(19) United States
(12) Patent Application Publication
(10) Pub. No.: US 2023/0017064 A1
(43) Pub. Date: Jan. 19, 2023

(54) METHODS FOR THE CHARACTERIZATION OF FLUID

(71) Applicant: NextGen Jane, Inc., Oakland, CA (US)

(72) Inventors: Ridhi TARIYAL, Oakland, CA (US); Stephen GIRE, Oakland, CA (US); Sarah BUSH, Oakland, CA (US); Margaret EISEN, Oakland, CA (US); Maya LATHI, Oakland, CA (US)

(21) Appl. No.: 17/772,832

(22) PCT Filed: Oct. 30, 2020
(86) PCT No.: PCT/US2020/058382

§ 371 Date: Apr. 28, 2022

Related U.S. Application Data
(60) Provisional application No. 63/061,709, filed on Aug. 5, 2020.
(60) Provisional application No. 62/930,465, filed on Nov. 4, 2019.
(60) Provisional application No. 62/929,579, filed on Nov. 1, 2019.

(51) Int. Cl. C12Q 1/6883; C12Q 1/689; G01N 33/68; A61B 5/15; G16H 50/30; G16H 50/20

(52) U.S. Cl. CPC C12Q 1/6883; C12Q 1/689; G01N 33/6893; A61B 5/150045; G16H 50/30; G16H 50/20; C12Q 2600/178; C12Q 2600/154; C12Q 2600/158; G01N 2800/361

(57) ABSTRACT

Provided herein are methods for detecting a menstrual cycle disorder. Menstrual cycle disorders is detected through analysis of a property of a collected menstrual fluid sample, such as gene expression, flow rate, protein content, nucleic acid content, biomarkers, or other properties.

### CROSS-REFERENCE

This application claims the benefit of U.S. Patent Application No. 62/929,579, filed Nov. 1, 2019, U.S. Patent Application No. 62/930,465, filed Nov. 4, 2019, and U.S. Patent Application No. 63/061,709, filed Aug. 5, 2020, which are hereby incorporated by reference in their entirety.

### BACKGROUND

Women are underrepresented in research. Diagnostic development for women's reproductive health has been hampered by a lack of understanding of uterine and menstrual physiology. To date, none of the peripheral blood biomarkers proposed for diagnosing women's reproductive health exhibit the accuracy required for clinical use.

Not only have period movements allowed for reduced stigma and national conversations about menstruation, but women are also gravitating to consumer health and digital recruitment platforms, creating new opportunities for research and diagnostic development. Menstrual blood provides direct access to reproductive tissue.

### SUMMARY

In certain aspects, disclosed herein are methods of using a predictive model to identify a condition of heavy menstrual bleeding (HMB). In some embodiments, the method comprises, using one or more processors in a computer server: (a) receiving data defining a plurality of subject digital biomarkers, each digital biomarker comprising a response by the subject to an associated inquiry in a computing data storage; (b) retrieving from computer data storage a combination of independent variables relating to the subject; (c) using a predictive model to determine a dependent variable representing a subject HMB risk score for the subject based on the combination of independent variables relating to the subject; (d) determining if the dependent variable representing the subject HMB score is above a threshold; and if the dependent variable representing the subject HMB score is above the threshold, sending information to be displayed. In some embodiments, the combination of independent variables includes objective data relating to the subject's menstrual bleeding. In some embodiments, the combination of independent variables includes survey data relating to the subject. In some embodiments, the survey data comprises at least one independent variable selected from the group consisting of a menstrual cycle phenotype, a physical body characteristic, a disease or condition, a medical treatment, demographic information, lifestyle information, ancestry, sexuality, menstrual management, health care usage and access. In some embodiments, the combination of independent variables includes health record data relating to the subject. In some embodiments, the objective data comprises a menstrual flow rate measurement. In some embodiments, the menstrual flow rate measurement comprises: (a) collecting menstrual fluid from a subject for a specified duration; (b) measuring the volume of collected menstrual fluid; (c) calculating a flow rate from the volume and the specified duration. In some embodiments, the method further comprises analyzing a biological marker from the collected menstrual fluid. In some embodiments, the biological marker is selected from a cell-type, a protein, a microorganism, a metabolite, a hematocrit level, or a nucleic acid. In some embodiments, the biological marker is unique to menstrual fluid.

Described herein, in some embodiments, are methods for generating a severity assessment of a menstrual bleeding state of a human female subject. In some embodiments, the method comprises: (a) presenting one or more questions to the subject about a first set of attributes related to the subject's menstrual history and a second set of attributes related to a subject's menstrual phenotype, wherein the one or more questions are presented to the subject on a display of a graphic user interface of an input device; (b) prompting the subject to enter a response to the one or more questions into the input device, wherein the input device transmits the response to a system comprising a processor and a computer-readable memory, wherein the system calculates an assessment score corresponding to menstrual bleeding state of the subject using the response to the one or more questions and stores the assessment score in the memory; (c) using an assay to perform one or more measurements on a menstrual fluid sample from the subject; (d) comparing the one or more measurements with one or more predetermined thresholds; and determine based on the calculated assessment score and the comparison with the one or more predetermined thresholds, the menstrual bleeding state of the subject; and generating a severity assessment of the subject's menstrual bleeding state.

Described herein, in certain aspects are methods of preparation of a biological sample. In some embodiments, the method comprises: (a) identifying a subject; (b) classifying the subject into a risk group based on one or more digital biomarkers; (c) collecting menstrual fluid from a subject for a specified duration; (d) measuring the volume of collected menstrual fluid €calculating a flow rate from the volume and the specified duration. In some embodiments, the collecting step is performed using a cup, a tampon, or a pad. In some embodiments, the menstrual fluid is collected in a manner that preserves at least one of intact cells from the vaginal-cervical space, protein, metabolite, DNA or RNA. In some embodiments, the collecting step comprises contacting the menstrual fluid or a portion thereof with a preserving solution. In some embodiments, the method further comprises extracting nucleic acid from the menstrual fluid and measuring at least one nucleic acid parameter. In some embodiments, the method further comprises measuring the presence of level of a metabolite in the menstrual fluid. In some embodiments, the nucleic acid parameter comprises the amount of nucleic acid, the diversity of nucleic acid, the presence of a miRNA, mRNA expression, copy number. In some embodiments, the method further comprises separating or isolating one or more cell types from the menstrual fluid. In some embodiments, the one or more cell types are selected from the group selected from immune cells, ovarian cells, fallopian tube cells, endometrial cells, and cervical cells. In some embodiments, the method further comprises repeating the collecting step for two or more longitudinal samples from the subject. In some embodiments, the method further comprises measuring an individual flow rate for each longitudinal sample and deriving a mean, average or progression of flow rate from the individual flow rates. In some embodiments, the digital biomarker comprises a factor listed in Table 4. Described herein, in certain aspects is a method for detecting a menstrual cycle disorder, comprising: (a) determining an expression level of one or more markers in a fluid sample obtained from the vaginal cavity of a subject, wherein the one or more markers are selected from Table 1, Table 2, and/or Table 3; and (b) comparing said expression level to a reference level of said one or more markers; wherein an increased or decreased expression level of said one or more markers relative to said reference expression level indicates that said subject has said menstrual cycle disorder. In some embodiments, the fluid sample is obtained from the subject during the subject's menstrual window. In some embodiments, the reference level is obtained from the subject in a time period outside subject's menstrual window. In some embodiments, the reference level is obtained from a healthy control subject or an average level from a group of healthy control subjects. In some embodiments, the one or more markers are protein expression markers selected from Table 1. In some embodiments, the one or more markers are gene expression markers selected from Table 2. In some embodiments, the one or more markers are gene expression markers selected from Table 3. In some embodiments, said menstrual cycle disorder is heavy menstrual bleeding. In some embodiments, the method further comprises determining a risk level for the menstrual cycle disorder in the subject. In some embodiments, the determining a risk level step comprises assessing one or more phenotypic or behavioral characteristics of the subject selected from Table 4 and/or Table 5. In some embodiments, the method further comprises stratifying the subject into a treatment group based on the risk level. In some embodiments, the method further comprises obtaining two or more fluid samples from the vaginal cavity of the same subject, wherein the two or more fluid samples are from different time points within the subject's menstrual cycle, wherein step (a) is repeated for each of the two or more fluid samples and wherein the expression level for each of the two or more fluid samples are compared in step (b). In some embodiments, the fluid sample comprises blood. In some embodiments, the fluid sample comprises shed endothelial cells and shed epithelial cells. In some embodiments, the fluid sample comprises a cell type selected from the group consisting of endothelial cells, epithelial cells, immune cells, and stem cells. In some embodiments, the two or more fluid samples are obtained during the subject's menstrual window. In some embodiments, at least one of the two or more fluid samples are obtained outside of the subject's menstrual window. In some embodiments, the one or more markers are selected from the group consisting of HLA-Aa (Major histocompatibility complex class I), IL-6ST (Interleukin 6 signal transducer), APCDD1L (Adenomatosis polyposis coli downregulated 1-like), TBX3 (T-box 3), UBN1a (Ubinuclein), DSPa (Desmoplakin), SDCBP2 (Syndecan binding protein (syntenin)), EZH2 (Enhancer of zeste 2 polycomb repressive complex 2 subunit), UHMK1 (U2AF homology motif (UHM) kinase), NR2C2 (Nuclear receptor subfamily 2, group C, member 2), MIR1282a (microRNA), TMED6a (Transmembrane p24 trafficking protein), VAV3 (Vav 3 guanine nucleotide exchange factor), CDC42BPA (CDC42 binding protein kinase alpha (DMPK-like)), C17orf75 (Chromosome 17 open reading frame), MB21D1 (Mab-21 domain containing 1), PTPRC (Protein tyrosine phosphatase, receptor type C), WISP1 (WNT1 inducible signaling pathway protein 1), CDC27 (Cell division cycle 27), FSD1L (Fibronectin type III and SPRY domain containing 1-like), BPIFB1 ((C20orf114) a BPI fold containing family B, member 1), SCGB3A1a (Secretoglobin, family 3A, member 1) TFF3a (Trefoil factor 3 (intestinal)), SCGB1D2a (Secretoglobin, family 1D, member 2), SCGB2A2a (Secretoglobin, family 2A, member 2), PRODH (Proline dehydrogenase (oxidase) 1), MFF (Mitochondrial fission factor), TSPAN8 (Tetraspanin), CXCL6a (Chemokine (C-X-C motif) ligand 6), SPDYE2 (Speedy/RINGO cell cycle regulator family member E2), FCGBP (Fc fragment of IgG binding protein), IRX6 (Iroquois homeobox 6), ADAM10 (ADAM metallopeptidase domain 10), MUC5ACa (Mucin 5AC, oligomeric mucus/gel-forming), TRHDE-AS1 (TRHDE antisense RNA (LOC283392)), CYP26A1 (Cytochrome P450, family 26, subfamily A, polypeptide 1), SAA2a (Serum amyloid A2), MMEL1 (Membrane metalloendopeptidase-like 1), GRIP2 (Glutamate receptor interacting protein 2), and SAA1a (Serum amyloid A1). In some embodiments, the method further comprises determining an increased expression level of one or more markers selected from the group consisting of HLA-Aa (Major histocompatibility complex class I), IL-6ST (Interleukin 6 signal transducer), APCDD1L (Adenomatosis polyposis coli downregulated 1-like), TBX3 (T-box 3), UBN1a (Ubinuclein), DSPa (Desmoplakin), SDCBP2 (Syndecan binding protein (syntenin)), EZH2 (Enhancer of zeste 2 polycomb repressive complex 2 subunit), UHMK1 (U2AF homology motif (UHM) kinase), NR2C2 (Nuclear receptor subfamily 2, group C, member 2), MIR1282a (microRNA), TMED6a (Transmembrane p24 trafficking protein), VAV3 (Vav 3 guanine nucleotide exchange factor), CDC42BPA (CDC42 binding protein kinase alpha (DMPK-like)), C17orf75 (Chromosome 17 open reading frame), MB21D1 (Mab-21 domain containing 1), PTPRC (Protein tyrosine phosphatase, receptor type C), WISP1 (WNT1 inducible signaling pathway protein 1), CDC27 (Cell division cycle 27), and FSD1L (Fibronectin type III and SPRY domain containing 1-like) relative to said reference expression level. In some embodiments, the method further comprises determining a decreased expression level of the one or more markers selected from the group consisting of BPIFB1 ((C20orf114) a BPI fold containing family B, member 1), SCGB3A1a (Secretoglobin, family 3A, member 1) TFF3a (Trefoil factor 3 (intestinal)), SCGB1D2a (Secretoglobin, family 1D, member 2), SCGB2A2a (Secretoglobin, family 2A, member 2), PRODH (Proline dehydrogenase (oxidase) 1), MFF (Mitochondrial fission factor), TSPAN8 (Tetraspanin), CXCL6a (Chemokine (C-X-C motif) ligand 6), SPDYE2 (Speedy/RINGO cell cycle regulator family member E2), FCGBP (Fc fragment of IgG binding protein), IRX6 (Iroquois homeobox 6), ADAM10 (ADAM metallopeptidase domain 10), MUC5ACa (Mucin 5AC, oligomeric mucus/gel-forming), TRHDE-AS1 (TRHDE antisense RNA (LOC283392)), CYP26A1 (Cytochrome P450, family 26, subfamily A, polypeptide 1), SAA2a (Serum amyloid A2), MMEL1 (Membrane metalloendopeptidase-like 1), GRIP2 (Glutamate receptor interacting protein 2), and SAA1a (Serum amyloid A1). relative to said reference expression level. In some embodiments, the method further comprises determining an expression pattern of said one or more genes or expression products thereof. In some embodiments, the fluid sample is disposed in a sample collector. In some embodiments, said sample collector is a pad, a tampon, a vaginal cup, a cervical cap, a menstrual disk, a cervical disk, a sponge, or an interlabial pad. In some embodiments, the sample collector comprises a chamber comprising a buffer for preserving intact cells, DNA, RNA, protein, metabolite(s), or any combination thereof. In some embodiments, the method further comprises the step of treating the subject for a heavy menstrual bleeding disorder if the increased or decreased expression level of said one or more markers relative to said reference expression level indicates that said subject has said menstrual cycle disorder. In some embodiments, the step of treating is selected from the group consisting of a therapeutic agent, a surgical intervention or a combination thereof. In some embodiments, the therapeutic agent is selected from the group consisting of an antifibrinolytic agent, a combined hormonal contraceptive, a progestogens, a progestogen-releasing intrauterine device, an androgen, a gonadotropin releasing hormone analogue or any combination thereof. In some embodiments, the surgical intervention is selected from the group consisting of surgical excision, n endometrial ablation, endometrial cryoablation, uterine artery embolization, myomectomy, hysterectomy, and any combination thereof. In some embodiments, the method further comprises placing the subject in a non-treatment category if the subject does not have increased or decreased expression level of said one or more markers relative to said reference expression level.

In certain aspects, disclosed herein are methods of producing a desired preparation for assessment of menstrual health In some embodiments, the method comprises: (a) receiving a fluid sample collected from the vaginal cavity of a subject, the fluid sample comprising one or more types of cells; (b) contacting the fluid sample with a buffer solution under conditions suitable to maintain one or more cell types in a substantially intact state; (c) separating one cell type in the fluid sample from the remaining fluid sample; (d) determining an expression level of one or more markers in the one cell type, wherein the one or more markers are selected from Table 1 and/or Table 2; and (e) comparing the expression level with a reference level to assess a level of menstrual health. In some embodiments, step (b) maintains at least 90%, 95%, or substantially 100% of said one or more types of cells in a substantially intact state. In some embodiments, the fluid sample is a menstrual fluid sample. In some embodiments, the one cell type is selected from the group consisting of endothelial cells, epithelial cells, mesenchymal cells, and leukocytes. In some embodiments, the one cell type is separated based on expression of a cell surface antigen. In some embodiments, the one cell type is an endothelial cell and the cell surface antigen is selected from the group consisting of CD31/PECAM-1, CD34, CD36/SR-B3, CD39, CD44, CD47, CD54/ICAM-1, CD61, CD62E, CD62P, CD80, CD86, CD93, CD102, CD105, CD106, CD112, CD117, ESAM, ENDOMUCIN, CXCL16, CD121a, CD141, CD142, CD143, CD144, CD146, CD147, CD151, CD160, CD201, CD213a, CD248, CD309, ADAMS 8-17, 33, ADAMTS-13, ADAMTS-18, VWF, TEM8, NOTCH, KLF4 and any combination thereof. In some embodiments, the one cell type is an epithelial cell and the cell surface antigen is selected from the group consisting of EpCAM, E-cadherin, CD326, and any combination thereof. In some embodiments, the one cell type is a leukocyte and the cell surface antigen is CD45. In some embodiments, the one cell type is a mesenchymal cell and the cell surface antigen is selected from the group consisting of N-cadherin, OB-cadherin, alpha-5, beta-1 integrin, alpha-V, beta-6 integrin, Syndecan-1, and any combination thereof. In some embodiments, the method further comprises using an antibody that binds the cell surface antigen to separate the one cell type. In some embodiments, the fluid sample is disposed in a sample collector. In some embodiments, said sample collector is a pad, a tampon, a vaginal cup, a cervical cap, a menstrual disk, a cervical disk, a sponge, or an interlabial pad. In some embodiments, the sample collector comprises the buffer solution.

Disclosed herein, in some embodiments are methods for assessing menstrual health in a subject. In some embodiments, the method comprises: (a) receiving a menstrual fluid sample collected from said subject; (b) contacting said menstrual fluid sample with a buffer solution under conditions suitable to preserve a sample component selected from the group consisting of intact cells, nucleic acid, protein, and any combination thereof; and (c) determining a sample component level from the menstrual fluid sample. In some embodiments, the sample component comprises intact cells, and wherein step (b) maintains at least 90%, 95%, or substantially 100% of said one or more types of cells in a substantially intact state. In some embodiments, step (c) comprises determining an amount of one or more cell types in the menstrual fluid sample. In some embodiments, the one or more cell types are selected from the group consisting of leukocytes, erythrocytes, endothelial cells, epithelial cells, stromal cells, stem cells, and any combinations thereof. In some embodiments, the method further comprises comparing the amount of one or more cell types to a predetermined threshold. In some embodiments, an increase in the amount as compared to the predetermined threshold indicates that the subject has a menstrual cycle disorder. In some embodiments, the sample component comprises nucleic acid. In some embodiments, the method further comprises determining an amount of nucleic acid present in the menstrual fluid sample. In some embodiments, the method further comprises comparing the amount of nucleic acid to a predetermined threshold. In some embodiments, an increase in the amount as compared to the predetermined threshold indicates that the subject has a menstrual cycle disorder. In some embodiments, the predetermined threshold is an amount of nucleic acid present in a reference menstrual fluid sample. In some embodiments, the reference menstrual fluid sample is obtained from a healthy control subject. In some embodiments, the nucleic acid comprises RNA, and wherein the method further comprises measuring the level of at least one RNA expression marker. In some embodiments, the nucleic acid comprises miRNA, and wherein the method further comprises measuring the level of at least one miRNA. In some embodiments, the method comprises comparing the level to a predetermined threshold. In some embodiments, the sample component comprises protein. In some embodiments, the method comprises determining the presence or level of at least one protein marker. In some embodiments, the method comprises comparing the level to a predetermined threshold. In some embodiments, the sample component comprises a microbial source present in the menstrual fluid sample. In some embodiments, the microbial source is a bacteria, a fungus, or a virus. In some embodiments, the method further comprises measuring a microbial source component, and wherein the component is selected from the group consisting of nucleic acid, protein, metabolite, cell, and any combination thereof. In some embodiments, the method further comprises measuring the bacterial diversity in the menstrual fluid sample. In some embodiments, said menstrual fluid sample is disposed in a sample collector. In some embodiments, said sample collector is a pad, a tampon, a vaginal cup, a cervical cap, a menstrual disk, a cervical disk, a sponge, or an interlabial pad. In some embodiments, the sample collector comprises the buffer solution. In some embodiments, said buffer solution has a pH greater than 7. In some embodiments, the method further comprises, prior to (c), storing said menstrual fluid sample in the presence of said buffer solution for at least 1 day. In some embodiments, the storage occurs at up to about 4° C. In some embodiments, the storage occurs at up to about −20° C. In some embodiments, the storage occurs at room temperature.

Disclosed herein in some embodiments, are methods for detecting a menstrual cycle disorder in a subject. In some embodiments, the method comprises: (a) determining a flow rate of a menstrual fluid sample collected from said subject within a predefined time period; and (b) comparing said flow rate of said menstrual fluid sample to a predetermined threshold; wherein an increased flow rate relative to said predetermined threshold indicates that said subject has said menstrual cycle disorder. In some embodiments, said predetermined threshold is a flow rate of a menstrual fluid sample obtained from a reference subject within said predefined time period. In some embodiments, said reference subject is a healthy control subject. In some embodiments, said predefined time period is greater than 15 minutes. In some embodiments, said predefined time period is less than 2 hours. In some embodiments, the method further comprises measuring a volume of said menstrual fluid sample collected from said subject within said predefined time period. In some embodiments, the method further comprises comparing said measured volume to a predetermined threshold. In some embodiments, said predetermined threshold is a volume of a menstrual fluid sample collected from a reference subject within said predefined time period. In some embodiments, said menstrual fluid sample is disposed in a sample collector. In some embodiments, said sample collector is a pad, a tampon, a vaginal cup, a cervical cap, a menstrual disk, a cervical disk, a sponge, or an interlabial pad.

Disclosed herein in some embodiments are methods for detecting a menstrual cycle disorder. In some embodiments, the method comprises: (a) determining an expression level of one or more markers in a fluid sample obtained from the vaginal cavity of a subject, wherein the one or more markers are selected from Table 1, Table 2 and/or Table 3; (b) applying a classifier algorithm to said expression level of one or more markers and a reference level of each of the one or more markers to calculate a metric that quantifies a difference between the expression level and the reference level for each of the one or more markers; and (c) determining a presence of a menstrual cycle disorder based on the metric. Disclosed herein, in some embodiments is a method of producing a desired preparation for assessment of menstrual health, comprising: (a) receiving a fluid sample collected from the vaginal cavity of a subject, the fluid sample comprising one or more types of cells; (b) contacting the fluid sample with a buffer solution under conditions suitable to maintain one or more cell types in a substantially intact state; (c) separating one cell type in the fluid sample from the remaining fluid sample; and (d) applying a classifier algorithm to said expression level of one or more markers in the one cell type to calculate a metric that quantifies the difference between said expression level and a reference level to assess a level of menstrual health, wherein the one or more markers are selected from Table 1 and/or Table 2.

Disclosed herein in some embodiments are methods for detecting a menstrual cycle disorder in a subject. In some embodiments, the method comprises: (a) determining a flow rate of a menstrual fluid sample collected from said subject within a predefined time period; and (b) applying a classifier algorithm to said flow rate of said menstrual fluid sample to calculate a metric that quantifies a difference between said metric and a predetermined threshold; wherein an increased flow rate relative to said predetermined threshold indicates that said subject has said menstrual cycle disorder.

Disclosed herein in some embodiments are methods comprising: (a) obtaining a fluid sample from a vaginal cavity of a subject, wherein the fluid sample is stabilized under conditions which preserve a component of the fluid sample (b) determining an expression level of one or more markers in the fluid sample; (c) comparing said expression level to a reference level of said one or more markers to detect an increased or decreased expression level of the one or more markers relative to the reference expression level, and (d) determining from the increased or decreased expression level whether the subject has a menstrual cycle disorder. In some embodiments, the component is preserved for at least 1 day. In some embodiments, the component is preserved at up to about 4° C. In some embodiments, the component is preserved at up to about −20° C. In some embodiments, the component is preserved at room temperature In some embodiments, the component is selected from the group consisting of a cell, a nucleic acid, a protein, a metabolite, and a microorganism. In some embodiments, the one or more markers are selected from the group consisting of the markers in Table 4. In some embodiments, the one or more markers are selected from the group consisting of the markers in Table 5. In some embodiments, the one or more markers are selected from the group consisting of the markers in Table 1. In some embodiments, the menstrual cycle disorder is HMB. In some embodiments, the menstrual cycle disorder is AUB.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. In some embodiments, the novel features of the invention are set forth with particularity in the appended claims. In some embodiments, a better understanding of the features and advantages of the present invention will be obtained by reference to the following detailed description that sets forth illustrative embodiments, in which the principles of the invention are utilized, and the accompanying drawings of which:

FIG. 1 depicts a pattern of menstrual blood over the days of a cycle.

FIG. 2 depicts the different colors of menstrual blood.

FIG. 3 depicts the different sizes of tampons.

FIG. 4 depicts the different sizes of pads.

FIG. 5 depicts the different types of menstrual products used.

FIG. 6 depicts different hair loss patterns.

FIG. 7 depicts possible locations for acne on a subject.

FIG. 8 depicts acne severity.

FIG. 9 depicts different types of acne.

FIG. 10 depicts different patterns of hair on the upper lip.

FIG. 11 depicts different patterns of hair on the arms.

FIG. 12 depicts different patterns of hair on the jawline.

FIG. 13 depicts different patterns of hair on the thighs.

FIG. 14 depicts different patterns of hair on the chest.

FIG. 15 depicts different patterns of hair on the stomach.

FIG. 16 depicts different patterns of hair on the upper back.

FIG. 17 depicts different patterns of hair on the lower back.

FIG. 18 depicts different patterns of hair on the pubic area.

FIG. 19 depicts the location of the uterus.

FIG. 20 depicts a method to test pelvic floor strength.

FIG. 21 depicts locations where a subject may store fat.

FIG. 22 depicts a method to identify body size.

FIG. 23 depicts different types of caffeine.

FIG. 24 illustrates the diversity of bacterial species among patients with endometriosis, polycystic ovarian syndrome, and among healthy patients.

FIG. 25A-25C illustrate the different types of primary cell types, reproductive issues, and stem cells found in menstrual blood over time.

FIG. 26 displays the amount of alkaline hematin found in two patient samples of menstrual blood.

FIG. 27A-27C illustrate potential data groupings by stratified patient.

FIG. 28 illustrates factors used to annotate a patient.

FIG. 29 is a schematic of the comparison of a patient value over time as compared to the mean value of a other patients with similar annotations.

### DETAILED DESCRIPTION

Provided in this disclosure are methods for detecting a menstrual cycle disorder in a subject. In some embodiments, a subject is suspected of having or at risk for a menstrual cycle disorder. In some embodiments, a subject is a demographic, lifestyle, or genetic risk factor for a menstrual cycle disorder. In some embodiments, a subject exhibits expression of RNA, protein, metabolites or other cellular products that is linked as a risk factor for a menstrual cycle disorder.

### Menstrual Fluid Samples

In some embodiments of methods herein, fluid from the vaginal cavity of a subject is collected from the subject, such as by using a collection device. In some embodiments, the collection device is placed inside or near the vagina and left in place for a period of time to allow fluid such as menstrual fluid to collect in or on the device. In some embodiments, the fluid is then removed or extracted from the device for further analysis. In some embodiments, the fluid is menstrual fluid, such as menstrual blood collected during the subject's menstrual window. In some embodiments, fluid is collected from the vaginal cavity outside of the subject's menstrual window.

In some embodiments, a menstrual cycle disorder is a disorder or abnormality associated with a menstrual cycle, which comprises heavy menstrual bleeding or abnormal bleeding, such as abnormal uterine bleeding. In some aspects, these methods comprise collecting a sample of fluid from the vaginal cavity of a subject (e.g., menstrual fluid) and analyzing an aspect of the fluid.

In some embodiments, aspects of fluid which are analyzed include, but are not limited to, flow rate, cell types, nucleic acid content, gene expression, protein biomarker expression, metabolites, and gene target expression. In some embodiments, these aspects are quantified, and in some cases, a change such as an increase or decrease in an aspect are correlated with a menstrual cycle disorder. In some embodiments, aspects of fluid which are analyzed include the biological markers (also referred to as “biomarkers”) described herein. In some embodiments, such a change are indicative of a menstrual cycle disorder.

In some cases, normal menstrual cycle occurs approximately every month and comprises the shedding of the lining of the uterus through the vagina. In some cases, normal menstrual flow lasts approximately 4 or 5 days, lasts up to 7 days, and occurs every 21 to 35 days.

In some embodiments, collection of fluid from the vaginal cavity, such as menstrual fluid, provides direct access to reproductive tissue. In some embodiments, this access allows for biopsy, biomarker detection, and assessment of health conditions. In some embodiments, knowledge gained from such information provides women with health information about reproductive conditions, which affects fertility or quality of life.

In some instances, menstrual cycle disorders are the most prevalent gynecologic health problems in the United States, and heavy menstrual bleeding (HMB) affects up to 30% of women at some time during their reproductive years. Many reproductive conditions present with changes to the menstrual cycle, for example, including light bleeding or heavy bleeding. In some embodiments, the menstrual cycle presents abnormal uterine bleeding (AUB). In some embodiments, AUB comprises bleeding or spotting between periods (e.g., off-cycle bleeding), bleeding or spotting after sex, heavy bleeding during menstruation, a menstrual cycle that is abnormally long (e.g., longer than 38 days), a menstrual cycle that is abnormally short (e.g., shorter than 24 days), irregular periods (e.g., periods in which the cycle length varies by at least 7, 8, or 9 days), or bleeding after menopause. In some instances, the societal and personal burden of AUB lies in its impact on quality of life, productivity, and health care use and costs.

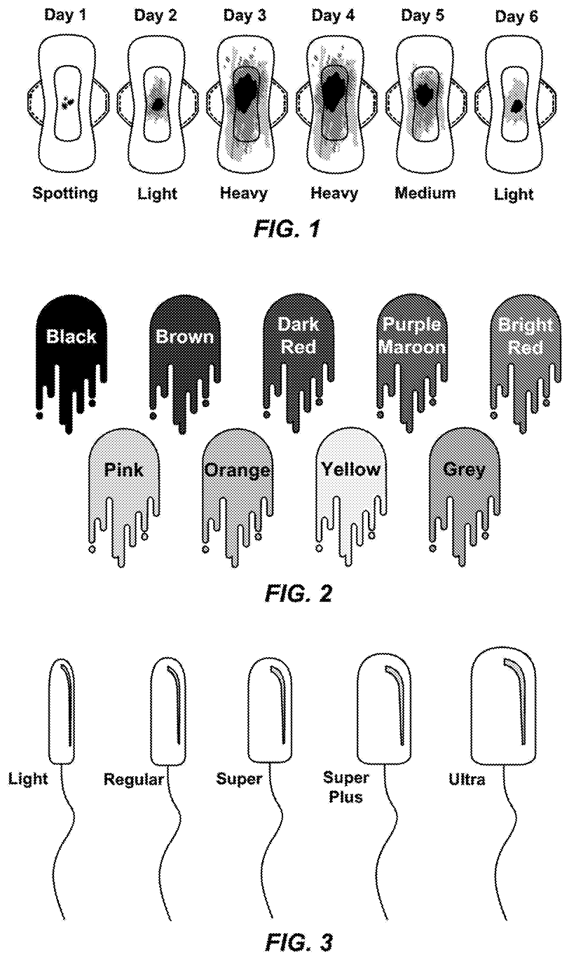

In some embodiments, heavy menstrual bleeding is diagnosed using a pictorial blood loss assessment chart, such as the one depicted in FIG. 1.

In some embodiments, the alkaline haematin test provides a quantitative assessment for heavy menstrual bleeding. In some embodiments, this involves a collection of sanitary products in a menstrual cycle and soaking them in sodium hydroxide.

In some embodiments, one or more blood markers provides a measurement for heavy menstrual bleeding. In some embodiments, the blood marker is hematocrit, hemoglobin, zinc protoporphyrin, or any combination thereof. In some embodiments, the blood marker level or amount in a collected menstrual fluid sample is compared to the blood marker level or amount in a whole blood sample (e.g., venous blood) from the same subject.

In some instances, a menstrual cycle disorder is indicative of an underlying disease or disorder, which affect the health, quality of life, fertility, or lifespan of a woman. In some instances, menstrual cycle disorders and other disorders which present with, or cause, HMB or AUB include eating disorders; extreme weight loss; excessive exercise; polycystic ovary syndrome (PCOS); ovarian cysts; premature ovarian failure; breast cancer; ovarian cancer; infertility; diminished ovarian reserve; chronic or frequent urinary tract infections; ectopic pregnancy; heart disease; type 1 diabetes; type 2 diabetes; an autoimmune condition such as lupus, multiple sclerosis, or rheumatoid arthritis; pelvic inflammatory disease (PID); fibroids (e.g., uterine fibroids); adenomyosis; cervical cancer; endometrial cancer; uterine cancer; or infection of the cervix or endometrium. In some embodiments, HMB or AUB accompanies a disease affecting the kidney, liver, thyroid, or adrenal glands.

In some embodiments, fluid collected from the vaginal cavity is a biological matrix comprising a plurality of cell types. In some instances, the cell types include immune cells, ovarian cells, fallopian tube cells, endometrial cells, and cervical cells. In some embodiments, the fluid is menstrual fluid, including menstrual blood. In some instances, comparison of endometrium and other reproductive tissues from the fluid collected from the vaginal cavity, such as menstrual fluid, of healthy women and those with dysfunction advances understanding of key areas of endometrial physiology, including infertility, receptivity, endometriosis, and cancer. In some embodiments, a typical gene expression is linked to cellular dysfunction. In some instances, endometrial mucosa undergoes dynamic, hormone-dependent alterations throughout the life of females. In some instances, detailed information about menstrual cycle-specific gene expression changes promotes understanding of the gene regulatory networks which underlie changes in the menstrual cycle. In some embodiments, such changes lead to preparation of uterus for embryo implantation. In further cases, such changes is analyzed to identify molecular differences between healthy and diseased endometrium.

In some instances, epigenetic factors such as methylation and the immune repertoire play a large role in initiating morphological and functional changes with the endometrium that promote or be essential for uterine receptivity. In some embodiments, these methylation changes is cycle dependent, and is associated with gene expression regulation. In some instances, the female reproductive tract comprise one or more immune cell populations, which is enriched for a particular cell type. In some embodiments, in some cases, an immune cell population becomes enriched in natural killer cells, which plays a role in endometrial receptivity or increase a risk for pregnancy loss.

New efforts have genomically characterized endometrium, allowing for single-cell resolution of tissue characterization for better disease genotyping. Studies have begun to shed light on the complex sample makeup of menstrual fluid, with a distinct immune repertoire. In some embodiments, eutopic endometrium or cells isolated from menstrual fluid has a genomic correlation with endometriosis.

Measurement of menstrual bleeding is one way to diagnose or monitor menstrual cycle disorders such as HMB or AUB. In some embodiments, heavy menstrual bleeding includes bleeding about 80 ml or more during a single menstrual period. However, almost half of all women reporting heavy menstrual bleeding has less than about 40 ml of menstrual bleeding per cycle. Thus, in some instances, accurate and reliable methods for quantifying menstrual bleeding, including heavy menstrual bleeding, is necessary to detect menstrual cycle disorders and to understand effects of HMB or AUB on health and discomfort of the patient.

In some embodiments, normal menstrual bleeding comprises a menstrual flow having a normal volume and flow rate. In some embodiments, normal menstrual bleeding presents with a typical expression of one or more genes, presence or absence of one or more cell types, a typical nucleic acid content, a typical flow rate, a typical protein biomarker expression, a metabolite, a hematocrit level, or a typical gene target biomarker expression.

In some embodiments, normal menstrual bleeding is in some cases be identified by a volume of menstrual fluid lost during a menstrual cycle or by a flow rate during a menstrual cycle. In some embodiments, normal menstrual bleeding comprises between 10 mL and 80 mL of menstrual blood during a single menstrual cycle. In some embodiments, normal menstrual bleeding comprises a maximum volume of at least 40 mL, at least 45 mL, at least 50 mL, at least 55 mL, at least 60 mL, at least 65 mL, at least 70 mL, at least 75 mL, at least 80 mL, at least 85 mL, at least 90 mL, at least 95 mL, or at least 100 mL of menstrual fluid lost during a menstrual cycle. In some embodiments, normal menstrual bleeding comprises a flow rate of up to 7 mL per day, up to 8 mL per day, up to 9 mL per day, up to 10 mL per day, up to 11 mL per day, up to 12 mL per day, up to 13 mL per day, up to 14 mL per day, up to 15 mL per day, up to 16 mL per day, up to 17 mL per day, up to 18 mL per day, up to 19 mL per day, or up to 20 mL per day. In some embodiments, normal menstrual bleeding comprises a flow rate of up to 0.05 mL per hour, up to 0.1 mL per hour, up to 0.2 mL per hour, up to 0.3 mL per hour, up to 0.4 mL per hour, up to 0.5 mL per hour, up to 0.6 mL per hour, up to 0.7 mL per hour, up to 0.8 mL per hour, up to 0.9 mL per hour, or up to 1.0 mL per hour.

In some cases, HMB is menstrual bleeding having a larger volume or higher flow rate than normal menstrual bleeding. In some cases, HMB present with an altered expression of one or more genes detectable in a menstrual fluid sample, presence or absence of one or more cell types detectable in a menstrual fluid sample, a high or low nucleic acid content detectable in a menstrual fluid sample, a high flow rate of menstrual fluid during at least a portion of a menstrual cycle, an altered protein or biomarker expression detectable in a menstrual fluid sample, an altered metabolite level in a menstrual fluid sample, an altered enzyme level in a menstrual fluid sample, an altered hematocrit level in a menstrual fluid level, or an altered gene target biomarker expression detectable in a menstrual fluid sample.

In some embodiments, HMB comprises at least 60 mL, at least 65 mL, at least 70 mL, at least 75 mL, at least 80 mL, at least 85 mL, at least 90 mL, at least 95 mL, at least 100 mL, at least 110 mL, at least 120 mL, or more of menstrual fluid lost during a single menstrual cycle. In some embodiments, HMB, such as HMB presenting as a high flow rate, comprises at least 30 mL, at least 40 mL, at least 50 mL, at least 60 mL, at least 70 mL, at least 80 mL, at least 90 mL, at least 100 mL, at least 110 mL, at least 120 mL, or more menstrual fluid lost during a single menstrual cycle. In some embodiments, HMB comprises a flow rate of at least 10 mL per day, at least 11 mL per day, at least 12 mL per day, at least 13 mL per day, at least 14 mL per day, at least 15 mL per day, at least 16 mL per day, at least 17 mL per day, at least 18 mL per day, at least 19 mL per day, or at least 20 mL per day of menstrual fluid lost during at least a portion of a single menstrual cycle. In some embodiments, HMB comprises a flow rate of at least 0.1 mL per hour, at least 0.2 mL per hour, at least 0.3 mL per hour, at least 0.4 mL per hour, at least 0.5 mL per hour, at least 0.6 mL per hour, at least 0.7 mL per hour, at least 0.8 mL per hour, at least 0.9 mL per hour, at least 1.0 mL per hour, at least 1.1 mL per hour, at least 1.2 mL per hour, at least 1.3 mL per hour, at least 1.4 mL per hour, or at least 1.5 mL per hour of menstrual fluid lost during a single menstrual cycle. In some embodiments, HMB, such as HMB presenting as a high flow rate, comprises a volume of menstrual fluid lost that is consistent with normal menstrual bleeding. In such cases, a volume of menstrual fluid lost is at least about 30 mL, 40 mL, 50 mL, 60 mL, 70 mL, 80 mL, 90 mL, 100 mL, 110 mL, 120 mL, or more menstrual fluid during a single menstrual cycle. In some embodiments, HMB lasts for up to 0.5, 1, 2, 3, 4, 5, 6, 7, 8, 9, 10, 11, 12, 13, 14, 15, 16, 17, 18, 19, 20, 21, 22, 23, 24, 25, 26, 27, 30, 29, or 30 days. In some embodiments, HMB lasts for at least 1, 2, 3, 4, 5, 6, 7, 8, 9, 10, 11, 12, 13, 14, 15, 16, 17, 18, 19, 20, 21, 22, 23, 24, 25, 26, 27, 30, 29, 30, or more days. HMB lasting longer than 7 days is menorrhagia.

AUB is menstrual bleeding having a volume or flow rate that is different than that of normal menstrual bleeding. In some embodiments, AUB is HMB. In some embodiments, AUB presents with an altered expression of one or more genes detectable in a menstrual fluid sample, presence or absence of one or more cell types detectable in a menstrual fluid sample, a high or low nucleic acid content detectable in a menstrual fluid sample, a high flow rate of menstrual fluid during at least a portion of a menstrual cycle, an altered protein or biomarker expression detectable in a menstrual fluid sample, an altered metabolite level in a menstrual fluid sample, an altered hematocrit level in a menstrual fluid level, or an altered gene target biomarker expression detectable in a menstrual fluid sample.

In some embodiments, AUB comprises at least 20 mL, at least 30 mL, at least 40 mL, at least 50 mL, at least 60 mL, at least 70 mL, at least 80 mL, at least 90 mL, at least 100 mL, at least 110 mL, at least 120 mL, or more menstrual fluid lost during a single menstrual cycle. In some embodiments, AUB comprises a flow rate of at least 7 mL per day, at least 8 mL per day, at least 9 mL per day, 10 mL per day, at least 11 mL per day, at least 12 mL per day, at least 13 mL per day, at least 14 mL per day, at least 15 mL per day, at least 16 mL per day, at least 17 mL per day, at least 18 mL per day, at least 19 mL per day, or at least 20 mL per day of menstrual fluid lost during at least a portion of a single menstrual cycle. In some embodiments, AUB comprises a flow rate of at least 0.1 mL per hour, at least 0.2 mL per hour, at least 0.3 mL per hour, at least 0.4 mL per hour, at least 0.5 mL per hour, at least 0.6 mL per hour, at least 0.7 mL per hour, at least 0.8 mL per hour, at least 0.9 mL per hour, at least 1.0 mL per hour, at least 1.1 mL per hour, at least 1.2 mL per hour, at least 1.3 mL per hour, at least 1.4 mL per hour, or at least 1.5 mL per hour of menstrual fluid lost during at least a portion of a single menstrual cycle. In some embodiments, AUB, such as AUB presenting as a high flow rate, comprises a volume of menstrual fluid lost that is consistent with normal menstrual bleeding. In such cases, a volume of menstrual fluid lost is at least about 30 mL, 40 mL, 50 mL, 60 mL, 70 mL, 80 mL, 90 mL, 100 mL, 110 mL, 120 mL, or more menstrual fluid during a single menstrual cycle. In some embodiments, AUB lasts for up to 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, AUB lasts for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days. In some embodiments, AUB is HMB.

In some embodiments, HMB presents as an alteration in the distribution, ratio or amounts of cell types in menstrual fluid. For example, HMB may present as an increase in red blood cells in the menstrual fluid of an HMB subject, or an alteration in the ratios between endothelial, epithelial and hematopoietic cells or any combination thereof.

Described herein are methods for detecting a menstrual cycle disorder in a subject. Briefly, a fluid sample is obtained from the vaginal cavity of a subject for analysis, and a property of the fluid is determined. In some embodiments, properties of the fluid sample comprises (i) gene expression detectable in a fluid sample, (ii) presence or absence of a cell type detectable in a fluid sample, (iii) amount of nucleic acid detectable in a fluid sample, (iv) flow rate of menstrual fluid during a portion or all of a menstrual cycle, (v) one or more protein biomarkers detectable in a fluid sample, (vi) one or more metabolite detectable in a fluid sample, (vii) one or more enzyme detectable in a fluid sample, and/or (viii) one or more gene target biomarkers detectable in a fluid sample. Such properties of a fluid sample is detected or measured in a fluid sample from the vaginal cavity during the menstrual window and/or outside the menstrual window. In some embodiments, samples is from a subject experiencing menstrual bleeding, such as normal menstrual bleeding, HMB, or AUB.

In some embodiments, a fluid sample, such as a menstrual fluid sample or a sample of another fluid, is collected from a subject using a sample collector which collects fluid from the vaginal cavity. In some embodiments, a sample collector is placed in the vagina or outside the vagina for sample collection. In some embodiments, a sample collector collects a sample by pooling, holding, catching, directing, or absorbing the sample. In some embodiments, a sample collector is absorbent, semi-absorbent, or non-absorbent. In some embodiments, a sample collector is soluble in a buffer. In some embodiments, a sample collector is broken down, for example by exposing the sample collector to an acidic environment, a basic environment, or an enzyme. In some embodiments, sample collectors comprise a pad, a tampon, a vaginal cup, a cervical cap, a menstrual disk, a cervical disk, a sponge, or an interlabial pad. In some embodiments, more than one type of sample collector are used.

In some embodiments, a sample collector is left in place for a pre-determined amount of time to collect a fluid sample. In some embodiments, at least 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses. In some embodiments, at most 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses while the sample collection device is left in place. In some embodiments, about 5 minutes, 10 minutes, 15 minutes, 30 minutes, 45 minutes, 1 hour, 1.5 hours, 2 hours, 3 hours, 4 hours, 5 hours, 6 hours, 7 hours, or 8 hours elapses while the sample collector is left in place

In some embodiments, a sample is collected during the menstrual window (the period) of a subject. In some embodiments, a sample collector is disposable. In some embodiments, a disposable sample collector is discarded or broken down after use. In some embodiments, a disposable sample collector is dissolvable, biodegradable, recyclable, or compostable. Typically, one disposable sample collector is used to collect one sample from one subject. In some embodiments, a sample collector is reusable. In some embodiments, a reusable sample collector is washable, sterilizable, or autoclavable. In some embodiments, a reusable sample collector is resistant to degradation, tearing, pore formation, or dissolution. In some embodiments, a reusable sample collector comprises anti-microbial, antibacterial, antiviral, or antifungal properties. In some embodiments, a reusable sample collector is used one or more times to collect one or more samples. In some embodiments, a reusable sample collector is used about 1, 2, 3, 4, 5, 6, 7, 8, 9, 10, 15, 20, 25, 30, or more times to collect one or more fluid samples. In some embodiments, a reusable sample collector is used to repeatedly collect fluid samples from one subject. In some embodiments, a reusable sample collector is used to collect samples from a plurality of subjects.

In some embodiments, one or more sample is collected during one or more periods (menstrual windows) of a subject. In some embodiments, 1 sample is collected during 1 period cycle, 2 samples is collected during 1 period cycle, 3 samples is collected during 1 period cycle, 4 samples is collected during 1 period cycle, more than 4 samples is collected during 1 period cycle, 2 samples is collected during 2 period cycles, 3 samples is collected during 2 period cycles, 4 samples is collected during 2 period cycles, 5 samples is collected during 2 period cycles, 6 samples is collected during 2 period cycles, 7 samples is collected during 2 period cycles, 8 samples is collected during 2 period cycles, more than 8 samples is collected during 2 period cycles, 3 samples is collected during 3 period cycles, 4 samples is collected during 3 period cycles, 5 samples is collected during 3 period cycles, 6 samples is collected during 3 period cycles, 7 samples is collected during 3 period cycles, 8 samples is collected during 3 period cycles, 9 samples is collected during 3 period cycles, 10 samples is collected during 3 period cycles, 11 samples is collected during 3 period cycles, 12 samples is collected during 3 period cycles, more than 12 samples is collected during 3 period cycles, 4 samples is collected during 4 period cycles, 5 samples is collected during 4 period cycles, 6 samples is collected during 4 period cycles, 7 samples is collected during 4 period cycles, 8 samples is collected during 4 period cycles, 9 samples is collected during 4 period cycles, 10 samples is collected during 4 period cycles, 11 samples is collected during 4 period samples, 12 samples is collected during 4 period cycles, 13 samples is collected during 4 period cycles, 14 samples is collected during 4 period cycles, 15 samples is collected during 4 period cycles, 16 samples is collected during 4 period cycles, or more than 16 samples is collected during 4 period cycles. In some embodiments, a plurality of samples is collected during more than 4 period cycles.

In some embodiments, a plurality of samples is collected during a single period cycle, and in some cases, in a single day. For example, samples are collected for different durations of collection on a single day or on multiple days, such as collections for 15 minutes, 30 minutes, 45 minutes, 1 hour, 2 hours, 3 hours, 4 hours, 5 hours or 6 hours.

In some embodiments, samples are collected outside the menstrual window, e.g., between the time of the subject's periods. In such cases, a non-menstrual fluid is collected using the sample collector. In some embodiments, non-menstrual fluid which is collected include vaginal secretions, cervical mucus, cervicovaginal fluid, spotting blood (i.e., from between periods), amniotic fluid, a mucus plug, or other vaginal discharge. In some embodiments, non-menstrual fluid is collected and analyzed using a protocol which is used to collect and analyze menstrual fluid.

In some embodiments, a sample is collected after a menstrual window has closed, e.g., after a period has ended. In some embodiments, a sample is collected on the same day a menstrual window closed. In some embodiments, a sample is collected about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days after a menstrual window has closed. In some embodiments, a sample is collected at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, or at least 30 days after a menstrual window has closed. In some embodiments, sample is collected not more than 1 day, not more than 2 days, not more than 3 days, not more than 4 days, not more than 5 days, not more than 6 days, not more than 7 days, not more than 8 days, not more than 9 days, not more than 10 days, not more than 11 days, not more than 12 days, not more than 13 days, not more than 14 days, not more than 15 days, not more than 16 days, not more than 17 days, not more than 18 days, not more than 19 days, not more than 20 days, not more than 21 days, not more than 22 days, not more than 23 days, not more than 24 days, not more than 25 days, not more than 26 days, not more than 27 days, not more than 28 days, not more than 29 days, or not more than 30 days after a menstrual window has closed. In some embodiments, a sample is collected between 1 day and 30 days, between 1 day and 25 days, between 1 day and 20 days, between 1 day and 15 days, between 1 day and 10 days, between 1 day and 5 days, between 5 days and 30 days, between 5 days and 25 days, between 5 days and 20 days, between 5 days and 15 days, between 5 days and 10 days, between 10 days and 30 days, between 10 days and 25 days, between 10 days and 20 days, between 10 days and 15 days, between 15 days and 30 days, between 15 days and 25 days, between 15 days and 20 days, between 20 days and 30 days, between 20 days and 25 days, or between 25 days and 30 days after a menstrual window has closed.

In some embodiments, non-menstrual fluid collected between two menstrual windows is collected during various points during the reproductive cycle. In some embodiments, non-menstrual fluid is collected during a pre-ovulation phase, during ovulation, or during a post-ovulation phase. In some embodiments, non-menstrual fluid is collected during a proliferative phase, or during a luteal or secretory phase. In some embodiments, a phase of the reproductive cycle is an abnormal phase. In some embodiments, menstrual fluid and non-menstrual fluid is collected from the same subject.

In some embodiments, a sample is collected between two menstrual windows. In some embodiments, a sample is collected about halfway between two menstrual windows, before the halfway point between two menstrual windows, or after the halfway point between two menstrual windows.

In some embodiments, multiple samples are collected between two menstrual windows. In some embodiments, 2, 3, 4, 5, 6, 7, or 8 samples is collected between two menstrual windows. In some such cases, the multiple samples is collected from different times between the two menstrual windows.

In some embodiments, a sample is collected between two menstrual windows, while a second sample is collected between a second two menstrual windows. In further cases, a third sample is collected between a third two menstrual windows. In a general case, an nth sample is collected between n two menstrual windows, where n is a positive integer which is equal to 1 or more.

In some embodiments, fluid samples are collected from a subject both during a menstrual window and between a menstrual window. In some embodiments, a fluid sample is collected from a subject during a menstrual window, and a second fluid sample is collected from the same subject between two menstrual windows. In some embodiments, a fluid sample is collected from a subject during a menstrual window and a second fluid sample is collected from the same subject after the end of that menstrual window, and before the next menstrual window. In some embodiments, a fluid sample is collected from a subject before the start of a menstrual window, and a second fluid sample is collected from the same subject during that menstrual window.

In some embodiments, a volume of fluid, such as menstrual fluid or other fluid collected from a vaginal cavity, is determined using the sample collector. In some embodiments, a volume of menstrual fluid in a sample collector is determined for example by reading graduations on the sample collector. In some embodiments, graduations are at least 0.01 mL, 0.02 mL, 0.03 mL, 0.04 mL, 0.05 mL, 0.06 mL, 0.07 mL, 0.08 mL, 0.09 mL, 0.1 mL, 0.2 mL, 0.3 mL, 0.4 mL, 0.5 mL, 0.6 mL, 0.7 mL, 0.8 mL, 0.9 mL, or 1.0 mL. In some embodiments, a volume of menstrual fluid in a sample collector is determined by measuring the mass of fluid inside the sample collector. In some embodiments, a volume of menstrual fluid in a sample collector is determined for example by compressing or otherwise expelling the fluid from the sample collector and measuring the volume (or mass) of the expelled fluid.

In some embodiments, collected fluid such as menstrual fluid is extracted from the sample collector. In some embodiments, extraction occur by pouring, pipetting, or suctioning of the fluid, which is appropriate, for example, when the sample collector comprises a menstrual cup or other non-absorbent reservoir. In some embodiments, extraction occur by dissolving or otherwise breaking down and removing the sample collector from the sample, which is appropriate, for example, when the sample collector comprises a sponge, a tampon, a pad, or another absorbent material.

Provided herein are also methods for collecting and preserving a sample such as a sample of fluid collected form a vaginal cavity (e.g., menstrual fluid). In some embodiments, such methods is used to accurately and/or reliably indicate a menstrual disorder, such as HMB or AUB, or another such disorder.

In some embodiments, such methods comprise collection of a sample of fluid from the vaginal cavity of a subject. In some embodiments, collection is for example be performed using a collection device such as a sponge, a tampon, a pad, or another absorbent material. In some embodiments, a further description of collection devices and methods is provided herein.

In some embodiments, the fluid is stabilized. In some embodiments, stabilization comprises adding the fluid to a buffer, such as a preservation buffer. In some embodiments, stabilization also comprises storage at a specific temperature (e.g., 4° C., −20° C., room temperature, or another acceptable temperature, including those described herein). In some embodiments, stabilization comprises moving the sample to a new vessel and placing a lid or covering on the vessel. In some embodiments, the vessel is vacuum sealed, or the sample is stored under argon gas or nitrogen gas.

In some embodiments, preservation and stabilization by such methods preserves a component of the sample, such that the component remains substantially unchanged, or that the component does not deteriorate significantly while the sample is stored. In some embodiments, components include a cell, a nucleic acid, a gene target biomarker, a protein biomarker, another protein, a microorganism (e.g., a pathogen or member of a microbiome), a small molecule, a metabolite, or another component.

In some embodiments, a component or property of a component is measured, either quantitatively or qualitatively. In some embodiments, an expression level of a marker is determined, an amount of a metabolite is measured, a small molecule is quantified, a microorganism is identified, a protein is measured, or a nucleic acid is quantified.

In some embodiments, the measured property or component is compared to a reference level. In some embodiments, a reference level is a level of the component or property in a sample from a vaginal cavity of a subject not experiencing HMB or AUB, or of a subject who is healthy. In some embodiments, an increased or decreased expression level of the one or more markers relative to the reference expression level. In some embodiments, such an increase or decrease indicates HMB or AUB. In some embodiments, the method comprises determining from the increased or decreased expression level whether the subject has a menstrual cycle disorder.

### Gene Expression

In some embodiments of methods herein, fluid from the vaginal cavity, such as menstrual fluid comprises a gene expression profile. In some embodiments, during HMB or AUB, this gene expression profile is altered. In some embodiments, alterations to the gene expression profile comprises an increase and/or a decrease in the expression of one or more genes in females having HMB or AUB compared with the menstrual fluid of females having normal menstrual bleeding.

In some embodiments, expression of certain genes in endothelial cells from an endometrium of a female diagnosed with HMB display a reduction in expression of UEA-1 and CD31 and overexpression of F8RA and CD34 when compared with endothelial cells from an endometrium of a female having normal menstrual bleeding. In some embodiments, distinct patterns of expression of osteopontin, laminin, fibronectin, and collagen IV is observed in a female with HMB. In some embodiments, an increase in endothelial cell proliferation occur in in the endometrium of a female with HMB.

In some methods, expression of one or more genes in a sample of fluid collected from a vaginal cavity menstrual fluid is detected or measured. In some embodiments, genes measured is genes which are normally expressed in fluid such as menstrual fluid or genes which are not normally expressed in fluid such as menstrual fluid. In some embodiments, expressed genes which are measured is genes which is expressed or absent during HMB or AUB. Genes has a higher or lower expression during HMB than during normal menstrual bleeding. In some embodiments, such genes include UEA-a, CD31, F8RA, CD34, osteopontin, laminin, fibronectin, collagen IV, or expression products thereof.

In some embodiments, expression or accumulation of protein and/or RNAs is upregulated during HMB or AUB such as, for example, one or more of HLA-Aa (Major histocompatibility complex class I), IL-6ST (Interleukin 6 signal transducer), APCDD1L (Adenomatosis polyposis coli downregulated 1-like), TBX3 (T-box 3), UBN1a (Ubinuclein), DSPa (Desmoplakin), SDCBP2 (Syndecan binding protein (syntenin)), EZH2 (Enhancer of zeste 2 polycomb repressive complex 2 subunit), UHMK1 (U2AF homology motif (UHM) kinase), NR2C2 (Nuclear receptor subfamily 2, group C, member 2), MIR1282a (microRNA), TMED6a (Transmembrane p24 trafficking protein), VAV3 (Vav 3 guanine nucleotide exchange factor), CDC42BPA (CDC42 binding protein kinase alpha (DMPK-like)), C17orf75 (Chromosome 17 open reading frame), MB21D1 (Mab-21 domain containing 1), PTPRC (Protein tyrosine phosphatase, receptor type C), WISP1 (WNT1 inducible signaling pathway protein 1), CDC27 (Cell division cycle 27), and FSD1L (Fibronectin type III and SPRY domain containing 1-like).

In some embodiments, expression or accumulation of protein and/or RNAs is downregulated during HMB or AUB such as, for example, one or more of BPIFB1 ((C20orf114) a BPI fold containing family B, member 1), SCGB3A1a (Secretoglobin, family 3A, member 1) TFF3a (Trefoil factor 3 (intestinal)), SCGB1D2a (Secretoglobin, family 1D, member 2), SCGB2A2a (Secretoglobin, family 2A, member 2), PRODH (Proline dehydrogenase (oxidase) 1), MFF (Mitochondrial fission factor), TSPAN8 (Tetraspanin), CXCL6a (Chemokine (C-X-C motif) ligand 6), SPDYE2 (Speedy/RINGO cell cycle regulator family member E2), FCGBP (Fc fragment of IgG binding protein), IRX6 (Iroquois homeobox 6), ADAM10 (ADAM metallopeptidase domain 10), MUC5ACa (Mucin 5AC, oligomeric mucus/gel-forming), TRHDE-AS1 (TRHDE antisense RNA (LOC283392)), CYP26A1 (Cytochrome P450, family 26, subfamily A, polypeptide 1), SAA2a (Serum amyloid A2), MMEL1 (Membrane metalloendopeptidase-like 1), GRIP2 (Glutamate receptor interacting protein 2), and SAA1a (Serum amyloid A1).

In some embodiments, genes expressed in a sample of fluid from the vaginal cavity, such as menstrual fluid, is measured using an acceptable method. In some embodiments, methods for measuring gene expression include polymerase chain reaction (PCR), quantitative real-time (q-RT-PCR), isothermal PCR, restriction enzyme analysis, RNA sequencing (e.g., sequencing mRNA), northern blotting, serial analysis of gene expression (SAGE), in situ hybridization (ISH), fluorescence ISH (FISH), microarray analysis.

In some embodiments, PCR-based systems use consensus or degenerate primer sequences to allow for amplification and identification of expressed RNA sequences.

In some embodiments, the presence or absence of an expressed gene is measured. Presence of an expressed gene is determined, for example, by detecting at least a trace amount of a gene in a sample. In some embodiments, an expressed gene is present if it is detectable after about 30, 31, 32, 33, 34, 35, 36, 37, 38, 39, or 40 cycles of PCR. In some embodiments, an expressed gene is present if it is detectable by sequencing. In some embodiments, an expressed gene is present if a detectable signal is produced in response to the expressed gene in an imaging experiment such as ISH or FISH. In some embodiments, at least 1, 2, 3, 4, 5, 6, 7, 8, 9, 10, 20, 30, 40, 50, 100, 150, 200, 300, 400, 500, 1000, or more copies of an expressed gene is detectable. In some embodiments, absence of an expressed gene is determined, for example, as failure to detect a gene after about 30, 31, 32, 33, 34, 45, 56, 37, 38, 39, or 40 cycles. In some embodiments, absence of an expressed gene is determined if it is not detected by sequencing in at least 1, 2, 3, 4, 5, or more samples. In some embodiments, absence of an expressed gene is determined if no detectable signal is produced in response to the expressed gene in an imaging experiment, such as ISH or FISH. In some embodiments, an expressed gene is absent if there are no copies in a sample of menstrual fluid. In some embodiments, an expressed gene is absent if the gene is expressed at a level below a threshold, such as a threshold of detection.

In some embodiments, gene expression in a sample is increased or decreased compared with a predetermined threshold. In some embodiments, a predetermined threshold of gene expression is determined from a reference sample, such as a reference sample collected from a subject experiencing normal menstrual bleeding, a subject experiencing HMB, or a subject experiencing AUB. In some embodiments, a threshold gene expression is an upper threshold, such as the maximum gene expression measured during normal menstrual bleeding. In some embodiments, a threshold gene expression is a lower threshold, such as the minimum gene expression measured during normal menstrual bleeding. In some embodiments, a threshold of gene expression is determined by measuring gene expression of a sample of menstrual fluid of at least 1, 2, 3, 4, 5, 10, 15, 20, 30, 40, or 50 subjects experiencing normal menstrual bleeding. In some embodiments, a threshold of gene expression is determined by measuring the gene expression of a sample of menstrual fluid of at least 1, 2, 3, 4, 5, 10, 15, 20, 30, 40, or 50 subjects experiencing HMB. In some embodiments, a threshold of gene expression is determined by measuring the gene expression of a sample of menstrual fluid of at least 1, 2, 3, 4, 5, 10, 15, 20, 30, 40, or 50 subjects experiencing AUB. In some embodiments, a threshold of a gene expression is determined in a sample of fluid collected from a vaginal cavity other than menstrual fluid of at least 1, 2, 3, 4, 5, 10, 15, 20, 30, 40, or 50 subjects. In some embodiments, a threshold of gene expression is determined by comparing the gene expression of a sample of menstrual fluid of subjects experiencing normal menstrual bleeding and HMB, or subjects experiencing normal menstrual bleeding and AUB. In some embodiments, a threshold of gene expression is an average gene expression, a mean gene expression, a mode gene expression, a maximum gene expression, a minimum gene expression, an average gene expression plus 1, 2, or 3 standard deviations of gene expression, or an average gene expression minus 1, 2, or 3 standard deviations of gene expression.

In some embodiments, gene expression is measured as an absolute expression or a relative expression. In some embodiments, absolute expression is measured as an amount of gene present in a tested sample, and is expressed for example as a number of copies, as a mass (e.g., mg), as a number of copies per volume, or as a mass per volume in a menstrual fluid sample. In some embodiments, relative expression is measured as an amount of a gene normalized to another value. In some embodiments, relative expression is normalized for example to a total amount of gene expression in a menstrual fluid sample, an amount of menstrual fluid in a menstrual fluid sample, an amount of cells in a menstrual fluid sample, an amount of a cell type in a menstrual fluid sample, to an amount of a protein biomarker in a menstrual fluid sample, to an amount of a gene target biomarker in a menstrual fluid sample, or to an expression of a same gene in a menstrual fluid sample from a subject experiencing normal menstrual bleeding.

In some embodiments, gene expression is compared to the expression level of the gene in a sample of fluid collected from a vaginal cavity such as menstrual fluid collected from a reference subject. In some embodiments, expression of a gene is increased or decreased in a sample of a subject experiencing HMB or AUB compared with a sample of a subject experiencing normal menstrual bleeding.

In some embodiments, absolute and/or relative expression levels of a gene measured using the methods herein is increased or decreased relative to normal levels of that gene. In some embodiments, this normal gene expression level is a reference expression level. In some embodiments, a reference expression level is the expression of a same gene in the fluid collected from the vaginal cavity of a control subject, such as menstrual fluid of a woman experiencing normal menstrual bleeding. In some embodiments, a reference expression level is the expression of a housekeeping gene, such as gapdh or β-actin. In some embodiments, using a reference expression level, one determines whether the expression of a given gene is increased or decreased in a sample.

In some embodiments, gene expression is measured on any day a subject is experiencing menstrual bleeding. In some embodiments, gene expression is measured more than once during menstrual bleeding. In some embodiments, gene expression is measured on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of menstrual bleeding. Sometimes, gene expression is measured after 30 days of menstrual bleeding. In some embodiments, gene expression is measured on a day a subject is not experiencing menstrual bleeding, such as between two menstrual windows.

In some samples, such as samples from a woman experiencing HMB or AUB, one or more genes is increased or decreased compared with a reference sample. In some embodiments, an increase in a gene expression is indicative of HMB, AUB, a menstrual cycle disorder, or another disorder. In some embodiments, a decrease in a gene expression is indicative of HMB, AUB, a menstrual cycle disorder, or another disorder.

In some embodiments, expression of one or more genes in a sample of a subject experiencing HMB or AUB compared with a sample of a subject experiencing normal menstrual bleeding is correlated or anti-correlated with the presence or severity of HMB or AUB. In some embodiments, an increase in gene expression is correlated with HMB or an increase in gene expression is correlated with AUB. In some embodiments, a decrease in gene expression is correlated with HMB or a decrease in gene expression is correlated with AUB. In some embodiments, an increase of expression of a first gene and a decrease expression of a second gene is correlated with HMB or AUB.

In some embodiments, expression of certain genes present at a given ratio in a normal sample is present in an increased or decreased ratio in a sample of a subject experiencing HMB or AUB. In some embodiments, the ratio of two genes is increased in a sample of a subject experiencing HMB or AUB compared with the ratio of the same two genes in a sample of a subject experiencing normal menstrual bleeding. In some embodiments, the ratio of two genes is decreased in a sample of a subject experiencing HMB or AUB compared with the ratio of the same two genes in a sample of a subject experiencing normal menstrual bleeding.

### Cell Types

In some embodiments of methods herein, fluid taken from the vaginal cavity is a complex, heterogeneous mixture of one or more cell populations and comprises one or more cell types. During HMB or AUB, the cell types found in fluid, such as menstrual blood, is altered. In some embodiments, a fluid sample from the vaginal cavity of a subject experiencing HMB or AUB comprises a different composition of cell types, more of at least one cell type, or less of at least one cell type than a subject experiencing normal menstrual bleeding.

In some embodiments, a cell type in a fluid sample from the vaginal cavity of a subject, such as menstrual fluid, comprises a leukocyte, an erythrocyte, an endothelial cell, an epithelial cell, a stromal cell, a stem cell, a mesenchymal cell, or a combination thereof. In some embodiments, a cell type is present in a sample from a subject experiencing HMB or AUB that is absent in a sample from a subject experiencing normal menstrual bleeding. In some embodiments, a cell type is absent in a sample from a subject experiencing HMB or AUB that is present in a sample from a subject experiencing normal menstrual bleeding. In some embodiments, a cell type is present in a higher quantity in a sample from a subject experiencing HMB or AUB than in a sample from a subject experiencing normal menstrual bleeding. In some embodiments, a cell type is present in a lower quantity in a sample from a subject experiencing HMB or AUB than in a sample from a subject experiencing normal menstrual bleeding.

In some embodiments, a cell type is determined based on the presence, absence, or expression of a cell surface antigen. In some embodiments, a cell type is determined based on a combination or relative expression of two or more cell surface antigens. In some embodiments, a cell is an endothelial cell. In some embodiments, cell surface antigens includes, CD31/PECAM-1, CD34, CD36/SR-B3, CD39, CD44, CD47, CD54/ICAM-1, CD61, CD62E, CD62P, CD80, CD86, CD93, CD102, CD105, CD106, CD112, CD117, ESAM, ENDOMUCIN, CXCL16, CD121a, CD141, CD142, CD143, CD144, CD146, CD147, CD151, CD160, CD201, CD213a, CD248, CD309, ADAMS 8-17, 33, ADAMTS-13, ADAMTS-18, VWF, TEM8, NOTCH, KLF4, or a combination thereof. In some embodiments, a cell is an epithelial cell. In some embodiments, cell surface antigens include, for example, EpCAM, E-cadherin, CD326, or a combination thereof. In some embodiments, a cell is a leukocyte. In some embodiments, cell surface antigens include CD45. In some embodiments, a cell is a mesenchymal cell. In some embodiments, cell surface antigens include, for example, N-cadherin, OB-cadherin, alpha-5, beta-1 integrin, alpha-V, beta-6 integrin, Syndecan-1, or a combination thereof. In some embodiments, a cell type comprises at least 1, at least 2, at least 3, at least 4, at least 5, at least 10, at least 15, or at least 20 cell surface antigens such as those disclosed herein. In some embodiments, a cell type comprises no more than 1, no more than 2, no more than 3, no more than 4, no more than 5, no more than 10, no more than 15, or no more than 20 cell surface antigens such as those disclosed herein.

In some embodiments, a cell type present in a menstrual sample is identified from menstrual fluid collected on any day a subject is experiencing menstrual bleeding. In some embodiments, cell types is identified in samples taken at more than one time point during the menstrual cycle. Cell types is identified in samples taken on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of menstrual bleeding. Sometimes, a cell type is identified in a menstrual fluid sample collected after 30 days of menstrual bleeding. In some embodiments, a cell type is identified when a subject is not experiencing menstrual bleeding, such as between two menstrual windows.

In some embodiments, a cell type of a cell in a sample is determined by an acceptable method, which includes, but is not limited to, flow cytometry, immunohistochemistry, immunocytochemistry, gene expression analysis, protein expression analysis, or metabolome analysis of the cell. In some embodiments, at least 1, 2, 3, 4, 5, 6, 7, 8, 9, 10, 20, 30, 40, 50, or more cell types is determined.

In some embodiments, normalization and cellular characterization analysis is used for identification of cell types. In some embodiments, such analysis requires granular genomic analysis or bioinformatic deconvolution.

In some embodiments, an amount of a cell type in a sample is determined by measuring the amount of that cell type using an acceptable method, which includes flow cytometry, immunohistochemistry, immunocytochemistry, gene expression analysis, protein expression analysis, or metabolome analysis of a plurality of cells. In some embodiments, an amount of a cell type is determined using whole sample. In some embodiments, an amount of a cell type is determined by measuring an amount of a type of cells in a subset of cells of the sample, such as a subset of cells that is removed from the whole sample.

In some embodiments, a presence of a cell type is determined. In some embodiments, a presence of a cell type is expressed as yes or no, wherein yes indicates that at least one of a cell type is identified in a menstrual fluid sample, and no indicates that none of a cell type is identified in a menstrual fluid sample. In some embodiments, a presence of a cell type is expressed semi-quantitatively (e.g. no expression, low expression, moderate expression, or high expression).

In some embodiments, an amount of a cell type is a total amount or a normalized amount of that cell type. In some embodiments, an amount is normalized for example to the total amount of cells in a sample, to a total amount of a subset of cells in the sample, to a biomarker, to a volume, to an amount of time. In some embodiments, an amount of a cell type is a relative amount of that cell type compared with another type of cell in the same sample. In some embodiments, an amount of a cell type is a relative amount of that cell type compared with the amount of the same cell type in a second sample. In some embodiments, the second sample is for example a sample of a woman experiencing a normal menstrual period.

In some embodiments, an amount of a cell type is compared with a predetermined threshold. In some embodiments, an amount of a cell type is increased or decreased by a predetermined threshold. In some embodiments, a predetermined threshold of an amount of a cell type is determined from a reference sample, such as a fluid collected from the vaginal cavity of a control subject, such as menstrual fluid collected from a subject experiencing normal menstrual bleeding, a subject experiencing HMB, or a subject experiencing AUB. In some embodiments, a threshold amount of a cell type is an upper threshold, such as the maximum amount of a cell type measured during normal menstrual bleeding. In some embodiments, a threshold amount of a cell type is a lower threshold, such as the minimum amount of a cell type measured during normal menstrual bleeding. In some embodiments, a threshold of an amount of a cell type is determined by measuring an amount of a cell type of a sample of menstrual fluid of at least 1, 2, 3, 4, 5, 10, 15, 20, 30, 40, or 50 subjects experiencing normal menstrual bleeding. In some embodiments, a threshold of an amount of a cell type is determined by measuring the amount of a cell type of a sample of menstrual fluid of at least 1, 2, 3, 4, 5, 10, 15, 20, 30, 40, or 50 subjects experiencing HMB. In some embodiments, a threshold of an amount of a cell type is determined by measuring the amount of a cell type of a sample of menstrual fluid of at least 1, 2, 3, 4, 5, 10, 15, 20, 30, 40, or 50 subjects experiencing AUB. In some embodiments, a threshold of an amount of a cell type is determined in a sample of fluid collected from a vaginal cavity other than menstrual fluid of at least 1, 2, 3, 4, 5, 10, 15, 20, 30, 40, or 50 subjects. In some embodiments, a threshold of an amount of a cell type is determined by comparing the amount of a cell type of a sample of menstrual fluid of subjects experiencing normal menstrual bleeding and HMB or subjects experiencing normal menstrual bleeding and AUB. In some embodiments, a threshold of an amount of a cell type is an average amount of a cell type, a mean amount of a cell type, a mode gene expression, a maximum gene expression, a minimum gene expression, an average gene expression plus 1, 2, or 3 standard deviations of gene expression, or an average gene expression minus 1, 2, or 3 standard deviations of gene expression.

In some embodiments, an amount of a cell type is increased compared to the normal amount of that cell type or compared with a predetermined threshold. In some embodiments, the amount of a cell type is increased by at least about 10%, 50%, 100%, 150%, or 200%.

In some embodiments, an amount of a cell type is decreased compared to the normal amount of that cell type or compared with a predetermined threshold. In some embodiments, an amount of a cell type is decreased by at least about 10%, 25%, 50%, 75%, or 90%.

In some embodiments, a ratio of cell types is increased compared to the normal amount of that cell type or compared with a predetermined threshold. In some embodiments, a ratio of cell types is increased by at least about 10%, 25%, 50%, 75%, or 90%. In some embodiments, a ratio of cell types is decreased compared to the normal amount of that cell type or compared with a predetermined threshold. In some embodiments, a ratio of cell types is decreased by at least about 10%, 25%, 50%, 75%, or 90%.

In some embodiments, a difference in one or more types of cells found in a fluid sample collected from a vaginal cavity such as menstrual fluid is indicative of HMB, AUB, a menstrual cycle disorder, or another disorder. In some embodiments, a presence of one or more types of cells found in menstrual fluid is indicative of HMB, AUB, a menstrual cycle disorder, or another disorder.

### Nucleic Acid Content

In some embodiments of methods herein, fluid from the vaginal cavity of a subject, such as menstrual blood, comprises a nucleic acid content, which is a total nucleic acid content. In some embodiments, a nucleic acid content (e.g., total nucleic acid) is used as a proxy for loss of material during menstruation. In some embodiments, loss of material describes cells or tissue which is lost during menstruation. In some embodiments, material lost comprises that of the subject, of a fetus, of a bacterium, of a virus, or of a fungus. In some embodiments, higher nucleic acid content in menstrual fluid indicates a higher loss of material, while lower nucleic acid content in menstrual fluid indicates a lower loss of material. During HMB or AUB, nucleic acid content is altered when compared with that of normal menstrual bleeding, indicating that during HMB or AUB, the amount of material lost through menstruation is different than during normal menstrual bleeding.

In some methods, a nucleic acid is measured from a fluid sample obtained from the vaginal cavity of a subject such as menstrual fluid. In some embodiments, nucleic acid is nucleic acid which is present in a collected sample of menstrual fluid from a subject. In some embodiments, nucleic acid is cellular nucleic acid or free nucleic acid. In some embodiments, nucleic acid is nucleic acid of the subject, or is nucleic acid of a virus, bacteria, or fungus present in the menstrual fluid of the subject.

In some embodiments, nucleic acid in such fluid sample comprises deoxyribonucleic acid (DNA) or ribonucleic acid (RNA). In some embodiments, DNA measured comprises chromosomal DNA or mitochondrial DNA. In some embodiments, RNA measured comprises rRNA, tRNA, mRNA, siRNA, or snRNA. In some embodiments, nucleic acid measured comprises maternal nucleic acid or fetal nucleic acid.

In some embodiments, total amount of nucleic acid, total DNA, or total RNA is measured. In some embodiments, a ratio of DNA and total nucleic acid, a ratio of DNA and RNA, or a ratio of RNA and total nucleic acid is measured or calculated from a menstrual fluid sample. In some embodiments, a ratio of a type of RNA to total RNA or total nucleic acid (e.g., mRNA to total RNA or mRNA to total nucleic acid) is measured or calculated from a menstrual fluid sample. In some embodiments, a ratio of a type of DNA to total DNA or total nucleic acid (e.g., mitochondrial DNA to total DNA or mitochondrial DNA to total nucleic acid) is measured or calculated from a menstrual fluid sample. In some embodiments, an amount or ratio of nucleic acid, DNA, or RNA is compared with an amount or ratio of nucleic acid, DNA, or RNA from menstrual fluid of a female with normal menstrual bleeding, from menstrual fluid of a female with HMB, or from menstrual fluid of a female with AUB.

In some embodiments, prior to quantification, nucleic acid, RNA, or DNA is extracted, removed, or purified from a sample for measurement. In some embodiments, extraction methods include organic extraction, Chelex extraction, and solid phase extraction. In some embodiments, organic extraction comprises the addition of one or more chemical solutions to a menstrual fluid sample, lysis, an extraction (e.g., a phenol chloroform extraction), and precipitation (e.g., ethanol precipitation). In some embodiments, Chelex extraction comprises adding a Chelex resin to a menstrual fluid sample, boiling, vortexing, centrifuging, and collecting a supernatant comprising DNA. In some embodiments, solid phase extraction comprises exploiting the ability of DNA to bind to silica by encouraging the binding of DNA to silica (e.g., a column comprising silica or beads comprising silica), perhaps using one or more chaotropic salts.

In some embodiments, extraction, removal, or purification of nucleic acid, RNA, or DNA comprises removal of protein, or other cellular components from a fraction comprising nucleic acid, RNA, or DNA. In some embodiments, extraction, removal, or purification of nucleic acid, RNA, or DNA comprises precipitating and subsequently re-dissolving the nucleic acid, RNA, or DNA. In some embodiments, gravity filtration, vacuum filtration, or centrifugal filtration is employed for extraction, removal, or purification of nucleic acid, RNA, or DNA, perhaps followed by an elution step. In some embodiments, nucleic acid, RNA, or DNA is gel-purified or purified using a filter paper based lysis and elution method.

In some embodiments, nucleic acid from a sample is sequenced using an acceptable sequencing method. In some embodiments, sequencing is performed on the total nucleic acid, or on a subset of the nucleic acid, such as RNA, rRNA, tRNA, mRNA, siRNA, snRNA, DNA, chromosomal DNA, or mitochondrial DNA. Nucleic acid sequenced is nucleic acid of the subject, nucleic acid of a microbiome of the subject (e.g., including viral, bacterial, and/or fungal nucleic acid), or a combination thereof. In some embodiments, basic sequencing methods include Maxam-Gilbert sequencing or chain termination methods. In some embodiments, advanced and de novo sequencing methods includes shotgun sequencing or bridge PCR. In some embodiments, high throughput sequencing methods includes massively parallel signature sequencing, Polony sequencing, 454 pyrosequencing, Illumina sequencing, Combinatorial probe anchor synthesis, SOLiD sequencing, Ion Torrent semiconductor sequencing, DNA nanoball sequencing, Heliscope single molecule sequencing, single molecule real time sequencing, nanopore DNA sequencing, or sequencing using microfluidic systems. In some embodiments, sequencing techniques includes tunneling currents DNA sequencing, sequencing by hybridization, sequencing with mass spectrometry, microfluidic Sanger sequencing, microscopy-based sequencing, RNAP sequencing, or in vitro virus high-throughput sequencing.

In some embodiments, nucleic acid in a sample, including total nucleic acid, DNA, or RNA, is quantified using an acceptable method. In some embodiments, a common method for quantifying nucleic acid, DNA, or RNA is spectrophotometric analysis. Briefly, a spectrophotometer is used to determine the average concentration and purity of DNA, RNA, or both in a sample, by exploiting the light absorbing properties of nucleic acids. In some embodiments, nucleic acids absorb ultraviolet light in a specific pattern. In some embodiments, a spectrophotometer exposes a sample comprising DNA, RNA, or both to ultraviolet light, which has a wavelength of about 260 nm, and measure the light which passes through the sample. In some cases, optical density of the sample is calculated from the intensity of the light which is transmitted through the sample and detected using Beer's law:

$$OD = Log ( \frac{{I}_{i}}{{I}_{t}} ) ,$$

wherein OD is the optical density of a sample, Iᵢ is the intensity of the incident light (e.g., the light applied to a sample), and Iₜ is the transmitted light (e.g., the light detected after passing through a sample). Concentration of nucleic acid in a sample is calculated from optical density as:

C=50 μg/mL×OD×df,

wherein C is the concentration of the DNA or RNA in the sample, OD is the optical density (e.g., as calculated by Beer's law), and df is a dilution factor of the sample.

In some embodiments, another common method for quantifying nucleic acid, DNA, or RNA is fluorescence tagging, e.g., in the presence of a nucleotide dye. Briefly, DNA or RNA is tagged with a fluorescent tag, which is a fluorescent nucleotide dye, and the nucleotide dye that binds to the RNA or DNA selectively fluoresces when bound. In some embodiments, dyes is UV fluorescent dyes. In some embodiments, a tagging method is modified to use a non-fluorescent nucleotide dye, such as a visual dye. In some embodiments, DNA dyes include ethidium bromide, SYBR gold, SYBR green, SYBR safe, Eva green, propidium iodide, crystal violet, dUTP-conjugated probes, 4′,6-diamidino-2-phenylindole, 7-aminoactinomycin D, Hoechst 33258 (33342, 34580), or YOYO-1/DiYO-1/TOTO-1/DiTO-1.

In some embodiments, nucleic acid is measured or quantified on any day a subject is experiencing menstrual bleeding. In some embodiments, nucleic acid is measured more than once during menstrual bleeding. In some embodiments, nucleic acid is measured on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of menstrual bleeding. In some embodiments, nucleic acid is measured after 30 days of menstrual bleeding. In some embodiments, nucleic acid is measured or quantified when a subject is not experiencing menstrual bleeding, such as between two menstrual windows.

In some embodiments, nucleic acid in a sample, including total nucleic acid, DNA, RNA, or a ratio or metric thereof, is increased or decreased compared with a predetermined threshold. In some embodiments, a predetermined threshold of nucleic acid is determined from a reference sample, such as a fluid collected from the vaginal cavity of a control subject, such as menstrual fluid collected from a subject experiencing normal menstrual bleeding, a subject experiencing HMB, or a subject experiencing AUB. In some embodiments, a threshold of nucleic acid is an upper threshold, such as the maximum nucleic acid measured during normal menstrual bleeding. In some embodiments, a threshold of nucleic acid is a lower threshold, such as the minimum nucleic acid measured during normal menstrual bleeding. In some embodiments, a threshold of nucleic acid is determined by measuring the nucleic acid of a sample of menstrual fluid of at least 1, 2, 3, 4, 5, 10, 15, 20, 30, 40, or 50 subjects experiencing normal menstrual bleeding. In some embodiments, a threshold of nucleic acid is determined by measuring the nucleic acid of a sample of menstrual fluid of at least 1, 2, 3, 4, 5, 10, 15, 20, 30, 40, or 50 subjects experiencing HMB. In some embodiments, a threshold of nucleic acid is determined by measuring the nucleic acid of a sample of menstrual fluid of at least 1, 2, 3, 4, 5, 10, 15, 20, 30, 40, or 50 subjects experiencing AUB. In some embodiments, a threshold of nucleic acid is determined in a sample of fluid collected from a vaginal cavity other than menstrual fluid of at least 1, 2, 3, 4, 5, 10, 15, 20, 30, 40, or 50 subjects. In some embodiments, a threshold of nucleic acid is determined by comparing the nucleic acid of a sample of menstrual fluid of subjects experiencing normal menstrual bleeding and HMB, or subjects experiencing normal menstrual bleeding and AUB. In some embodiments, a threshold of nucleic acid is an average amount of nucleic acid, a mean amount of nucleic acid, a mode amount of nucleic acid, a maximum amount of nucleic acid, a minimum amount of nucleic acid, an average amount of nucleic acid plus 1, 2, or 3 standard deviations of an amount of nucleic acid, or an average amount of nucleic acid minus 1, 2, or 3 standard deviations of an amount of nucleic acid.

In some embodiments, an increase in nucleic acid is an increase of at least about 1%, about 5%, about 10%, about 15%, about 25%, about 25%, about 35%, about 40%, about 45%, about 50%, about 55%, about 60%, about 65%, about 70%, about 75%, about 80%, about 85%, about 90%, about 95%, about 100%, about 150%, about 200%, or more compared with a predetermined threshold. In some embodiments, a decrease in nucleic acid is a decrease of at least about 1%, about 5%, about 10%, about 15%, about 25%, about 30%, about 35%, about 40%, about 45%, about 50%, about 55%, about 60%, about 65%, about 70%, about 75%, about 80%, about 85%, about 90%, about 95%, about 100% compared with a predetermined threshold. In some embodiments, an increase in nucleic acid in a sample is indicative of HMB, AUB, a menstrual cycle disorder, or another disorder. In some embodiments, decrease in nucleic acid in a sample is indicative of HMB, AUB, a menstrual cycle disorder, or another disorder.

### Biomarkers

In some embodiments of methods herein, a fluid sample from the vaginal cavity of a subject comprises a biological marker or biomarker profile. In some embodiments, during HMB or AUB, this biomarker profile is altered. In some embodiments, biomarkers is of a tissue or cell, such as a fallopian, ovarian, uterine, endometrial, cervical, or vaginal tissue or cell that is found in a menstrual fluid sample of a subject. In some embodiments, a biomarker is of a component of blood, such as an erythrocyte, plasma, a leukocyte, a platelet, or other component. In some embodiments, a biomarker is common to any set of subjects or a set of all subjects. In some embodiments, a biomarker is common to a set of subjects experiencing normal menstrual bleeding, HMB, or AUB. In some embodiments, a biomarker is unique to a set of subjects experiencing normal menstrual bleeding, HMB, or AUB. One or biomarkers of a menstrual fluid sample is indicative of HMB, AUB, a menstrual cycle disease or disorder, or another disease or disorder.

In some embodiments, a biomarker is measured on any day a subject is experiencing menstrual bleeding. In some embodiments, a biomarker is measured more than once during menstrual bleeding. Flow rate is measured on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of menstrual bleeding. In some embodiments, a biomarker is measured between days 1 and 30, between days 5 and 30, between days 10 and 30, between days 20 and 30, between days 1 and 20, between days 5 and 20, between days 10 and 20, between days 1 and 10, between days 5 and 10, between days 1 and 7, between days 1 and 6, between days 1 and 5, between days 1 and 4, between days 1 and 3, between days 1 and 2, between days 2 and 7, between days 2 and 6, between days 2 and 5, between days 2 and 4, between days 2 and 3, between days 3 and 7, between days 3 and 6, between days 3 and 5, between days 3 and 4, between days 4 and 7, between days 4 and 6, between days 4 and 5, between days 5 and 7, between days 5 and 6, or between days 6 and 7 of menstrual bleeding. Sometimes, a biomarker is measured after 30 days of menstrual bleeding. In some embodiments, a biomarker is measured 1, 2, 3, 4, 5, or more times during a menstrual cycle.

In some embodiments, one or more biomarkers is assayed in a sample collected when a subject is not menstruating, such as between two menstrual windows, such as any time a sample may be collected as described herein. In some embodiments, a biomarker assayed when a subject is not menstruating is a biomarker that is measured when a subject is menstruating, or a biomarker that cannot be measured when a subject is menstruating. In some embodiments, a biomarker measured from a vaginal fluid sample outside the menstrual window is indicative of a disorder. In some embodiments, a biomarker measured from a vaginal sample outside the menstrual window is used in combination with a biomarker measured from a menstrual fluid sample to indicate a menstrual cycle disorder, HMB, or AUB.

In some methods, one or more biomarkers in a sample collected from a vaginal cavity such as menstrual fluid is detected or measured. In some embodiments, biomarkers comprise protein biomarkers or gene target biomarkers. In some embodiments, a protein biomarker is a protein which is found in menstrual blood. In some embodiments, a protein biomarker is either present or absent in normal menstrual blood or in menstrual blood from a subject experiencing HMB or AUB. In some embodiments, a presence or absence of a protein biomarker in a sample of menstrual blood is indicative of a menstrual cycle disorder.

In some embodiments, proteomic analysis of a sample collected from a vaginal cavity such as menstrual fluid identify protein biomarkers which is found in a sample such as a menstrual fluid sample. In some embodiments, a protein biomarkers is unique to menstrual fluid. In some embodiments, a protein biomarkers is unique to another fluid collected from a vaginal cavity. In some embodiments, a proteins are not be found in whole blood or other vaginal discharge or fluid. In some embodiments, identification or measurement of such proteins in a sample provides insight into the health, e.g., menstrual health or reproductive health of a female. In some instances, at least 1, 2, 3, 4, 5 6, 7, 8, 9, 10, 50, 100, 200, 300, 400, or 500 protein biomarkers is identified as unique to menstrual fluid or another fluid collected from a vaginal cavity. In some embodiments, a set of about 385 proteins makes up a unique set of protein biomarkers in menstrual fluid or another fluid collected from a vaginal cavity. In some embodiments, the protein biomarkers are integral to the menstrual cycle, at times including the endometrial cycle and is used to assess normal menstrual bleeding, HMB, or AUB. In some embodiments, a list of protein biomarkers which is found in a menstrual fluid sample is presented in Table 1.

In some embodiments, a protein biomarker is measured using techniques such as enzyme linked immunosorbent assay (ELISA), western blot, LC-MS, flow-cytometry, immunohistochemistry, or other technique capable of detecting or measuring a protein biomarker.

In some embodiments, an amount of a protein biomarker is measured and is expressed as an absolute amount or a relative amount. In some embodiments, an absolute amount of a protein biomarker is measured as an amount of a biomarker present in a tested sample and is expressed for example as a number of molecules, as a mass (e.g., mg), as a number of molecules per volume, or as a mass per volume in a menstrual fluid sample. In some embodiments, a relative amount of a protein biomarker is measured as an amount of a protein biomarker normalized to another value. In some embodiments, a relative amount of a protein is normalized for example to a total amount of protein or protein biomarker in a menstrual fluid sample, to a total amount of a specific protein, protein biomarker, set of proteins, or set of protein biomarkers in a menstrual fluid sample, to an amount of menstrual fluid in a menstrual fluid sample, to an amount of cells in a menstrual fluid sample, to an amount of a cell type in a menstrual fluid sample, to a gene expression in a menstrual fluid sample, to a gene target biomarker in a menstrual fluid sample, or to an amount of a same protein biomarker in a menstrual fluid sample from a subject experiencing normal menstrual bleeding.

In some embodiments, a protein biomarker is increased or decreased in a subject experiencing a disorder such as a menstrual cycle disorder, HMB or AUB. In some embodiments, an increase or decrease is determined by comparing the measured level of a protein biomarker to the measured level of a protein biomarker in a reference sample, such as fluid collected from the vaginal cavity of a control subject, such as a sample of menstrual fluid from a reference subject or subject experiencing normal menstrual bleeding.

In some embodiments, a protein biomarker is increased or decreased compared with a predetermined threshold amount. In some embodiments, a predetermined threshold of a protein biomarker is determined from a reference sample, such as a reference sample collected from a subject having a menstrual cycle disorder, a subject experiencing normal menstrual bleeding, a subject experiencing HMB, or a subject experiencing AUB. In some embodiments, a threshold of a protein biomarker is determined by measuring an amount of a protein biomarker in samples of menstrual blood of at least 1, 2, 3, 4, 5, 10, 15, 20, 30, 40, or 50 subjects experiencing normal menstrual bleeding. In some embodiments, a threshold value is determined by measuring the amount of a protein biomarker of the menstrual blood of at least 1, 2, 3, 4, 5, 10, 15, 20, 30, 40, or 50 subjects experiencing HMB. In some embodiments, a threshold value is determined by measuring the amount of a protein biomarker of the menstrual blood of at least 1, 2, 3, 4, 5, 10, 15, 20, 30, 40, or 50 subjects experiencing AUB. In some embodiments, a threshold of a protein biomarker is determined in a sample of fluid collected from a vaginal cavity other than menstrual fluid of at least 1, 2, 3, 4, 5, 10, 15, 20, 30, 40, or 50 subjects. In some embodiments, a threshold value is determined by comparing an amount of a protein biomarker in samples of subjects experiencing normal menstrual bleeding and HMB or subjects experiencing normal menstrual bleeding and AUB. In some embodiments, a threshold of a protein biomarker is an average amount of a protein biomarker, a mean amount of a protein biomarker, a mode amount of a protein biomarker, a maximum amount of a protein biomarker, a minimum amount of a protein biomarker, an average amount plus 1, 2, or 3 standard deviations of a protein biomarker, or an average amount minus 1, 2, or 3 standard deviations of a protein biomarker.

In some embodiments, an amount of a protein biomarker can increase at least 10%, 20%, 30%, 40%, 50%, 60%, 70%, 80%, 90%, 100%, 150%, 200%, 300%, 400%, or 500% compared with a predetermined threshold. In some embodiments, an amount of a protein biomarker can decrease by at least 10%, 20%, 30%, 40%, 50%, 60%, 70%, 80%, 90%, or 100%, compared with a predetermined threshold. In some embodiments, an increase or decrease in an amount of a protein biomarker is associated with a disorder such as a menstrual cycle disorder, HMB or AUB. In some embodiments, an increase or decrease in a level of a protein biomarker in a sample of menstrual blood is indicative of a menstrual cycle disorder.

In some embodiments, relative levels of two or more protein biomarkers is determined. In some embodiments, a ratio of two protein biomarkers is determined and compared with the ratio of the same two protein biomarkers in a reference sample. In some embodiments, an increase or decrease in said ratio is detected in a subject experiencing HMB or AUB. In some embodiments, an increase or decrease in said ratio in a sample of menstrual fluid is indicative of a menstrual cycle disorder.

In some embodiments, a panel of protein biomarkers is measured. In some embodiments, a panel of protein biomarkers comprises at least 2, 5, 10, 20, 30, 40, 50, 60, 70, 80, 90, or 100 protein biomarkers. In some embodiments, at least 1, 2, 5, 10, 20, 30, 40, 50, 60, 70, 80, 90, or 100 protein biomarkers in the panel is altered (increased or decreased) in a sample collected from a subject experiencing HMB or AUB.

In some embodiments, one or more markers from a panel of biomarkers is measured in menstrual fluid. In some embodiments, some such biomarkers is measured in a sample of a fluid collected from a vaginal cavity that is not menstrual fluid. In some embodiments, proteins which is biomarkers in menstrual fluid may be found in Table 1.

TABLE 1
Menstrual fluid protein biomarkers

| Accession | | Accession | |
|---|---|---|---|
| # | Protein Name | # | Protein Name |
| P15428 | 15-hydroxyprostaglandin dehydrogenase | Q9H293 | Interleukin-25 |
| | [NAD+] | Q96CT2 | Kelch-like protein 29 |
| O15120 | “1-acyl-sn-glycerol-3-phosphate | P02533 | Keratin, type I cytoskeletal 14 |
| | acyltransferase beta 1-acyl-sn-glycerol-3- | P08727 | Keratin, type I cytoskeletal 19 |
| | phosphate acyltransferase beta” | P08779 | Keratin, type I cytoskeletal 16 |
| P62979 | 40S ribosomal protein S27a | Q2M2I5 | Keratin, type I cytoskeletal 24 |
| Q8NHW5 | 60S acidic ribosome protein P0-like | P13645 | Keratin, type I cytoskeletal 10 |
| Q96L21 | 60S ribosomal protein L10-like | P13646 | Keratin, type I cytoskeletal 13 |
| P83881 | 60S ribosomal protein L36a | P35527 | Keratin, type I cytoskeletal 9 |
| Q969Q0 | 60S ribosomal protein L36a-like | Q9NSB4 | Keratin, type II cuticular Hb2 |
| P40429 | 60S ribosome protein L13A | O95678 | Keratin, type II cytoskeletal 75 |
| Q9Y3U8 | 60S ribosome protein L36 | P05787 | Keratin, type II cytoskeletal 8 |
| O95450 | A disintegrin and metalloproteinase with | P08729 | Keratin, type II cytoskeletal 7 |
| | thromb | P12035 | Keratin, type II cytoskeletal 3 |
| Q9UP79 | A disintegrin and metalloproteinase with | P19013 | Keratin, type II cytoskeletal 4 |
| | thromb | Q01546 | Keratin, type II cytoskeletal 2 oral |
| P07510 | Acetylcholine receptor subunit gamma | Q14CN4 | Keratin, type II cytoskeletal 72 |
| Q9BWD1 | Acetyl-CoA acetyltransferase, cyt | Q5XKE5 | Keratin, type II cytoskeletal 79 |
| P68133 | Actin, alpha skeletal muscle | Q6KB66 | Keratin, type II cytoskeletal 80 |
| P63267 | Actin, gamma-enteric smooth muscle | Q86Y46 | Keratin, type II cytoskeletal 73 |
| Q9C0K3 | Actin-related protein 11 | Q8N1N4 | Keratin, type II cytoskeletal 78 |
| Q9P1U1 | Actin-related protein 3B | P04264 | Keratin, type II cytoskeletal 1 |
| Q9BZ11 | ADAM 33 | P35908 | Keratin, type II cytoskeletal 2 |
| Q9H6B4 | Adipocyte adhesion molecule | | epidermal |
| P12235 | ADP/ATP translocase 1 | P13647 | Keratin, type II cytoskeletal 5 |
| Q9H0C2 | ADP/ATP translocase 4 | P02538 | Keratin, type II cytoskeletal 6A |
| P61204 | ADP-ribosylation factor 3 | P04259 | Keratin, type II cytoskeletal 6B |
| P05187 | Alkaline ph | P48668 | Keratin, type II cytoskeletal 6C |
| Q9BQI0 | Allograft inflammatory factor 1-like | Q5T7P3 | Late cornified envelope protein 1B |
| P61647 | Alpha-2,8-sialyltransferase 8F | Q5TA79 | Late cornified envelope protein 2A |
| P35609 | Alpha-actinin-2 | O14633 | Late cornified envelope protein 2B |
| Q6S8J3 | ANKRD26-like family C member 1A | Q5TA81 | Late cornified envelope protein 2C |
| A5A3E0 | ANKRD26-like family C member 1B | Q5TA82 | Late cornified envelope protein 2D |
| Q5VYY1 | Ankyrin repeat domain-containing protein 22 | Q5TA76 | Late cornified envelope protein 3A |
| Q5VT79 | Annexin A8-like protein 2 | Q9BYE3 | Late cornified envelope protein 3D |
| Q16671 | Anti-Muellerian hormone type-2 receptor | Q5TA78 | Late cornified envelope protein 4A |
| P53680 | AP-2 complex subunit sigma-1 | Q2I0M4 | Leucine-rich repeat-containing protein |
| P02654 | Apolipoprotein C-I | | 26 |
| P08519 | Apolipoprotein(a) | Q8N423 | Leukocyte immunoglobulin-like |
| P20292 | Arachidonate 5-lipoxygenase-activating | | receptor subfamily B member 2 |
| | protein | O75197 | Low-density lipoprotein receptor- |
| P15848 | Arylsulfatase B | | related protein 5 |
| Q15121 | Astrocytic ph | Q14114 | Low-density lipoprotein receptor- |
| Q8IZY2 | ATP-binding cassette sub-family A member 7 | | related protein 8 |
| O00571 | ATP-dependent RNA helicase DDX3X | P01229 | Lutropin subunit beta |
| O15523 | ATP-dependent RNA helicase DDX3Y | P07333 | Macrophage colony-stimulating factor |
| P16066 | Atrial natriuretic peptide receptor A | | 1 receptor |
| Q8NFJ9 | Bardet-Biedl syndrome 1 protein | Q9ULC4 | Malignant T call amplified sequence 1 |
| Q9BYX7 | Beta-actin-like protein 3 | Q8WXG6 | MAP kinase-activating death domain |
| P35612 | Beta-adducin | | protein |
| Q9Y2P5 | Bile acyl-CoA synthetase | Q495T6 | Membrane metallo-endopeptidase-like |
| O14514 | Brain-specific angiogenesis inhibitor 1 | | 1 |
| Q8WY22 | BRI3-binding protein | Q15049 | Membrane protein MLC1 |
| Q6PL45 | BRICH | Q14696 | Mesoderm development candidate 2 |
| O95696 | Bromodomain-containing protein 1 | P10620 | Microsomal glutathione S-transferase 1 |
| Q8WVV5 | Butyrophilin subfamily 2 member A2 | Q96DA6 | Mitochondrial import inner membrane |
| Q9HCU4 | Cadherin EGF LAG seven-pass G-type | | translocase subunit TIM14 |
| | receptor 2 | P27361 | Mitogen-activated protein kinase 3 |
| Q9UJ99 | Cadherin-22 | O14950 | Myosin regulatory light chain 12B |
| Q9Y6N3 | Calcium-activated chloride channel regulator | Q76KP1 | N-acetyl-beta-gluc |
| | family member 3 | P51970 | NADH dehydrogenase [ubiquinone] 1 |
| P07451 | Carbonic anhydrase 3 | | alpha subcomplex subunit 8 |
| P49747 | Cartilage oligomeric matrix protein | Q96PH1 | NADPH oxidase 5 |
| Q8N126 | Cell adhesion molecule 3 | O95944 | Natural cytotoxicity triggering receptor |
| O95833 | Chloride intracellular channel protein 3 | | 2 |
| P11597 | Cholesteryl ester transfer protein | Q9P2S2 | Neurexin-2-alpha |
| P01233 | Choriogonadotropin subunit beta | P58401 | Neurexin-2-beta |
| A6NKQ9 | Choriogonadotropin subunit beta variant 1 | Q99466 | Neurogenic locus notch homolog |
| Q6NT52 | Choriogonadotropin subunit beta variant 2 | | protein 4 |
| Q9P2D1 | Chromodomain-helicase-DNA-binding | P48645 | Neuromedin-U |
| | protein 7 | Q8N729 | Neuropeptide W |
| Q9Y281 | Cofilin-2 | P12838 | Neutrophil defensin 4 |
| Q8N6L0 | Coiled-coil domain-containing protein 155 | O15381 | Nuclear val |
| A1A5D9 | Coiled-coil domain-containing protein 64B | Q8NH81 | Olfactory receptor 10G6 |
| Q14050 | Collagen alpha-3(IX) chain | O43612 | Orexin |
| Q96CG8 | Collagen triple helix repeat-containing protein | Q9HC10 | Otoferlin |
| | 1 | P50542 | Peroxisomal targeting signal 1 receptor |
| Q9NPY3 | Complement component Clq receptor | Q9BTY2 | Plasma alpha-L-fuc |
| Q9HCH3 | Copine-5 | Q9UIW2 | Plexin-A1 |
| O95741 | Copine-6 | Q9Y4D7 | Plexin-D1 |
| Q9UBL6 | Copine-7 | Q7Z5L7 | Podocan |
| Q86YQ8 | Copine-8 | Q6PEZ8 | Podocan-like protein 1 |
| Q9P0M6 | Core histone macro-H2A.2 | Q92692 | Poliovirus receptor-related protein 2 |
| Q9BYD5 | Cornifelin | Q96NY8 | Poliovirus receptor-related protein 4 |
| Q9H2A7 | C-X-C motif chemokine 16 | O15354 | Probable G-protein coupled receptor 37 |
| O14949 | Cytochrome b-c1 complex subunit 8 | Q8N0Y7 | Probable phosphoglycerate mutase 4 |
| P09669 | Cytochrome c oxidase subunit 6C | O43555 | Progonadoliberin-2 |
| Q02928 | Cytochrome P450 4A11 | Q9HC23 | Prokineticin-2 |
| Q5TCH4 | Cytochrome P450 4A22 | Q8IVL6 | Prolyl 3-hydroxylase 3 |
| Q14008 | Cytoskeleton-associated protein 5 | Q16647 | Prostacyclin synthase |
| Q96GG9 | DCNl-like protein 1 | Q8TAA3 | Proteasome subunit alpha type-7-like |
| Q9BTC0 | Death-inducer obliterator 1 | Q86UD0 | Protein C9orf140 |
| Q08495 | Dematin | Q9BT09 | Protein canopy homolog 3 |
| Q8TEH3 | DENN domain-containing protein 1A | P05129 | Protein kinase C gamma type |
| Q8IV53 | DENN domain-containing protein 1C | Q8N114 | Protein shisa-5 |
| Q9H4A9 | Dipeptidase 2 | Q93097 | Protein Wnt-2b |
| Q9H4B8 | Dipeptidase 3 | Q9Y6F9 | Protein Wnt-6 |
| O95886 | Disks large-associated protein 3 | Q9UM07 | Protein-arginine deiminase type-4 |
| O95147 | Dual specificity protein ph | Q9UN74 | Protocadherin alpha-4 |
| Q9P225 | Dynein heavy chain 2, axonemal | Q9Y5H6 | Protocadherin alpha-8 |
| Q96JB1 | Dynein heavy chain 8, axonemal | Q9Y5I4 | Protocadherin alpha-C2 |
| Q5XPI4 | E3 ubiquitin-protein ligase RNF123 | Q14517 | Protocadherin Fat 1 |
| P84090 | Enhancer of rudimentary homolog | Q96JQ0 | Protocadherin-16 |
| P52797 | Ephrin-A3 | Q2VWP7 | Protogenin |
| P52798 | Ephrin-A4 | P07949 | Proto-oncogene tyr |
| Q08345 | Epithelial discoidin domain-containing | Q5JNZ5 | Putative 40S rib |
| | receptor 1 | A6NMY6 | Putative annexin A2-like protein |
| Q9NZJ5 | Eukaryotic translation initiation factor 2-alpha | A8MZA4 | Putative golgin subfamily A member |
| | kinase 3 | | 6-like protein 6 |
| Q9UKV8 | Eukaryotic translation initiation factor 2C 2 | P48741 | Putative heat shock 70 kDa protein 7 |
| Q16658 | Fascin | Q58FF7 | Putative heat shock protein HSP 90- |
| Q9Y3I1 | F-box only protein 7 | | beta-3 |
| Q9NYL4 | FK506-binding protein 11 | Q6DN03 | Putative histone H2B type 2-C |
| Q5SZK8 | FRAS1-related extracellular matrix protein 2 | Q6DRA6 | Putative histone H2B type 2-D |
| O15552 | Free fatty acid receptor 2 | Q92928 | Putative Ras-related protein Rab-1C |
| Q9H479 | Fructosamine-3-kinase | Q5VYJ4 | Putative small nuclear |
| O75899 | Gamma-aminobutyric acid type B receptor | | ribonucleoprotein polypeptide E-like |
| | subunit 2 | | protein 1 |
| Q9GZZ7 | GDNF family receptor alpha-4 | A6NGW2 | Putative stereocilin-like protein |
| Q8N2G8 | GH3 domain-containing protein | Q9H853 | Putative tubulin-like protein alpha-4B |
| P47871 | Glucagon receptor | A6NJS3 | Putative V-set and immunoglobulin |
| O76003 | Glutaredoxin-3 | | domain-containing-like protein |
| P08263 | Glutathione S-transferase A1 | | ENSP00000303034 |
| P09210 | Glutathione S-transferase A2 | P35241 | Radixin |
| Q16772 | Glutathione S-transferase A3 | A6NIZ1 | Ras-related protein Rap-1b-like protein |
| Q9NZD2 | Glycolipid transfer protein | Q92932 | Receptor-type tyr |
| P02724 | Glycophorin-A | Q9NZ71 | Regulator of telomere elongation |
| Q8N3Y3 | Glycosyltransferase-like protein LARGE2 | | helicase 1 |
| Q8N7Z2 | Golgin subfamily A member 6-like protein 1 | Q96D15 | Reticulocalbin-3 |
| Q96HH9 | GRAM domain-containing protein 3 | Q02846 | Retinal guanylyl cyclase 1 |
| O15496 | Group 10 secretory ph | P11908 | Ribose-phosphate pyrophosphokinase |
| P01116 | GTPase KRas | | 2 |
| Q9UBI6 | Guanine nucleotide-binding protein | P21108 | Ribose-phosphate pyrophosphokinase |
| | G(I)/G(S)/G(O) subunit gamma-12 | | 3 |
| Q9HAV0 | Guanine nucleotide-binding protein subunit | Q15050 | Ribosome biogenesis regulatory |
| | beta-4 | | protein homolog |
| Q6B0K9 | Hemoglobin subunit mu | O94885 | SAM and SH3 domain-containing |
| P09105 | Hemoglobin subunit theta-1 | | protein 1 |
| P02008 | Hemoglobin subunit zeta | P10523 | S-arrestin |
| Q8TDQ0 | Hepatitis A virus cellular receptor 2 | Q96GP6 | Scavenger receptor class F member 2 |
| Q04756 | Hepatocyte growth factor activator | P09683 | Secretin |
| P81172 | Hepcidin | Q13018 | Secretory ph |
| P55795 | Heterogeneous nuclear ribonucleoprotein H2 | Q9NPR2 | Semaphorin-4B |
| Q6UXD1 | Histidine-rich carboxyl terminus protein 1 | Q9H3T3 | Semaphorin-6B |
| Q92522 | Histone H1x | Q9H3T2 | Semaphorin-6C |
| P04908 | Histone H2A type 1-B/E | Q9UQ35 | Serine/arginine repetitive matrix |
| P20671 | Histone H2A type 1-D | | protein 2 |
| Q96KK5 | Histone H2A type 1-H | Q9BZL6 | Serine/threonine-protein kinase D2 |
| Q99878 | Histone H2A type 1-J | Q9UL54 | Serine/threonine-protein kinase TAO2 |
| Q6FI13 | Histone H2A type 2-A | Q8IYT8 | Serine/threonine-protein kinase ULK2 |
| Q16777 | Histone H2A type 2-C | P30154 | Serine/threonine-protein ph |
| Q9BTM1 | Histone H2A.J | P36873 | Serine/threonine-protein ph |
| P16104 | Histone H2A.x | P62714 | Serine/threonine-protein ph |
| P58876 | Histone H2B type 1-D | Q5TCZ1 | SH3 and PX domain-containing protein |
| Q93079 | Histone H2B type 1-H | | 2A |
| P06899 | Histone H2B type 1-J | Q96PQ1 | Sialic acid-binding Ig-like lectin 12 |
| Q99879 | Histone H2B type 1-M | Q9NYZ4 | Sialic acid-binding Ig-like lectin 8 |
| P23527 | Histone H2B type 1-O | Q9NQ36 | Signal peptide, CUB and EGF-like |
| Q16778 | Histone H2B type 2-E | | domain-containing protein 2 |
| Q5QNW6 | Histone H2B type 2-F | P49771 | SL cytokine |
| P57053 | Histone H2B type F-S | Q96A28 | SLAM family member 9 |
| P84243 | Histone H3.3 | Q8N130 | Sodium-dependent phosphate transport |
| Q6NXT2 | Histone H3-like | | protein 2C |
| Q99525 | Histone H4-like protein type G | Q9UMY4 | Sorting nexin-12 |
| P78426 | Homeobox protein Nkx-6.1 | Q7RTU9 | Stereocilin |
| P01742 | Ig heavy chain V-I region EU | P78539 | Sushi repeat-containing protein SRPX |
| P06326 | Ig heavy chain V-I region Mot | Q7RTX1 | Taste receptor type 1 member 1 |
| P01761 | Ig heavy chain V-I region SIE | Q7RTX0 | Taste receptor type 1 member 3 |
| P01760 | Ig heavy chain V-I region WOL | Q9BZG2 | Testicular acid ph |
| P01777 | Ig heavy chain V-III region TEI | Q9UI38 | Testis-specific protease-like protein 50 |
| P01765 | Ig heavy chain V-III region TIL | Q9UPZ6 | Thrombospondin type-1 domain- |
| P01763 | Ig heavy chain V-III region WEA | | containing protein 7 A |
| P01594 | Ig kappa chain V-I region AU | P01266 | Thyroglobulin |
| P04432 | Ig kappa chain V-I region Daudi | Q9UPV9 | Trafficking kinesin-binding protein 1 |
| P01598 | Ig kappa chain V-I region EU | Q969E4 | Transcription elongation factor A |
| P01599 | Ig kappa chain V-I region Gal | | protein-like 3 |
| P01600 | Ig kappa chain V-I region Hau | Q6IPX3 | Transcription elongation factor A |
| P01601 | Ig kappa chain V-I region HK101 (Fragment) | | protein-like 6 |
| P01607 | Ig kappa chain V-I region Rei | P30536 | Translocator protein |
| P01608 | Ig kappa chain V-I region Roy | P43307 | Translocon-associated protein subunit |
| P04431 | Ig kappa chain V-I region Walker | | alpha |
| P80362 | Ig kappa chain V-I region WAT | Q9BVT8 | Transmembrane and ubiquitin-like |
| P01610 | Ig kappa chain V-I region WEA | | domain-containing protein 1 |
| P06309 | Ig kappa chain V-II region GM607 | Q9Y3Q3 | Transmembrane emp24 domain- |
| | (Fragment) | | containing protein 3 |
| P06310 | Ig kappa chain V-II region RPMI 6410 | A6NL71 | Transmembrane protease, serine 11E2 |
| P01619 | Ig kappa chain V-III region B6 | Q9UK28 | Transmembrane protein 59-like |
| P04206 | Ig kappa chain V-III region GOL | A6NKL6 | Transmembrane protein TTMA |
| P01621 | Ig kappa chain V-III region NG9 (Fragment) | Q13428 | Treacle protein |
| P01624 | Ig kappa chain V-III region POM | Q9HCJ0 | Trinucleotide repeat-containing gene |
| P04434 | Ig kappa chain V-III region VH (Fragment) | | 6C protein |
| P01623 | Ig kappa chain V-III region WOL | P28289 | Tropomodulin-1 |
| P06314 | Ig kappa chain V-IV region B17 | P07477 | Trypsin-1 |
| P06313 | Ig kappa chain V-IV region JI | P07478 | Trypsin-2 |
| P06316 | Ig lambda chain V-I region BL2 | P35030 | Trypsin-3 |
| P01700 | Ig lambda chain V-I region HA | Q96A98 | Tuberoinfundibular peptide of 39 |
| P01699 | Ig lambda chain V-I region VOR | | residues |
| P01715 | Ig lambda chain V-IV region Bau | Q13748 | Tubulin alpha-3C/D chain |
| O95373 | Importin-7 | Q6PEY2 | Tubulin alpha-3E chain |
| Q8WX77 | Insulin-like growth factor-binding protein-like | Q9NY65 | Tubulin alpha-8 chain |
| | 1 | Q13885 | Tubulin beta-2A chain |
| P19827 | Inter-alpha-trypsin inhibitor heavy chain H1 | Q9BVA1 | Tubulin beta-2B chain |
| P19823 | Inter-alpha-trypsin inhibitor heavy chain H2 | Q13509 | Tubulin beta-3 chain |
| Q06033 | Inter-alpha-trypsin inhibitor heavy chain H3 | P08138 | Tumor necr |
| Q9UHA7 | Interleukin-1 family member 6 | P41273 | Tumor necrosis factor ligand |
| Q13261 | Interleukin-15 receptor subunit alpha | | superfamily member 9 |
| Q9HBE5 | Interleukin-21 receptor | Q13454 | Tumor suppressor candidate 3 |
| Q5T200 | Zinc finger CCCH domain-containing protein | Q12792 | Twinfilin-1 |
| | 13 | Q9Y4E8 | Ubiquitin carboxyl-terminal hydrolase |
| Q12836 | Zona pellucida sperm-binding protein 4 | | 15 |
| Q9HCY8 | Protein S100-A14 | Q8TEY7 | Ubiquitin carboxyl-terminal hydrolase |
| | | | 33 |
| | | Q5VVQ6 | Ubiquitin thioesterase OTU1 |
| | | P51668 | Ubiquitin-conjugating enzyme E2 D1 |
| | | P61077 | Ubiquitin-conjugating enzyme E2 D3 |
| | | Q9Y2X8 | Ubiquitin-conjugating enzyme E2 D4 |
| | | P62253 | Ubiquitin-conjugating enzyme E2 G1 |
| | | Q14376 | UDP-glucose 4-epimerase |
| | | Q6UX73 | Uncharacterized protein C16orf89 |
| | | Q6ICL3 | Uncharacterized protein C22orf25 |
| | | Q08AI8 | Uncharacterized protein C2orf54 |
| | | Q5BLP8 | Uncharacterized protein C4orf48 |
| | | Q5TF21 | Uncharacterized protein C6orfl74 |
| | | Q9Y2F5 | Uncharacterized protein KIAA0947 |
| | | Q6ZSJ9 | UPF0626 protein B |
| | | Q969E3 | Urocortin-3 |
| | | P10746 | Uroporphyrinogen-III synthase |
| | | P54725 | UV excision repair protein RAD23 |
| | | | homolog A |
| | | Q9H0V9 | VIP36-like protein |
| | | Q8TEU8 | WAP, kazal, immunoglobulin, kunitz |
| | | | and NTR domain-containing protein 2 |

In some embodiments, a gene target biomarker is a gene target which is present in a sample collected from a vaginal cavity such as menstrual fluid. In some embodiments, a gene target biomarker is either present or absent in a fluid collected from a vaginal cavity, normal menstrual fluid, or in menstrual fluid from a subject experiencing HMB or AUB. In some embodiments, a presence, absence, or value of a gene target biomarker in a sample collected from a vaginal cavity menstrual fluid is indicative of a menstrual cycle disorder.

In some embodiments, a gene target biomarker in a sample collected from a vaginal cavity such as menstrual fluid is measured using an acceptable method In some embodiments, methods for measuring gene expression include polymerase chain reaction (PCR), quantitative real-time (q-RT-PCR), isothermal PCR, restriction enzyme analysis, RNA sequencing (e.g., sequencing mRNA), northern blotting, serial analysis of gene expression (SAGE), in situ hybridization (ISH), fluorescence ISH (FISH), and microarray analysis, and microarray analysis.

In some embodiments, an amount of a gene target biomarker is measured, and is expressed as an absolute amount or a relative amount. In some embodiments, absolute expression is measured as an amount of a gene target biomarker present in a tested sample, and is expressed for example as a number of copies, as a mass (e.g., mg), as a number of copies per volume, or as a mass per volume in a menstrual fluid sample. In some embodiments, relative expression is measured as an amount of a gene target biomarker normalized to another value. In some embodiments, relative expression is normalized for example to a total amount of one or more gene target biomarkers in a sample collected from a vaginal cavity such as a menstrual fluid sample, an amount of menstrual fluid in a menstrual fluid sample, an amount of cells in a menstrual fluid sample, an amount of a cell type in a menstrual fluid sample, to an amount of a protein in a menstrual fluid sample, or to an amount of a same gene target biomarker in a menstrual fluid sample from a subject experiencing normal menstrual bleeding.

In some embodiments, a gene target biomarker is increased or decreased in a subject experiencing a menstrual cycle disorder, HMB, or AUB. In some embodiments, an increase or decrease is determined by comparing the measured level of a gene target biomarker to the measured level of a protein biomarker in a reference sample fluid collected from the vaginal cavity of a control subject, such as a sample of menstrual fluid from a reference subject or subject experiencing normal menstrual bleeding.

In some embodiments, a gene target biomarker is increased or decreased compared with a predetermined threshold. In some embodiments, a predetermined threshold of a gene target biomarker is determined from a reference sample, such as a reference sample collected from a subject experiencing normal menstrual bleeding, a subject experiencing HMB, or a subject experiencing AUB. In some embodiments, a threshold gene target biomarker is an upper threshold, such as the maximum gene expression measured during normal menstrual bleeding. In some embodiments, a threshold gene target biomarker is a lower threshold, such as the minimum gene expression measured during normal menstrual bleeding. In some embodiments, a threshold of a gene target biomarker is determined by measuring a gene target biomarker of a sample of a fluid collected from a vaginal cavity such as menstrual fluid of at least 1, 2, 3, 4, 5, 10, 15, 20, 30, 40, or 50 subjects experiencing normal menstrual bleeding. In some embodiments, a threshold of gene target biomarker is determined by measuring the gene expression of a sample of menstrual fluid of at least 1, 2, 3, 4, 5, 10, 15, 20, 30, 40, or 50 subjects experiencing HMB. In some embodiments, a threshold of a gene target biomarker is determined by measuring the gene expression of a sample of menstrual fluid of at least 1, 2, 3, 4, 5, 10, 15, 20, 30, 40, or 50 subjects experiencing AUB. In some embodiments, a threshold of a gene target biomarker is determined in a sample of fluid collected from a vaginal cavity other than menstrual fluid of at least 1, 2, 3, 4, 5, 10, 15, 20, 30, 40, or 50 subjects. In some embodiments, a threshold of a gene target biomarker is determined by comparing the gene expression of a sample of menstrual fluid of subjects experiencing normal menstrual bleeding and HMB or subjects experiencing normal menstrual bleeding and AUB. In some embodiments, a threshold of a gene target biomarker is an average amount of a gene target biomarker, a mean amount of a gene target biomarker, a mode amount of a gene target biomarker, a maximum amount of a gene target biomarker, a minimum amount of a gene target biomarker, an average amount of a gene target biomarker, plus 1, 2, or 3 standard deviations of gene expression, or an average amount of a gene target biomarker, minus 1, 2, or 3 standard deviations of gene expression.

In some embodiments, an amount of a gene target biomarker can increase at least 10%, 20%, 30%, 40%, 50%, 60%, 70%, 80%, 90%, 100%, 150%, 200%, 300%, 400%, or 500% compared with a predetermined threshold. In some embodiments, an amount of a gene target biomarker can decrease by at least 10%, 20%, 30%, 40%, 50%, 60%, 70%, 80%, 90%, or 100% compared with a predetermined threshold. In some embodiments, an increase or decrease in an amount of a protein biomarker is associated with HMB or AUB. In some embodiments, an increase or decrease in a level of a protein biomarker in a sample of menstrual fluid is indicative of a menstrual cycle disorder.

In some embodiments, relative levels of two or more gene target biomarkers is determined. In some embodiments, a ratio of two gene target biomarkers is determined and compared with the ratio of the same two gene target biomarkers in a reference sample. In some embodiments, an increase or decrease in said ratio is detected in a subject experiencing HMB or AUB. In some embodiments, an increase or decrease in said ratio in a sample of menstrual fluid is indicative of a menstrual cycle disorder.

In some embodiments, a panel of gene target biomarkers is measured. In some embodiments, a panel of gene target biomarkers comprises at least 2, 5, 10, 20, 30, 40, 50, 60, 70, 80, 90, or 100 gene target biomarkers. In some embodiments, at least 1, 2, 5, 10, 20, 30, 40, 50, 60, 70, 80, 90, or 100 gene target biomarkers in the panel is altered (increased or decreased) in a sample collected from a subject experiencing HMB or AUB.

In some embodiments, one or more genes is used as target biomarkers for a menstrual fluid sample from a subject. In some embodiments, gene targets which is biomarkers in menstrual fluid may be found in Table 2.

TABLE 2
Menstrual blood gene target biomarkers
Gene Name

| | | | | | | |
|---|---|---|---|---|---|---|
| GNB1 | MPZL2 | KRT13 | RPS21 | DEK | RPL27A | ARL6IP1 |
| RPL22 | MLL | KRT16 | PPDPF | C6orf62 | IPO7 | SCNN1B |
| ENO1 | RPS25 | EIF1 | PTK6 | FAM65B | EIF4G2 | IL4R |
| PLOD1 | ARHGEF12 | PTRF | DNAJC5 | HIST1H2AC | COPB1 | MVP |
| EPHA2 | HSPA8 | RPL27 | MIR3648 | HLA-A | RPS13 | CORO1A |
| CDC42 | ETS1 | SLC25A39 | MIR3687 | HLA-E | LDHA | PRSS8 |
| CLIC4 | APLP2 | ARHGAP27 | NRIP1 | TUBB | HIPK3 | CYLD |
| MED18 | CCND2 | CDC27 | APP | MUC21 | CAPRIN1 | RBL2 |
| PHACTR4 | SCARNA10 | ITGB3 | CCT8 | HLA-C | CD44 | GFOD2 |
| EPB41 | GAPDH | MMD | C21orf7 | HLA-B | PTPRJ | KARS |
| PTP4A2 | CHD4 | SRSF1 | BACH1 | CLIC1 | DDB1 | COX4I1 |
| KHDRBS1 | RIMKLB | CLTC | MORC3 | CFB | EEF1G | CYBA |
| EIF3I | A2ML1 | MED13 | TTC3 | HLA-DRA | SLC3A2 | RPL13 |
| RBBP4 | ARHGDIB | DDX5 | BRWD1 | HLA-DRB1 | FAU | PRPF8 |
| SFPQ | LDHB | GNA13 | TMPRSS2 | HLA-DQB1 | CFL1 | SPNS2 |
| MACF1 | TWF1 | PRKAR1A | SIK1 | HLA-DPA1 | GSTP1 | PFN1 |
| YBX1 | ANO6 | RPL38 | CSTB | HLA-DPB1 | CCND1 | ALOX12 |
| SLC2A1 | TUBA1B | EVPL | PFKL | RPS18 | CTTN | EIF5A |
| RPS8 | TMBIM6 | 9-Sep | COL18A1 | RPL10A | RAB6A | RPL26 |
| PRDX1 | DAZAP2 | MYL12A | COL6A2 | SRSF3 | RPS3 | NCOR1 |
| CMPK1 | KRT80 | MYL12B | MAPK1 | C6orf132 | PICALM | UBB |
| RNF11 | KRT4 | ANKRD12 | MIF | HSP90AB1 | BIRC3 | C17orf76-AS1 |
| NRD1 | KRT78 | RAB31 | FBXO7 | CRISP3 | BIRC2 | RPL23A |
| USP24 | KRT8 | ROCK1 | MYH9 | EEF1A1 | TMEM123 | RPL23 |
| JAK1 | EIF4B | ATP5A1 | EIF3D | NT5E | MMP1 | RPL19 |
| PDE4B | PFDN5 | C18orf25 | LGALS1 | SYNCRIP | ATM | NR1D1 |
| SERBP1 | ITGA5 | SMAD2 | EIF3L | UBE2J1 | CD24 | CHD1 |
| SH3GLB1 | RPL41 | RAB27B | DDX17 | AIM1 | ARPC2 | PJA2 |
| GBP2 | MYL6 | FECH | JOSD1 | SNX3 | DOCK10 | CDC42SE2 |
| RPL5 | PTGES3 | LMAN1 | RPL3 | CD164 | TRIP12 | SKP1 |
| SCARNA2 | NACA | CD226 | ATF4 | SERINC1 | SP100 | PITX1 |
| SORT1 | LRP1 | HMHA1 | XRCC6 | VNN1 | NCL | TGFB1 |
| CAPZA1 | CTDSP2 | GPX4 | ARL8B | RPS12 | PTMA | HSPA9 |
| RHOC | USP15 | MIDN | EMC3 | SGK1 | C2orf54 | MATR3 |
| HIPK1 | RAP1B | RPS15 | RPL32 | UTRN | HDLBP | PAIP2 |
| CSDE1 | LYZ | OAZ1 | ANKRD28 | SASH1 | 2-Sep | SPINK5 |
| ATP1A1 | NAP1L1 | EEF2 | UBE2E1 | SYNE1 | RASSF2 | GRPEL2 |
| MAN1A2 | OSBPL8 | UBXN6 | RPL15 | SOD2 | XRN2 | CD74 |
| FAM46C | BTG1 | RPL36 | TOP2B | WTAP | RBM39 | RPS14 |
| TXNIP | SLC25A3 | RPS28 | TGFBR2 | IGF2R | LINC00657 | ANXA6 |
| ECM1 | HSP90B1 | ZNF426 | CMTM6 | QKI | TGM2 | GM2A |
| MCL1 | C12orf75 | CDC37 | GOLGA4 | RNASET2 | TOP1 | SPARC |
| CDC42SE1 | GLTP | ILF3 | RPSA | MAFK | SRSF6 | CYFIP2 |
| S100A10 | RPL6 | CALR | RPL14 | ACTB | PKIG | CLINT1 |
| FLG | PTPN11 | RPL18A | CTNNB1 | EIF2AK1 | YWHAB | DOCK2 |
| CRNN | RPLP0 | FKBP8 | HIGD1A | HNRNPA2B1 | STK4 | NPM1 |
| CRCT1 | RAN | UBA52 | GPX1 | CBX3 | MMP9 | ATP6V0E1 |
| LCE3E | ZMYM2 | UBA2 | RHOA | C7orf41 | FAM210B | HNRNPH1 |
| LCE3D | USP12 | SPINT2 | RPL29 | 7-Sep | RTFDC1 | CANX |
| SPRR3 | HMGB1 | NCCRP1 | TKT | PPIA | RBM38 | GNB2L1 |
| SPRR2D | HSPH1 | RPS19 | ARF4 | UPP1 | RAB22A | CDYL |
| SPRR2A | UFM1 | ARHGEF1 | ARL6IP5 | CHCHD2 | GNAS | F13A1 |
| SPRR2E | ELF1 | CNFN | FOXP1 | AKAP9 | TUBB1 | SDPR |
| S100A8 | TSC22D1 | VASP | CGGBP1 | GNG11 | RPS6KA3 | STK17B |
| S100A6 | TPT1 | SNRPD2 | PCNP | GNB2 | EIF2S3 | SF3B1 |
| RPS27 | LCP1 | CALM3 | RPL24 | SERPINE1 | CYBB | HSPD1 |
| TPM3 | LMO7 | GLTSCR2 | BBX | PRKAR2B | DDX3X | CLK1 |
| ASH1L | MYCBP2 | FTL | FSTL1 | LAMB1 | SLC9A7 | ACSL1 |
| ARHGEF2 | DNAJC3 | RPL13A | CSTA | TES | TIMP1 | CCT5 |
| LMNA | IPO5 | RPS11 | ITGB5 | CAPZA2 | RBM3 | SUB1 |
| CCT3 | COL4A2 | VSIG10L | SEC61A1 | SND1 | MAGED2 | IL7R |
| HDGF | CUL4A | ZNF83 | RPN1 | HILPDA | ALAS2 | RPL37 |
| TAGLN2 | RPPH1 | RPS9 | CNBP | CALU | MSN | IL6ST |
| COPA | PNP | EPS8L1 | CDV3 | CPA4 | OGT | MAP3K1 |
| UHMK1 | HNRNPC | RPL28 | COPB2 | MKLN1 | RPS4X | KIF2A |
| PRRC2C | ARHGAP5 | RPS5 | ZBTB38 | CALD1 | XIST | TNPO1 |
| RABGAP1L | BAZ1A | RPS7 | XRN1 | JHDM1D | ATRX | BTF3 |
| TOR1AIP2 | RPS29 | YWHAQ | PLOD2 | MKRN1 | SH3BGRL | VCAN |
| QSOX1 | RPL36AL | ODC1 | TSC22D2 | ATG9B | MORF4L2 | PTGS1 |
| GLUL | NIN | PDIA6 | SERP1 | INSIG1 | ACSL4 | RPL35 |
| LAMC1 | KTN1 | LPIN1 | SIAH2 | CTSB | DOCK11 | RPL12 |
| IVNS1ABP | SYNE2 | OST4 | MBNL1 | PCM1 | PGRMC1 | SET |
| TPR | ACTN1 | LBH | SEC62 | ASAH1 | RPL39 | AIF1L |
| RGS18 | KIAA0247 | BIRC6 | TBLIXR1 | BNIP3L | LAMP2 | SETX |
| NUCKS1 | TTC9 | LTBP1 | AP2M1 | DPYSL2 | STAG2 | RPL7A |
| C1orf116 | MAP3K9 | EML4 | PSMD2 | CLU | MBNL3 | GPSM1 |
| CD55 | PCNX | CALM2 | EIF4A2 | TMEM66 | FLNA | PLCXD1 |
| CAPN2 | TMED10 | PSME4 | LPP | PCMTD1 | RPL10 | SLC25A6 |
| ARF1 | TC2N | SPTBN1 | ATP13A3 | RPS20 | MPP1 | TMSB4X |
| GUK1 | PAPOLA | RPS27A | MUC4 | SDCBP | GLUD1 | RPS24 |
| TOMM20 | PPP2R5C | SMEK2 | TFRC | ASPH | PTEN | GHITM |
| LYST | DYNC1H1 | USP34 | FYTTD1 | TRAM1 | PCGF5 | FAM25A |
| HNRNPU | EIF5 | XPO1 | RPL35A | RPL7 | TM9SF3 | MORF4L1 |
| TRIM58 | THBS1 | ACTR2 | MRFAP1 | CA2 | GSTO1 | BCL2A1 |
| PFKP | CHAC1 | ANXA4 | WDR1 | UQCRB | SHOC2 | RPS15A |
| NET1 | SNAP23 | MXD1 | RPL9 | RPL30 | ACSL5 | C2orf88 |
| GDI2 | PDIA3 | ZNF638 | PDS5A | EIF3E | ABLIM1 | GLS |
| ATP5C1 | SERF2 | MOB1A | FRYL | EIF3H | RPLP2 | MYO1B |
| OPTN | B2M | MTHFD2 | TMEM165 | FAM83A | CTSD | RPS3A |
| FAM107B | DUOX2 | TMSB10 | IGFBP7 | FBXO32 | HBB | MFAP3L |
| RSU1 | DUOXA2 | TGOLN2 | TMPRSS11B | PSCA | KIAA1199 | HPGD |
| VIM | COPS2 | MAL | MOB1B | EEF1D | RHCG | AKNA |
| PIP4K2A | ARPP19 | RPL31 | PF4 | PLIN2 | CIB1 | ATP6V1G1 |
| YME1L1 | CGNL1 | LIMS1 | PPBP | RPS6 | IQGAP1 | STOM |
| ZEB1 | ANXA2 | RANBP2 | CCNG2 | VCP | HBA2 | XRCC5 |
| ITGB1 | VPS13C | SOWAHC | HNRPDL | RMRP | HBA1 | RPL37A |
| HNRNPF | TPM1 | ACTR3 | SNCA | FAM108B1 | RPS2 | TNS1 |
| NCOA4 | RAB8B | GYPC | NFKB1 | VPS13A | PRSS22 | VCL |
| SRGN | HERC1 | CXCR4 | UBE2D3 | HNRNPK | PPL | PKM |
| PPA1 | PPIB | ZEB2 | RPL34 | ISCA1 | SNN | NCKAP1 |
| PSAP | DENND4A | SP3 | PDE5A | CTSL1 | GSPT1 | SMARCA5 |
| P4HA1 | RPL4 | HNRNPA3 | ANXA5 | HEMGN | FAM126B | PTBP3 |
| SYNPO2L | RPLP1 | NFE2L2 | KIAA1109 | RAD23B | TRAK2 | EEF1B2 |

Additional gene targets that is used as biomarkers for use with a fluid sample from the vaginal cavity of a subject are provided in Table 3.

TABLE 3
Additional gene targets
ADDITIONAL GENE TARGETS

| | | | | | | | |
|---|---|---|---|---|---|---|---|
| VEGFA | ANGPTL6 | CLEC12A | ELFN2 | IFNA16 | LOXL1 | PLXNB3 | SERPINI1 |
| VEGFB | ANGPTL7 | CLEC12B | EMCN | IFNA17 | LOXL2 | PLXNC1 | SERPINI2 |
| TGFB1 | ANXA1 | CLEC14A | EPGN | IFNA2 | LOXL3 | PLXND1 | SFRP1 |
| EGF | ANXA10 | CLEC17A | EPO | IFNA21 | LOXL4 | PPBP | SFRP2 |
| PTGES2 | ANXA11 | CLEC18A | EREG | IFNA4 | LPA | PRL | SFRP4 |
| PTGFR | ANXA13 | CLEC18B | F10 | IFNA5 | LTA | PROL1 | SFRP5 |
| MMP1 | ANXA2 | CLEC18C | F12 | IFNA6 | LTB | PRSS1 | SFTA2 |
| MMP2 | ANXA3 | CLEC19A | F13A1 | IFNA7 | MASP1 | PRSS12 | SFTA3 |
| MMP3 | ANXA4 | CLEC1A | F13B | IFNA8 | MASP2 | PRSS2 | SFTPA1 |
| ILF3 | ANXA5 | CLEC1B | F2 | IFNB1 | MBL2 | PRSS3 | SFTPA2 |
| MMP24 | ANXA6 | CLEC2A | F7 | IFNE | MDK | PSPN | SFTPB |
| MMP25 | ANXA7 | CLEC2B | F9 | IFNG | MEGF10 | PTN | SFTPC |
| MMP7 | ANXA8 | CLEC2D | FAM20A | IFNK | MEGF11 | PZP | SFTPD |
| MMP8 | ANXA8L1 | CLEC2L | FAM20B | IFNW1 | MEGF6 | REG1A | SHH |
| MMP9 | ANXA9 | CLEC3A | FAM20C | IGF1 | MEGF8 | REG1B | SLPI |
| MMP10 | AREG | CLEC3B | FASLG | IGF2 | MEGF9 | REG3A | SPAM1 |
| MMP11 | ARTN | CLEC4A | FCN1 | IHH | MEP1A | REG3G | ST14 |
| MMP12 | ASTL | CLEC4C | FCN2 | IL10 | MEP1B | REG4 | SULF1 |
| MMP19 | BDNF | CLEC4D | FCN3 | IL11 | MMP13 | RPTN | SULF2 |
| MMP26 | BMP1 | CLEC4E | FGF1 | IL12A | MMP14 | S100A1 | TCHH |
| HIF1A | BMP10 | CLEC4F | FGF10 | IL12B | MMP15 | S100A10 | TCHHL1 |
| FGF2 | BMP15 | CLEC4G | FGF11 | IL13 | MMP16 | S100A11 | TDGF1 |
| SERPINE1 | BMP2 | CLEC4M | FGF12 | IL15 | MMP17 | S100A12 | TGFA |
| PLAU | BMP3 | CLEC5A | FGF13 | IL16 | MMP20 | S100A13 | TGFB2 |
| COL1A1 | BMP4 | CLEC6A | FGF14 | IL17A | MMP21 | S100A14 | TGFB3 |
| COL2A1 | BMP5 | CLEC7A | FGF16 | IL17B | MMP23B | S100A16 | TGM1 |
| COL1A2 | BMP6 | CLEC9A | FGF17 | IL17C | MMP27 | S100A2 | TGM2 |
| COL3A1 | BMP7 | CNTF | FGF18 | IL17D | MMP28 | S100 A3 | TGM3 |
| COL6A1 | BMP8A | COLEC10 | FGF19 | IL17F | MST1 | S100A4 | TGM4 |
| COL4A1 | BMP8B | COLEC11 | FGF20 | IL18 | MST1L | S100A5 | TGM5 |
| COL4A2 | BRINP2 | COLEC12 | FGF21 | IL19 | MSTN | S100A6 | TGM6 |
| COL17A | BRINP3 | CPAMD8 | FGF22 | IL1A | MUC1 | S100A7 | TGM7 |
| COL6A2 | BTC | CPN2 | FGF23 | IL1B | MUC12 | S100A7A | THPO |
| LAMA1 | C17orf58 | CRHBP | FGF3 | IL1F10 | MUC13 | S100A7L2 | TIMP1 |
| LAMB1 | C1QA | CRLF1 | FGF4 | IL1RN | MUC15 | S100A8 | TIMP2 |
| LAMC1 | C1QB | CRLF3 | FGF5 | IL2 | MUC16 | S100A9 | TIMP3 |
| LAMA4 | C1QC | CRNN | FGF6 | IL20 | MUC17 | S100B | TIMP4 |
| FN1 | C1QL1 | CSF1 | FGF7 | IL22 | MUC19 | S100G | TLL1 |
| HSPG2 | C1QL2 | CSF2 | FGF8 | IL23A | MUC2 | S100P | TLL2 |
| ANG | C1QL3 | CSF3 | FGF9 | IL24 | MUC20 | S100Z | TMPRSS15 |
| SCGB1A1 | C1QL4 | CSH1 | FGFBP1 | IL25 | MUC21 | SCUBE1 | TNF |
| A2M | C1QTNF1 | CSH2 | FGFBP2 | IL26 | MUC22 | SCUBE2 | TNFSF10 |
| A2ML1 | C1QTNF2 | CSHL1 | FGFBP3 | IL3 | MUC3A | SCUBE3 | TNFSF11 |
| ADAM10 | C1QTNF3 | CSPG4 | FIGF | IL34 | MUC4 | SDC1 | TNFSF12 |
| ADAM11 | C1QTNF4 | CSPG5 | FLG | IL36A | MUC5AC | SDC2 | TNFSF13 |
| ADAM12 | C1QTNF5 | CST1 | FLG2 | IL36B | MUC5B | SDC3 | TNFSF13B |
| ADAM15 | C1QTNF6 | CST11 | FLT3LG | IL36G | MUC6 | SDC4 | TNFSF14 |
| ADAM17 | C1QTNF7 | CST2 | FREM1 | IL36RN | MUC7 | SEMA3A | TNFSF15 |
| ADAM18 | C1QTNF8 | CST3 | FREM2 | IL37 | MUC8 | SEMA3B | TNFSF18 |
| ADAM19 | C1QTNF9 | CST4 | FREM3 | IL4 | MUCL1 | SEMA3C | TNFSF4 |
| ADAM2 | C1QTNF9B | CST5 | FRZB | IL5 | NGF | SEMA3D | TNFSF8 |
| ADAM20 | CBLN1 | CST6 | FST | IL6 | NGLY1 | SEMA3E | TNFSF9 |
| ADAM21 | CBLN2 | CST7 | FSTL1 | IL7 | NODAL | SEMA3F | TPO |
| ADAM22 | CBLN3 | CST8 | FSTL3 | IL9 | NRG1 | SEMA3G | VEGFC |
| ADAM23 | CBLN4 | CST9 | GDF1 | INHA | NRG2 | SEMA4A | VWC2 |
| ADAM28 | CCBE1 | CST9L | GDF10 | INHBA | NRG3 | SEMA4B | VWC2L |
| ADAM29 | CCL1 | CSTA | GDF11 | INHBB | NRG4 | SEMA4C | WFIKKN1 |
| ADAM30 | CCL11 | CSTB | GDF15 | INHBC | NRTN | SEMA4D | WFIKKN2 |
| ADAM32 | CCL13 | CSTL1 | GDF2 | INHBE | NTF3 | SEMA4F | WIF1 |
| ADAM33 | CCL14 | CTF1 | GDF3 | INS | NTF4 | SEMA4G | WNT1 |
| ADAM7 | CCL15 | CTSA | GDF5 | INS-IGF2 | OGFOD1 | SEMA5A | WNT10A |
| ADAM8 | CCL16 | CTSB | GDF6 | INSL3 | OGFOD2 | SEMA5B | WNT10B |
| ADAM9 | CCL17 | CTSC | GDF7 | INSL5 | OSM | SEMA6A | WNT11 |
| ADAMDEC1 | CCL18 | CTSD | GDF9 | INSL6 | OVGP1 | SEMA6B | WNT16 |
| ADAMTS1 | CCL19 | CTSE | GDNF | ISM1 | P3H1 | SEMA6C | WNT2 |
| ADAMTS10 | CCL2 | CTSF | GH1 | ISM2 | P3H2 | SEMA6D | WNT2B |
| ADAMTS12 | CCL20 | CTSG | GH2 | ITIH1 | P3H3 | SEMA7A | WNT3 |
| ADAMTS13 | CCL21 | CTSH | GPC1 | ITIH2 | P4HA1 | SERPINA1 | WNT3A |
| ADAMTS14 | CCL22 | CTSK | GPC2 | ITIH3 | P4HA2 | SERPINA10 | WNT4 |
| ADAMTS15 | CCL23 | CTSL | GPC3 | ITIH4 | P4HA3 | SERPINA11 | WNT5A |
| ADAMTS16 | CCL24 | CTSO | GPC4 | ITIH5 | P4HTM | SERPINA12 | WNT5B |
| ADAMTS17 | CCL25 | CTSS | GPC5 | ITIH6 | PAMR1 | SERPINA2 | WNT6 |
| ADAMTS18 | CCL26 | CTSV | GPC6 | ITLN1 | PAPPA | SERPINA3 | WNT7A |
| ADAMTS19 | CCL27 | CTSW | GREM1 | ITLN2 | PAPPA2 | SERPINA4 | WNT7B |
| ADAMTS2 | CCL28 | CTSZ | GRIFIN | KAZALD1 | PARM1 | SERPINA5 | WNT8A |
| ADAMTS20 | CCL3 | CX3CL1 | HABP2 | KITLG | PCSK5 | SERPINA6 | WNT8B |
| ADAMTS3 | CCL3L3 | CXCL1 | HBEGF | KNG1 | PCSK6 | SERPINA7 | WNT9A |
| ADAMTS4 | CCL4 | CXCL10 | HCFC1 | KY | PDGFA | SERPINA9 | WNT9B |
| ADAMTS5 | CCL4L1 | CXCL11 | HCFC2 | LEFTY1 | PDGFB | SERPINB1 | XCL1 |
| ADAMTS6 | CCL4L2 | CXCL12 | HGF | LEFTY2 | PDGFC | SERPINB10 | XCL2 |
| ADAMTS7 | CCL5 | CXCL13 | HGFAC | LEP | PDGFD | SERPINB11 | ZFP91 |
| ADAMTS8 | CCL7 | CXCL14 | HHIP | LGALS1 | PF4 | SERPINB12 | |
| ADAMTS9 | CCL8 | CXCL2 | HMSD | LGALS12 | PF4V1 | SERPINB13 | |
| ADAMTSL1 | CD109 | CXCL3 | HPSE | LGALS13 | PGF | SERPINB2 | |
| ADAMTSL2 | CD209 | CXCL5 | HPSE2 | LGALS14 | PI3 | SERPINB3 | |
| ADAMTSL3 | CELA1 | CXCL6 | HPX | LGALS16 | PIK3IP1 | SERPINB4 | |
| ADAMTSL4 | CELA2A | CXCL8 | HRG | LGALS2 | PLAT | SERPINB5 | |
| ADAMTSL5 | CELA2B | CXCL9 | HRNR | LGALS3 | PLG | SERPINB6 | |
| AGT | CELA3A | DHH | HTRA1 | LGALS4 | PLOD1 | SERPINB7 | |
| AMBP | CELA3B | EBI3 | HTRA3 | LGALS7 | PLOD2 | SERPINB8 | |
| AMH | CFC1 | EDA | HTRA4 | LGALS8 | PLOD3 | SERPINB9 | |
| ANGPT1 | CFC1B | EGFL6 | HYAL1 | LGALS9 | PLXDC1 | SERPINC1 | |
| ANGPT2 | CHRD | EGFL7 | HYAL2 | LGALS9B | PLXDC2 | SERPIND1 | |
| ANGPT4 | CHRDL1 | EGFL8 | HYAL3 | LGALS9C | PLXNA1 | SERPINE2 | |
| ANGPTL1 | CHRDL2 | EGLN1 | HYAL4 | LGALSL | PLXNA2 | SERPINE3 | |
| ANGPTL2 | CLC | EGLN2 | IFNA1 | LIF | PLXNA3 | SERPINF1 | |
| ANGPTL3 | CLCF1 | EGLN3 | IFNA10 | LMAN1 | PLXNA4 | SERPINF2 | |
| ANGPTL4 | CLEC10A | ELANE | IFNA13 | LMAN1L | PLXNB1 | SERPING1 | |
| ANGPTL5 | CLEC11A | ELFN1 | IFNA14 | LOX | PLXNB2 | SERPINH1 | |

miRNA target

| | | | | | | |
|---|---|---|---|---|---|---|
| hsa-let-7a-5p | hsa-miR-563 | hsa-miR-519a-5p | hsa-miR-4269 | hsa-miR-4663 | hsa-miR-513c-3p | hsa-miR-6801-3p |
| hsa-let-7b-5p | hsa-miR-566 | hsa-miR-519b-5p | hsa-miR-4277 | hsa-miR-4666a-5p | hsa-miR-548t-3p | hsa-miR-6803-3p |
| hsa-let-7c-5p | hsa-miR-569 | hsa-miR-520c-5p | hsa-miR-4279 | hsa-miR-4680-5p | hsa-miR-548x-5p | hsa-miR-6805-3p |
| hsa-let-7e-5p | hsa-miR-573 | hsa-miR-518d-5p | hsa-miR-4283 | hsa-miR-4680-3p | hsa-miR-548o-5p | hsa-miR-6810-5p |
| hsa-let-7f-5p | hsa-miR-579-3p | hsa-miR-1224-3p | hsa-miR-4284 | hsa-miR-4685-3p | hsa-miR-548aj-5p | hsa-miR-6811-5p |
| hsa-miR-17-3p | hsa-miR-584-5p | hsa-miR-1225-3p | hsa-miR-4329 | hsa-miR-4686 | hsa-miR-548am-5p | hsa-miR-6814-5p |
| hsa-miR-20a-5p | hsa-miR-586 | hsa-miR-1228-5p | hsa-miR-3605-3p | hsa-miR-4687-5p | hsa-miR-3529-3p | hsa-miR-6817-5p |
| hsa-miR-21-5p | hsa-miR-600 | hsa-miR-1229-3p | hsa-miR-3613-5p | hsa-miR-4690-5p | hsa-miR-365b-3p | hsa-miR-6819-5p |
| hsa-miR-24-3p | hsa-miR-609 | hsa-miR-1231 | hsa-miR-3613-3p | hsa-miR-4703-3p | hsa-miR-1227-5p | hsa-miR-6823-5p |
| hsa-miR-26a-5p | hsa-miR-612 | hsa-miR-1264 | hsa-miR-3620-3p | hsa-miR-4704-3p | hsa-miR-1229-5p | hsa-miR-6828-5p |
| hsa-miR-26b-5p | hsa-miR-622 | hsa-miR-320b | hsa-miR-3622b-3p | hsa-miR-4710 | hsa-miR-1236-5p | hsa-miR-6780b-5p |
| hsa-miR-29a-3p | hsa-miR-626 | hsa-miR-320c | hsa-miR-3651 | hsa-miR-4721 | hsa-miR-1238-5p | hsa-miR-6839-5p |
| hsa-miR-32-5p | hsa-miR-628-3p | hsa-miR-1323 | hsa-miR-3656 | hsa-miR-4723-5p | hsa-miR-3620-5p | hsa-miR-6839-3p |
| hsa-miR-93-5p | hsa-miR-630 | hsa-miR-1301-3p | hsa-miR-3657 | hsa-miR-4725-3p | hsa-miR-4743-3p | hsa-miR-6853-3p |
| hsa-miR-98-5p | hsa-miR-631 | hsa-miR-1179 | hsa-miR-3658 | hsa-miR-4731-3p | hsa-miR-6068 | hsa-miR-6858-5p |
| hsa-miR-29b-3p | hsa-miR-642a-5p | hsa-miR-1182 | hsa-miR-3663-3p | hsa-miR-4735-5p | hsa-miR-6071 | hsa-miR-6866-3p |
| hsa-miR-199a-5p | hsa-miR-646 | hsa-miR-1200 | hsa-miR-3670 | hsa-miR-3591-3p | hsa-miR-6080 | hsa-miR-6872-3p |
| hsa-miR-181a-5p | hsa-miR-647 | hsa-miR-1207-5p | hsa-miR-3679-5p | hsa-miR-4745-5p | hsa-miR-6083 | hsa-miR-6874-5p |
| hsa-miR-181c-5p | hsa-miR-648 | hsa-miR-548j-5p | hsa-miR-3679-3p | hsa-miR-4746-3p | hsa-miR-6084 | hsa-miR-6876-3p |
| hsa-miR-199b-5p | hsa-miR-548d-3p | hsa-miR-1297 | hsa-miR-3682-3p | hsa-miR-4747-5p | hsa-miR-6089 | hsa-miR-6879-5p |
| hsa-miR-214-3p | hsa-miR-653-5p | hsa-miR-1244 | hsa-miR-3684 | hsa-miR-4748 | hsa-miR-6124 | hsa-miR-6879-3p |
| hsa-miR-216a-5p | hsa-miR-656-3p | hsa-miR-1248 | hsa-miR-3909 | hsa-miR-499b-3p | hsa-miR-6128 | hsa-miR-6882-5p |
| hsa-let-7g-5p | hsa-miR-549a | hsa-miR-548n | hsa-miR-3924 | hsa-miR-4758-5p | hsa-miR-6132 | hsa-miR-6884-5p |
| hsa-let-7i-5p | hsa-miR-660-5p | hsa-miR-1268a | hsa-miR-3935 | hsa-miR-4760-5p | hsa-miR-6133 | hsa-miR-6884-3p |
| hsa-miR-1 | hsa-miR-363-5p | hsa-miR-548i | hsa-miR-3938 | hsa-miR-4762-5p | hsa-miR-6134 | hsa-miR-6886-3p |
| hsa-miR-138-5p | hsa-miR-767-5p | hsa-miR-1252-5p | hsa-miR-374c-5p | hsa-miR-4763-5p | hsa-miR-548ay-5p | hsa-miR-6894-5p |
| hsa-miR-140-5p | hsa-miR-769-3p | hsa-miR-664a-3p | hsa-miR-548aa | hsa-miR-4764-3p | hsa-miR-6505-5p | hsa-miR-6895-5p |
| hsa-miR-141-3p | hsa-miR-802 | hsa-miR-1197 | hsa-miR-1268b | hsa-miR-4769-5p | hsa-miR-6512-3p | hsa-miR-6895-3p |
| hsa-miR-143-3p | hsa-let-7a-3p | hsa-miR-320d | hsa-miR-548ab | hsa-miR-4769-3p | hsa-miR-6515-5p | hsa-miR-7113-3p |
| hsa-miR-145-5p | hsa-let-7f-1-3p | hsa-miR-1908-5p | hsa-miR-4419a | hsa-miR-4774-3p | hsa-miR-6715b-3p | hsa-miR-7114-5p |
| hsa-miR-152-3p | hsa-miR-27a-5p | hsa-miR-1910-5p | hsa-miR-4425 | hsa-miR-4775 | hsa-miR-6721-5p | hsa-miR-7153-3p |
| hsa-miR-9-5p | hsa-miR-92a-1-5p | hsa-miR-2113 | hsa-miR-4429 | hsa-miR-4778-3p | hsa-miR-6722-3p | hsa-miR-7156-3p |
| hsa-miR-206 | hsa-miR-92a-2-5p | hsa-miR-759 | hsa-miR-4430 | hsa-miR-4436b-3p | hsa-miR-892c-5p | hsa-miR-7157-3p |
| hsa-miR-320a | hsa-miR-29b-2-5p | hsa-miR-2114-5p | hsa-miR-4434 | hsa-miR-4789-3p | hsa-miR-134-3p | hsa-miR-7159-5p |
| hsa-miR-29c-3p | hsa-miR-105-3p | hsa-miR-2115-3p | hsa-miR-4435 | hsa-miR-4800-3p | hsa-miR-383-3p | hsa-miR-7159-3p |
| hsa-miR-200a-3p | hsa-miR-16-2-3p | hsa-miR-2276-3p | hsa-miR-4436a | hsa-miR-5047 | hsa-miR-597-3p | hsa-miR-7160-5p |
| hsa-miR-365a-3p | hsa-miR-214-5p | hsa-miR-2278 | hsa-miR-4437 | hsa-miR-5001-3p | hsa-miR-605-3p | hsa-miR-7161-3p |
| hsa-miR-302c-5p | hsa-miR-23b-5p | hsa-miR-711 | hsa-miR-4445-5p | hsa-miR-5002-5p | hsa-miR-653-3p | hsa-miR-7845-5p |
| hsa-miR-369-3p | hsa-miR-135a-3p | hsa-miR-2681-5p | hsa-miR-4448 | hsa-miR-5004-5p | hsa-miR-548f-5p | hsa-miR-7849-3p |
| hsa-miR-374a-5p | hsa-miR-127-5p | hsa-miR-3115 | hsa-miR-4450 | hsa-miR-548ap-5p | hsa-miR-1537-5p | hsa-miR-7855-5p |
| hsa-miR-377-3p | hsa-miR-149-3p | hsa-miR-3120-3p | hsa-miR-4452 | hsa-miR-548ap-3p | hsa-miR-2276-5p | hsa-miR-8055 |
| hsa-miR-379-5p | hsa-miR-150-3p | hsa-miR-3126-5p | hsa-miR-4456 | hsa-miR-5008-5p | hsa-miR-500b-3p | hsa-miR-8057 |
| hsa-miR-330-3p | hsa-miR-188-3p | hsa-miR-3127-5p | hsa-miR-3155b | hsa-miR-5009-3p | hsa-miR-6720-5p | hsa-miR-8068 |
| hsa-miR-324-5p | hsa-miR-195-3p | hsa-miR-3132 | hsa-miR-4483 | hsa-miR-5094 | hsa-miR-6730-3p | hsa-miR-8070 |
| hsa-miR-339-5p | hsa-miR-194-3p | hsa-miR-466 | hsa-miR-4491 | hsa-miR-5190 | hsa-miR-6736-3p | hsa-miR-8072 |
| hsa-miR-433-3p | hsa-miR-296-3p | hsa-miR-544b | hsa-miR-4493 | hsa-miR-5196-5p | hsa-miR-6737-5p | hsa-miR-8082 |
| hsa-miR-452-5p | hsa-miR-130b-5p | hsa-miR-3140-3p | hsa-miR-4498 | hsa-miR-5572 | hsa-miR-6737-3p | hsa-miR-8089 |
| hsa-miR-409-3p | hsa-miR-335-3p | hsa-miR-3141 | hsa-miR-4419b | hsa-miR-548ar-5p | hsa-miR-6738-3p | hsa-miR-1199-5p |
| hsa-miR-410-3p | hsa-miR-146b-3p | hsa-miR-3143 | hsa-miR-4500 | hsa-miR-548as-5p | hsa-miR-6743 -5p | hsa-miR-7973 |
| hsa-miR-485-5p | hsa-miR-505-5p | hsa-miR-3145-3p | hsa-miR-4509 | hsa-miR-5579-3p | hsa-miR-6744-5p | |
| hsa-miR-491-5p | hsa-miR-513a-3p | hsa-miR-3148 | hsa-miR-1587 | hsa-miR-664b-3p | hsa-miR-6750-5p | |
| hsa-miR-492 | hsa-miR-574-5p | hsa-miR-3155a | hsa-miR-3120-5p | hsa-miR-5580-3p | hsa-miR-6752-5p | |
| hsa-miR-496 | hsa-miR-548b-5p | hsa-miR-3157-5p | hsa-miR-3156-3p | hsa-miR-5581-5p | hsa-miR-6753-5p | |
| hsa-miR-515-5p | hsa-miR-548a-5p | hsa-miR-3158-3p | hsa-miR-3173-5p | hsa-miR-548at-5p | hsa-miR-6755-3p | |
| hsa-miR-519e-5p | hsa-miR-548c-5p | hsa-miR-3163 | hsa-miR-3682-5p | hsa-miR-5582-3p | hsa-miR-6760-5p | |
| hsa-miR-519c-5p | hsa-miR-625-3p | hsa-miR-1193 | hsa-miR-3913-3p | hsa-miR-5584-5p | hsa-miR-6764-5p | |
| hsa-miR-520a-5p | hsa-miR-548d-5p | hsa-miR-3179 | hsa-miR-3925-3p | hsa-miR-5584-3p | hsa-miR-6766-5p | |
| hsa-miR-525-5p | hsa-miR-654-3p | hsa-miR-320e | hsa-miR-3942-3p | hsa-miR-5585-3p | hsa-miR-6768-5p | |
| hsa-miR-518f-5p | hsa-miR-874-3p | hsa-miR-4296 | hsa-miR-3960 | hsa-miR-5586-3p | hsa-miR-6769a-3p | |
| hsa-miR-526a | hsa-miR-891b | hsa-miR-4298 | hsa-miR-3972 | hsa-miR-548au-5p | hsa-miR-6773-5p | |
| hsa-miR-518c-3p | hsa-miR-875-3p | hsa-miR-4300 | hsa-miR-3973 | hsa-miR-5591-3p | hsa-miR-6776-5p | |
| hsa-miR-520g-3p | hsa-miR-744-5p | hsa-miR-4302 | hsa-miR-3977 | hsa-miR-5682 | hsa-miR-6777-3p | |
| hsa-miR-518d-3p | hsa-miR-885-3p | hsa-miR-4311 | hsa-miR-4643 | hsa-miR-548aw | hsa-miR-6782-5p | |
| hsa-miR-520h | hsa-miR-877-5p | hsa-miR-4323 | hsa-miR-4648 | hsa-miR-5703 | hsa-miR-6783-5p | |
| hsa-miR-499a-5p | hsa-miR-924 | hsa-miR-4259 | hsa-miR-4651 | hsa-miR-211-3p | hsa-miR-6784-5p | |
| hsa-miR-504-5p | hsa-miR-944 | hsa-miR-4251 | hsa-miR-4653-5p | hsa-miR-514a-5p | hsa-miR-6790-5p | |
| hsa-miR-455-5p | hsa-miR-523-5p | hsa-miR-4326 | hsa-miR-4653-3p | hsa-miR-548g-5p | hsa-miR-6792-5p | |
| hsa-miR-539-5p | hsa-miR-518e-5p | hsa-miR-4262 | hsa-miR-4657 | hsa-miR-1277-5p | hsa-miR-6798-3p | |
| hsa-miR-561-3p | hsa-miR-522-5p | hsa-miR-4268 | hsa-miR-4658 | hsa-miR-1306-5p | hsa-miR-6801-5p | |

### Other Properties of Fluid Samples

In some embodiments of method herein, other properties or contents of samples from a vaginal cavity, such as menstrual fluid, is measured. In some embodiments, the properties or contents include a small molecule, a microbiome, a pathogen, a metabolic marker or metabolome, an epigenetic marker or modification or epigenome, or a hormone.

In some embodiments, a fluid sample from the vaginal cavity of a subject, such as menstrual fluid, comprises a small molecule or chemical compound. In some embodiments, the small molecule differs in content, amount, or concentration in menstrual fluid from a subject experiencing HMB or AUB compared with a sample of menstrual fluid from a subject experiencing normal menstrual bleeding.

In some embodiments, the small molecule in such a sample is a molecule having a low molecular weight. In some embodiments, the small molecule has a molecular weight less than 1000 Da, less than 900 Da, less than 800 Da, less than 700 Da, less than 600 Da, less than 500 Da, less than 400 Da, less than 300 Da, less than 200 Da, or less than 100 Da. In some embodiments, the small molecule has a size less than 100 nm, less than 50 nm, less than 10 nm, or less than 1 nm.

In some embodiments, a small molecule is a drug, a supplement, a metabolite, or a monomer (e.g., a ribonucleotide, a deoxyribonucleotide, an amino acid, or a monosaccharide). In some embodiments, a small molecule is an organic molecule or an inorganic molecule. In some embodiments, a small molecule is able to regulate a biological process.

In some embodiments, a small molecule is a secondary metabolite. In some embodiments, a secondary metabolite is a natural metabolite or product. In some embodiments, a secondary metabolite is produced by a microorganism, such as a bacterium or fungus, such as a microorganism in a microbiome of the subject or a pathogen of the subject. In some embodiments, secondary metabolites includes alkaloids, glycosides, lipids, nonribosomal peptides (e.g., actinomycin-D), phenazines, natural phenols (e.g., flavonoids), polyketide, terpenes (e.g., steroids), and tetrapyrroles. In some embodiments, a microorganism responsible for producing such a secondary metabolite is in the sample, in the vaginal cavity of the subject, or elsewhere in or on the subject.

In some embodiments, a small molecule of a fluid sample from a vaginal cavity of a subject differs from another fluid sample from a vaginal cavity of another subject. In some embodiments, a small molecule of a fluid sample of a vaginal cavity of a subject experiencing HMB or AUB differed from a small molecule of that of a subject experiencing normal menstrual bleeding. In some embodiments, a small molecule of a fluid sample from a vaginal cavity of a subject experiencing HMB or AUB has a higher or lower concentration in a fluid sample from the vaginal cavity of the subject than in that of a subject experiencing normal menstrual bleeding. In some embodiments, a fluid sample from a vaginal cavity of a subject experiencing HMB or AUB comprises one or more small molecules which are not present in that of a subject experiencing normal menstrual bleeding. In some embodiments, a fluid sample from a vaginal cavity of a subject experiencing HMB or AUB lacks a small molecule which is found in that of a subject experiencing normal menstrual bleeding. In some embodiments, a fluid sample from the vaginal cavity of a subject experiencing HMB or AUB comprises a different small molecule composition, such as a different ratio of small molecules or a different set of small molecules, than that of a subject experiencing normal menstrual bleeding.

In some embodiments, a small molecule is detected using any acceptable method, including mass spectrometry (MS), gas chromatography (GC), liquid chromatography (LC), high performance liquid chromatography (HPLC), HPLC-MS, HPLC-MS/MS, GC-MS, GC-MS/MS, LC-MS, LC-MS/MS, or by an assay such as ELISA or an enzymatic assay.

In some embodiments, a microbiome detectable in a fluid sample from the vaginal cavity of a subject, such as menstrual fluid, comprises an ecological community of microorganisms, which comprises commensal, symbiotic, pathogenic microorganisms, or a combination thereof. In some embodiments, a microbiome comprises bacteria, fungi (e.g., yeast), viruses, archaea, protists, or a combination thereof. In some embodiments, a microbiome comprises members of an endometrial microbiome, uterine microbiome, or a vagino-cervical microbiome of a subject.

In some embodiments, a microbiome detectable in a fluid sample from the vaginal cavity of a subject, such as a menstrual fluid, is pathogen free or comprises one or more pathogens. In some embodiments, pathogens comprises microorganisms such as bacteria, fungi (e.g., yeast), viruses, archaea, protists, or a combination thereof. In some embodiments, a fluid sample has one type of pathogen or more than one type of pathogen. In some embodiments, a fluid sample has 2, 3, 4, 5, or more types of pathogens. In some embodiments, a pathogen found in a sample of a subject experiencing HMB or AUB is absent in a sample from a subject experiencing normal menstrual bleeding.

In some embodiments, a pathogen in a fluid sample from the vaginal cavity of a subject is indicative of HMB, AUB, a menstrual cycle disorder, or another disorder. In some embodiments, a pathogen in a fluid sample from the vaginal cavity of a subject is causative of an infection, which is causative of a disease or condition. In some embodiments, such a pathogen causes HMB, AUB, a menstrual cycle disorder, or another disorder. In some embodiments, a pathogen causes a disease associated with HMB, AUB, a menstrual cycle disorder, or another disorder. In some embodiments, such a pathogen alters the symptoms of HMB, AUB, a menstrual cycle disorder or another disorder in a subject. In some embodiments, a pathogen in a fluid sample from the vaginal cavity of a subject is unrelated to HMB, AUB, a menstrual cycle disorder, or another disorder of the subject.

A pathogen is disruptive of a microbiome of a subject. In some embodiments, a pathogen reduces the quantity or variety of one or more microorganisms present in the microbiome detectable in a fluid sample from the vaginal cavity of a subject, such as menstrual fluid. In some embodiments, a pathogen causes such a reduction in microorganism population for example by out-competing one or more microorganisms of such a microbiome.

In some embodiments, a microbiome of a subject, such as an endometrial microbiome, uterine microbiome, or a vagino-cervical microbiome, which is detectable in a menstrual fluid sample, provides a potential source of novel biomarkers for detection of HMB or AUB.

In some embodiments, a microbiome of a fluid sample from a vaginal cavity of a subject differs from another fluid sample from a vaginal cavity of another subject. In some embodiments, the microbiome of a fluid sample from a vaginal cavity of a subject experiencing HMB or AUB differs from a microbiome of that of a subject experiencing normal menstrual bleeding. In some embodiments, a microbiome of a fluid sample from a vaginal cavity of a subject experiencing HMB or AUB has a higher or lower total amount of one or more microbes than that of a subject experiencing normal menstrual bleeding. In some embodiments, a microbiome of a fluid sample from a vaginal cavity of a subject experiencing HMB or AUB comprises one or more microbes which are not present in that of a subject experiencing normal menstrual bleeding. In some embodiments, a microbiome of a fluid sample from a vaginal cavity of a subject experiencing HMB or AUB lacks a microbe which is found in that of a subject experiencing normal menstrual bleeding. In some embodiments, a microbiome of a fluid sample from a vaginal cavity of a subject experiencing HMB or AUB comprises a different composition, such as a different ratio of microbes or a different set of microbes, than that of a subject experiencing normal menstrual bleeding. In some embodiments, the difference between two or more microbiome, such as the difference between the microbiome of the a fluid sample from a vaginal cavity of a first subject and a second subject, wherein the second subject is experiencing normal menstrual fluid, is referred to as beta-diversity, and is described or indexed, for example, using unweighted or weighted UniFrac distance metrics or a Bray-Curtis dissimilarity.

In some embodiments, an alteration in the microbiome of a subject compared with that of a subject experiencing normal menstrual bleeding is indicative of HMB, AUB, a menstrual cycle disorder, or another disorder. In some embodiments, one or more microbes in a microbiome is measured, quantified, or detected in a sample of menstrual fluid by an acceptable method, which comprises a sequencing technique, such as 16s rRNA sequencing, followed by analysis. In some embodiments, a property of a microbiome is measured or detected, such as one or more protein biomarkers, gene target biomarkers, expressed genes, genotype of organisms, phenotype of organisms, diversity of organisms, number of organisms, or other property is measured from a menstrual fluid sample of a subject.

In some embodiments, a fluid sample from the vaginal cavity, such as menstrual fluid, comprises one or more metabolic markers, which differs in content, amount, or concentration in a fluid sample from a vaginal cavity of a subject experiencing HMB or AUB compared with a sample of menstrual fluid from a subject experiencing normal menstrual bleeding. Metabolic markers found in such a fluid sample comprises one or more drug metabolites, one or more endogenous metabolites, such as an amino acid, organic acid, nucleic acid, fatty acid, amine, sugar, vitamin, co-factor, pigment, antibiotic, other metabolites, or one or more exogenous metabolites, such as an environmental contaminant or xenobiotic.

In some embodiments, an amount or presence of a metabolite of a fluid sample from a vaginal cavity of a subject differs from another fluid sample from a vaginal cavity of another subject. In some embodiments, an amount or presence of a metabolite in a fluid sample from a vaginal cavity of a subject experiencing HMB or AUB differs from an amount or presence of the same metabolite in the menstrual fluid of a subject experiencing normal menstrual bleeding. In some embodiments, an amount or presence of a metabolite in a fluid sample from a vaginal cavity of a subject experiencing HMB or AUB is higher or lower in the menstrual fluid of a subject experiencing normal menstrual bleeding. In some embodiments, a fluid sample from a vaginal cavity of a subject experiencing HMB or AUB comprises one or more metabolites which are not present in that of a subject experiencing normal menstrual bleeding. In some embodiments, a fluid sample from a vaginal cavity of a subject experiencing HMB or AUB lacks a metabolite which is found in that of a subject experiencing normal menstrual bleeding. In some embodiments, the metabolic composition (e.g., metabolome) of a fluid sample from a vaginal cavity of a subject experiencing HMB or AUB comprises a different composition, such as a different ratio of metabolites or a different set of metabolites than that of a subject experiencing normal menstrual bleeding.

In some embodiments, metabolites is detected using any acceptable method, including MS, GC, LC, HPLC, HPLC-MS, HPLC-MS/MS, GC-MS, GC-MS/MS, LC-MS, LC-MS/MS, or by an assay such as ELISA or an enzymatic assay. In some embodiments, a metabolomic analysis is performed to detect a plurality of metabolites in a fluid sample from a vaginal cavity of a subject.

In some embodiments, one or more epigenetic modifications to nucleic acid of a fluid sample from a vaginal cavity of a subject differs from another fluid sample from a vaginal cavity of another subject. In some embodiments, one or more epigenetic modifications to nucleic acid in a fluid sample from a vaginal cavity of a subject experiencing HMB or AUB is altered compared with a fluid sample from a vaginal cavity of a subject from a subject experiencing normal menstrual bleeding. In some embodiments, an epigenetic modification not present in a sample from a subject experiencing normal menstrual bleeding is present in a sample from a subject experiencing HMB or AUB. In some embodiments, a modification which is present in a fluid sample from a vaginal cavity of a subject from a subject experiencing normal menstrual bleeding is not present in a sample from a subject experiencing HMB or AUB. Sometimes, a given epigenetic modification is present to a greater or lesser extent in a sample from a subject experiencing HMB or AUB than in a sample from a subject experiencing normal menstrual bleeding.

In some embodiments, epigenetic modifications to nucleic acid is measured in a fluid sample collected from a vaginal cavity of a subject such as a menstrual fluid sample. Epigenetic modifications referred to changes to a DNA strand, which are not changes to the DNA sequence. In some embodiments, epigenetic changes comprises methylation of the DNA as well as histone modification, acetylation, methylation, ubiquitylation, phosphorylation, sumoylation, ribosylation, or citrullination. In some embodiments, epigenetic modifications is of known gene targets. More than one type of epigenetic modification is present in a sample. In some embodiments, epigenetic modifications occur with other changes, such as changes in protein biomarkers, changes in gene target biomarkers, changes in gene expression, changes in nucleic acid content, changes in cell types present, and changes in flow rate of menstrual fluid.

In some embodiments, epigenetic modifications is detected using one or more acceptable methods. In some embodiments, for example when methylation has occurred, methylome sequencing is performed. Methylated DNA methylation sites is uncovered for example by bisulfite conversion followed by sequencing or microarray analysis, or by genome wide methylation quantification combined with HPLC-UV, LC-MS/MS, and/or ELISA. In some embodiments, the extent of methylation of nucleic acid in a sample, such as the extent of methylation of one or more known gene targets, is determined for example by bisulfite conversion followed by q-RT-PCR or PCR and sequencing, or by performing a DNA enzyme digest based on a target sequence followed by q-RT-PCR or PCR and sequencing.

In some embodiments, one or more hormones of a fluid sample from a vaginal cavity of a subject differs from another fluid sample from a vaginal cavity of another subject. In some embodiments, one or more hormones in a fluid sample from a vaginal cavity of a subject experiencing HMB or AUB is altered compared with a sample of menstrual fluid from a subject experiencing normal menstrual bleeding. In some embodiments, a hormone not present in a fluid sample from a vaginal cavity of a subject experiencing normal menstrual bleeding is present in a sample from a subject experiencing HMB or AUB. In some embodiments, a hormone which is present in a sample from a fluid sample from a vaginal cavity of a subject experiencing normal menstrual bleeding is absent in a sample from a subject experiencing HMB or AUB. In some embodiments, a hormone is present to a greater or lesser extent (e.g., higher concentration or lower concentration) in a sample from a subject experiencing HMB or AUB than in a sample from a subject experiencing normal menstrual bleeding.

In some embodiments, a hormone in a fluid sample from a vaginal cavity of a subject is estrogen, follicle stimulating hormone, progesterone, human chorionic gonadotropin (hCG), or luteinizing hormone (LH). In some embodiments, hormone levels vary throughout the menstrual cycle in menstrual fluid or non-menstrual fluid samples of subjects

In some embodiments, a hormone is measured using any acceptable method. In some embodiments, a hormone is measured using GC-MS, GC-MS/MS, LC-MS, LC-MS/MS, HPLC, HPLC-MS, Western blotting, ELISA, a dot blot, an immunoassay, or another method.

### Other Tests

In some embodiments, other tests are performed in parallel with analysis of the menstrual fluid. In some embodiments, parallel testing allows for a more complete analysis of the health or disease status of a subject in some cases. In some embodiments, analysis of another test in addition to analysis of a menstrual fluid sample or cervicovaginal fluid sample is used to optimize the collection system or analysis of the fluid sample. In some embodiments, another test is used to confirm or support the results of analysis on a fluid sample or the results of the analysis on a fluid sample is used to confirm or supplement the results of another test. In some embodiments, other tests comprise a blood test. In some embodiments, blood tests can include a glucose test, a measurement of the level of one or more hormones, genomic analysis, or sequencing analysis. In some embodiments, other tests comprise a test on a tissue sample. In some embodiments, tissue samples include a vaginal sample, an endometrial sample, a fallopian sample, an ovarian sample, an ovum sample, a cervical sample, a labial sample, a placenta sample, or a skin sample. In some embodiments, a test on a tissue sample comprises genomic analysis, sequencing analysis, or histological analysis.

### Flow Rate

In some cases of methods provided herein, menstrual blood is expelled from the body at a given flow rate. In some cases, subjects experiencing normal menstrual bleeding has a normal flow rate. During HMB or AUB, this flow rate is altered. In some embodiments, during HMB, flow rate is increased during a portion of up to the entire duration of the menstrual cycle. In some embodiments, some subjects experience a normal flow rate for a portion (e.g., a first portion) of the menstrual cycle and experience an increased flow rate during another portion (e.g., a second portion) of the menstrual cycle. In some embodiments, subjects has an increased flow rate for at least about 1 hour, at least about 2 hours, at least about 3 hours, at least about 4 hours, at least about 5 hours, at least about 6 hours, at least about 12 hours, at least about 18 hours, at least about 24 hours, at least about 2 days, at least about 3 days, at least about 4 days, at least about 5 days, at least about 6 days, at least about 7 days, or more.

In some methods, a flow rate of menstrual blood is measured. In some embodiments, a flow rate is measured as a normal flow rate, a high flow rate, or a low flow rate. In some embodiments, a normal flow rate is indicative of normal menstrual bleeding. In some embodiments, a high flow rate is indicative of HMB or AUB. In some embodiments, a low flow rate is indicative of AUB.

In some embodiments, a flow rate is compared with a pre-determined threshold flow rate to determine whether it is decreased or increased or normal compared with the threshold flow rate. In some embodiments, a predetermined threshold flow rate is determined from a reference sample, such as fluid collected from the vaginal cavity of a control subject, such as menstrual fluid collected from a subject experiencing normal menstrual bleeding, a subject experiencing HMB, or a subject experiencing AUB. In some embodiments, a threshold flow rate is a flow rate experienced by a subject during normal menstrual bleeding. In some embodiments, a threshold flow rate is an upper threshold, such as a maximum flow rate measured during normal menstrual bleeding. In some embodiments, a threshold flow rate is determined from measuring the flow rate of at least 1, 2, 3, 4, 5, 10, 15, 20, 30, 40, or 50 subjects experiencing normal menstrual bleeding.

In some embodiments, a flow rate is measured as the volume of menstrual blood collected from a subject over a time period. In some embodiments, the time period is pre-determined, and is a portion of the duration of menstrual bleeding up to the entire duration of menstrual bleeding. In some embodiments, the time period is at least 5 minutes, 10 minutes, 15 minutes, 30 minutes, 45 minutes, 1 hour, 1.5 hours, 2 hours, 3 hours, 4 hours, 5 hours, 6 hours, 7 hours, 8 hours, 9 hours, 10 hours, 11 hours, 12 hours, 18 hours, 24 hours, 48 hours, or 72 hours. In some embodiments, the time period is no more than 15 minutes, 30 minutes, 45 minutes, 1 hour, 1.5 hours, 2 hours, 3 hours, 4 hours, 5 hours, 6 hours, 7 hours, 8 hours, 9 hours, 10 hours, 11 hours, 12 hours, 18 hours, 24 hours, 48 hours, or 72 hours. In some embodiments, a flow rate is measured as a maximum, median, mode, or average flow rate during a given menstrual cycle. In some embodiments, a range of flow rates is measured during a given menstrual cycle. In some embodiments, volume of menstrual blood collected during an elapsed time period is compared with a threshold value to determine normal menstrual bleeding, HMB, or AUB.

In some embodiments, a flow rate is measured by determining the amount of time required for a predetermined volume of menstrual blood to be collected from the subject. In some embodiments, a predetermined volume of blood is at least about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.5 mL, about 2.0 mL, about 2.5 mL, about 3.0 mL, about 3.5 mL, about 4.0 mL, about 4.5 mL, about 5.0 mL, or more. In some embodiments, once a predetermined volume of blood has been detected, a flow rate is calculated for example by determining the ratio of the volume collected of a menstrual fluid sample to the time taken to collect the menstrual fluid sample. In some embodiments, the time elapsed during collection of a menstrual fluid sample of a predetermined volume is compared with a threshold value to determine normal menstrual bleeding, HMB, or AUB.

In some embodiments, a flow rate is measured on any day a subject is experiencing menstrual bleeding. In some embodiments, a flow rate is measured more than once during menstrual bleeding. Flow rate is measured on day 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of menstrual bleeding. In some embodiments, a flow rate is measured after 30 days of menstrual bleeding. In some embodiments, a flow rate is measured 1, 2, 3, 4, 5, or more times during a menstrual cycle.

In some embodiments, a flow rate is at least 0.01 mL per hour, at least 0.5 mL per hour, at least 0.1 mL per hour, at least 0.5 mL per hour, at least 1.0 mL per hour, at least 1.5 mL per hour, at least 2.0 mL per hour, at least 2.5 mL per hour, at least 3.0 mL per hour, at least 3.5 mL per hour, at least 4.0 mL per hour, at least 4.5 mL per hour, or at least 5.0 mL per hour. In some embodiments, a flow rate measured in a subject during one menstrual cycle is different than a flow rate measured in a subject during a different menstrual cycle. In some embodiments, a flow rate varies during a single menstrual cycle in a subject. In some embodiments, a flow rate measured at one time point in a menstrual cycle is the same or different than a flow rate measured at a different time point in the same menstrual cycle. In some embodiments, a subject experiencing HMB or AUB during a menstrual cycle experiences normal menstrual bleeding during a different part of the same menstrual cycle or during a different menstrual cycle. In some embodiments, a subject experiencing normal menstrual bleeding during a menstrual cycle experiences HMB or AUB during a different part of the same menstrual cycle or during a different menstrual cycle.

In some embodiments, an increased flow rate is a flow rate that is higher than a threshold flow rate. In some embodiments, a threshold flow rate is determined using data from subjects or other experimental data. In some embodiments, a threshold flow rate is a predicted value or a measured value. In some embodiments, a threshold flow rate is determined by measuring the flow rate of the menstrual fluid of at least 1, 2, 3, 4, 5, 10, 15, 20, 30, 40, or 50 subjects experiencing normal menstrual bleeding. In some embodiments, a threshold flow rate is determined by measuring the flow rate of the menstrual fluid of at least 1, 2, 3, 4, 5, 10, 15, 20, 30, 40, or 50 subjects experiencing HMB. In some embodiments, a threshold flow rate is determined by measuring the flow rate of the menstrual fluid of at least 1, 2, 3, 4, 5, 10, 15, 20, 30, 40, or 50 subjects experiencing AUB. In some embodiments, a threshold flow rate is determined by comparing the flow rate of subjects experiencing normal menstrual bleeding and HMB, or subjects experiencing normal menstrual bleeding and AUB. In some embodiments, a threshold flow rate is an average flow rate, a mean flow rate, a mode flow rate, a maximum flow rate, a minimum flow rate, an average flow rate plus 1, 2, or 3 standard deviations of a flow rate, or an average flow rate minus 1, 2, or 3 standard deviations of a flow rate.

In some embodiments, an increased flow rate is at least 10%, 20%, 30%, 40%, 50%, 60%, 70%, 80%, 90%, 100%, 150%, 200% higher than a threshold flow rate. In some embodiments, an increased flow rate is indicative of HMB or AUB. In some embodiments, a decreased flow rate is at least 10%, 20%, 30%, 40%, 50%, 60%, 70%, 80%, or 90% lower than a threshold flow rate. In some embodiments, a decreased flow rate is a maximum rate of flow of a menstrual cycle which is lower than a threshold rate. In some embodiments, a decreased flow rate is indicative of AUB. In some embodiments, an increased or decreased flow rate indicates a menstrual cycle disorder.

In some embodiments, a flow rate of a fluid which is not menstrual fluid, such as cervicovaginal fluid or amniotic fluid, is measured using these methods. Buffer solutions

In some embodiments, fluid taken from the vaginal cavity is a complex, heterogeneous mixture of one or more cell populations and comprises one or more cell types. During HMB or AUB, the cell types found in fluid, such as menstrual blood, is altered. In some embodiments, a fluid sample from the vaginal cavity of a subject experiencing HMB or AUB comprises a different composition of cell types, more of at least one cell type, or less of at least one cell type than a subject experiencing normal menstrual bleeding. In some embodiments, prior to measuring an amount of a type of a cell in a sample, the sample is contacted with a buffer solution, e.g., by layering, pipetting, pouring, stirring, vortexing, or otherwise exposing the sample to the buffer solution.

In some embodiments, the buffer solution is a buffer which preserves one or more cell types found in a fluid sample collected from a vaginal cavity such as menstrual fluid or a buffer which holds one or more cell types found in menstrual fluid without affecting the integrity of the one or more cell types. In some embodiments, a buffer solution comprises LBgard (Biomatrica, San Diego), RNAgard (Biomatrica, San Diego), or other commercially available buffer solution. In some embodiments, a buffer solution is designed to preserve the integrity of protein, nucleic acid, lipids, metabolites, cell membranes, or whole cells.

In some embodiments, a buffer solution is at an acceptable pH. In some embodiments, a buffer solution is at a pH which is exactly 7. In some embodiments, a buffer solution is at a pH which is between 6.99 and 7.01, between 6.95 and 7.05, between 6.9 and 7.1, between 6.85 and 7.15, between 6.8 and 7.2, between 6.75 and 7.25, or between 6.7 and 7.3.

In some embodiments, a buffer solution has an acceptable osmolality. In some embodiments, a buffer solution has an osmolality of between 260 mOsm/kg and 320 mOsm/kg, between 260 mOsm/kg and 300 mOsm/kg, between 260 mOsm/kg and 280 mOsm/kg, between 280 mOsm/kg and 320 mOsm/kg, between 280 and 300 mOsm/kg, or between 300 mOsm/kg and 320 mOsm/kg. In some embodiments, a buffer solution has an osmolality of about 260 mOsm/kg, about 270 mOsm/kg, about 280 mOsm/kg, about 290 mOsm/kg, about 300 mOsm/kg, about 310 mOsm/kg, or about 320 mOsm/kg. In some embodiments, a buffer solution has an osmolality of more than 320 mOsm/kg or less than 260 mOsm/kg.

In some embodiments, a buffer solution has an acceptable viscosity. In some embodiments, a buffer solution has a viscosity of about 1×10⁻⁴ Pa·s, about 2×10⁻⁴ Pa·s, about 3×10⁻⁴ Pa·s, about 4×10⁻⁴ Pa·s, about 5×10⁻⁴ Pa·s, about 6×10⁻⁴ Pa·s, about 7×10⁻⁴ Pa·s, about 8×10⁻⁴ Pa·s, about 9×10⁻⁴ Pa·s, about 1×10⁻³ Pa·s, about 2×10⁻³ Pa·s, about 3×10⁻³ Pa·s, about 4×10⁻³ Pa·s, about 5×10⁻³ Pa·s, about 6×10⁻³ Pa·s, about 7×10⁻³ Pa·s, about 8×10⁻³ Pa·s, or about 9×10⁻³ Pa·s. In some embodiments, a buffer solution has a viscosity of between 1×10⁻⁴ Pa·s and 1×10⁻² Pa·s, between 1×10⁻⁴ Pa·s and 5×10⁻³ Pas, between 1×10⁻⁴ Pa·s and 1×10⁻³ Pas, between 1×10⁻⁴ Pa·s and 5×10⁻⁴ Pa·s, between 5×10⁻² Pa·s and 1×10⁻³ Pa·s, between 5×10⁻⁴ Pa·s and 5×10⁻³ Pas, between 5×10⁻⁴ Pa·s and 1×10⁻³ Pas, between 1×10⁻³ Pas and 1×10⁻² Pa·s, between 1×10⁻³ Pas and 5×10⁻³ Pas, or between 5×10⁻³ Pas and 1×10⁻² Pa·s. In some embodiments, a buffer solution has an osmolality that is approximately the same as the osmolality of water, the osmolality of blood, the osmolality of cervicovaginal fluid, or the osmolality of menstrual fluid.

In some embodiments, a buffer comprises a preservation solution. In some embodiments, a preservation solution is formulated to preserve a sample in whole or in part. In some embodiments, a preservation solution is formulated to preserve one or more cell types within a sample.

In some embodiments, the preservation solution described herein comprises at least one of: a preservation agent, a dissociation agent, or a combination thereof. In some embodiments, the preservation agent is a zwitterionic compound, an osmoprotectant, an apoptosis inhibitor, a non-reducing sugar or polyol, a disaccharide derivative, a chelating agent, a pH buffer, a phosphatase inhibitor, a protease inhibitor, or a combination thereof. In some embodiments, the dissociation agent is a mucolytic, an expectorant, a surfactant, a nuclease, a protease, or a combination thereof. In some embodiments, the preservation solution further comprises a spike-in. In some embodiments, the preservation solution consists essentially of: a zwitterionic compound, an osmoprotectant, an apoptosis inhibitor, a non-reducing sugar or polyol, a chelating agent, a pH buffer, a phosphatase inhibitor, a protease inhibitor, a mucolytic, an expectorant, a surfactant, a nuclease, a protease, a spike-in, or any combination thereof. In some embodiments, the preservation solution comprises an agent for selective lysis of non-endometrial cells but not of endometrial cells in the sample. In some embodiments, the preservation solution comprises an agent for selective lysis of a cell that is not an endometrial cell. In some embodiments, the agent for selective lysis is a dissociation agent. In some embodiments, the agent for selective lysis is the nuclease, the protease, or a combination thereof. In some embodiments, the preservation solution selectively lysed about 10%, 20%, 30%, 40%, 50%, 60%, 70%, 80%, 90%, 95%, or 99% of the non-endometrial cells in the sample. In some embodiments, the preservation solution selectively lyses about 10%, 20%, 30%, 40%, 50%, 60%, 70%, 80%, 90%, 95%, or 99% of the cells which are not endometrial cells in the sample. In some embodiments, the preservation solution further comprises a binding agent.

In some embodiments, the preservation solution comprises a zwitterionic compound. In some embodiments, the zwitterionic compound is a betaine or a betaine analog. In some embodiments, the zwitterionic compound is trimethylamino N-oxide (TMAO). In some embodiments, the zwitterionic compound is N-Tris(hydroxymethyl)methyl-2-aminoethanesulfonic acid; 3-(N,N-bis[2-hydroxyethyl]amino)-2-hydroxypropanesulphonic acid; 3-(N-morpholino)propanesulfonic acid, 4-(2-hydroxyethyl)-1-piperazineethanesulfonic acid; Tris(hydroxymethyl)aminomethane; piperazine-N,N′-bis(2-ethanesulfonic acid); 2-(N-Morpholino)ethanesulfonic acid hydrate; N,N-Bis(2-hydroxyethyl)-2-aminoethanesulfonic acid; N-[Tris(hydroxymethyl)methyl]glycine; 3-((3-acrylamidopropyl)-dimethylammonio)-propane-1-sulfonate; hydroxyectoine; ectoine; homoectoine; L-carnitine; sarcosine; N,N-Dimethylglycine triethylammonium acetate; glycerol phosphate; tricine; pentaerythritol; N-ethyl-N,N-bis-(2-hydroxyethyl)ammonium-N-4-butyl sulfonate; 3-morpholino-2-hydroxypropanesulfonic acid; 4-(2-ethoxy-2-oxoethyl)-4-ethylmorpholin-4-ium bromide; N-(2-ethoxy-2-oxoethyl)-3-hydroxy-N,N-bis(2-hydroxyethyl)propan-1-aminium bromide; 2-ethoxy-N,N,N-triethyl-2-oxoethanaminium bromide; 2-((3-hydroxypropyl)dimethylammonio)acetate; 2-((2-hydroxypropyl)dimethylammonio) acetate; 2-(2-(hydroxymethyl)-1-methylpiperidinium-1-yl)acetate; 2-((2-hydroxyethyl)dimethylammonio)acetate; 2-((2,3-dihydroxypropyl) dimethylammonio)acetate; 1-(2-ethoxy-2-oxoethyl)-4-hydroxy-1-methylpiperidinium bromide; 2-(4-hydroxy-1-methylpiperidinium-1-yl)acetate; 2-ethoxy-N-(2-(2-hydroxyethoxy)ethyl)-N,N-dimethyl-2-oxoethanaminium bromide; 2-((2-(2-hydroxyethoxy)ethyl)dimethylammonio)acetate; 2-(bis(2-hydroxyethyl)-(methyl)ammonio)acetate; 4-(2-hydroxyethyl)-4-methyl-2-oxomorpholin-4-ium bromide; 2-(bis(2-hydroxyethyl)-(methyl)ammonio)acetate; 2-(4-(2-hydroxyethyl)morpholino-4-ium)acetate; 4-(2-ethoxy-2-oxoethyl)-4-methylmorpholin-4-ium bromide; 1-(2-ethoxy-2-oxoethyl)-1-methylpyrrolidinium bromide; 2-(benzyl(2-hydroxy-ethyl)(methyl)ammonio)acetate; 3-(2,3-dihydroxypropyl)-1-methyl-1H-imidazol-3-ium chloride; 1,3-dimethyl-1H-imidazol-3-ium methyl sulfate; N-benzyl-2-ethoxy-N,N-dimethyl-2-oxoethanaminium bromide; 1-(2-ethoxy-2-oxoethyl)-1-methylpiperidi-nium bromide; N-(2-ethoxy-2-oxoethyl)-N,N-dimethylbenzenaminium bromide; 1-(2-ethoxy-2-oxoethyl)-3-hydroxy-1-methylpiperidinium bromide; 3-(2-(2-hydroxyethoxy)ethyl)-1-methyl-1H imidazol-3-ium chloride; 3-(2-(2-(2-hydroxyethoxy)ethoxy)ethyl)-1-methyl-1H-imidazol-3-ium chloride; 1-methyl-3-tetradecyl-1H-imidazol-3-ium bromide; N-(2-ethoxy-2-oxoethyl)-N,N-dimethylcyclo-hexanaminium bromide; 3-((2-hydroxy-ethyl)dimethyl-ammonio)pro-panoate; or any combination thereof. In some embodiments, the zwitterionic compound is a polyzwitterion. In some embodiments, the polyzwitterion is carboxybetaine methacrylate-1; carboxybetaine methacrylate-1-tertiary amine; carboxybetaine methacrylate-2; carboxybetaine acrylamide-2; carboxybetaine acrylamide-2-ethyl ester; carboxybetaine acrylamide-2-RGD; carboxybetaine diacrylamide crosslinker; glycine betaine; poly-sulfobetaine; or any combination thereof.

In some embodiments, the preservation solution comprises an osmoprotectant. In some embodiments, an osmoprotectant is for example trimethylammonium acetate; glycerol phosphate; diglycerol phosphate, N-(2-hydroxy-1,1-bis(hydroxymethyl)ethyl)glycine; 3-(N-morpholino)-2-hydroxypropanesulfonic acid; pentaerythritol; glyceric acid; malic acid; tartaric acid; lactic acid; glycolic acid; 2-hydroxybutyric acid; 3-hydroxybutyric acid; 4-amino-3-hydroxybutyric acid; 3-(1-azoniabicyclo[2.2.2]oct-1-yl)propane-1-sulfonate; 1-(2-carboxylatoethyl)-1-azabicyclo[2.2.2]octan-1-ium; or any combination thereof.

In some embodiments, the preservation solution comprises an apoptosis inhibitor. In some embodiments, the apoptosis inhibitor is PERK-eIF2-a inhibitor, ASK1 inhibitor, NRF2-KEAP1 inhibitor, JNK inhibitor, p38 MAP kinase inhibitor, IRE1 inhibitor, GSK3 inhibitor, PIK3 pathway inhibitor, MEK inhibitor, calpain inhibitor, caspase-1 inhibitor, or any combination thereof.

In some embodiments, the preservation solution comprises a non-reducing sugar or polyol. In some embodiments, the non-reducing sugar or polyol is glycol, glycerol, erythritol, threitol, arabitol, xylitol, ribitol, adonitol, mannitol, sorbitol, galactitol, fucitol, iditol, inositol, adonitol, sucralfate, sucrose octasulfate, sucrose, trehalose, or any combination thereof. In some embodiments, the preservation solution comprises a disaccharide derivative. In some embodiments, the disaccharide derivative comprises sucralose, trichloronated maltose, or a combination thereof.

In some embodiments, the preservation solution comprises a chelating agent. In some embodiments, the chelating agent is diethylenetriaminepentaacetic acid (DTPA); ethylenediaminetetraacetic acid (EDTA); ethylene glycol tetraacetic acid (EGTA); trans-1,2-diaminocyclohexane-N,N,N′,N′-tetraacetic acid (CDTA); 1,2-bis(2-aminophenoxy)ethane-N,N,N′,N′-tetraacetic acid (BAPTA); 1,4,7,10-tetraazacyclododecane-1,4,7,10-tetraacetic acid (DOTA); N-(2-hydroxyethyl)ethylenediamine-N,N′,N′-triacetic acid; sodium gluconate; nitrilotriacetic acid (NTA); or a combination thereof.

In some embodiments, the preservation solution comprises a pH buffer. In some embodiments, the pH buffer comprises citric acid; tartaric acid; malic acid; sulfosalicylic acid; sulfoisophthalic acid; oxalic acid; borate; CAPS (3-(cyclohexylamino)-1-propanesulfonic acid); CAPSO (3-(cyclohexylamino)-2-hydroxy-1-propanesulfonic acid); EPPS (4-(2-hydroxyethyl)-1-piperazinepropanesulfonic acid); HEPES (4-(2-hydroxyethyl)piperazine-1-ethanesulfonic acid); MES (2-(N-morpholino)ethanesulfonic acid); MOPS (3-(N-morpholino)propanesulfonic acid); MOPSO (3-morpholino-2-hydroxypropanesulfonic acid); PIPES (1,4-piperazinediethanesulfonic acid); TAPS (N[tris(hydroxymethyl)methyl]-3-aminopropanesulfonic acid); TAPSO (2-hydroxy-3-[tris(hydroxymethyl)methylamino]-1-propanesulfonic acid); TES (N-[tris(hydroxymethyl)methyl]-2-aminoethanesulfonic acid); bicine (N,N-bis(2-hydroxyethyl)glycine); tricine (N-[tris(hydroxymethyl)methyl]glycine); tris (tris(hydroxymethyl)aminomethane); bis-tris (2-[bis(2-hydroxyethyl)amino]-2-(hydroxymethyl)-1,3-propanediol); or a combination thereof.

In some embodiments, the preservation agent comprises a phosphatase inhibitor. In some embodiments, the phosphatase inhibitor comprises beta-Glycerophosphate, aprotinin, bestatin, EDTA, leupeptin, pepstatin A, or a combination thereof.

In some embodiments, the preservation agent comprises a protease inhibitor. In some embodiments, the protease inhibitor is (2R)-2-Mercaptomethyl-4-methylpentanoyl-beta-(2-naphthyl)-Ala-Ala Amide; 2-Antiplasmin; 3,4-Dichloroisocoumarin; 4-(2-Aminoethyl) benzenesulfonyl fluoride hydrochloride; 5-(R,S)-T-trans-Cinnamido-7-methyl-4-oxo-octanoyl-L-prolyl-L-proline; a1-Antchymotrypsin; a1-Antitrypsin; a2-Antiplasmin; a2-Macroglobulin; Antithrombin III; Aprotinin; Bromoenol lactone; BTEE; C1 Esterase inhibitor; Chymostatin; Complement C1 esterase inhibitor; Dichloromethylenediphosphonic acid disodium salt; Diisopropyl fluorophosphate; e-Amino-n-caproic acid; Ecotin; EDTA; Eglin C fragment 60-63 methyl ester; Gabexate mesylate; Histatin 5; Ile-Pro-Ile; Isoamylphosphonyl-Gly-L-Pro-L-Ala; Leupeptin; N a-p-Tosyl-L-lysine chloromethyl ketone hydrochloride; N-Acetyl-eglin C; N-Tosyl-L-phenylalanine chloromethyl ketone; p-Chloromercuribenzoic acid Free Acid; Phenylmethylsulfonyl fluoride; Trypsin Inhibitor; Trypsin-chymotrypsin inhibitor; Z-L-Phe chloromethyl ketone; Boc-Asp(OMe)-fluoromethyl ketone; Z-Ala-Glu(OMe)-Val-Asp(OMe)-fluoromethyl ketone; Antipain dihydrochloride from microbial source protease inhibitor; CA-074 methyl ester; Calpain Inhibitor I; Calpain Inhibitor II; Cystatin; E-64 protease inhibitor; Leupeptin trifluoroacetate salt; α2-Macroglobulin; Procathepsin B; Z-Leu-Leu-Leu-fluoromethyl ketone; Z-Phe-Phe-fluoromethyl ketone; or a combination thereof.

In some embodiments, the preservation solution comprises a spike-in. In some embodiments, as used herein, a “spike-in” is a molecule, such as a nucleic acid, a cell, or a set of molecules or cells added to a sample, wherein the spike-in is used to quantitatively or qualitatively assess or to normalize a sample. In some embodiments, the spike-in is a nucleic acid spike-in. In some embodiments, the nucleic acid spike-in is a DNA spike-in, an RNA spike-in, a bacterial spike-in, or a combination thereof. In some embodiments, the DNA spike-in is a synthetic DNA or a plurality of synthetic DNAs. In some embodiments, the RNA spike-in is a synthetic RNA or a plurality of synthetic RNAs. In some embodiments, the RNA spike-in is a set of RNA transcripts developed by the External RNA Controls Consortium (ERCC).

In some embodiments, the preservation solution comprises a mucolytic agent. In some embodiments, the mucolytic agent dissociates (e.g., “unclump”) at least a portion of cellular aggregations in the cervicovaginal sample. In some embodiments, the mucolytic is acetylcysteine, ambroxol, bromhexine, carbocisteine, domiodol, dornase alfa, eprazinone, erdosteine, letosteine, mannitol, mesna, neltenexine, sobrerol, stepronin, tiopronin, N-acetyl-L-cysteine, L-acetyl cysteine/Liberase™, or a combination thereof.

In some embodiments, the preservation solution comprises an expectorant. In some embodiments, the expectorant is althea root, antimony pentasulfide, creosote, guaiacolsulfonate, guaifenesin (+oxomemazine), ipecacuanha, levoverbenone, potassium iodide, senega, tyloxapol, ammonium chloride, or a combination thereof.

In some embodiments, the preservation solution comprises a surfactant. In some embodiments, the surfactant is polyoxyethylene glycol octylphenol ethers; polyoxyethylene glycol alkylphenol ethers; polyoxyethylene glycol sorbitan alkyl esters; sorbitan alkyl esters; polyethylene glycol; polypropylene glycol; carboxylates; sulphonates; petroleum sulphonates; alkylbenzenesulphonates; naphthalenesulphonates; olefin sulphonates; alkyl sulphates; sulphates; sulphated esters; sulphated alkanolamides; alkylphenols; ethoxylated aliphatic alcohol; polyoxyethylene surfactants; carboxylic esters; polyethylene glycol esters; anhydrosorbitol esters; glycol esters; carboxylic amide; monoalkanolamine condensates; polyoxyethylene fatty acid amides; quaternary ammonium salts; polyoxyethylene alkyl and alicyclic amines; N,N,N′,N′ tetrakis substituted ethylenediamines; 2-alkyl 1-hydroxethyl 2-imidazolines; or a combination thereof.

In some embodiments, the preservation solution comprises a nuclease. In some embodiments, the nuclease is a Benzonase®, DNAse I, DNAse II, Exonuclease III, Micrococcal Nuclease, Nuclease P1, Nuclease Si, Phosphodiesterase I, Phosphodiesterase II, RNAse A, RNAse H, RNAse Ti, or a combination thereof.

In some embodiments, the preservation solution comprises a protease. In some embodiments, the protease is adispase II, trypsin, pronase, collagenase 1, collagenase 2, collagenase 3, collagenase 4, hyaluronidase, pepsin, papain, chemotrypsin, chymase, clostripain, complement C1r, complement C1s, complement factor D, complement factor I, cucumisin, dipeptidyl peptidase, elastase, endoproteinase, enterokinase, Factor X Activated, caspase, cathepsin, matrix metalloprotease, or a combination thereof.

In some embodiments, the osmolality of the preservation solution is from about 310 to about 410 mOsm kg⁻¹. In some embodiments, the osmolality of the preservation solution is from about 95 to about 210 mOsm kg⁻¹.

In some embodiments, the preservation solution does not comprise a fixative. In some embodiments, the fixative comprises an alcohol, an aldehyde, an oxidizing agent, a metallic fixative or a combination thereof. In some embodiments, the alcohol is methanol, ethanol, propanol, isopropanol, butanol, or a combination thereof. In some embodiments, the aldehyde is formaldehyde, glutaraldehyde, or a combination thereof. In some embodiments, the oxidizing agent is an osmium tetraoxide, potassium permanganate, potassium dichromate, or a combination thereof. In some embodiments, the metallic fixative is a mercuric chloride, a picric acid, or a combination thereof. In some embodiments, the preservation solution does not comprise an alcohol, an aldehyde, an oxidizing agent, a metallic fixative, or a combination thereof.

In some embodiments, the preservation solution comprises a binding agent. In some embodiments, the binding agent selectively binds to an endometrial cell, a non-endometrial cell of the individual, spermatozoa, bacterial cell, fungal cell, or a combination thereof. In some embodiments, the binding agent selectively binds to at least one protein or fragment thereof. In some embodiments, the at least one protein or fragment thereof is a biomarker of endometriosis. In some embodiments, the binding agent selectively binds to nucleic acid. In some embodiments, the nucleic acid is a biomarker of endometriosis. In some embodiments, the binding agent is immobilized, for example, to a bead or to a surface of a component of the systems described herein. In some embodiments, the binding agent is coupled to the bead or the surface of the system. In some embodiments, the binding agent is reversibly or irreversibly coupled to the bead or the surface of the system. In some embodiments, the binding agent comprises a cleavable moiety, for example, a cleavable linker. In some embodiments, the cleavable linker is cleaved photolytically, chemically, thermally, or enzymatically.

In certain embodiments, for example, about 0.5 ml of preservation solution (Biomatrica® LBgard™) is diluted in about 8.5 ml of distilled water to form a diluted preservation solution. In other embodiments, for example, for example, about a volume of preservation solution (Biomatrica® RNAgard™) is diluted in a volume of distilled water to form a diluted preservation solution. In some embodiments, such a diluted preservation solution is used in the methods and/or systems provided herein. In some embodiments, the diluted preservation solution is added to a tampon at about 3 ml to about 5 ml of diluted preservation solution per gram of fluid that is absorbed into the tampon. In some embodiments, a light absorbency tampon absorbs up to 6 g of fluid, thus, about 18 ml to about 30 ml of diluted preservation solution is added to the light absorbency tampon. In some embodiments, the diluted preservation solution is added to the tampon in the system described herein, following the rupture of the disruptable member. In some embodiments, accordingly, as the absorbency of the tampon increases, the amount of diluted preservation solution to be added increases. In some embodiments, the sample is incubated in the buffer solution prior to analysis. In some embodiments a sample is incubated in a buffer solution at about 4° C., about 10° C., about 15° C., about 20° C., about 25° C., about 30° C., about 35° C., about 40° C., about 45° C., about 50° C., about 55° C., about 60° C., about 55° C., about 60° C., about 65° C., about 70° C., about 75° C., about 80° C., about 85° C., about 90° C., about 95° C., or about 100° C. In some embodiments, a sample is incubated in a buffer solution at between 4° C. and 100° C., between 4° C. and 50° C., between 4° C. and 30° C., 4° C. and 20° C., between 4° C. and 15° C., between 4° C. and 10° C., between 10° C. and 20° C., between 10° C. and 15° C., between 15° C. and 20° C., between 20° C. and 100° C., between 20° C. and 50° C., between 20° C. and 40° C., between 20° C. and 35° C., between 20° C. and 30° C., between 20° C. and 25° C., between 25° C. and 40° C., between 25° C. and 35° C., between 25° C. and 30° C., between 30° C. and 40° C., between 30° C. and 35° C., between 35° C. and 40° C., between 30° C. and 100° C., between 30° C. and 90° C., between 30° C. and 80° C., between 30° C. and 70° C., between 30° C. and 60° C., between 30° C. and 50° C., between 40° C. and 100° C., between 40° C. and 90° C., between 40° C. and 80° C., between 40° C. and 70° C., between 40° C. and 60° C., between 40° C. and 50° C., between 50° C. and 70° C., between 50° C. and 60° C., between 60° C. and 100° C., between 60° C. and 90° C., between 60° C. and 80° C., between 60° C. and 70° C., between 70° C. and 100° C., between 70° C. and 90° C., between 70° C. and 80° C., between 80° C. and 100° C., between 80° C. and 90° C., or between 90° C. and 100° C.

In some embodiments, incubation of a sample lasts for at least about 30 seconds, about 1 minute, about 2 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, overnight, for about 24 hours, or more. In some embodiments, incubation of a sample lasts for between 30 seconds and 24 hours, between 30 seconds and 12 hours, between 30 seconds and 6 hours, between 30 seconds and 1 hour, between 30 seconds and 30 minutes, between 30 seconds and 1 minute, between 1 minute and 1 hour, between 1 minute and 45 minutes, between 1 minutes and 30 minutes, between 1 minute and 10 minutes, between 1 minute and 5 minutes, between 5 minutes and 1 hour, between 5 minutes and 45 minutes, between 5 minutes and 30 minutes, between 5 minutes and 15 minutes, between 5 minutes and 10 minutes, between 10 minutes and 1 hour, between 10 minutes and 45 minutes, between 10 minutes and 30 minutes, between 10 minutes and 15 minutes, between 15 minutes and 1 hour, between 15 minutes and 45 minutes, between 15 minutes and 30 minutes, between 30 minutes and 24 hours, between 30 minutes and 12 hours, between 30 minutes and 6 hours, between 30 minutes and 1 hour, between 30 minutes and 45 minutes, between 45 minutes and 1 hour, between 1 hour and 24 hours, between 1 hour and 12 hours, between 1 hour and 6 hours, between 1 hour and 5 hours, between 1 hour and 4 hours, between 1 hour and 3 hours, between 1 hour and 2 hours, between 2 hours and 6 hours, between 2 hours and 5 hours, between 2 hours and 4 hours, between 2 hours and 3 hours, between 3 hours and 6 hours, between 3 hours and 5 hours, between 3 hours and 4 hours, between 4 hours and 6 hours, between 4 hours and 5 hours, between 5 hours and 6 hours, between 6 hours and 24 hours, between 6 hours and 12 hours, or between 12 hours and 24 hours.

In some embodiments, the sample is incubated in the buffer solution prior to analysis. In some embodiments, the buffer solution is incubated at a given temperature for at least 1 day, 2 days, 3 days, 4 days, 5 days, 6 days, 1 week, 2 weeks, 4 weeks, or more. In some embodiments, the sample is stored at room temperature, in a refrigerator, in a freezer, in a −80° C. freezer, on dry ice, or in liquid nitrogen.

In some embodiments, the sample is contacted with the buffer solution at a ratio of about 10% v/v, 20% v/v, 30% v/v, 40% v/v, 50% v/v, 60% v/v, 70% v/v, 80% v/v, 90% v/v, 100% v/v, 150% v/v, 200% v/v, 250% v/v, 300% v/v, 400% v/v, 500% v/v, 600% v/v, 700% v/v, 800% v/v, 900% v/v, or 1000% v/v, where v/v indicates the ratio of the volume of the buffer solution to the volume of the sample). In some embodiments, a sample is contacted with the buffer solution at a ratio of less than 10% v/v or greater than 1000% v/v. In some embodiments, the sample is contacted with the buffer solution at a ratio of between 10% v/v and 1000% v/v, between 10% v/v and 900% v/v, between 10% v/v and 800% v/v, between 10% v/v and 700% v/v, between 10% v/v and 600% v/v, between 10% v/v and 500% v/v, between 10% v/v and 400% v/v, between 10% v/v and 300% v/v, between 10% v/v and 200% v/v, between 10% v/v and 100% v/v, between 100% v/v and 1000% v/v, between 100% v/v and 900% v/v, between 100% v/v and 800% v/v, between 100% v/v and 700% v/v, between 100% v/v and 600% v/v, between 100% v/v and 500% v/v, between 100% v/v and 400% v/v, between 100% v/v and 300% v/v, between 100% v/v and 200% v/v, between 200% v/v and 100% v/v, between 200% v/v and 900% v/v, between 200% v/v and 800% v/v, between 200% v/v and 700% v/v, between 200% v/v and 600% v/v, between 200% v/v and 500% v/v, between 200% v/v and 400% v/v, between 200% v/v and 300% v/v, between 300% v/v and 1000% v/v, between 300% v/v and 900% v/v, between 300% v/v and 800% v/v, between 300% v/v and 700% v/v, between 300% v/v and 600% v/v, between 300% v/v and 500% v/v, between 300% v/v and 400% v/v, between 400% v/v and 1000% v/v, between 400% v/v and 900% v/v, between 400% v/v and 800% v/v, between 400% v/v and 700% v/v, between 400% v/v and 600% v/v, between 400% v/v and 500% v/v, between 500% v/v and 1000% v/v, between 500% v/v and 900% v/v, between 500% v/v and 800% v/v, between 500% v/v and 700% v/v, between 500% v/v and 600% v/v, between 600% v/v and 1000% v/v, between 600% v/v and 900% v/v, between 600% v/v and 800% v/v, between 600% v/v and 700% v/v, between 700% v/v, and 1000% v/v, between 700% v/v and 900% v/v, between 700% v/v and 800% v/v, between 800% v/v and 1000% v/v, between 800% v/v and 900% v/v, or between 900% v/v and 1000% v/v. In some embodiments, the sample is contacted with the buffer solution at a ratio of between 10% v/v and 100% v/v, between 10% v/v and 90% v/v, between 10% v/v and 80% v/v, between 10% v/v and 70% v/v, between 10% v/v and 60% v/v, between 10% v/v and 50% v/v, between 10% v/v and 40% v/v, between 10% v/v and 30% v/v, between 10% v/v and 20% v/v, between 20% v/v and 100% v/v, between 20% v/v and 90% v/v, between 20% v/v and 80% v/v, between 20% v/v and 70% v/v, between 20% v/v and 60% v/v, between 20% v/v and 50% v/v, between 20% v/v and 40% v/v, between 20% v/v and 30% v/v, between 30% v/v and 100% v/v, between 30% v/v and 90% v/v, between 30% v/v and 80% v/v, between 30% v/v and 70% v/v, between 30% v/v and 60% v/v, between 30% v/v and 50% v/v, between 30% v/v and 40% v/v, between 40% v/v and 100% v/v, between 40% v/v and 90% v/v, between 40% v/v and 80% v/v, between 40% v/v and 70% v/v, between 40% v/v and 60% v/v, between 40% v/v and 50% v/v, between 50% v/v and 100% v/v, between 50% v/v and 90% v/v, between 50% v/v and 80% v/v, between 50% v/v and 70% v/v, between 50% v/v and 60% v/v, between 60% v/v and 100% v/v, between 60% v/v and 90% v/v, between 60% v/v and 80% v/v, between 60% v/v and 70% v/v, between 70% v/v and 100% v/v, between 70% v/v and 90% v/v, between 70% v/v and 80% v/v, between 80% v/v, and 100% v/v, between 80% v/v and 90% v/v, or between 90% v/v and 100% v/v.

In some embodiments, upon contact with the buffer, cell membranes of at least about 80%, 85%, 90%, 95%, 99%, or 100% of cells present in the sample remains in a substantially intact state. In some embodiments, an intact state in some cases comprises an absence of pores or tears in the cell membrane, an absence of disruption of glycosylation (e.g., glycosylation of surface molecules), or an absence of denaturing (e.g. denaturation of surface proteins).

In some embodiments, upon contact with the buffer, the cell membranes of at least 70%, 75%, 80%, 85%, 90%, 95%, 99%, or 100% of the types of cells in the sample is maintained in a substantially intact state. In some embodiments, a cell membrane of a type of cell is considered to be maintained in a substantially intact state if at least about 80%, 85%, 90%, 95%, 99%, or 100% of cells of that type present in the sample remain in a substantially intact state. Device for collection or measurement of menstrual fluid

In some embodiments of methods provided herein, a sample collection device is used for collection of a menstrual fluid sample from a subject. In some embodiments, any device capable of collecting a sample from a subject is used. In some embodiments, a sample collection device comprises means of collecting menstrual fluid. In some embodiments, a sample collection device additionally comprises other components to measure, store, preserve, or ship a sample. In some embodiments, a sample collection device is provided with one or more lab accessories, such as a syringe or a vacutainer, to allow for removal of a sample from the device or preparation or analysis of the sample.

In some embodiments, a sample collection device hold a volume of menstrual fluid. In some embodiments, a sample collection device has a capacity of at least 0.5 mL, 1.0 mL, 1.5 mL, 2.0 mL, 2.5 mL, 3.0 mL, 3.5 mL, 4.0 mL, 4.5 mL, or 5.0 mL. In some embodiments, a volume of fluid collected in a sample collection device is equal to or less than the capacity of the sample collection device. In some embodiments, up to 0.5 mL, 1.0 mL, 1.5 mL, 2.0 mL, 2.5 mL, 3.0 mL, 3.5 mL, 4.0 mL, 4.5 mL, or 5.0 mL of menstrual fluid is collected in a sample collection device. In some embodiments, at least 0.5 mL, 1.0 mL, 1.5 mL, 2.0 mL, 2.5 mL, 3.0 mL, 3.5 mL, 4.0 mL, 4.5 mL, or 5.0 mL of menstrual fluid is collected in a sample collection device.

In some embodiments, a sample collection device comprises a means of collecting menstrual fluid from a subject, such as a tampon. In some embodiments, a tampon is inserted into the vagina of a subject, and menstrual fluid collects in or on the tampon. In some embodiments, after insertion into the vagina of a subject, time is allowed to elapse such that a sufficient volume of menstrual fluid is collected. In some embodiments, after an amount of time has passed, the tampon is removed.

In some embodiments, another means of collecting menstrual fluid, either inside the vagina or external to the vagina, is used, such as a pad, a tampon, a vaginal cup, a cervical cap, a menstrual disk, a cervical disk, a sponge, or an interlabial pad. In some embodiments, these, or other means, of collecting menstrual fluid is interchangeable.

In some embodiments, a sample collection device further comprises a collection packet. In some embodiments, a collection packet is a device used to receive a tampon or other means of collecting menstrual fluid from a subject. In some embodiments, a collection packet is a cylindrical device or is any shape which accommodates a tampon or other means of collecting menstrual fluid inside.

In some embodiments, a sample collection packet has a capacity of at least the volume of the means of collecting menstrual fluid. In some embodiments, a sample collection packet has a capacity of at least 0.5 mL, 1.0 mL, 1.5 mL, 2.0 mL, 2.5 mL, 3.0 mL, 3.5 mL, 4.0 mL, 4.5 mL, 5.0 mL, 6.0 mL, 7.0 mL, 8.0 mL, 9.0 mL, 10.0 mL, or more.

In some embodiments, a sample collection packet comprises a lid. In some embodiments, a lid is snapped closed, screwed closed, closed using a vacuum force, taped closed, glued closed, or crimped closed. In some embodiments, a lid is removable or non-removable once it is closed. In some embodiments, a sample collection packet is closed without a lid, for example by pinching the top of the sample collection packet or by using other closing means such as a zipper mechanism or an adhesive.

In some embodiments, a sample collection device comprise a device or kit as described in WO 2016/025332 A1 or WO 2017/180909 A1. In some embodiments, a representative and exemplary system includes at least some of the following components: a specialized sample collector which optimizes collection of fluid from a vaginal cavity (e.g., menstrual fluid) for testing; a biological matrix extractor comprising a compression top which pull fluid, vaginal mucosa, or semen into an assay delivery reservoir, through a filter; an assay cartridge which evaluates the biological content of a biological matrix; a cartridge reader which automates assay development, result capture, and/or result interpretation; or a mobile app interface that interprets and/or track a user's results and curates validated recommendations for health and behavior. In some embodiments, the cartridge reader electronically interfaced with a mobile phone by plugging into a headphone jack on the mobile phone. Optionally, a web-based interface provides access to easy interventions like food shopping, vitamin stores, and health facilities for therapeutics, and provides a positive behavioral feedback loop to increase prevention adherence.

In some embodiments, a sample collection device comprises a system for collecting a biological sample from a subject, which comprises a comprising a sample collector that non-invasively collects the biological sample from the subject. In some embodiments, the sample collector is inserted into to subject's vaginal cavity to collect the biological sample. In some embodiments, the system described herein collects a volume of biological sample comprising menstrual fluid, cervicovaginal fluid, secreted mucus, shed uterus cells, shed ovary cells, or other cells, tissue, or fluid. In some embodiments, the sample collector is made of materials that are capable of collecting and/or retaining the biological sample. In some embodiments, the sample collector is made of highly absorbent materials that absorb a liquid sample rapidly. In some embodiments, the sample collector is made of materials that release absorbed liquid samples rapidly, such as when a compression mechanism (e.g., pressure, force) is applied to the sample collector. In various embodiments, disposing the menstrual fluid sample in a preservation solution to form the mixture comprises placing a sample collector into a first central cavity of a system wherein the sample collector is compressed or squeezed, for example, to remove at least a portion of the sample from the sample collector. In some embodiments, the system comprises an extractor for extracting the biological sample from the sample collector. In some embodiments, the extractor comprises a component for applying a compression mechanism to the sample collector. In some embodiments, components for applying compression mechanisms include but are not limited to a spring, threaded screw, lever, air-tight plunger, or roller-based compression. In some embodiments, the liquid sample absorbed on a sample collector is extracted by applying a compression mechanism to the sample collector. In some embodiments, the system comprises the compression mechanism. In some embodiments, the system does not comprise a compression mechanism. In some embodiments, the compression mechanism is compressed outside of the system. In some embodiments, closing or sealing the system activates the compression mechanism. In some embodiments, closing or sealing the system does not activate the compression mechanism. In some embodiments, the compression mechanism is activated separately from closing or sealing the system. In some embodiments, the liquid sample absorbed on a sample collector is extracted without a compression mechanism. In some embodiments, the liquid sample absorbed on a sample collector is eluted into a buffer described herein. In some instances, compression of the sample collector in a manner that compresses the sample collector is carried out by the individual from whom the menstrual fluid sample was collected. In some instances, compression of the sample collector is carried out by at a laboratory or other location which processes the sample collector for assaying the collected sample. In some embodiments, the extractor comprises a sample receptacle that receives the sample collector via an opening, and a reservoir that is in fluid communication with the sample receptacle for receiving the biological sample released from the sample collector. In some embodiments, the reservoir and/or receptacle contains a solution comprising one or more reagents for analyzing, preserving, storing, or transporting the collected biological sample. In some instances, placing the sample collector into the first central cavity is carried out by a medical professional, such as an obstetrician or nurse. In some embodiments, the one or more reagents are necessary for hydrolyzing, diffusing, or releasing the biological sample. In some embodiments, the one or more reagents are necessary for analyzing, preserving, or extracting deoxyribonucleic acid, ribonucleic acid, or protein in the biological sample. In some embodiments, the one or more reagents are necessary for reducing analysis background noise. In some embodiments, the one or more reagents are necessary for precipitating or removing a contaminant in the biological sample. In some embodiments, the one or more reagents are necessary for testing the biological sample for a presence or absence of an analyte in the biological sample. In some embodiments, the receptacle contains a reagent that are necessary for dissolving the sample collector upon coming in contact with the sample collector. In some embodiments, accordingly, the sample collector is made of materials that dissolve upon contact with the reagent stored in the receptacle, thereby releasing the biological sample into the reservoir. In some embodiments, the system furthers comprise a cartridge comprising a chamber, wherein the cartridge and/or the chamber is connected to the reservoir via a docking unit, such that upon the cartridge and/or the chamber coming in contact with the reservoir, the released biological sample flows into the cartridge and/or the chamber. In some embodiments, the docking unit comprises a one-way pressure valve. In some embodiments, the docking unit comprises a resealable slit. In some embodiments, the cartridge containing the collected biological sample is covered or sealed. In some embodiments, the cartridge containing the collected biological sample is transported without causing damage or degradation to the collected biological sample. In some embodiments, the system further comprises software or bioinformatics for analyzing the presence of a pathology or disease, and recommending a treatment. In some embodiments, the software is an FDA-approved software. In some embodiments, the system comprises recommending a diet to a subject indicated of nutrition deficiency in the test results, without involving a dietitian or any health care professional.

### Subjects and Prognostic Risk Factors

Described herein, in some embodiments, are methods performed to detect a menstrual cycle disorder in a subject. In some embodiments, phenotypic or behavioral characteristics described herein (also referred to as “features” or “digital biomarkers”) of the subject are utilized to identify a subject at risk for a menstrual cycle disorder. In some embodiments, the features described herein are identified using the survey described herein.

In some embodiments, a subject is experiencing symptoms of a menstrual cycle disorder such as HMB; AUB; cramping, including cramping which is extreme; fatigue; prolonged menstruation; breast tenderness, which is extreme; migraine; back pain; weight loss; weight gain; one or more irregular cycles; or other symptoms. In some embodiments, the methods described herein are performed on a subject at risk for a menstrual cycle disorder. In some embodiments, an at-risk subject is a subject having HMB, a subject having AUB, a subject previously diagnosed with a menstrual cycle disorder, a subject having a family history of HMB, a subject having a family history of AUB, or a subject having a family history of a menstrual cycle disorder.

In some embodiments, phenotypic and behavioral characteristics is used to construct a profile of a subject, such as to predict or provide a risk factor of having a menstrual cycle disorder such as HMB or AUB. In some embodiments, inverse probability weighting is applied to adjust for potential confounders or to identify which factors is causative and/or closely associated with having and/or being at risk for a menstrual cycle disorder such as HMB or AUB. In some embodiments, longitudinal data is collected from a subject, such as through one or more surveys to collect phenotypic and behavioral characteristics of the subject. In some embodiments, such surveys are collected a single time, or is collected over days, weeks, months or years. In some embodiments, such surveys are collected from a single subject at least 1, 2, 3, 4, 5, 6, 7, 8, 9, or 10 times over any time period. In some embodiments, such surveys are collected from a single subject not more than 1, 2, 3, 4, 5, 6, 7, 8, 9, or 10 times over any time period. In some embodiments, such surveys are collected from a single subject about 1, 2, 3, 4, 5, 6, 7, 8, 9, or 10 times over any time period.

In some embodiments, surveys are collected from a number of different subjects or groups of subjects, and data compared. In some embodiments, survey data from one or more normal subjects (e.g., subjects without a menstrual cycle disorder) is compared with survey data from one or more subjects having HMB and/or AUB, or one or more subjects not having HMB or AUB but having other menstrual cycle disorders. In some embodiments, survey data from one or more subjects is compared with survey data from one or more subjects in the same group (e.g., survey data from a subject having HMB is compared with survey data from other subjects having HMB).

In some embodiments, survey data is collected and/or compared from at least 1, 5, 10, 50, 100, 500, or 1000 subjects. In some embodiments, survey data is collected and/or compared from not more than 1, 5, 10, 50, 100, 500, or 1000 subjects. In some embodiments, survey data is collected and/or compared from about 1, 5, 10, 50, 100, 500, or 1000 subjects. In some embodiments, survey data is collected and/or compared from between 1 and 1000 subjects, between 1 and 500 subjects, between 1 and 100 subjects, between 1 and 50 subjects, between 1 and 10 subjects, between 1 and 5 subjects, between 5 and 1000 subjects, between 5 and 500 subjects, between 5 and 100 subjects, between 5 and 50 subjects, between 5 and 10 subjects, between 10 and 1000 subjects, between 10 and 500 subjects, between 10 and 100 subjects, between 10 and 50 subjects, between 50 and 1000 subjects, between 50 and 500 subjects, between 50 and 100 subjects, between 100 and 1000 subjects, between 100 and 500 subjects, or between 500 and 1000 subjects. Subjects from whom survey data is collected is in the same group (e.g., normal menstrual bleeding, HMB, AUB, or another group) or in different groups. In some embodiments, a subject is in more than one group at the time of a single survey. In some embodiments, a subject is in one group at the time of a first survey, and in a different group at the time of a second survey. In some embodiments, a subject in a normal menstrual bleeding group is later in an HMB or AUB group, or a subject in an HMB or AUB group is later in a normal menstrual bleeding group.

In some embodiments, a survey collects data describing one or more phenotypic and/or behavioral characteristics (“digital biomarker”) of subjects. In some embodiments, phenotypic and/or behavioral characteristics of subjects includes characteristics listed in Table 4. In some embodiments, a survey collects data describing a phenotypic or behavioral characteristic (“digital biomarker”) that is not listed in Table 4. In some embodiments, the survey collects the information listed in FIGS. 1-23.

TABLE 4
Characteristics of a subject that is utilized to identify a subject at risk for a
menstrual cycle disorder
CHARACTERISTICS OF SUBJECTS

| | | | |
|---|---|---|---|
| Age | location of residence | Natural hair color | Annual income |
| Level of education | Relationship status | Frequency of | Current living situation |
| | | consensual physical | |
| | | touch (outside of | |
| | | sexual contact) | |
| Religious background | Current Religious | Military service | Mental health |
| | affiliation | | |
| menstruation phenotype | pregnancy state | number of pregnancies | No. and type of tampons or |
| | | | other menstrual products |
| | | | used during period cycle |
| height | weight | relationship status | birth sex |
| race | ethnicity | sexual orientation | gender |
| hormone therapy | alcohol consumption | cigarette consumption | marijuana consumption |
| hemp/CBD consumption | exercise | diet | stress level |
| sleep | fertility treatments | infertility | preeclampsia |
| pre-term labor | miscarriage | abortion | vaginal birth |
| C-section | live births | mother’s pregnancy | age of first menstruation |
| | | history | |
| mother’s age of first | Polycystic Ovarian | Ovarian cysts | Fibroids |
| menstruation | Syndrome | | |
| Breast Cancer | Ovarian Cancer | Endometrial Cancer | Pelvic Inflammatory |
| | | | Disease |
| Chronic or frequent UTIs | Ectopic Pregnancy | Heart Disease | disability |
| Type I Diabetes | Type II Diabetes | Autoimmune | Diminished Ovarian |
| | | conditions (Lupus, | Reserve |
| | | MS, rheumatoid | |
| | | arthritis) | |
| Endometriosis/Adenomyosis | family disease history | position of uterus | Painful periods |
| Heavy bleeding during period | Irregular bleeding on | Short interval between | Chronic pelvic pain |
| | or off period | periods | |
| Lower abdominal or back pain | Pain during | Pain while defecating, | Bloating, nausea, and |
| | penetrative sex | often with cycles of | vomiting |
| | | diarrhea and | |
| | | constipation | |
| Groin pain | Pain during exercise | vaginal yeast, viral, or | vaginal infection treated |
| | | bacterial infection | with antibiotics |
| medication usage | freeze preservation of | strength of pelvic floor | loss of bladder control |
| | eggs | | |
| birth control usage and type | duration of hormonal | sexual history | likelihood of orgasm during |
| | birth control usage | | sex |
| medication or supplement or | frequency of sex | occurrence of sexually | abnormal pap smear |
| device usage for sexual | | transmitted infection | |
| enhancement | | | |
| age of first sexual intercourse | number of sexual | sex during period | usage of emergency |
| | partners | | contraception |
| current menstrual bleeding | typical menstrual | cycle length | number of days of bleeding |
| pattern | bleeding pattern | | |
| consistency of bleeding pattern | ovulation cycle | pain during menstrual | type and level of pain |
| over menstrual cycles | | window | experienced during |
| | | | menstrual window |
| pain outside menstrual | type and level of pain | diet interaction with | activity or treatment used |
| window | experienced outside | menstrual-associated | for pain relief for |
| | menstrual window | pain | menstrual-associated pain |
| spasms in uterus not during | one-sided, low belly | rectal pain during | Number of sanitary |
| menstrual window | pain during ovulation | period or ovulation | products used together for |
| | (mittelschmerz) | | menstrual bleeding |
| passing blood clots during | menstrual bleeding | menstrual bleeding | interference of menstruation |
| menstruation | level during sleep | level during day | with typical daily activities |
| days of heavy flow during | pattern of bleeding | pattern of bleeding | pattern of bleeding |
| menstruation | during heavy flow | during lighter flow | throughout menstrual |
| | days | days | window |
| waist size | hip size | bust size | weight gain potential |
| weight loss potential | muscle mass | potential to increase or | areas of fat gain or |
| | | decrease muscle mass | accumulation |
| body areas having coarse hair | Color of menstrual | Consistency of | Odor of Menstrual fluid |
| | fluid | menstrual fluid | |
| Feel of menstrual fluid | Hair loss | acne | Body hair |
| History of environmental | Family history of | Use of antibiotics | Sex education |
| exposure | disease | | |
| Attitudes toward menstruation | Health insurance | ER visits | Preventative care practices |

In some embodiments, one or more of the characteristics listed in Table 4 is linked to HMB, AUB, or a menstrual cycle disorder. In some embodiments, a characteristic is linked with HMB, AUB, or a menstrual cycle disorder if it correlates with HMB, AUB, or a menstrual cycle disorder (e.g., is present with a disorder more often than a random association), if the characteristic causes HMB, AUB, or a menstrual cycle disorder, or if HMB, AUB, or a menstrual cycle disorder causes the characteristic. In some embodiments, a quantity or level of a characteristic is linked to HMB, AUB, or a menstrual cycle disorder. In some embodiments, a presence or absence of a characteristic is linked to HMB, AUB, or a menstrual cycle disorder. In some embodiments, a link between a characteristic and HMB, AUB, or a menstrual cycle disorder is statistically significant (e.g., p<0.1, p<0.05, or p<0.01).

In some embodiments, at least 1, at least 2, at least 3, at least 4, at least 5, at least 10, at least 15, at least 20, at least 30, or at least 40 characteristics is linked to HMB, AUB, or a menstrual cycle disorder. In some embodiments, no more than 1, no more than 2, no more than 3, no more than 4, no more than 5, no more than 10, no more than 15, no more than 20, no more than 30, or no more than 40 characteristics is linked to HMB, AUB, or a menstrual cycle disorder. In some embodiments, about 1, about 2, about 3, about 4, about 5, about 10, about 15, about 20, about 30, or about 40 characteristics is linked to HMB, AUB, or a menstrual cycle disorder. In some embodiments, between 1 and 40, between 1 and 30, between 1 and 20, between 1 and 10, between 1 and 5, between 5 and 40, between 5 and 30, between 5 and 20, between 5 and 10, between 10 and 40, between 10 and 30, between 10 and 20, between 20 and 40, between 20 and 30, or between 30 and 40 characteristics is linked to HMB, AUB, or a menstrual cycle disorder.

One or more phenotypic or behavioral characteristics is linked with a specific menstrual cycle disorder, such as endometriosis. In some embodiments, these phenotypic or behavioral characteristics is a subset of the characteristics listed in Table 4. In some embodiments, these phenotypic or behavioral characteristics is an additional characteristic not included in Table 4. In some embodiments, a list of phenotypic and behavioral characteristics that is linked with endometriosis is provided in Table 5.

TABLE 5
Characteristics of subjects which is indicative of Endometriosis
CHARACTERISTICS OF SUBJECTS WHICH CAN BE INDICATIVE OF ENDOMETRIOSIS

| | | | |
|---|---|---|---|
| endometriosis or | surgical diagnosis | age of diagnosis | years since diagnosis |
| adenomyosis diagnosis | | | |
| Ovarian endometrioma | Peritoneal superficial | Deep infiltrating | stage of endometriosis |
| diagnosis | endometriosis diagnosis | endometriosis diagnosis | |
| surgical treatment | reproductive status | removal of endometrial | retention of ovarian |
| | | implants | function/loss of |
| | | | reproductive ability |
| removal of uterus and | recurrence | Number of surgeries for | treatment of endometriosis |
| ovaries | | endometriosis | |
| Combination oral | Danazol treatment | GnRH agonist treatment | Progestins treatment |
| contraceptive pills | | | |
| treatment | | | |
| pelvic pain | pain killer usage for | hormone treatment for | pain type |
| | pelvic pain | pelvic pain | |
| pain severity | pain frequency | activities or treatments | activities or treatments |
| | | that lessen pelvic pain | that increase pelvic pain |

In some embodiments, a subject experiencing HMB or AUB experiences HMB or AUB during some or all of their menstrual cycles. In some embodiments, a subject experiencing HMB or AUB experiences HMB or AUB during at least 10%, 20%, 30%, 40%, 50%, 60%, 70%, 80%, 90%, or 100% of their menstrual cycles. In some embodiments, a subject experienced HMB or AUB one or more times in the past, is experiencing HMB or AUB for the first time, experience HMB or AUB sporadically, experience HMB or AUB regularly, or experience HMB or AUB during each menstrual cycle.

In some embodiments, a subject is a human subject. In some embodiments, a subject is a female subject. In some embodiments, the female subject has a chromosomal disorder such as a sex chromosome aberration. In some embodiments, a subject is transgender. In some embodiments, the female subject is experiencing puberty, in her reproductive years, experiencing menopause, or post-menopausal.

In some embodiments, a subject is experienced their first period. In some embodiments, a subject experiences their first period at 9 years of age, 10 years of age, 11 years of age, 12 years of age, 13 years of age, 14 years of age, 15 years of age, 16 years of age, 17 years of age, 18, years of age 19 years of age, or 20 years of age. In some embodiments, some subjects have experienced their first period earlier than 9 years of age or later than 20 years of age. In some embodiments, a subject's first period has been induced, for example, via a hormone therapy.

In some embodiments, a risk level is assigned to or calculated for a subject. In some embodiments, a risk level is dependent on the measurement of one or more markers (e.g., expression level of the one or more markers), a phenotypic or behavioral characteristic of the subject, or both. In some embodiments, a risk level of a subject is a quantitative or qualitative assessment of the risk that the subject. In some embodiments, a risk level of a subject indicates a likelihood that the subject has or will develop a condition, such as HMB, AUB, or another menstrual cycle disorder.

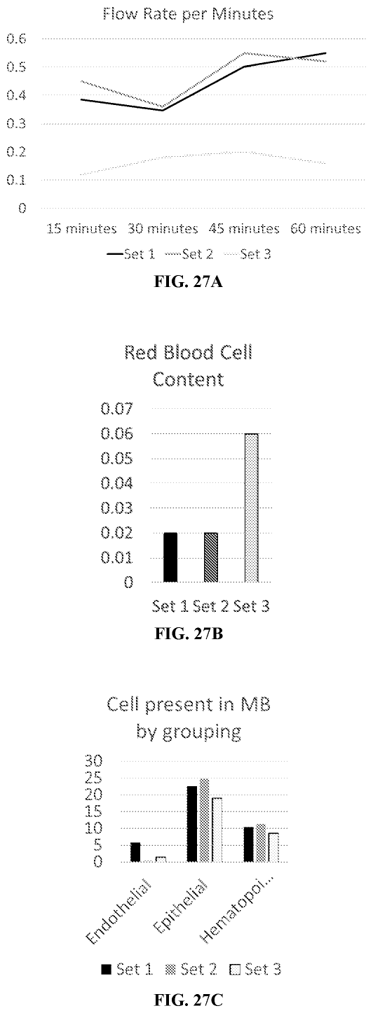

In some embodiments, a subject is stratified into a treatment group based on a risk level assigned to or calculated for them. In some embodiments, stratifying includes assigning a subject to a treatment group or excluding a subject from a treatment group. In some embodiments, stratification is described in further detail below, including in FIGS. 27-29 and in the examples.

In some embodiments, a reference subject is a subject who is experiencing HMB or AUB. In some embodiments, a reference subject is a subject with a diagnosis of a menstrual cycle disorder or another disorder or disease. In some embodiments, a sample from such a reference subject is a positive control.

In some embodiments, the subject has a household income of less than $25,000; $25,001-$50,000; $50,001-$75,000; $75,001-$100,000; $100,001-$150,000; $150,001-$200,000; or greater than $200,000. In some embodiments, the subject has a highest level of education of no high school, some high school, high school diploma (or equivalent), some college, trade school/technical school certification, associate's degree, bachelor's degree, master's degree, or doctorate or professional degree. In some embodiments, the survey asks the subject to list locations where the subject has lived.

### Menstrual Cycle

In some embodiments of methods provided herein, the digital biomarker comprises a property of a menstrual cycle. In some embodiments, menstrual fluid or other fluid from the vaginal cavity is collected from a reference subject to provide a reference sample using the methods described herein. In some embodiments, a reference subject is a healthy control subject. In some embodiments, a reference subject experiences normal menstrual bleeding during collection of the menstrual fluid. In some embodiments, a reference subject experiences normal menstrual bleeding during at least 50%, 60%, 70%, 80%, 90%, 95%, 99%, or 100% of their menstrual cycles. In some embodiments, menstrual fluid is collected from more than one reference subject to provide more than one reference sample. In some embodiments, the one or more reference samples serves as control samples. In some embodiments, a reference sample from a reference subject provides a baseline measurement of a property of a fluid sample, such as fluid from the vaginal cavity, such as menstrual fluid, against which a sample from another subject is compared. In some embodiments, the one or more reference samples is used to provide an average or a mean for comparison to one or more other subjects, or to provide a baseline or predetermined threshold for use in comparisons.

In some embodiments, the survey asks the subject questions about the subject's menstrual cycle and flow. In some embodiments, the survey asks the subject how long, in days, was the subject's last cycle (from Day 1 of the subject's period to the first day of the subject's next period). In some embodiments, the survey asks a subject that doesn't use an app or calendar to track the subject's menstrual cycle to approximate the length of the cycle, including without limitations, noticeably shorter than 28 days, shorter than 28 days but not by much, about 28 days, longer than 28 days but not by much, noticeably longer than 28 days, or don't know.

In some embodiments, the survey asks a subject whether during the subject's cycle, the period start date is completely predictable, somewhat predictable, or not at all predictable. In some embodiments, the survey asks a subject whether during the subject's cycle, the period end date is completely predictable, somewhat predictable, or not at all predictable. In some embodiments, the survey asks the subject to approximate the frequency of the subject's period, including without limitations, once a month, more than once a month, every three months, irregular, no identifiable pattern, infrequently, or never. In some embodiments, the survey asks the subject to describe how often the subject changes menstrual products, including without limitations, every hour, every 2 hours, every 4 hours, every 6 hours, every 8 hours, or every 10 hours. In some embodiments, the subject had a menstrual cycle during the past month. In some embodiments, the subject had most recent menstrual bleeding that was typical of what is normal for the subject. In some embodiments, the subject did not have a menstrual cycle due to birth control, pregnancy, or being perimenopausal. In some embodiments, the subject had bleeding when not on the subject's period.

In some embodiments, information on a typical menstrual cycle comprises the length of a menstrual cycle, the number of days of bleeding of a menstrual cycle, the frequency of a menstrual cycle. In some embodiments, the color of the menstrual fluid is black, brown, dark red, purple maroon, bright red, pink, orange, yellow, or gray, as depicted in FIG. 2. In some embodiments, the texture of the menstrual fluid is classified as runny liquid, thick consistency, clots/thick chunks, slippery with mucus, stringy, or a combination thereof. In some embodiments, the odor of the menstrual fluid is metallic/coppery, rotten, fishy, like onions, gym sweat, tangy/fermented, or sweet. In some embodiments, the feel of the menstrual flow is a drip, a trickle, gushing, a flood, or nothing. In some embodiments, the survey asks the subject to describe their period as spotting, very light, light, moderate, or heavy. An example of this is seen in FIG. 1. In some embodiments, the survey asks the subject how many days (including spotting) did the subject bleed for during the subject's period. In some embodiments, the survey asks the subject to chronologically list the sequence of spotting, light flow days, medium flow days and heavy flow days the subject experiences during menstruation. In some embodiments, the survey asks the subject if the subject's menstrual flow (heaviness) is nearly identical, somewhat similar, or completely different month to month.

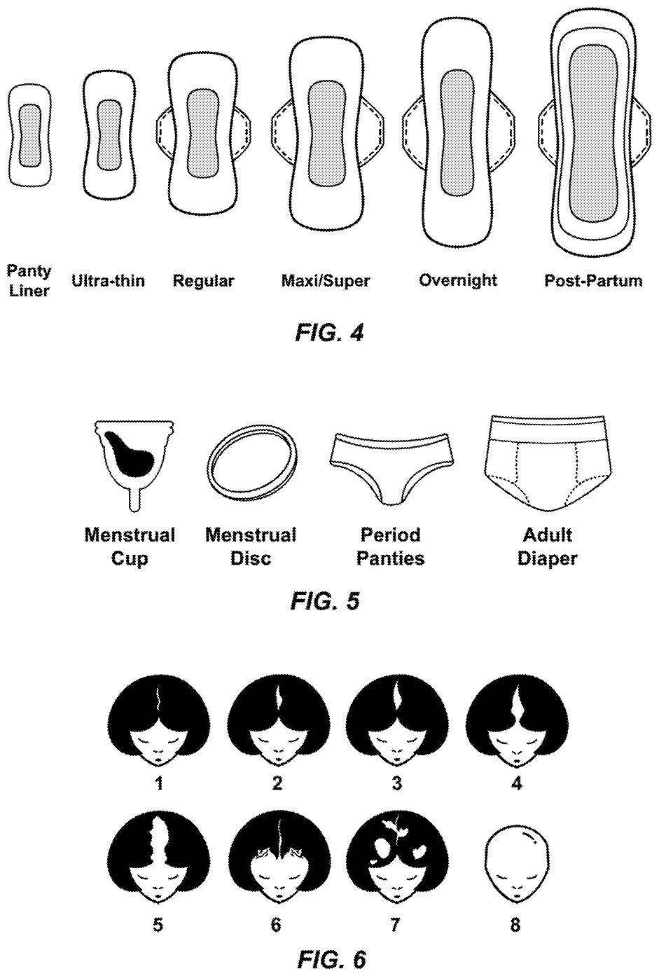

In some embodiments, the survey asks a subject how many times the subject bleeds through an outfit (not underwear) during a heavy flow, a medium flow, or a light flow, even when using typical menstrual products. In some embodiments, the survey asks a subject how many times the subject gets up, after the subject has gone to bed, to change menstrual products during a heavy flow, a medium flow, or a light flow. In some embodiments, the survey asks the subject how many light, regular, super, super plus, and ultra tampons the subject fully soaks on a given day (24 hours). In some embodiments, the survey asks the subject how many light, regular, super, super plus, and ultra tampons the subject fully soaks on a heavy flow, a medium flow, or a light flow day during the daytime and nighttime. An example of tampons sizes is seen in FIG. 3. In some embodiments, the survey asks the subject how many pantyliner, ultra-thin, regular, maxi/super, overnight, and post-partum pads the subject fully soaks on a heavy flow, a medium flow, or a light flow day (24 hours). In some embodiments, the survey asks the subject how many pantyliner, ultra-thin, regular, maxi/super, overnight, and post-partum pads the subject fully soaks on a heavy flow, a medium flow, or a light flow day during the daytime and nighttime. In some embodiments, an example of pad sizes is seen in FIG. 4. In some embodiments, the survey asks the subject to estimate how many times the subject empties or changes a menstrual cup, menstrual disk, period panties, or adult diaper per heavy flow, medium flow, or light flow day (24 hours). In some embodiments, the survey asks the subject to estimate how full the subject's menstrual cup, menstrual disk, period panties, or adult diaper was when changed. In some embodiments, these products are shown in FIG. 5.

In some embodiments, the survey collects information on how many menstrual products are fully used. In some embodiments, the survey collects information on how many menstrual products are used on a heavy flow day, a medium flow day, or a light flow day. In some embodiments, a survey collects information on how many products are used during the daytime or during the nighttime. In some embodiments, the menstrual products is a tampon, a pad, a menstrual cup, a menstrual disc, period panties, or an adult diaper. In some embodiments, the tampons is light, regular, super, super plus, or ultra tampons. In some embodiments, the pad is a panty-liner, an ultra-thing pad, a regular pad, a maxi/super pad, an overnight pad, or a post-partum pad.

In some embodiments, the survey collects information about menstrual product use. In some embodiments, the survey collects information about current use or past use of menstrual products. In some embodiments, the menstrual product is organic tampons, nonorganic tampons, organic pads, nonorganic pads, menstrual cup, menstrual disc, period panties, adult diaper, or bladder control pads. In some embodiments, the survey asks the subject which of the following menstrual products the subject currently uses, including without limitations, organic tampons, nonorganic tampons, organic pads, nonorganic pads, menstrual cup, menstrual disc, period panties, adult diaper, or bladder control pads. In some embodiments, the survey asks the subject which of the following menstrual products the subject has ever used, including without limitations, organic tampons, nonorganic tampons, organic pads, nonorganic pads, menstrual cup, menstrual disc, period panties, adult diaper, or bladder control pads.

In some embodiments, the survey asks the subject how many years the subject has used organic tampons. In some embodiments, the survey asks the subject how many years the subject has used nonorganic tampons. In some embodiments, the survey asks the subject how many years the subject has used organic pads. In some embodiments, the survey asks the subject how many years the subject has used nonorganic pads. In some embodiments, the survey asks the subject how many years the subject has used a menstrual cup. In some embodiments, the survey asks the subject how many years the subject has used a menstrual disk. In some embodiments, the survey asks the subject how many years the subject has used period panties. In some embodiments, the survey asks the subject how many years the subject has used adult diapers. In some embodiments, the survey asks the subject how many years the subject has used bladder control pads.

In some embodiments, the subject has a pre-existing condition/disability or physical limitation that influences the products the subject uses during the subject's period. In some embodiments, the survey asks the subject to explain why and how the subject manages the subject's menses. In some embodiments, the survey asks if religious or cultural norms have influenced the subject's menstrual management routine and to explain how.

In some embodiments, the survey collects information on cycle tracking. In some embodiments, the survey collects information on ovulation. In some embodiments, the survey collects information on methods of tracking ovulation. In some embodiments, the methods are spotting, mittelschmerz (lower belly pain, usually one-sided), LH urine test, PdG urine test, testing consistency of cervico-vaginal fluid, or, basal body temperature. In some embodiments, the survey collects information about methods of tracking a period. In some embodiments, the subject tracks aspects of the subject's menstrual cycle and/or ovulation using a paper calendar, a mobile app, or another method. If the subject tracks aspects of the subject's menstrual cycle and/or ovulation using a mobile app, the survey asks the subject which mobile app(s) the subject uses and why the subject likes one app more than another. If the subject tracks aspects of the subject's menstrual cycle and/or ovulation using a mobile app, the survey asks how accurate the mobile app is in predicting the start date of the subject's period, with the options being within 1, 2, 3, 4, 5, 6, 7, 8, 9, or 10 days of the start date of the subject's period.

In some embodiments, the survey collects information on missed days of work or school, reduced work capacity, impact on life, or impact on career due to menstruation. In some embodiments, the survey asks a subject how many days of work or school the subject misses due to a typical period. In some embodiments, the survey asks a subject how many days the subject shows up to work or school but is unable to be fully present or perform to full capacity due to a typical period. In some embodiments, the survey asks a subject to rate how much the subject's period has affected the subject's life or career on a scale of 1 to 10 (1=no impact, 10=failed classes or lost promotions due to period). In some embodiments, a subject experiences pain during menstruation. In some embodiments, menstrual pain is rated between 0 and 10 on the Mankoski pain scale.

In some embodiments, the survey collects information on menstrual clots. In some embodiments, the survey asks, during a typical period, how many times does the subject notice menstrual clots (blobs of blood, tissue, and mucus which can look gel-like or clumpy) whether it's seeing it or feeling it. If the subject experiences menstrual clots, the survey asks the subject to describe the size of the clots, including without limitations, smaller than a dime, size of a dime, size of a nickel, size of a quarter, or size of a ping-pong ball.

In some embodiments, the survey collects information on habits during menstruation. In some embodiments, the survey asks if finances affect how a subject manages the subject's period. In some embodiments, if finances do affect how a subject manages a period, the survey asks the subject to rate the following statements on a scale of 1 to 10 (1 being “I can't relate to this statement” and 10 being “this statement strongly resonates with me”): The survey asks if during a period, the subject wears specific clothing to hide or minimize leaks or packs a change of clothes in case of leaks. In some embodiments, the survey asks if as the start date of the subject's period approaches, the subject starts carrying spare menstrual products; pre-emptively changes their diet or exercise routine; pre-emptively takes pain medication in anticipation of pain; starts avoiding essential activities like grocery shopping; starts avoiding leisure activities like hanging out with friends; or reschedules appointments. In some embodiments, the survey asks a subject how often the subject checks the back of a seat or clothing for leaks during a typical period.

In some embodiments, a subject is experience symptoms either chronically, consistently, or occasionally. In some embodiments, symptoms include painful period, heavy bleeding during a period, irregular bleeding on or off a period, short interval between periods, chronic pelvic pain, lower abdominal or back pain, pain during penetrative sexual intercourse, pain during defecation (including normal defecation, diarrhea, and constipation), bloating, nausea, vomiting, groin pain, pain during exercise, migraine, fatigue, throbbing veins in legs, shooting rectal pain, acne, pale skin, increased body odor, or greasy hair. In some embodiments, a subject experiences spasms in their uterus when menstruating and/or when not menstruating. In some embodiments, a subject experiences one-sided, low belly pain during ovulation, i.e., mittelschmerz. In some embodiments, a patient experiences shooting rectal pain during a menstrual cycle or during ovulation. In some embodiments, the survey asks the subject to describe the role pain plays in the subject's life.

The survey asks which of the following helps relieve subject's menstrual pain: pain medication (over the counter), pain medication (prescription), Cannabis, ice, kava, hot bath, black cohosh, emptying bladder, relaxation, music, heating pad, meditation, sex/orgasm, acupuncture, lying down, massage, bowel movement, laxatives/enema, TENS unit (electrical stimulus), exercise, or other. In some embodiments, the survey asks the subject if eating sugar, animal products, alcohol, junk food, spicy food, salty food, or any other foods increases menstrual pain. In some embodiments, the survey asks the subject if eating salmon, banana, dark chocolate, citrus, spicy foods, dark leafy vegetables, watermelon, chamomile, herbal teas, or other foods helps relieve menstrual pain.

In some embodiments, the survey collects information on attitudes towards menstruation. In some embodiments, the survey collects information on the age in which a subject first learned about menstruation, how a subject first learned about menstruation, childhood communication on menstruation, shame around menstruation, influences on thoughts on menstruation, or availability of menstrual products. In some embodiments, the survey asks how old a subject was when the subject first learned about menstruation. In some embodiments, the subject is first learned about menstruation from, including without limitations, parents/family, friends/peers, puberty & self-help books, online resources, or other resources. In some embodiments, the survey asks a subject to rate how openly the subject's family communicated about periods when growing up on a scale of 1-10 (1=no discussions of menstruation, hiding menstrual products and waste; 10=encouraging and celebrating menstruation, menstrual products stored openly). In some embodiments, the survey asks what kinds of messages the subject received around menstruation while growing up. In some embodiments, the subject regularly hid period products from view when going to the bathroom in public in the past. In some embodiments, the subject currently hides period products from view when going to the bathroom in public. In some embodiments, the survey asks if a subject has ever felt shame around the subject's period. If the subject has felt shame in the past, the survey asks the subject to rate the shame on a scale of 1-10. In some embodiments, the survey may also ask the subject to rate the shame the subject currently feels on a scale of 1-10. In some embodiments, the survey asks the substitute to identify the major influences that shaped the subject's current views on menstruation, including without limitations, family, friends/peers, religious beliefs, cultural norms, menstruation advocacy groups or nonprofits, entertainment media (e.g. books, movies, TV shows), social media (e.g. influencers, threads on social networks), or other.

In some embodiments, the survey asks a subject to describe the availability of menstrual products in bathrooms outside the subject's home on a scale of 1-10. In some embodiments, the survey asks if a subject's school or workplace provides any of the following period-related accommodations: paid menstrual leave, ability to work from home during the subject's period, menstrual products available in bathrooms, freely available pain management products, such as pain medication or heating pads, or any other accommodations.

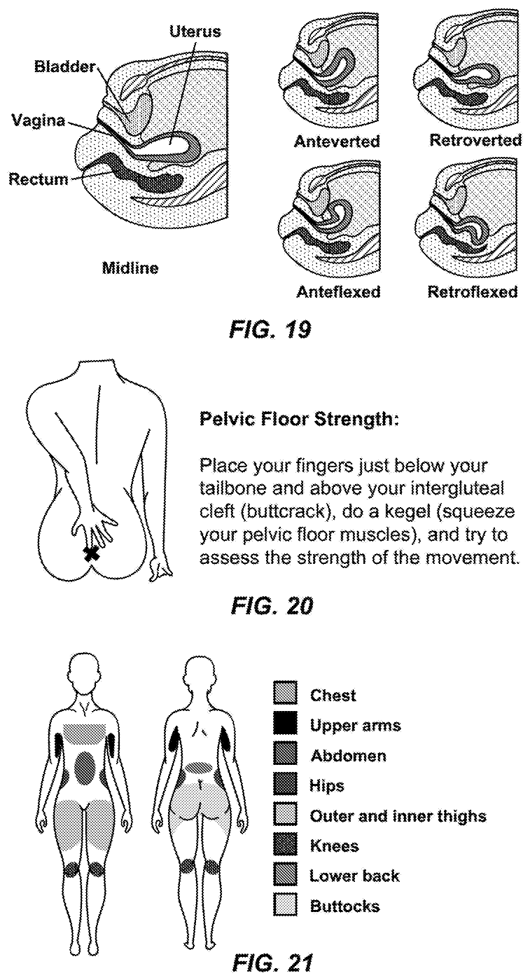

In some embodiments, a subject has a uterus which is anteverted (i.e., tipped forward or aimed toward the belly), retroverted (i.e., tipped backwards, or aimed toward the rectum), anteflexed (i.e., top of uterus points forward relative to cervix while front of uterus is concave), or retroflexed (i.e., top of uterus points backward relative to cervix while front of uterus is convex). In some embodiments, the survey asks the subject to indicate the position of the subject's uterus using FIG. 19 as a reference.

### Menstrual Cycle Disorders and Autoimmune Disorders

In some embodiments of methods herein, the digital biomarker comprises a property related to a menstrual cycle disorder or other autoimmune disorder. In some embodiments, a subject has a menstrual cycle disease or disorder, or other disorder which presents with, or cause, HMB or AUB. In some embodiments, diseases and disorders that a subject has include eating disorders; extreme weight loss; excessive exercise; polycystic ovary syndrome (PCOS); ovarian cysts; premature ovarian failure; breast cancer; ovarian cancer; infertility; diminished ovarian reserve; chronic or frequent urinary tract infections; ectopic pregnancy; heart disease; type 1 diabetes; type 2 diabetes; an autoimmune condition such as lupus, multiple sclerosis, or rheumatoid arthritis; pelvic inflammatory disease (PID); endometriosis; fibroids (e.g., uterine fibroids); adenomyosis; cervical cancer; endometrial cancer; uterine cancer; or infection of the cervix or endometrium. In some embodiments, HMB or AUB accompanies a disease affecting the kidney, liver, thyroid, or adrenal glands.

In some embodiments, the survey collects information on diagnosis with endometriosis or adenomyosis, age of diagnosis, tests involved in diagnosis, subtypes of endometriosis, stage of endometriosis, medication to treat endometriosis, surgical treatment to endometriosis, reoccurrence of endometriosis, pain associated with endometriosis, pain relief strategy, or pelvic pain. In some embodiments, a subject has endometriosis. In some embodiments, the subject has been surgically diagnosed with endometriosis or adenomyosis. In some embodiments, the subject, has been diagnosed with adenomyosis by either ultrasound or Mill. In some embodiments, the subject is suspected of having endometriosis or adenomyosis. In some embodiments, the survey asks subjects that have been surgically diagnosed with either endometriosis or adenomyosis by a doctor how old the subjects were when diagnosed and how many years has the subject lived with endometriosis. In some embodiments, the survey may also ask what tests the doctor used to diagnosis the subject, which could include ultrasound, pelvic exam, surgery, or other tests.

Endometriosis is a condition when endometrium grows on other pelvic organs. In some cases, types of endometriosis include ovarian endometrioma, peritoneal superficial endometriosis, deep infiltrating endometriosis, or adenomyosis. In some embodiments, endometriosis is stage 1 (minimal), stage 2 (mild), stage 3 (moderate), or stage 4 (severe). In some embodiments, a subject had endometriosis for at least 1 month, 6 months, 1 year, 2 years, 3 years, 4 years, 5 years, or more. In some embodiments, a subject has had surgery for endometriosis. In some embodiments, the surgery is conservative, semiconservative, or radical. In some embodiments, the medication is combination oral contraceptive pills (COCs), Danazol, GnRH agonist, or progestins.

In some embodiments, a subject has adenomyosis. Adenomyosis is a condition where the endometrium breaks through or invades the muscle wall of the uterus. In some cases, symptoms of adenomyosis include menstrual cramps, lower abdominal pressure, bloating before periods, or HMB. In some embodiments, a subject has had adenomyosis for at least 1 month, 6 months, 1 year, 2 years, 3 years, 4 years, 5 years, or more.

In some embodiments, the survey collects information on fibroids. In some embodiments, the information is age at diagnosis, years since diagnosis, method of diagnosis, type of fibroids, surgical treatment, age at treatment, reoccurrence at treatment, medication to treat fibroids, pelvic pain, pain relief, and frequency of pelvic pain. in some embodiments, the type of fibroid is intramural (in the uterine wall), subserosal (on the outside of the uterine wall), submucosal (under the lining of the uterine cavity), or pedunculated (a stalk like growth that can attach to many places). In some embodiments, the surgical treatment is myomectomy (laparoscopic/open/hysteroscopic), laparoscopic myomectomy, open myomectomy, hysteroscopic myomectomy, hysterectomy, uterine fibroid embolization (ufe), endometrial ablation, myolysis, partial hysterectomy, MM-guided focused ultrasound surgery, watching/wait and see/waiting for menopause, or complementary and/or alternative medicine (CAM). In some embodiments, the medication is combination oral contraceptive pills (COCs), GnRH agonist, Progestins, Tranexamic acid (Lysteda, Cyklokapron), or Ulipristal acetate.

In some embodiments, the survey asks how much pelvic pain the subject had during the subject's last period, including without limitations, no pain, mild cramps (medication never or rarely needed to continue daily activities), moderate cramps (medication usually needed to continue daily activities), or severe cramps (medication and bed rest needed to continue daily activities). In some embodiments, the survey asks how often the subject had pelvic pain during the subject's period, including without limitations, seldom (less than a quarter of my periods), often (a quarter to half of my periods), usually (more than half of my periods), or always (every period). In some embodiments, the subject is taken pain-killers or hormones for pelvic pain during the subject's last period. In some embodiments, the painkillers may be prescribed by a doctor, over-the-counter (e.g. aspirin, ibuprofen, paracetamol/acetaminophen, naproxen), hormones, or a cannabinoid based product.

The survey asks if during the subject's last period, the pelvic pain prevented the subject from going to work or school or carrying out daily activities (even if taking pain-killers). In some embodiments, the survey asks the subject to rate how severe the subject's pelvic pain was at its worst during the subject's last period from 0-10 on the Mankoski Pain Scale.

Th subject may have had pelvic pain within the last 3 months that felt, including without limitations, throbbing, shooting, stabbing, sharp, cramping, gnawing, hot-burning, aching, heavy, tender, splitting, tiring-exhausting, sickening, fearful, punishing-cruel, or other. In some embodiments, the survey asks the subject to identify factors that makes the pelvic pain worse such as sitting, full bladder or urinating, time of day, stress, bowel movement, constipation, full meal, intercourse or orgasm, standing or walking, exercise, weather, contact with clothing, or coughing/sneezing. In some embodiments, the survey asks what helps the subject's pelvic pain, including without limitations, prescription pain medication, over the counter pain medication, relaxation, lying down, music, massage, ice, heating pad, bowel movement, hot bath, meditation, laxatives/enema, emptying bladder, cannabinoid based products, herbal remedies, black cohosh, kava, or other.

In some embodiments, the survey collects information on an autoimmune disease. In some embodiments, the autoimmune disease is Alopecia areata, Antiphospholipid antibody syndrome (APL), Autoimmune hepatitis, Celiac disease, Crohn's disease, Diabetes type 1, Graves' disease, Guillain-Barre syndrome, Hashimoto's disease, Hemolytic anemia, Idiopathic thrombocytopenic purpura (ITP), Inflammatory bowel disease (IBD), Inflammatory myopathies, Multiple sclerosis (MS), Myasthenia gravis (MG), Primary biliary cirrhosis, Psoriasis, Rheumatoid arthritis, Scleroderma, Sjogren's syndrome, Systemic lupus erythematosus, Vitiligo, or a combination thereof. In some embodiments, the survey collects information on age of first diagnosis, years since diagnosis, or time to diagnosis. In some embodiments, the survey collects information on medications or complementary or alternative medicine used to treat an autoimmune disorder. In some embodiments, the medication is Corticosteroids (e.g. Prednisone); NSAIDS (e.g. Advil); DMARDs (e.g. Methotrexate, Plaquenil); Janus kinase inhibitor (e.g. Xeljanz); Calcineurin inhibitor (e.g. Neoral, Astagraf XL); mTOR inhibitor (e.g. Rapamune, Afinitor); IMDH inhibitor (e.g. Imuran, CellCept); DMTs (e.g. Lemtrada, Aubagio); Hormones (e.g. Levothyroxine); Biologic (e.g. Humira, Rituxan, Stelara); Monoclonal antibodies (e.g. Simulect, Zinbryta); IVIG (e.g. Immunoglobulin therapy); Plasmapheresis; surgery; or other treatments. In some embodiments, the complementary or alternative medicine is vitamins and supplements (i.e. Vitamin D, probiotics), yoga/exercise, acupuncture, diet, elimination of toxins, functional medicine, nutritionist, physical therapist, or a combination thereof. In some embodiments, the survey collects information on reducing flareups, managing symptoms, a relationship between menstruation and an autoimmune disorder, or impact on quality of life.

In some embodiments, the survey asks what symptoms a subject experiences during a flare-up, including without limitations, fatigue, joint pain, digestive issues, dermatological issues (i.e. rash, irritation), pain, hair loss, weight loss, weight gain, muscle weakness, brain fog, anemia, numbness and tingling, swollen glands, or other symptoms. In some embodiments, the survey asks how a subject knows a flare up is imminent, including without limitations, unknown, stress, eating a “trigger” food, a major life change, overdoing it, not taking prescribed medicine, change in weather, change in seasons, bacterial or viral infection, or lack of sleep.

In some embodiments, the subject believes there is a correlation between the subject's period and symptoms/flares of the autoimmune disease(s). In some embodiments, the subject's period is impacted during an autoimmune disease(s) flare up, including without limitations, heavier flow than normal, lighter flow than normal, irregular or increased frequency of cramping, increased intensity of cramping, or increase in clotting. In some embodiments, the survey asks how a subject's autoimmune disease(s) is impacted when the subject has their period, including without limitations, more likely to experience a flare, increased fatigue, increased pain, autoimmune symptoms worsen, or autoimmune symptoms improve. In some embodiments, the survey asks if during the subject's last period, the symptoms (either period related or autoimmune related) prevented the subject from going to work or school or carrying out daily activities. In some embodiments, the subject's ability to work full-time, ability to work part-time, ability to attend school, social life, romantic life, recreational travel, parenting, hobbies, or exercise is impacted by the subject's autoimmune disease greatly, somewhat, or not at all.

In some embodiments, the subject has been or is currently pregnant. In some embodiments, if the subject has been or is pregnant, the subject's autoimmune disease(s) was impacted during pregnancy, including without limitations, autoimmune disease(s) went into a non-drug-induced remission, experienced a flare of autoimmune disease(s), autoimmune symptoms improved, autoimmune symptoms worsened, or other. In some embodiments, the subject has been on treatment prior to getting pregnant, and the survey asks if the subject stayed on this treatment regimen through the pregnancy. In some embodiments, the subject's autoimmune diseases have been impacted post-partum, including without limitations, autoimmune disease(s) went into a non-drug-induced remission, experienced a flare of autoimmune disease(s), autoimmune symptoms improved, autoimmune symptoms worsened, or other.

### Sexual Behaviors and Education

In some embodiments of methods provided herein, the digital biomarker comprises a property related to a sexual behavior or education of the subject as described herein. In some embodiments, the survey collects information on sexual education. In some embodiments, the sexual education is abstinence, consent, how to say “no” to sex, different birth control methods, STIs, how to use a condom, how to prevent HIV, HPV vaccine, or a combination thereof. In some embodiments, the sexual education is formal or informal. In some embodiments, the survey collects information about the age of sexual education or the source of sexual education. In some embodiments, the survey collects information about communication about sex.

In some embodiments, the survey asks the subject to identify the sex-ed topics for which the subject received “formal” or informal instruction before the age of 18, including without limitations, abstinence, consent, how to say “no” to sex, different birth control methods, STIs, how to use a condom, how to prevent HIV, or the HPV vaccine. In some embodiments, if the subject received formal sex-ed instruction, the subject received it prior to the subject's first experience of intercourse.

In some embodiments, the survey asks the subject to identify the sex-ed topics for which the subject received “informal” instruction (from parents or guardians) before the age of 18, including without limitations, abstinence, consent, how to say “no” to sex, different birth control methods, STIs, how to use a condom, how to prevent HIV, or the HPV vaccine. If the subject received informal sex-ed instruction. In some embodiments, the subject is received it prior to the subject's first experience of intercourse.

In some embodiments, the survey asks which sources of sex education the subject received outside of school, including without limitations, puberty & self-help books, bird & the bees conversation with my parents, friends and siblings, pornographic videos & photos, romance novels, or other.

In some embodiments, the subject speak with the subject's partner about sex, sexual needs and desires often, when necessary, or never. In some embodiments, the subject speaks with friends about the subject's sex life often, when necessary, or never.

In some embodiments, the survey collects information on being sexually active, frequency of sex, STIs, age of first vaginal intercourse, sex during a period, emergency contraception, sexual pleasure, masturbation, orgasm, orgasm supplements, sex toys, erotic literature, or other materials to enhance sexual arousal. In some embodiments, the subject has been diagnosed with dyspareunia (painful sexual intercourse) or experienced genital pain just before, during or after sexual intercourse. In some embodiments, the subject has been treated for a sexually transmitted infection. In some embodiments, the survey asks how old the subject was during the subject's first sexual encounter. In some embodiments, the survey asks how old the subject was when the subject first had vaginal intercourse.

In some embodiments, a subject is sexually active. In some embodiments, a subject has sexual intercourse less than 10 times per year, about 10 times per year, about 1-3 times per month, about 1-3 times per week, or more than 3 times per week. In some embodiments, a subject had their first sexual encounter (e.g., sexual intercourse) at age 13, 14, 15, 16, 17, 18, 19, 20, 21, 22, 23, 24, 25, older than age 25, or younger than age 13. In some embodiments, a subject has sexual intercourse during their period or not have sexual intercourse during their period.

In some embodiments, the survey asks the subject what frequency best describes how often the subject receives welcome and consensual physical touch (outside of sexual contact), including without limitations, rarely, occasionally, regularly, or frequently.

In some embodiments, a subject had 0, 1, 2, 3, 4, 5, 6, 7, 8, 9, 10, or more sexual partners during their lifetime. In some embodiments, a subject had 0, 1, 2, 3, 4, 5, 6, 7, 8, 9, 10, or more sexual partners at the time a sample is collected.

In some embodiments, the subject has sex during menstruation. If the subject does not have sex during menstruation, the survey asks if this is due to the subject's preference or the preference of the subject's partner. In some embodiments, the survey asks how many times the subject has used emergency contraception (Plan B, Ella, etc). In some embodiments, the subject knows that Plan B is ordered through Amazon.

In some embodiments, the survey asks about a subject's views on masturbation. In some embodiments, the survey asks if the subject typically reaches orgasm while masturbating; if the subject has ever had an orgasm with a sexual partner; or if the subject has ever taken over-the-counter medications or supplements to enhance orgasm and which medications or supplements were taken; if the subject uses “toys” (such as vibrators) to achieve sexual pleasure; if the subject uses visual material (pictures/videos) to enhance sexual arousal; if the subject uses aural material to enhance sexual arousal (music, erotic podcasts); if the subject uses erotic literature to enhance sexual arousal; if the subject uses any apps to improve the subject's sex life, and if so, the survey asks what apps the subject uses.

In some embodiments, a subject has a sexually transmitted infection or has had a sexually transmitted infection in the past. In some embodiments, a sexually transmitted infection is bacterial vaginosis, Chlamydia, gonorrhea, genital herpes, hepatitis, human immunodeficiency virus, acquired immunodeficiency syndrome, human papillomavirus, pelvic inflammatory disease, syphilis, or trichomoniasis.

### Pregnancy and Childbirth

In some embodiments of methods provided herein, the digital biomarker is a property related to pregnancy, fertility, and childbirth of the subject. In some embodiments, the survey collets information about fertility. In some embodiments, the survey collects information on attempts to get pregnant, time to get pregnant, fertility treatment, infertility, egg freezing, egg donor, gestational carrier, embryo freezing, pregnancy, pre-term labor, pre-eclampsia, miscarriage, abortion, or frequency of medical care during pregnancy. In some embodiments, the survey collects information on giving birth, number of times giving birth, C-sections, vaginal delivery, breastfeeding, formula feeling, mothers age at birth of first child, mother's age at birth of last child, or a combination thereof. In some embodiments, the survey collects information about medical treatment after birth, time off work after birth before returning to work, childcare after birth, stiches after birth, postpartum hemorrhaging, postpartum bleeding, passing small clots, passing clots bigger than the size of a quarter, headaches, high blood pressure, swelling in hands and feet, blurred vision, sudden weight gain, leg pain, pain that felt like a pulled muscle, swelling in legs, painful urination, difficulty urinating, constipation, chills or fever, vaginal discharge with no odor, vaginal discharge with odor, heart palpitations, chest pain, difficulty breathing, afterpains (abdominal pains which can be especially feel while breastfeeding), increasingly worsening lower belly pain, back pain, incontinence, soreness in the perineal and/or vaginal area, anxiety, stress, depression, obsessive-compulsive behavior, emotional instability, sadness, diastasis recti (a gap between the right and left abdominal wall muscles which can give the appearance of a pregnant belly months after birth), postpartum preeclampsia, stroke, deep vein thrombosis, c-section infection, episiotomy infection, hemorrhoids, baby blues (overwhelmed, fragile, sad for a few weeks after birth), postpartum depression (mood swings, sadness, insomnia, irritability, suicidal thoughts, inability to care for new born that lasts longer than a few weeks), lactation consultant, experiences breastfeeding, pelvic therapy, sex drive, weight gain, changes in shoe size, hair loss, stretch marks, return of period after pregnancy, or a combination thereof. In some embodiments, the subject is experienced postpartum hemorrhaging to an extent that the subject filled more than one pad every hour.

In some embodiments, a subject is pregnant. In some embodiments, the pregnancy is a normal pregnancy or a complicated pregnancy. In some embodiments, complications comprise pre-eclampsia, eclampsia, stillbirth, a history of stillbirth, miscarriage, a history of miscarriage, high blood pressure, gestational diabetes, preterm labor, nausea and vomiting, hyperemesis gravidarum, or iron deficiency anemia. In some embodiments, a pregnant subject is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 weeks pregnant.

In some embodiments, a subject is a post-partum subject. In some embodiments, a postpartum subject is at least 1, 2, 3, 4, 5, 6, 7, 8, 9, 10, 11, 12, or more weeks post-partum.

In some embodiments, the survey asks if a subject has ever given birth. If the subject has given birth, the survey asks how many times the subject has given birth and the age at which the subject first gave birth. In some embodiments, the survey asks if a subject has ever had a C-section or vaginal delivery. In some embodiments, a subject has a baby. In some embodiments, a baby is 0 months, 1 month, 2 months, 3 months, 4 months, 5 months, 6 months, 7 months, 8 months, 9 months, 10 months, 11 months, 12 months, 18 months, or 24 months of age.

In some embodiments, a subject has been pregnant before. In some embodiments, a subject has had 0, 1, 2, 3, 4, 5, 6, 7, 8, 9, 10, or more pregnancies. In some embodiments, a subject has had their first pregnancy at age 13, 14, 15, 16, 17, 18, 19, 20, 21, 22, 23, 24, 25, 26, 27, 28, 29, 30, 31, 32, 33, 34, 35, 36, 37, 38, 39, or 40. In some embodiments, a subject has had their first pregnancy earlier than age 13 or later than age 40. In some embodiments, a subject has had 0, 1, 2, 3, 4, 5, 6, 7, 8, 9, 10, or more live births. In some embodiments, a subject has had 0, 1, 2, 3, 4, 5, 6, 7, 8, 9, 10, or more stillbirths. In some embodiments, a subject has had 0, 1, 2, 3, 4, 5, 6, 7, 8, 9, 10, or more miscarriages. In some embodiments, a birth (e.g., a live birth or a still birth) is via a Cesarean section or a vaginal delivery.

In some embodiments, the survey asks how often the subject saw a doctor during pregnancy, including without limitations, once a month until week 28, then once a month until week 36, then every week in the last month of pregnancy; once a trimester; once before birth; at birth; or other. In some embodiments, the survey asks how long after giving birth was the first time the subject saw a doctor again, including without limitations, within 3 weeks of birth for a normal checkup, for a 6 week normal checkup, or before a normal scheduled checkup for an emergency. In some embodiments, the subject has taken leave for 1 week, 2 weeks, 1 month, 2 months, 3 months, 4 to 6 months, 6 to 12 months, or longer than 12 months before returning to work. In some embodiments, the subject does not return to work.

In some embodiments, a subject is having or has a history of miscarriage. In some embodiments, a subject has had a miscarriage in the past 1, 2, 3, 4, 5, 6, 7, 8, 9 10, 11, or 12 months. In some embodiments, a subject has had a miscarriage in the past 1, 2, 3, 4, 5, 6, 7, 8, 9, or 10 years. In some embodiments, a subject has had multiple miscarriages. Some subjects has a family history of miscarriage. If the subject has had one or more miscarriages, the survey asks how many weeks pregnant the subject was during the last miscarriage and how long it took for the subject's period to return after the last miscarriage.

In some embodiments, a subject has had an abortion in the past. In some embodiments, a subject has had more than one abortion. In some embodiments, a subject has had 0, 1, 2, 3, 4, or more abortions. If the subject has had one or more abortions, the survey asks how many weeks pregnant the subject was during the last abortion and how long it took for the subject's period to return after the last abortion.

In some embodiments, a subject has frozen their eggs. In some embodiments, a subject has gone through 1, 2, 3, 4, or more cycles of egg freezing. In some embodiments, a subject is frozen their embryos. In some embodiments, a subject has gone through 1, 2, 3, 4, or more cycles of egg freezing. In some embodiments, the subject has been an egg donor or gestational carrier for another individual or couple. In some embodiments, the subject is frozen the subject's embryos. If so, the survey asks how many cycles the subject has gone through.

In some embodiments, a subject is exclusively breastfeeding their baby, mostly breastfeed their baby, breastfeed their baby about half the time, breastfeed their baby occasionally, or never breastfeed their baby. In some embodiments, the survey asks how many months the subject pursed this newborn feeding strategy. In some embodiments, the subject has had a partner to help with nighttime feedings or wakings. In some embodiments, the survey asks if the subject had extended family, friends or nanny care to help with baby care and household care after birth. In some embodiments, the subject had help for at least about 2 weeks, 1 month, 2 months, 3 months, 4 to 6 months, 6 to 12 months, or longer than 12 months.

In some embodiments, the subject used a lactation consultant. In some embodiments, the survey asks the subject to rate how stressful the subject found the experience of getting the newborn to latch on a scale of 1-10. In some embodiments, the survey asks the subject to rate how frustrating the subject found the experience of nursing to be the first time. In some embodiments, the subject is experienced sore nipples and breasts after birth, and if so, the survey asks how many weeks the soreness lasted after the subject first started breast feeding. In some embodiments, the survey asks if the soreness resolved after the subject's baby latched correctly. In some embodiments, the subject is developed painful cracks on the nipples, mastitis (inflammation of the breasts), or breast abscesses (painful build-up of pus in breasts). In some embodiments, the subject has seen a pelvic therapist during pregnancy to prepare for giving birth or after birth for pelvic massage, back pain, incontinence or other pregnancy/postpartum issues,

In some embodiments, the survey asks which of the following items the subject used in postpartum care: ibuprofen/acetaminophen, sitz bath, perineal spray, healing foam, maxi pads, upside down squirt bottle to wash perineal area, postpartum underwear, ice packs for vaginal/perineal area, kegels, nipple balm, or other. In some embodiments, the subject is found it easy to learn about the over the counter products (such as postpartum underwear, “MomWasher” peri bottles) which were most useful in the subject's postpartum care. In some embodiments, the survey asks from whom did the subject find out about the most useful products to use during postpartum care, including without limitations, doctor, nurse, lactation consultant, pregnancy preparation classes, family and friends, books, pregnancy/childbirth/parent Facebook groups, Instagram accounts, Googling, or other. In some embodiments, the subject is bought most products for postpartum care at big box retailers (Target, Walmart), pharmacies (CVS, Walgreens etc.), Amazon, independent vendors specializing in baby care with store fronts, or independent vendors specializing in baby care on-line.

In some embodiments, the survey asks how long it took after childbirth for the subject's sex drive to return, including without limitations, within a month, 1 to 3 months, 3 to 6 months, 6 to 12 months, or longer than 12 months. In some embodiments, the survey asks how much weight the subject gained during pregnancy and if the subject returned to a pre-pregnancy weight after giving birth. If the subject did return to the subject's pre-pregnancy weight, the survey asks the subject to rate on a scale of 1 to 10, 1 being easy and 10 being very difficult, how hard it was to return to a pre-pregnancy weight. In some embodiments, the survey asks how long it took for the subject to return to a pre-pregnancy weight. In some embodiments, the subject experienced an increase in shoe size during pregnancy, and if so, the survey asks how long it took the subject to return to the subject's pre-pregnancy shoe size, including without limitations, within a month, 1 to 3 months, 3 to 6 months, 6 to 12 months, longer than 12 months, or it did not revert. In some embodiments, the subject is experienced hair loss after pregnancy, and if so, the survey asks if the hair loss stopped within a month, 1 to 3 months, 3 to 6 months, 6 to 12 months, longer than 12 months, or it did not stop. In some embodiments, the subject experienced stretch marks during pregnancy, and if so, the survey asks how long it took the stretch marks to go away, including without limitations, within a month, 1 to 3 months, 3 to 6 months, 6 to 12 months, longer than 12 months, or they did not go away.

In some embodiments, the survey asks how long after giving birth did it take for the subject's period to return, including without limitations, within a month, 1 to 3 months, 3 to 6 months, 6 to 12 months, longer than 12 months, or other. In some embodiments, the subject's period may be different following childbirth.

In some embodiments, a subject is experiencing or at risk for infertility. Infertility comprises an inability to become pregnant (conceive) after one year of trying to conceive. In some embodiments, subjects experiencing or at risk for infertility is experiencing HMB or AUB, is at risk for HMB or AUB, and/or has or be at risk for a menstrual cycle disorder. In some embodiments, the subject is tried to get pregnant, tried to get pregnant for more than 6 months in a row without succeeding, sought treatment for fertility in any clinic, had a fertility work up, been diagnosed with infertility, or had a partner diagnosed with infertility.

In some embodiments, a subject has a family history of fertility or infertility. In some embodiments, the mother of the subject's age when they had their first child is at least 13, at least 15, at least 20, at least 25, at least 30, at least 35, or at least 40 years of age. In some embodiments, the mother of the subject's age when they had their last child is at least 13, at least 15, at least 20, at least 25, at least 30, at least 35, or at least 40 years of age.

### Menopause and Perimenopause

In some embodiments of method provided herein, the digital biomarker comprises a phenotypical or behavioral property related to menopause or perimenopause. In some embodiments, a subject is experiencing menopause. In some embodiments, a subject experiencing menopause has entered menopause early. Early menopause is menopause which begins before a threshold age, such as 40 years of age. Early menopause is caused by genetic factors, chemical factors (e.g., chemotherapy), a combination of genetic and chemical factors, or unknown factors. In some embodiments, a subject has entered menopause at 25 years of age, 26 years of age, 27 years of age, 28 years of age, 29 years of age, 30 years of age, 31 years of age, 32 years of age, 33 years of age, 34 years of age, 35 years of age, 36 years of age, 37 years of age, 38 years of age, 39 years of age, 40 years of age, 41 years of age, 42 years of age, 43 years of age, 44 years of age, 45 years of age, 46 years of age, 47 years of age, 48 years of age, 49 years of age, 50 years of age, or over 50 years of age.

In some embodiments, the survey collects information on menopause and perimenopause. In some embodiments, the survey collects information on Premature Ovarian Failure, use of hormone tests, changes to menstrual cycle, vasomotor symptoms, sleep issues, mood changes, physical symptoms, genitourinary symptoms, appearance changes, life changes due to menopause, hormone replacement therapy, bone loss, osteoporosis, or treatments for osteoporosis. In some embodiments, the hormone test is for Follicle-stimulating hormone (FSH), Estrogen (estradiol), Thyroid-stimulating hormone (TSH), or Anti-Mullerian hormone (AMH). In some embodiments, the treatments for osteoporosis comprise Vaginal estrogen, Low-dose antidepressants, Gabapentin (Neurontin, Gralise, others), Clonidine (Catapres, Kapvay, others), or other medications to prevent or treat osteoporosis. In some embodiments, the survey asks the subject if the hormone tests indicated any of the following: currently in menopause, not in menopause, or not sure (test results were unclear).

In some embodiments, the subject recently noticed changes that the subject think might indicate movement towards menopause, with the options being yes, no, or not sure. In some embodiments, these changes occurred within 1, 2, 3, 4, 5, 6, or 7 years.

In some embodiments, the subject's menstrual cycle recently become less regular than it used to be. In some embodiments, the cycle changed such that the subject loses more blood, loses less blood, the blood loss is irregular, there are more days in between periods, there are less days in between periods, the amount of flow has changed, the consistency of flow has changed.

In some embodiments, the subject experienced any of the following vasomotor symptoms (due to constriction or dilation of blood vessels): hot flashes, night sweats, feeling flushed, racing heart, perspiration, chills, or heart palpitations. In some embodiments, the subject experienced sleep issues such as insomnia, waking up during the night, sweating in the night, difficulty falling asleep due to physical discomfort, or daytime drowsiness. In some embodiments, the subject experienced mood changes such as irritability, fatigue, confusion, anger, tension, depression, anxiety, mood swings, or changes in libido (sex drive). In some embodiments, the subject experienced physical symptoms such as headaches, sore/tender breasts, muscle aches and pains, joint pain, or digestive issues. In some embodiments, the subject experienced genitourinary (vaginal & urethra) symptoms such as vaginal dryness, vaginal itchiness, vaginal discharge, burning with urination, frequent urination, recurrent UTIs, urinary incontinence, pain with intercourse, or shortening and/or tightening of the vaginal canal. In some embodiments, the subject experienced changes to appearance such as undesired weight gain, changes to the subject's silhouette (i.e. wider waist), appearance of acne, thinner hair on the top of the subject's head, thicker facial/body hair, less-even skin tone, or loss of skin elasticity.

In some embodiments, the subject made any of the following life changes in anticipation of the transition to menopause, or to counteract the apparent effects of menopause: increased calcium intake, increased vitamin D intake, quit smoking, quit drinking alcohol, reduced alcohol intake, increased physical activity, adopted dietary changes aimed toward weight loss, increased my performance of high impact weight-bearing exercises (i.e. running, skipping, dancing), started to take non-hormone medications, or started to use topic hormone therapies (normally an estrogen cream/insert/gel that is applied to the vagina).

In some embodiments, the subject sought hormone replacement therapy (HRT) in association with menopause. HRT may have started within 1 year, 2, years, 3, years, 4, years, 5, years, 6, years, 7 years, 8 years, 9 years or 10 years. In some embodiments, the subject experienced adverse outcomes after completing HRT such as bloating/swelling/tenderness in breasts, headaches and/or migraines, nausea, indigestion, unexpected vaginal bleeding, mood swings, acne, breast cancer, ovarian cancer, uterine cancer, blood clots, heart disease, or stroke.

In some embodiments, the subject experienced bone loss. In some embodiments, the subject is (or suspect to have) osteoporosis. In some embodiments, the subject has been diagnosed with osteoporosis by a doctor. In some embodiments, the subject used any of the following treatments: vaginal estrogen, low-dose antidepressants, Gabapentin (Neurontin, Gralise, others), Clonidine (Catapres, Kapvay, others), or medications to prevent or treat osteoporosis. In some embodiments, the subject used alternative treatments such as acupuncture, yoga, bioidentical hormones, plant estrogens (phytoestrogens), black cohosh, aromatherapy, meditation & mindfulness, hypnosis, vigorous exercise, cognitive behavioral therapy program, therapeutic massage, or isoflavens (soy).

### Medical History

In some embodiments of method provided herein, the digital biomarker comprises a phenotypical or behavioral property related to the medical history of the subject. In some embodiments, the survey collects information on medical history. In some embodiments, the medical history is the age of first period or the age of mother's first period. In some embodiments, the information is a diagnosis of abnormal menstrual bleeding, abnormal pap, anemia, asthma, premature menarche, delayed menarche, hypothalamic amenorrhea, dysmenorrhea, eating disorder, endometriosis, polycystic ovarian syndrome, premature menopause, ovarian failure, ovarian cysts, fibroids, genital prolapse, genital warts, hepatitis, HIV/AIDS, STIs, migraine headaches, obesity, substance abuse, cervical cancer, breast cancer, fibrocystic breast disease, dense breasts, ovarian cancer, endometrial cancer, pelvic inflammatory disease, infertility, diminished ovarian reserve, chronic or frequent UTIs, ectopic pregnancy, heart disease, Type I Diabetes, Type II Diabetes, hypothyroidism, hyperthyroidism, Hashimoto's, autoimmune conditions (lupus, MS, rheumatoid arthritis), or a combination thereof. In some embodiments, the information is a family history of clotting disorders, factor v leiden, hemophilia, endometriosis/adenomyosis, ovarian cysts, polycystic ovarian syndrome, fibroids, breast cancer, ovarian cancer, endometrial cancer, pelvic inflammatory disease, infertility, diminished ovarian reserve, chronic or frequent UTIs, ectopic pregnancy, pre-eclampsia, heart disease, Type I Diabetes, Type II Diabetes, or autoimmune conditions (Lupus, MS, rheumatoid arthritis). In some embodiments, the survey collects information on a past surgery. In some embodiments, the past surgery is an abdominal surgery, appendectomy, bladder suspension, breast biopsy, breast lumpectomy, cervical conization, cervical cryosurgery, laser treatment, cervix, cesarean section (C-section), cholecystectomy, colposcopy, diagnostic laparoscopy, dilation and curettage, endometrial ablation, hemorrhoidectomy, hernia repair, hysterectomy, hysteroscopy, myomectomy, oophorectomy, ovarian cystectomy, prolapse surgery, therapeutic laparoscopy, LEEP procedure (Loop electrosurgical excision), low back pain surgery, mastectomy, partial colectomy, releasing of peritoneal adhesions (scar tissue removal), tonsillectomy, tubal ligation, or a combination thereof. In some embodiments, the medical history is a test for BRCA1/BRCA2. If the subject has been tested for BRCA1/BRCA2 gene mutations, the survey asks which provider the subject used, including without limitations, Myriad Genetics, Color Genomics, Invitae, 23andme, Don't Know, or other provider. In some embodiments, the subject is BRCA1/BRCA2 positive (found to have mutations linked to breast and ovarian cancer in the subject's BRCA genes). In some embodiments, a subject takes medications, including over-the-counter medications or prescription medications. In some embodiments, a medication is paracetamol, acetaminophen, aspirin, ibuprofen, a COX-2 inhibitor (e.g., celecoxib or rofecoxib), an anti-inflammatory drug (e.g., naproxen mefanamix acid, aleve, 101anax101101101, 101anax101101, ketoprofen, anaprox), a narcotic drug (e.g., hydrocodone, codeine, morphine, oxycontin, hydrocodone, Demerol, or meperidine), another pain-killing drug such as one aimed at the nerves or central nervous system (e.g., amitriptyline, nortriptyline, gabapentin, pregabalin, or lamotrigine), a muscle relaxant (e.g., diazepam, temazepam, soma, lorzone, fexmid, or buscopan), an anti-anxiety or anti-depressant drug (e.g., citalopram, escitalopram, fluoxetine, fluvoxamine, paroxetine, diazepam, 102anax102102, 102anax102, luvox, paxil, 102anax102, 102anax, valium, amitriptyline, nortriptyline, SSRIs including sertraline), a stimulant (e.g., Adderall, Ritalin, Vyanese or methylphenidate), an HPV vaccine (e.g., Gardasil), estrogen-blockers, an herbal medicine or supplement, a vitamin or mineral supplement, or a multivitamin supplement. In some embodiments, a subject is currently taking one or more medications. In some embodiments, a subject has taken one or more medications within the past 1, 2, 3, 4, 5, or 6 months.

In some embodiments, the survey collects information about access to health care. In some embodiments, the survey collects information about health insurance, complementary and alternative medicine, costs associated with seeking health care, access to a vehicle, healthcare providers, most recent healthcare visit, preventative care, access to a medical professional.

In some embodiments, the complementary and alternative medicine is acupuncture, Ayurveda, homeopathy, naturopathy, traditional Chinese medicine, chiropractic and osteopathic medicine, dietary supplements, energy therapies (e.g. reiki), meditation, or body movement therapies (e.g. yoga, tai chi).

In some embodiments, the preventative care is a flu shot, a mammogram, a Pap smear, an HPV test, or a physical exam. In some embodiments, the subject receives preventative care at least about every 1, 2, 3, 4, 5, 6, 7, 8, or 10 years. In some embodiments, the subject needs to schedule an appointment for preventative care at least about 1 week, 2 weeks, 3 weeks, 4, weeks, 1 month, 2 months, 3, mores, 5 moths, 5 months, 6 months, or more than 6 months in advance of an appointment.

In some embodiments, the medical professional is a primary care physician or an OBGYN. In some embodiments, the subject is a Primary Care Physician to call when the subjects is sick or has a question about health. In some embodiments, the subject is an OBGYN (Obstetrician/Gynecologist) for the subject's reproductive needs. In some embodiments, the Primary Care Physician or OBGYN is located a distance away from the subject, including without limitations, less than 5 miles away, 5-10 miles away, 11-20 miles away, more than 20 miles away, more than 50 miles away, or more than 100 miles away. In some embodiments, the subject has a copay for an appointment. In some embodiments, the subject is regular access (owns or leases) to a vehicle. In some embodiments, the survey asks how far away, in miles, is the closest hospital to the subject. In some embodiments, the subject is normally travel to a doctor's appointment, including without limitations, by walking, public transportation, bike, drive, ask a family member/friend with a car to take me, shared ride service, or other. In some embodiments, the friend or family member joins the subject during doctor's visits.

In some embodiments, the subject is a budget in mind for annual healthcare costs and if so, the survey asks about the subject's budget. In some embodiments, the survey asks which of the following providers the subject is most likely to interact with during a medical appointment: nurse, PA (Physician's Assistant), MA (Medical Assistant), or Doctor (MD). In some embodiments, the survey asks a subject how easy it is to get a referral from the subject's primary care doctor for a specialist.

In some embodiments, the survey asks when the last time a subject went to the ER (Emergency Room). In some embodiments, the survey asks how many times a subject has been to the ER. In some embodiments, the survey asks when the last time a subject went to Urgent Care. In some embodiments, the survey asks how many times a subject has been to Urgent Care.

In some embodiments, the survey asks what the subject would be most likely to do if the subject had some abnormal symptoms related to reproductive health, including without limitations, call a primary care doctor's office to schedule an appointment; call a gynecologist's office to schedule an appointment; find the nearest Planned Parenthood for a consult; find the nearest minute clinic for a consult; wait to see if it gets worse, and if it does, find an urgent care clinic for a walk-in; wait to see if it gets worse, and if it does, find the closest hospital with an Emergency Room; seek on-line resources and wait to see how it develops; seek alternative at-home remedies to see if it resolves without escalating to a doctor; or other.

In some embodiments, the survey asks on a scale of 1 to 5, 1 being no consideration, 5 being heavy consideration, when the subject thinks about seeking medical care, how much consideration does the subject give to the following factors: co-pay, out of pocket costs before deductible, uncertainty around cost of tests prescribed, difficulty communicating with the doctor, wait time at doctor's office, commute to doctor's office, anxiety about results, costs of possible medications prescribed, having to take time off of work to go to a doctor, finding childcare to go to a doctor, or other factors.

In some embodiments, the survey asks the subject which plan best describes the subject's health insurance status, including without limitations, Health maintenance organization; Preferred provider organization; Point of service plan; Exclusive provider organization plan; High deductible health plan; Health Savings Account; Individual health plan purchased on the health insurance marketplace; VA care; Medicaid; Medicare; COBRA; or a combination thereof.

In some embodiments, a subject has a strong pelvic floor or a weak pelvic floor. Strength of pelvic floor muscles is determined by placing one or more fingers just above the tailbone and above the intergluteal cleft, squeezing the pelvic floor muscles, and feeling the strength of the movement felt.

In some embodiments, a subject has control of their bladder, sometimes loses control of their bladder, or regularly lose control of their bladder. In some embodiments, a subject experiences leaking with physical activity such as exercise, bending, sneezing, or coughing. In some embodiments, a subject experiences a sudden, intense urge to urinate, which present with a loss of control of the bladder or an uncontrollable flow of urine. In some embodiments, a subject experiences a frequent or constant need to urinate, which present without the ability to completely empty the bladder. In some embodiments, a subject experiences a slow leak of urine even when they believe their bladder has been emptied. In some embodiments, the survey asks if a physical therapist or doctor has ever confirmed the strength of the subject's pelvic floor. In some embodiments, the subject respond in the following ways: strong; weak; somewhere-in-between; or no. In some embodiments, the survey asks the subject to describe subject's pelvic floor strength as strong, weak, somewhere-in-between, or don't know, using FIG. 20 as a reference.

In some embodiments, a subject has had a vaginal yeast, viral, or bacterial infection in the past. In some embodiments, a subject has had a vaginal yeast, viral, or bacterial infection in the past 1, 2, 3, 4, 5, or 6 months. In some embodiments, a subject is a vaginal yeast, viral, or bacterial infection at the time the sample is taken. In some embodiments, a subject has recurrent vaginal yeast, viral, or bacterial infections. In some embodiments, the subject has had a vaginal yeast, viral, or bacterial infection in the past 3 months. If the subject has had a vaginal yeast, viral, or bacterial infection in the past 3 months, the survey asks if this infection occurred while the subject had a period. In some embodiments, the subject is ever had an abnormal Pap smear.

In some embodiments, the patient has been prescribed antibiotics in the past. In some embodiments, an antibiotic is amoxicillin, doxycycline, cephalexin, ciprofloxacin, clindamycin, metronidazole, azithromycin, sulfamethoxazole, trimethoprim, clavulanate, levofloxacin, penicillin, or another antibiotic. In some embodiments, antibiotics has been prescribed for a vaginal bacterial infection or for another infection. In some embodiments, a patient has taken a course of antibiotics at least 1, 5, 10, 15, 20, 25, 30, or more times in their life. In some embodiments, the subject has been prescribed a course of antibiotics in the last 3 months. In some embodiments, the survey asks the subject how many times the subject has been prescribed a course of antibiotics in the subject's lifetime. In some embodiments, the survey asks the subject to list any other medications the subject might be taking for other conditions. In some embodiments, the subject has been on any long-course antibiotics and if so, how many total months the subject spent using them.

In some embodiments, the survey collects information about therapy. In some embodiments, the subject has had a therapist currently or in the past. If the subject has seen a therapist, the survey asks what kind of therapist the subject has seen, including without limitations, Talk therapist, Psychologist, App-based therapy, Psychiatrist, Group therapy, or other.

### Body Composition

In some embodiments of method provided herein, the digital biomarker comprises a phenotypical or behavioral property of the subject's body composition as described herein.

In some embodiments, the subject's natural hair color is brown, black, blonde, red, grey, or white.

In some embodiments, the survey collects information about body composition. In some embodiments, the survey collects information on weight loss, weight gain, muscle mass, muscle composition, body fat location, waist circumference, bust circumference, hip circumference, bra band size, bra cup size, or US jean size. In some embodiments, the survey asks questions to further understand how the subject's body holds weight. In some embodiments, the survey asks the subject to describe the subject's ability to lose or gain weight. In some embodiments, the subject gains fat quickly and have a hard time taking it off; put on fat easily, but can lose it when wanted; not fluctuate a lot; or barely put on fat. In some embodiments, the survey asks the subject to describe the subject's ability to put on muscle mass. In some embodiments, the subject is put on muscle mass quickly and easily; have a hard time putting on muscle mass; or have muscle mass.

In some embodiments, the survey asks the subject to describe the subject's muscle as either lean muscle but not a lot of definition, defined muscle, or not well-defined muscle. In some embodiments, the survey asks the subject where the subject stores fat according to FIG. 21, including without limitations, chest, upper arms, abdomen, hips, outer and inner thighs, knees, lower back, or buttocks.

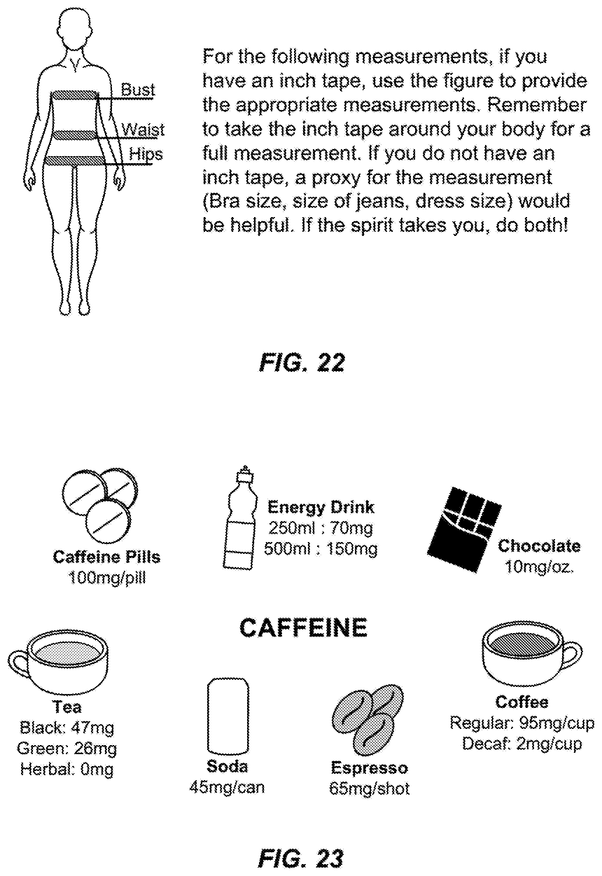

In some embodiments, the survey asks the subject to measure the circumference of the subject's waist, hips, and bust in accordance with FIG. 22. In some embodiments, the survey asks for the subject's bra band size. In some embodiments, the survey asks for the subject's bra cup size. In some embodiments, the survey asks for the subject's US jean size. In some embodiments, the survey asks for the subject's US dress size.

In some embodiments, a subject is short, average height, or tall. In some embodiments, a short subject is less than 5 feet tall, less than 5 feet and 1 inch tall, less than 5 feet and 2 inches tall, less than 5 feet and 3 inches tall, less than 5 feet and 4 inches tall, less than 5 feet and 5 inches tall, or less than 5 feet and 6 inches tall. In some embodiments, a tall subject is more than 5 feet and 6 inches tall, more than 5 feet and 7 inches tall, more than 5 feet and 8 inches tall, more than 5 feet and 9 inches tall, more than 5 feet and 10 inches tall, more than 5 feet and 11 inches tall, or more than 6 feet tall. In some embodiments, an average height subject has a height between that of a short subject and that of a tall subject.

In some embodiments, a subject is underweight, normal weight, overweight, obese, or morbidly obese. In some embodiments, an underweight subject is less than 115 pounds, less than 110 pounds, less than 105 pounds, or less than 100 pounds. In some embodiments, an underweight subject has a body mass index (BMI) of less than 19, less than 18.5, less than 18, or less than 17.5. In some embodiments, a normal weight subject is between 105 and 170 pounds, between 105 and 165 pounds, between 105 and 160 pounds, between 105 and 155 pounds, between 105 and 150 pounds, between 105 and 145 pounds, between 110 and 170 pounds, between 110 and 165 pounds, between 110 and 160 pounds, between 110 and 155 pounds, between 110 and 150 pounds, between 110 and 145 pounds, between 115 and 170 pounds, between 115 and 165 pounds, between 115 and 160 pounds, between 115 and 155 pounds, between 115 and 150 pounds, or between 115 and 145 pounds. In some embodiments, a normal weight subject has a BMI between 17.5 and 25.5, between 18 and 25.5, between 18.5 and 25.5, between 19 and 25.5, between 17.5 and 25, between 18 and 25, between 18.5 and 25, between 19 and 25, between 17.5 and 24.5, between 18 and 24.5, between 18.5 and 24.5, or between 19 and 24.5. In some embodiments, an overweight subject is between 145 and 190 pounds, between 145 and 185 pounds, between 145 and 180 pounds, between 145 and 175 pounds, between 145 and 170 pounds, between 150 and 190 pounds, between 150 and 185 pounds, between 150 and 175 pounds, between 150 and 170 pounds, between 155 and 190 pounds, between 155 and 185 pounds, between 155 and 180 pounds, between 155 and 175 pounds, or between 155 and 170 pounds. In some embodiments, an overweight subject has BMI of between 24.5 and 30.5, between 24.5 and 30, between 24.5 and 29.5, between 25 and 30.5, between 25 and 30, between 25 and 29.5, between 25.5 and 30.5, between 25.5 and 30, or between 25.5 and 29.5. In some embodiments, an obese subject is between 175 and 220 pounds, between 175 and 215 pounds, between 175 and 210 pounds, between 175 and 205 pounds, between 180 and 220 pounds, between 180 and 215 pounds, between 180 and 210 pounds, between 180 and 205 pounds, between 185 and 220 pounds, between 185 and 215 pounds, between 185 and 210 pounds, or between 185 and 205 pounds. In some embodiments, an obese subject has a BMI of between 29 and 41, between 29 and 40.5, between 29 and 40, between 29 and 39.5, between 29 and 39, between 29.5 and 41, between 29.5 and 40, between 29.5 and 39.5, between 29.5 and 39, between 30 and 41, between 30 and 40.5, between 30 and 40, between 30 and 39.5, between 30 and 39, between 30.5 and 41, between 30.5 and 40.5, between 30.5 and 40, between 30.5 and 39.5, between 30.5 and 39, between 31 and 41, between 31 and 40.5, between 31 and 40, between 31 and 39.5, or between 31 and 39. In some embodiments, a morbidly obese subject is more than 205 pounds, more than 210 pounds, more than 215 pounds, more than 220 pounds, or more than 225 pounds. In some embodiments, a morbidly obese subject has a BMI of more than 39, more than 39.5, more than 40, more than 40.5, or more than 41. BMI is calculated using any acceptable formula.

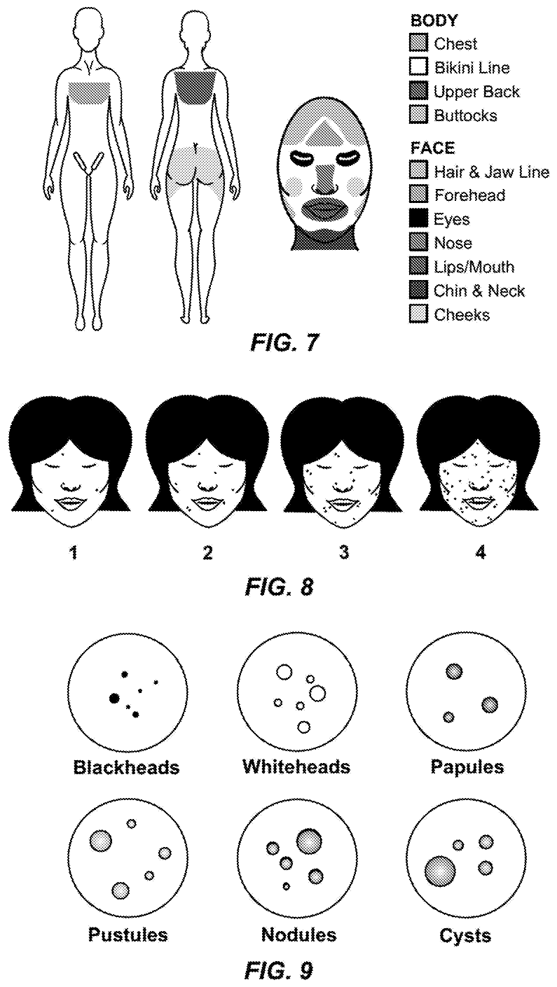

In some embodiments, the survey collects information about hair-loss. In some embodiments, the survey collects information about acne. In some embodiments, the survey collects information about the location, severity, or type of acne. In some embodiments, the type of acne comprises blackheads, whiteheads, pustules, nodules, or cysts. In some embodiments, the subject currently has acne. If the subject has acne, the survey asks them to indicate where the acne generally presents, including without limitations, chest, bikini line, upper back, buttocks, hair & jaw line, forehead, eyes, nose, lips/mouth, chin & neck, or cheeks. In some embodiments, an example of this is seen in FIG. 7. In some embodiments, the survey asks the subject to indicate the severity of the subject's acne using the image in FIG. 8 as reference. In some embodiments, the survey asks the subject which of the following options, shown in FIG. 9, best describes the subject's type of acne: blackheads, whiteheads, papules, pustules, nodules, or cysts.

In some embodiments, the survey collects information about the quantity of body hair. In some embodiments, the body hair is on the upper lip, upper arms, chin/neck, thighs, chest, stomach, upper back, lower back, or pubic region. In some embodiments, the survey asks the subject which of the following options, shown in FIG. 6, best describes the subject's hair. In some embodiments, the subject classifies the subject's hair as: 1— Thick and full hair; 2—Slightly reduced hair with widening part; 3—Reduced hair with widening part and some scalp showing; 4—Significantly reduced hair and more scalp showing; 5—Scalp mostly visible; 6—Traction alopecia; 7—Alopecia areata; or 8—Alopecia totalis.

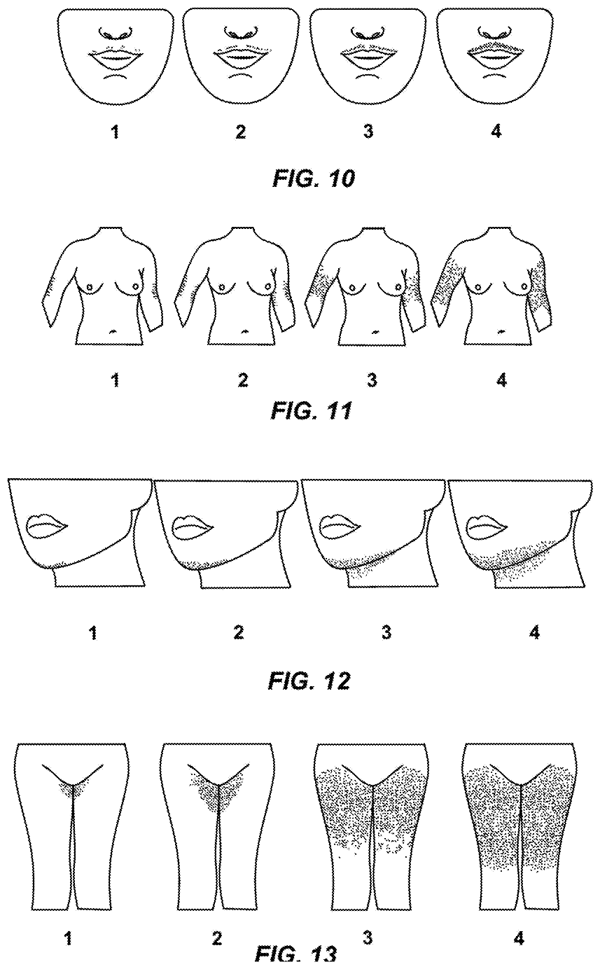

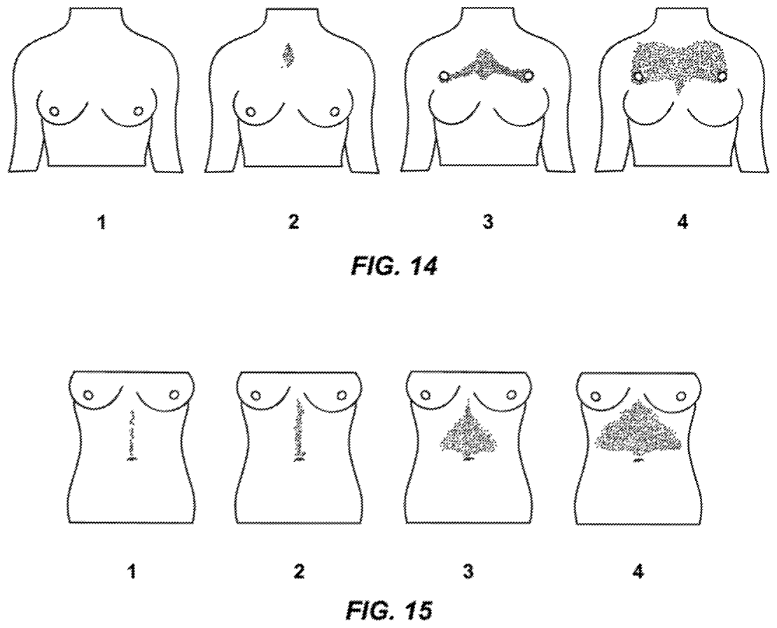

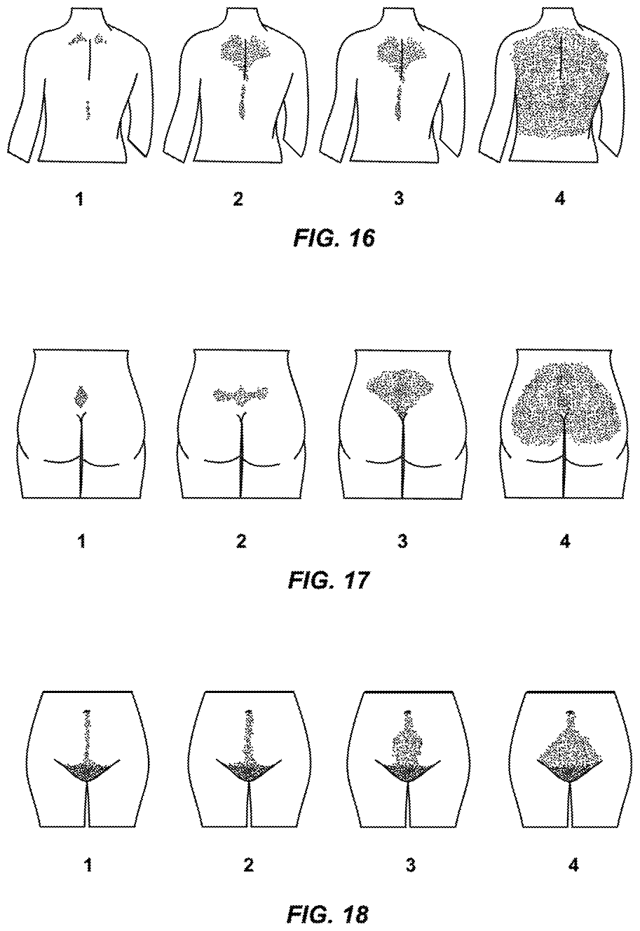

In some embodiments, the survey asks the subject to indicate the location and quantity of hair growth on the subject's upper lip using FIG. 10 as a reference. In some embodiments, the survey asks the subject to indicate the location and quantity of hair growth on the subject's upper arms using FIG. 11 as a reference. In some embodiments, the survey asks the subject to indicate the location and quantity of hair growth on the subject's chin/neck using FIG. 12 as a reference. In some embodiments, the survey asks the subject to indicate the location and quantity of hair growth on the subject's thighs using FIG. 13 as a reference. In some embodiments, the survey asks the subject to indicate the location and quantity of hair growth on the subject's chest using FIG. 14 as a reference. In some embodiments, the survey asks the subject to indicate the location and quantity of hair growth on the subject's stomach using FIG. 15 as a reference. In some embodiments, the survey asks the subject to indicate the location and quantity of hair growth on the subject's upper back using FIG. 16 as a reference.

In some embodiments, the survey asks the subject to indicate the location and quantity of hair growth on the subject's lower back using FIG. 17 as a reference. In some embodiments, the survey asks the subject to indicate the location and quantity of hair growth on the subject's pubic area using FIG. 18 as a reference.

In some embodiments, a subject consumes a vegan, ketogenic, Kosher, gluten-free, dairy-free, vegetarian, paleo, halal, low-fat, pescatarian, raw food, low-sugar, low-carb, Mediterranean, or typical Western diet. In some embodiments, a subject has a history of disordered eating or an eating disorder. In some embodiments, eating disorders include anorexia nervosa, bulimia nervosa, binge-eating disorder, one or more food aversions, or one or more food addictions.

In some embodiments, the survey asks the subject to describe where the subject gets most of the food the subject consumes, including without limitations, Order In/Take Out; Eat at Medium to Expensive Restaurants; Eat at Fast Food Restaurants (e.g. McDonalds, Burger King, Taco Bell, KFC, Pizza Hut etc.); Eat a Fast Fresh Restaurants (Chipotle, Affordable local); Whole Foods; Trader Joe's; Sprouts; Other National grocery chains (Safeway, Kroger); Farmer's Markets; Convenience stores; Grow my own food; or other. In some embodiments, the survey asks the subject how far away (in miles) is the closest grocery store or place where the subject regularly buys food.

In some embodiments, the survey collects information about caffeine intake. In some embodiments, the survey asks the subject to indicate how many units of each of the following caffeinated products the subject consumes on a daily basis: 8 oz Cups of regular coffee, 8 oz Cups of decaf coffee, Shots of espresso, 8 oz Cups of black tea, 8 oz Cups of green tea, 8 oz Cups of herbal tea, 12 oz cans of soda, 8 oz cans of energy drink, Number of 200 mg caffeine pills, 1 oz of milk chocolate (˜⅓ of a chocolate bar), and 1 oz of dark chocolate (˜⅓ of a chocolate bar). In some embodiments, the caffeinated products is seen in FIG. 23.

In some embodiments, a subject is of any race or ethnicity. In some embodiments, ethnicity or ancestry comprises Native American (indigenous Americans across North, Central and South America), Pacific Islander) Micronesia, Melanesia, Polynesia), South Asian (Afghanistan, Pakistan, India, Bangladesh, Nepal, Bhutan, Sri Lanka), Southeast Asian (Thailand, Vietnam, Malaysia, Singapore, the Philippines, Laos, Indonesia, Brunei, Myanmar, Cambodia and Timor-Leste), East Asian (China, Korea, Japan, Taiwan or Mongolia), Levantine (Lebanon, Syria, Jordan, Israel and Palestine), Peninsular Arab (Bahrain, Kuwait, Oman, Qatar, Saudi Arabia, United Arab Emirates, Yemen), Northern West Asian (Iran, Iraq, Armenia, Azerbaijan, Georgia), North African (Algeria, Egypt, Libya, Morocco, Sahrawi Arab Democratic Republic, Sudan, Tunisia), East Africa (Burundi, Comoros, Djibouti, Eritrea, Ethiopia, French Southern and Antarctic Lands, Kenya, Madagascar, Malawi, Mauritius, Mayotte, Mozambique, Reunion, Rwanda, Seychelles, Somalia, Somaliland, South Sudan, Tanzania, Uganda, Zambia, Zimbabwe), Central Africa (Angola, Cameroon, Central African Republic, Chad, Democratic Republic of the Congo, Republic of the Congo, Equatorial Guinea, Gabon, Sao Tome and Principe), West Africa (Benin, Burkina Faso, Cape Verde, Ivory Coast, Gambia, Ghana, Guinea, Guinea-Bissau, Liberia, Mali, Mauritania, Niger, Nigeria, Saint Helena, Ascension and Tristan da Cunha, Senegal, Sierra Leone, Togo), Southern Africa (Botswana, Swaziland, Lesotho, Namibia, South Africa), Broadly Sub-Saharan African, Afro-Caribbean (Anguilla, Antigua & Barbuda, Aruba, The Bahamas, Barbados, British Virgin Islands, Cayman Islands, Cuba, Dominica, Dominican Republic, Grenada, Guadeloupe, Haiti, Jamaica, Martinique, Montserrat, Netherlands Antilles, Puerto Rico, Saint Barthelemy, Saint Kitts & Nevis, Saint Lucia, St Martin, Saint Vincent, Trinidad & Tobago, Turks & Caicos Islands, US Virgin Islands), Western European (Belgium, France, Germany, Luxembourg, the Netherlands, the Republic of Ireland, and the United Kingdom, Austria and Switzerland), Northern Europe (Denmark, Finland, Greenland, Iceland, Norway, and Sweden), Eastern European (Czech Republic, Estonia, Latvia, Lithuania, Poland, Slovakia, Albania, Bosnia-Herzegovina, Bulgaria, Croatia, Hungary, Romania, Slovenia, and Serbia-Montenegro), Southern European (Greece, Italy, Portugal, and Spain), Broadly European, Broadly Latin American or a combination thereof. In some embodiments, the survey asks questions about how the US census would classify the subject, including without limitations, Alaska Native/Native American—Navajo Nation, Blackfeet Tribe, Mayan, Aztec, Native Village of Barrow Inupiat Traditional Government, Nome Eskimo Community; Black or African American—African American, Jamaican, Haitian, Nigerian, Ethiopian, Somali; (Of) Hispanic, Latino or Spanish origin—Mexican, Mexican American, Chicano, Puerto Rican, Cuban, Salvadoran, Dominican, Colombian, Guatemalan, Spaniard, Ecuadorian; Other Asian—Pakistani, Cambodian, Hmong; Other Pacific Islander—Tongan, Fijian, Marshallese; White—German, Irish, English, Italian, Lebanese, Egyptian; or some other race.

In some embodiments, the survey collects information on the subject's relationship status. In some embodiments, the relationship status is single, monogamous relationship, polyamorous relationship, open relationship, engaged, married, widowed, separated, divorced, civil union, a domestic partnership, it's complicated, or other. In some embodiments, the survey collects data on contentment with relationship status. In some embodiments, the survey asks the subject to rank the subject's contentment with the subject's current relationship status on a scale of 1 to 10, 10 being optimal.

In some embodiments, the current living situation is living on one's own, living with parents, living with a partner or spouse, living with children, living with roommates or a combination thereof. In some embodiments, the religious background or current religious affiliation is spiritual, Christian Protestant, Evangelical Christian, Catholic, LDS/Mormon, Jehovah's Witness, Nondenominational Christian, Nondenominational Jewish, Reform Jewish, Orthodox Jewish, Other Jewish, Shiite Muslim, Sunni Muslim, Other Islamic, Hindu, Sikh, Jain, Buddhist, Zoroastrian, Atheist, Agnostic, Taoist, Confucianist, Bahai, Lucumi practitioner (Santeria), Native American system of spirituality, or another religious affiliation.

### Gender

In some embodiments of methods provided herein, the digital biomarker comprises a property related to the subject's gender or sexual orientation. In some embodiments, a subject is of any sexual orientation. In some embodiments, a sexual orientation is asexual, bisexual, gay, heterosexual/straight, lesbian, pansexual or queer. In some embodiments, a subject is cisgender, non-binary, two-spirit, transgender, or agender. In some embodiments, the survey asks the subject what term best encapsulates the subject's sexual orientation, including without limitations, heterosexual/straight (sexual or emotional attraction to the opposite gender); gay (sexual or emotional attraction to the same gender); lesbian (sexual or emotional attraction to the same gender, typically female to female); bisexual (an umbrella term for people who experience sexual and/or emotional attraction to more than one gender); asexual (the lack of sexual attraction, and one identifying with this orientation); pansexual (capable of being attracted to many/any gender(s)); queer (general term for gender and sexual minorities who are not cisgender and/or heterosexual); or other sexual orientation.

In some embodiments, the survey asks the subject what term best encapsulates the subject's gender identity, including without limitations, cisgender (identify with sex assigned at birth); nonbinary (a spectrum of gender expressions that fall outside of the gender binary “male/female”); two-spirit (a term indexing North American indigenous gender identities, including third-gender and other gender variants); transgender (identify differently than sex assigned at birth); agender (encompassing many different genders of people who commonly do not have a gender and/or have a gender that they describe as neutral); or other gender identity.

In some embodiments, a subject is assigned female at birth. In some embodiments, a subject is assigned male at birth.

In some embodiments, a subject is non-binary or trans. In some embodiments, the survey collects information on age of first internal gender discordance, age of social transition, diagnosis of gender dysphoria, hormone use, birth control, gender confirmation surgery, fertility preservation, contraindications to hormone therapy, testosterone therapies, steroid use, bone mineral density, hematocrit levels, lipid levels, access to medical professionals, mammogram, or cervical cancer screening.

In some embodiments, the survey asks at what age did the subject first feel a gender discordance internally, at what age did the subject know the subject's gender identity differed from the subject's assigned sex, and at what age did the subject begin to socially transition (pronoun, dress, hair, binding) in the following areas: home (with family), at work, at school with peers/friends, and full social transition. In some embodiments, the survey asks how long the subject has identified as gender non-binary or transgender. In some embodiments, the subject is received a diagnosis of “gender dysphoria” from a therapist.

In some embodiments, the subject is used hormones to achieve gender congruence with the subject's affirmed gender. If the subject has used hormones, the survey asks at what age did the subject begin to use hormones to achieve gender congruence with the subject's affirmed gender. In some embodiments, the subject has been on hormones for gender affirming purposes for less than about 1, 2, 3, 4, 5, 6 7, 8, 9, or 10 years.

In some embodiments, the subject is used birth control to suppress the dysphoric nature of menstruation. In some embodiments, the subject has had surgery to achieve gender congruence with the subject's affirmed gender, and if the subject has had such a surgery, the survey asks at what age did the subject begin to use surgery to achieve gender congruence with the subject's affirmed gender. In some embodiments, the subject is considered fertility preservation prior to a medical or social transition. In some embodiments, the survey asks if gender affirming hormone therapy is covered by the subject's insurance.

In some embodiments, the subject has had transition included being given GnRH analogs (Lupron or some other brand) or a progesterone shot (like Depo-Provera) to suppress puberty or stop the subject's period completely. In some embodiments, the subject has been diagnosed with any of the following contraindications (a reason to not take a particular therapy) to hormone therapy: AIS (androgen insensitivity syndrome), Androgen-sensitive epilepsy, Migraines, elevated red blood cells/hematocrit, severe high blood pressure, kidney failure, heart failure, liver disease, coronary artery disease, or other contraindications.

In some embodiments, the subject is increased testosterone levels to average male physiological levels by administering testosterone as part of the subject's transition. If so, the subject may have taken oral testosterone undecanoate, Testosterone undecanoate Injection, Testosterone enanthate or cypionate Injection, Testosterone 1% gel through the skin, or Testosterone patch. In some embodiments, the survey asks about the route of administration for the subject's hormone therapy, which could be a pill, patch, cream, gel, implant, or pellets/rods. In some embodiments, the subject is practiced “stacking” (taking another type of steroid in addition to testosterone). In some embodiments, the subject is experienced any side effects from taking testosterone such as aggression at first application when T peaks, fatigue/irritability when T dips, psychiatric problems, elevations in blood pressure, increased libido, changes in emotions, elevated hematocrit levels, worsening lipid profile, elevations in glucose, acne, or cessation of menstruation.

In some embodiments, the subject has been monitored every 3 months for the first year of testosterone therapy for adverse effects. In some embodiments, the subject has been monitored every 6 to 12 months after the first year of testosterone therapy for adverse effects. In some embodiments, the subject is monitored serum testosterone while taking testosterone therapy. In some embodiments, the subject is acquired testosterone at a pharmacy with a doctor's prescription, through an on-line pharmacy without a prescription, deep net on-line resources, or other source. In some embodiments, the subject is monitored for bone mineral density on a regular basis. In some embodiments, the subject has had hematocrit levels and lipid levels are checked on a regular basis.

In some embodiments, the subject sees a medical professional every 3 months, every 6 months, annually, as needed, rarely, or never. In some embodiments, the survey asks a subject that does not regularly see a doctor for care, which of the following most closely describes why: fear of discrimination, access, affordability, finding a provider who understands trans care, or other reason.

In some embodiments, the survey asks what types of doctors/specialists the subject sees, including without limitations, General Practitioner, Endocrinologist, GYN, Urologist, Psychologist, Psychiatrist, Cardiologist, or other medical professional. In some embodiments, the subject is regularly screened for the following: heart disease, bone loss, stroke risk, depression, substance abuse, psychological distress, metabolic risk (diabetes, obesity), blood pressure, or other conditions.

In some embodiments, the subject still receives mammograms for breast cancer. In some embodiments, the survey asks about the subject's attitude regarding participation in breast cancer screening. In some embodiments, the subject still receives a pap smears for cervical cancer. In some embodiments, the survey asks about the subject's attitude regarding participation in cervical cancer screening.

In some embodiments, the subject has had any of the following surgeries: Hysterectomy (uterus removed), Oophorectomy (ovaries removed), Bilateral mastectomy (breasts removed), Metoidioplasty (clitoral release), Vaginectomy (removal of vaginal tissue), Scrotoplasty (construction of scrotum), Phalloplasty (construction of phallus), or Urethroplasty (creation of urethral canal, usually after construction of phallus, to allow urination standing up).

### Birth Control

In some embodiments of methods herein, the digital biomarker comprises a property related to birth control as described herein. In some embodiments, a subject takes a prescribed hormone. In some embodiments, prescribed hormones includes hormonal birth control, estrogen, GnRH agonists, testosterone, progesterone, anti-androgens, or other hormones. Prescribed hormones is used to prevent pregnancy, correct a hormone imbalance, to regulate or improve the cosmetic appearance of the subject, to regulate the weight of a subject, to regulate an irregular or painful period, or for gender transition.

In some embodiments, a subject is using birth control, such as a hormonal birth control or a non-hormonal birth control. In some embodiments, birth control is used for contraceptive purposes or non-contraceptive purposes. In some embodiments, birth control includes combination pills (e.g., Apri, Estrostep, Levlen, Loestri, Ortho Tri-Cyclen, Yasmin, or Yaz), progestin-only pills (e.g., Camila, Errin, Heather, Jolivette, Micronor, Nor-Q.D., or Orvette), spermicide, an injectable birth control (e.g., Depo-Provera), an upper arm implant (e.g., Nexplanon), a copper intrauterine device (e.g., Paraguard), a copper intrauterine device (e.g., Mirena, Kyleena, Liletta, or Skyla), a contraceptive patch, a vaginal ring (e.g., NuvaRing), plan B (e.g., levonorgestrel), surgical sterilization, partner vasectomy, abstinence, abstinence during ovulation, withdrawal, or condoms. In some embodiments, a subject uses birth control currently or has used birth control in the past 1 month, 2 months, 3 months, 4 months, 5 months, 6 months, 1 year, 2 years, 3 years, 4 years, or 5 years, or longer than 5 years ago. In some embodiments, a subject stopped using birth control or has switched methods of birth control within the past 1 month, 2 months, 3 months, 4 months, 5 months, 6 months, 1 year, 2 years, 3 years, 4 years, or 5 years. In some embodiments, reasons for stopping or switching birth control methods include difficulty in accessing, cost, wanting to get pregnant, unpleasant side effects, cultural reasons, a desire for change, a concern for health, or an interference with another medication. In some embodiments, the survey collects information on the purposes for using hormonal birth control, the start date of using birth control, the satisfaction level of using a method of birth control, method of birth control used in the past, reasons for stopping birth control, or changes experienced after stopping birth control.

In some embodiments, the survey asks a subject about the subject's purpose for using hormonal birth control, including without limitations, for contraception, to treat menstrual cramps, to regulate irregular periods, to control heavy periods, to treat acne/skin issues, to control symptoms of perimenopause (hot flashes, night sweats), to treat a reproductive disorder, or other. In some embodiments, the subject is very satisfied, satisfied, neutral, unsatisfied, or very unsatisfied with the subject's method of contraception. In some embodiments, the survey asks a subject for additional comments about the subject's experience with any type of birth control at any point in the subject's life. In some embodiments, the subject is changed their method of birth control because of the subject's partner's preference. In some embodiments, the survey asks the subject to elaborate on what motivated the partner to request a change in birth control method.

In some embodiments, the subject is previously decided to get off of hormonal birth control or had a doctor remove a hormonal/non-hormonal implant or IUD. If the subject has previously decided to get off of hormonal birth control or had a doctor remove a hormonal/non-hormonal implant or IUD, the survey asks the subject which hormonal birth controls or non-hormonal IUDs the subject used in the best, including without limitations, Combination Pills, Progestin-only Pills, Pills, Injection, Upper Arm Implant, Copper IUD, Hormonal IUD, Contraceptive Patch, Vaginal Ring, or other form of birth control.

### Military Service

In some embodiments of methods provided herein, the digital biomarker comprises a feature related to the subject's military service as described herein. In some embodiments, the survey collects information on military service. In some embodiments, the survey collects information on branch of the military, role of the military, rank in the military, deployment, UTIs, bacterial vaginosis, pregnancy, abortion, medical care, infertility, exposure to chemical hazards, exposure to biological hazards, exposure to environmental hazards, Post-Traumatic Stress Disorder, sexual trauma, disability compensation, health care services, mental health care services, or a combination thereof.

In some embodiments, the subject served in the Army, Navy, Air Force, Marines, or Coast Guard. In some embodiments, the subject's current status is described as Active Duty, Reserve, National Guard, or Veteran. In some embodiments, the subject has been (or is currently) enlisted personnel or an officer in the armed forces. In some embodiments, the highest paygrade the subject reached in the military is: Warrant Officer W-1, W-2, or W-3; Warrant Officer W-4 or W-5; Commissioned Officer O-1, O-2, or O-3; Commissioned Officer O-4, O-5, or O-6; Commissioned Officer O-7 or above; Enlisted member E-1, E-2, or E-3; Enlisted member E-4, E-5, or E-6; or Enlisted member E-7, E-8, or E-9.

In some embodiments, the survey asks the subject to describe the roles the subject played while deployed (e.g. logistics, cybersecurity, engineering, administrative, supply etc.). In some embodiments, the survey asks how many months total the subject has been/was deployed. In some embodiments, the survey asks the subject to list the places the subject has been deployed (City or country level. Separate places by comma.). In some embodiments, the subject has been deployed overseas to a region where the subject qualified for hazardous duty pay. In some embodiments, the subject has had daily duties require the subject to wear full body armor. In some embodiments, the subject is spent time living at a location where the infrastructure negatively impacted the subject's ability to conduct proper regular hygiene, and if so, the survey asks the subject to share how long the subject lived there and describe how the subject attempted to keep clean.

In some embodiments, the survey asks the subject which birth control method was used during deployment, including the birth control described herein. In some embodiments, the subject is deliberately sought hormonal birth control in order to stop the subject's period completely during deployment, and if so, the survey asks the subject which of the following reasons best describes why: could not guarantee access to period products, could not guarantee access to water for proper hygiene during period, could not guarantee access to privacy for managing period, did not want to deal with the hassle of managing period while deployed, or other reasons. In some embodiments, the survey asks if the circumstances of subject's deployment ever influenced the subject's choice of birth control, and if so, the survey asks how this influenced the subject's choice of birth control. In some embodiments, the survey asks the subject what period products the subject uses/used while deployed, including without limitations, pads, tampons, menstrual cups, menstrual discs, diapers, or other.

In some embodiments, the survey asks how frequently the subject experienced urinary tract infections or bacterial vaginosis during deployment, including without limitations, frequently, regularly, rarely, or never.

In some embodiments, the subject has had an unintended pregnancy during deployment. In some embodiments, the subject is sought to get an abortion outside of the medical facilities provided to military personnel, and if so, the subject had complications from this abortion. In some embodiments, the survey asks where the subject had to seek care for the complications, including without limitations, continued to seek care outside of military facilities, military facilities, avoided further care, or other.

In some embodiments, the subject has been in a situation where the subject needed an invasive reproductive exam (pap smear, for example) while deployed, and if so, the was comfortable receiving the reproductive exam from the military personnel available. In some embodiments, the subject received an abnormal Pap smear while deployed, and if so, the subject felt like there were adequate resources to understand and react to the diagnosis. In some embodiments, the subject has had deployment interrupt preventative care measures the subject wanted to participate in such as cervical cancer screening, on-going treatment for endometriosis, on-going treatment for menorrhagia, or on-going treatment for uterine fibroids.

In some embodiments, the subject has been diagnosed with infertility while on Active Duty, and if so, the subject was able to access treatment options for infertility at a military hospital. In some embodiments, the survey asks what treatment options the subject was able to use and to what extent the cost of the infertility treatments were covered, including without limitations, partially, fully, not at all, or other.

In some embodiments, the subject sought reproductive care outside of the resources provided by the military during deployment. In some embodiments, the subject has been exposed to contaminated water, overheated plastic water bottles, burn pits, or oversized body armor while deployed. In some embodiments, the subject spent any time living at a location where exposed to other chemical, biological or other environmental hazards during deployment.

In some embodiments, the subject has acute symptoms of exposure. In some embodiments, the subject has been treated for acute symptoms, and if so, the survey asks what treatment was administered. In some embodiments, the subject has recurring symptoms of exposure, and the survey asks the subject to list any recurring symptoms. In some embodiments, the subject has been treated for recurring symptoms of exposure, and if so, the survey asks what treatment was administered. In some embodiments, the subject is still experiencing symptoms of exposure. In some embodiments, the subject is currently seeking treatment for exposure and if so, the survey asks what treatments the subject is currently using. In some embodiments, the subject is experienced an onset of symptoms or a worsening of symptoms (asthma, pain, trouble breathing) in a particular location while deployed, and if so, the survey asks the subject to describe the circumstances and/or location of the onset of symptoms.

In some embodiments, the subject experienced military sexual trauma. In some embodiments, the survey asks if the subject reported it and if the subject sought medical attention after experiencing the trauma. If the subject did not seek medical attention, the survey asks the subject to explain why they chose not to. In some embodiments, the subject sought treatment for PTSD or treatment for sexual trauma associated with military duty while on Active Duty.

The survey asks what year the subject separated from the military. In some embodiments, the survey asks the subject to share the subject's VA disability rating if applicable. In some embodiments, the subject received service connected disability compensation, and if so, the survey asks which service connected condition the subject sought disability compensation for, with the options being post-traumatic stress disorder, migraines, lower back pain, or other.

In some embodiments, the subject is enrolled in the Veterans Health Administration, and if so, the subject uses VA health care or pursue medical services elsewhere, including without limitations, always VA facilities, always other non-VA healthcare facilities, or a combination of VA and non-VA facilities. In some embodiments, the subject uses VA outpatient mental health services regularly, infrequently, or never. In some embodiments, the subject participated in the Vocational Rehabilitation and Employment program. In some embodiments, the subject used Montgomery GI Bill benefits.

In some embodiments, the subject has had to use a non-VA facility to get checked, diagnosed or treated for the following conditions because the VA facility the subject has access to was not adequately staffed or equipped to provide the services the subject needed: mammogram, pap smear, breast cancer treatment, gynecological cancer treatment, PTSD Therapy, in-vitro fertilization treatment, or other condition.

In some embodiments, the subject has been able to access treatment options for infertility at a VA hospital, and if so, the survey asks what options the subject was able to use. In some embodiments, the survey asks if the cost of the infertility treatments were partially, fully, or not at all covered by the subject's Veteran's insurance. In some embodiments, the survey asks if the reproductive care provided by the VA meets the subject's needs, and if not, the survey asks the subject to elaborate on how the VA facilities/benefits could be more targeted to the subject's reproductive needs. In some embodiments, the subject sought treatment for PTSD or treatment for sexual trauma associated with military duty.

### Assault

In some embodiments of method provided herein, the digital biomarker comprises a feature related to sexual assault as described herein. In some embodiments, the survey collects information on assault. In some embodiments, the survey collects information on domestic violence, intimate partner violence, sexual assault, contraceptive use, or health services used after sexual assault.

In some embodiments, the subject is a survivor of domestic violence, and if so, the survey asks who perpetrated the violence, including without limitations, mother, father, siblings, extended family member, family friend, domestic partner, significant other, or other. In some embodiments, the subject is a survivor of intimate partner violence, and if so, the survey asks if the circumstances have changed or if they are ongoing and whether the subject is now safe or still affected by intimate partner violence.

In some embodiments, the subject is a survivor of sexual assault, and if so, the survey asks who perpetrated the violence, including without limitations, family member, boyfriend/girlfriend/partner, friend, acquaintance, stranger, uncertain, or other. In some embodiments, the survey asks if the perpetrator utilized any coercive methods such as physical force, threats (including threats to end relationship), alcohol or drugs, social capital, position of authority, withholding of payment/other resources, manipulation, or other. In some embodiments, the survey asks if the perpetrator used any sort of protective/contraceptive method such as dental dam, spermicide, provided Plan B afterwards, unknown, or other contraception.

In some embodiments, the subject is shared what happened with somebody afterwards, and if so, the survey asks the subject to identify who the story was shared with, including without limitations, mother, father, siblings, extended family, partner, friend(s), crisis/support workers (in person, over the phone, or over text), medical provider, therapist, deans or other figures who would provide accommodations at school or work, school's Title IX office or employer's HR office (formal accusation), legal authorities (criminal charges), or others. In some embodiments, the survey asks how long the subject waited to tell the first person about the assault, including without limitations, shared immediately, 1 week, 1 month, 2-6 months, 6-12 months, 1-2 years, 2-5 years, or more than 5 years. In some embodiments, the subject found the support or response that was needed. In some embodiments, the survey asks if the perpetrator's role in the subject's social network affected how and when the subject shared the story. In some embodiments, the subject experienced retaliation after choosing to report the experience, either within an institution or for criminal proceedings. In some embodiments, the subject sought reproductive care after the sexual assault, and if so, the survey asks what services the subject accessed, including without limitations, SANE/SART or other evidence collection processes, pelvic examination, STI/STD testing, HIV/AIDs testing, mental health services, emergency contraceptive services, abortion services, religious or spiritual counseling, or other reproductive care.

In some embodiments, the subject's experience of sexual assault have caused the subject to access reproductive health care and other medical services more frequently, the same amount, or less frequently. In some embodiments, the subject has had a non-consensual sexual experience (whether you considered it non-consensual at the time or not), and if so, the survey asks who perpetrated the violence, including without limitations, family member, boyfriend/girlfriend/partner, friend, acquaintance, stranger, uncertain, or other. In some embodiments, the survey asks if the perpetrator utilized any coercive methods such as physical force, threats (including threats to end relationship), alcohol or drugs, social capital, position of authority, withholding of payment/other resources, manipulation, or other. In some embodiments, the survey asks if the perpetrator used any sort of protective/contraceptive method such as dental dam, spermicide, provided Plan B afterwards, unknown, or other contraception.

In some embodiments, the subject shared what happened with somebody afterwards, and if so, the survey asks the subject to identify who the story was shared with, including without limitations, mother, father, siblings, extended family, partner, friend(s), crisis/support workers (in person, over the phone, or over text), medical provider, therapist, deans or other figures who would provide accommodations at school or work, school's Title IX office or employer's HR office (formal accusation), legal authorities (criminal charges), or others. In some embodiments, the subject waited 1 week, 1 month, 2-6 months, 6-12 months, 1-2 years, 2-5 years, or 5 or more years to tell the first person about the assault, with the options being. In some embodiments, the subject found the support or response that was needed. In some embodiments, the survey asks if the perpetrator's role in the subject's social network affected how and when the subject shared the story. In some embodiments, the subject experienced retaliation after choosing to report the experience, either within an institution or for criminal proceedings. In some embodiments, the subject sought reproductive care after the non-consensual sexual experience, and if so, the survey asks what services the subject accessed, including without limitations, SANE/SART or other evidence collection processes, pelvic examination, STI/STD testing, HIV/AIDs testing, mental health services, emergency contraceptive services, abortion services, religious or spiritual counseling, or other reproductive care.

“Sexual initiation” is defined as the first experience of vaginal intercourse, penetrative or otherwise. In some embodiments, the subject experienced force or coercion during the subject's sexual initiation, regardless of how the subject felt at the time. In some embodiments, the age difference was between the subject and the perpetrator was 1-3 years, 3-6 years, 6-9 years, 9 or more years, or unknown. In some embodiments, the survey asks how many years passed between the forced initiation and the subject's first voluntary sexual experience, with the options being 1 year, 2 years, 3 years, or 4 or more years.

In some embodiments, the survey asks the subject if the question of sexual trauma and/or assault has ever come up during a regular physical exam or during a medical encounter not related to the trauma/assault itself. In some embodiments, the subject sought medical care for deep dyspareunia (pain with deep vaginal penetration).

### Habits

In some embodiments of methods provided herein, the digital biomarker comprises a feature related to the subject's behavioral features as described herein. In some embodiments, a subject drinks alcohol. In some embodiments, a subject drinks at least 0, 1, 2, 3, 4, 5, 6, 7, 8, 9, or 10 alcoholic beverages on average per week. In some embodiments, a subject drinks no more than 0, 1, 2, 3, 4, 5, 6, 7, 8, 9, or 10 alcoholic beverages on average per week.

In some embodiments, a subject is a cigarette smoker. In some embodiments, a subject smokes at least 0, 1, 2, or 3 packs of cigarettes on average per day. In some embodiments, a subject smokes no more than 0, 1, 2, or 3 packs of cigarettes on average per day. In some embodiments, a subject is a former cigarette smoker. In some embodiments, a former cigarette smoker has quit at least 1 day, 1 week, 1 month, 6 months, 1 year, 2 years, 3 years or more prior to collection of menstrual fluid. In some embodiments, a former cigarette smoker has quit no more than 1 day, 1 week, 1 month, 6 months, 1 year, 2 years, or 3 years prior to collection of menstrual fluid. In some embodiments, a subject consumes other nicotine containing products such as chewing tobacco or electronic cigarettes. In some embodiments, the survey asks if a subject drinks alcohol, and if so, how many drinks on average the subject consumes a week. In some embodiments, the survey asks how many packs a subject did/does smoke per week. In some embodiments, the survey asks if a subject has smoked more than 100 cigarettes during the subject's lifetime. In some embodiments, the survey asks if a subject has ever been an e-cigarette smoker or currently smokes e-cigarettes. In some embodiments, the survey asks the subject the date in which the subject stopped smoking e-cigarettes. In some embodiments, the survey asks the subject to describe the subject's e-cigarette usage, including without limitations, occasional (a few times per year), regular (more than once per week), or daily (once or multiple times per day).

In some embodiments, the survey asks how frequently the subject has been passively exposed to second hand smoke over a long period of time, including without limitations, never, rarely (once per year), occasional (a few times per year), regular (more than once per week), or daily (once or multiple times per day).

In some embodiments, a subject used marijuana products. Marijuana use is never, occasional (e.g., not more than 1, 2, 3, 4, 5, 6, 7, 8, 9, 10, 11, or 12 times per year, or less than regular), regular (e.g., not more than 1, 2, 3, 4, 5, 6, or 7 times per months, or less than daily), or daily (average at least once per day or more than once per day). In some embodiments, a subject used other products comprising tetrahydrocannabinol (THC), cannabidiol (CBD), or a combination of THC and CBD. In some embodiments, the survey asks if a subject uses marijuana (THC) or hemp (CBD) products. In some embodiments, the survey asks the subject to describe the frequency of the subject's THC usage, including without limitations, never, occasional (a few times per year), regular (more than once per week), or daily (once or multiple times per day). In some embodiments, the survey asks how a subject consumes THC products, including without limitations, inhalation, ingestion, or topical. In some embodiments, the survey asks the subject to describe the frequency of the subject's CBD usage, including without limitations, never, occasional (a few times per year), regular (more than once per week), or daily (once or multiple times per day). In some embodiments, the survey asks how a subject consumes CBD products, including without limitations, inhalation, ingestion, or topical

In some embodiments, a subject exercises or does not exercise. In some embodiments, a subject who exercises occasionally, frequently, or daily. In some embodiments, a subject s exercises on average 0 days per week, 1-2 days per week, 3-4 days per week, 5-7 days per week. In some embodiments, a subject exercises multiple times per day. In some embodiments, exercise is low intensity (e.g., walking or hiking), moderate intensity (e.g., power walking, jogging, bicycling, yoga), or high intensity (e.g., weightlifting, running, sprinting, or recreational sports). In some embodiments, a subject exercises for at least 15 minutes, 20 minutes, 25 minutes, 30 minutes, 35 minutes, 40 minutes, or 45 minutes at a time. In some embodiments, the survey asks if the subject exercises and if so, which of the following determines how frequently the subject exercises: level of stress, level of free time, level of interest, accessibility of gym, how comfortable the subject feels at the gym, or other factors. In some embodiments, the survey asks the subject on average each week, how often (days per week) the subject performs low intensity exercise (walking, hiking, etc.) for at least 30 minutes. In some embodiments, the survey asks the subject on average each week, how often (days per week) the subject performs moderate exercise (power walking, jogging, bicycling, yoga, etc.) for at least 30 minutes. In some embodiments, the survey asks the subject on average each week, how often (days per week) the subject performs high intensity exercise (weightlifting, running, sprinting, recreational sports, or other high intensity exercise) for at least 30 minutes. In some embodiments, the subject has had exercise level impact the subject's period. In some embodiments, the subject has started or stopped the subject's period by changing the intensity of exercise. In some embodiments, the subject tracks steps with an app, and the survey asks on average how many steps the subject walks per day.

In some embodiments, a subject has stress, such as a stressful job. In some embodiments, a subject has many high-pressure demands on them daily, few high-pressure demands on them daily, good control of their day and/or priorities, minimal control over their day and/or priorities, frustration at their job, or satisfaction with their job and/or the role their job has in their life. In some embodiments, subjects work less than 30 hours per week between 30 and 40 hours per week, between 40 and 60 hours per week, between 60 and 80 hours per week, or more than 80 hours per week. In some embodiments, a subject does not have a job. In some embodiments, the employment status is full time, part time, contractor, salaried, multiple part-time, shift work (outside of the typical 9 to 5 business day), 9 am to 5 pm, Sam to 8 pm, night shift, self-employed, student, work from home, requires frequent crossing of time zones (pilot/flight attendant), or unemployed. In some embodiments, the survey collects information about job satisfaction or job security. In some embodiments, the survey asks the subject to choose which best describes the subject's type of employment, including without limitations, full time, part time, contractor, salaried, multiple part-time, shift work (outside of the typical 9 to 5 business day), 9 am to 5 pm, 8 am to 8 pm, night shift, self-employed, student, work from home, requires frequent crossing of time zones (pilot/flight attendant), unemployed, or other. In some embodiments, the survey asks how satisfied the subject is with the subject's job on a scale of 1 to 10. In some embodiments, the subject is concerned about job security.

In some embodiments, a subject has good (e.g., feeling refreshed in the morning) or poor (e.g., sleep can impact the quality of life) quality sleep. In some embodiments, a subject is satisfied with their sleep quantity and/or quality. In some embodiments, a subject is dissatisfied with their sleep quality and/or quantity. In some embodiments, the survey collects information about quantity of sleep, quality of sleep, medications involved in sleep. In some embodiments, the survey collects information using the Pittsburgh Sleep Quality Index. In some embodiments, the survey asks the subject how often they have had trouble sleeping in the path month because the subject woke up in the middle of the night or early morning, because the subject had to get up to take care of a child or other person living in the home, because the subject could not breathe comfortably, because the subject coughed or snored loudly, because the subject felt too cold, because the subject felt too hot, because the subject felt uncomfortable or in pain, because the subject had bad dreams, because the subject didn't have a place conducive to sleep (due to safety concerns, light, noise, or a combination thereof), because the subject had to get up to use the bathroom, because the subject could not get to sleep within 30 minutes, or because of any other reason. In some embodiments, the reason for trouble sleeping occurred at least 1, 2, 3, 4, 5, 6, or 7 times a week. In some embodiments, the reason for trouble sleeping have occurred at least 1, 2, 3, or 4 times during the past month.

In some embodiments, the survey asks how often the subject has had trouble staying awake while driving, eating meals, or during social activity. In some embodiments, the survey asks how often the subject has had a problem keeping up enthusiasm to get things done. In some embodiments, the survey asks the subject to rate the subject's sleep quality overall during the past month as very good, fairly good, fairly bad, or very bad.

In some embodiments, the survey collects information about caregiving. In some embodiments, the subject is a sole caregiver, a primary caregiver, an assistance caregiver, an occasional caregiver, or a professional caregiver. In some embodiments, the subject is a caregiver for a spouse/partner, parent, mother-in-law or father-in-law, child with special needs (under 18 years of age), child with special needs (over 18 years of age), sibling, grandparent, or a friend or neighbor. In some embodiments, the subject has been providing care for 1, 2, 3, 4, 5, 6, 7, 8, 9, 10, 11, or 12 months, or 1, 2, 3, 4, 5 years or more.

### Treatments and Subject Stratification

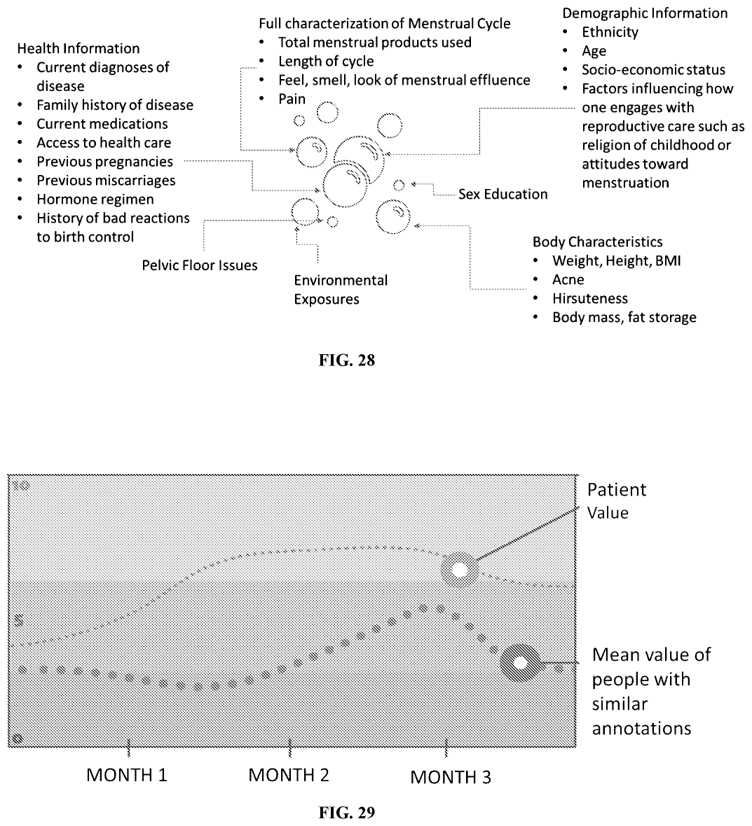

In some embodiments of methods provided herein, a biological marker or parameter from a fluid sample collected from a vaginal cavity of a subject as described herein and a feature from the survey as described herein are used to diagnose a subject. In some embodiments, the features from the survey are used to annotate a subject. In some embodiments, a feature or risk factor from the survey described herein is used to annotate a subject, as described in FIG. 28. In some embodiments, at least one feature from the survey described herein, a biological marker or a parameter from a fluid sample as described herein, or a combination thereof is used to stratify the subject into a treatment group. In some embodiments, a predictive model comprising at least one feature from the survey described herein, a biological marker or a parameter from a fluid sample as described herein, or a combination thereof is used to identify a condition of heavy menstrual bleeding. In some embodiments at least one feature from the survey described herein, a biological marker or a parameter from a fluid sample as described herein, or a combination thereof is used to generate a severity assessment of a menstrual bleeding state of a subject described herein.

In certain aspects, described herein is a method of using a predictive model to identify a condition of heavy menstrual bleeding, the method comprising using one or more processors in a computer server: receiving data defining a plurality of subject digital biomarkers, each digital biomarker comprising a response by the subject to an associated inquiry in a computing data storage; retrieving from computer data storage a combination of independent variables relating to the subject; using a predictive model to determine a dependent variable representing a subject HMB risk score for the subject based on the combination of independent variables relating to the subject; determining if the dependent variable representing the subject HMB score is above a threshold; and if the dependent variable representing the subject HMB score is above the threshold, sending information to be displayed. In certain aspects, described herein is a method for generating a severity assessment of a menstrual bleeding state of a human female subject, the method comprising: presenting one or more questions to the subject about a first set of attributes related to the subject's menstrual history and a second set of attributes related to a subject's menstrual phenotype, wherein the one or more questions are presented to the subject on a display of a graphic user interface of an input device; prompting the subject to enter a response to the one or more questions into the input device, wherein the input device transmits the response to a system comprising a processor and a computer-readable memory, wherein the system calculates an assessment score corresponding to menstrual bleeding state of the subject using the response to the one or more questions and stores the assessment score in the memory; using an assay to perform one or more measurements on a menstrual fluid sample from the subject; comparing the one or more measurements with one or more predetermined thresholds; and determine based on the calculated assessment score and the comparison with the one or more predetermined thresholds, the menstrual bleeding state of the subject; and generating a severity assessment of the subject's menstrual bleeding state. In certain aspects, described herein is a method of preparation of a biological sample comprising: identifying a subject; classifying the subject into a risk group based on one or more digital biomarkers; collecting menstrual fluid from a subject for a specified duration; measuring the volume of collected menstrual fluid; and calculating a flow rate from the volume and the specified duration.

In some embodiments, at least one biological marker or parameter from the fluid sample described herein is used to identify a condition in a subject. In some embodiments, the subject has been annotated with a feature from the survey. In some embodiments, the subject has been annotated into

In some embodiments, a marker or parameter from one or more fluid samples collected from a vaginal cavity of a subject, such as menstrual fluid, is used to diagnose a subject, for example, with HMB, AUB, or another menstrual cycle disorder. In some embodiments, a cell type, gene expression, gene target biomarker, nucleic acid, a flow rate, or a combination thereof is used to diagnose a subject.

In some embodiments, a marker or parameter from one or more fluid samples collected from a vaginal cavity of a subject, such as menstrual fluid, is used to identify a condition in a subject, for example, with HMB, AUB, or another menstrual cycle disorder. In some embodiments, a cell type, gene expression, gene target biomarker, nucleic acid, a flow rate, or a combination thereof is used to identify a condition in a subject.

In some embodiments, a diagnosis is a probability or likelihood that a subject has HMB, AUB, or another menstrual cycle disorder. In some embodiments, a diagnosis is expressed as a risk score, a percentage, a fraction, or another analog measurement. In some embodiments, a severity assessment of a menstrual bleeding state is calculated. In some embodiments, a measurement such as a risk score is based on a single marker or parameter. In some embodiments, a measurement such as a risk score is based on more than one marker or parameter. In some embodiments, a diagnosis is a positive diagnosis (i.e., the subject has or is likely to have HMB, AUB, or another menstrual cycle disorder) or a negative diagnosis (i.e., the subject does not have or is not likely to have HMB, AUB, or another menstrual cycle disorder). In some embodiments, a subject is a positive diagnosis of more than one of HMB, AUB, or another menstrual cycle disorder. In some embodiments, a subject is a negative diagnosis of more than one of HMB, AUB, or another menstrual cycle disorder. In some embodiments, a subject is a both a positive diagnosis of HMB, AUB, or another menstrual cycle disorder and a negative diagnosis of a different of HMB, AUB, or another menstrual cycle disorder. In some embodiments, a subject has a positive diagnosis of AUB and a negative diagnosis of HMB. In some embodiments, a subject has a positive diagnosis of HMB and a positive diagnosis of endometriosis.

In some embodiments, a diagnosis is expressed as a “yes” or “no” diagnosis. In some embodiments, a diagnosis is achieved, e.g., by determining that a marker or parameter is above or below a predetermined threshold as described herein.

In some embodiments, a subject is stratified into a treatment group based on one or more markers or parameters of one or more fluid samples collected from the vaginal cavity of the subject. In some embodiments, stratification is based on a diagnosis of HMB, AUB, one or more menstrual cycle disorders, or a combination thereof. In some embodiments, a subject is stratified into one treatment group, more than one treatment group, or no treatment groups.

In some embodiments, stratification is based on a risk factor. In some embodiments, a risk factor that is used for stratification is a risk factor for HMB, AUB, or another menstrual cycle disorder, or a risk factor for an adverse effect of a treatment that a treatment group might receive. In some embodiments, a subject having a menstrual cycle disorder such as endometriosis is stratified into a treatment group or not depending on if they have a risk factor (e.g., allergies, other drugs which interacts with treatment, pregnancy, etc.) that negatively affect either treatment or the health of the subject.

In some embodiments, risk factors include but are not limited to age, gender, history of heart disease, family history of heart disease, history of cancer, family history of cancer, allergies, other medications, activity level, weight, body mass index, cholesterol level, blood pressure, or other factors. In some embodiments, risk factors comprises a digital biomarker or features as described herein.

In some embodiments, subjects is stratified into one treatment group. In some embodiments, subjects is stratified into two or more treatment groups. In cases, where there are two or more treatment groups, treatment groups differ by dose of treatment, type of treatment (different drugs, drugs vs. alternate therapies, etc.), dietary regimen, supplements, exercise regimen, etc. In some embodiments, a treatment group comprises no treatment.

In some embodiments, a classification system comprising one or more classifiers is implemented to compare one or more features of menstrual fluid or cervicovaginal fluid of a subject suspected of having HMB, AUB, or another menstrual cycle disorder to a control or other reference sample. In some embodiments, features that are compared using a classifier include protein biomarker expression, gene biomarker expression, RNA expression, nucleic acid content, flow rate, microbiome, or another feature.

In some embodiments, a classifier calculates a metric and compare the metric to a reference value to determine whether a subject has HMB, AUB, or another menstrual cycle disorder. In some embodiments, a classifier algorithm further comprises a correction for age. In some embodiments, the correction for gestational age comprises a LOESS correction. In some embodiments, the classifier algorithm comprises a logistic regression. In some embodiments, the classifier algorithm comprises a logistic regression with elastic-net regularization. In some embodiments, the classifier algorithm comprises a Random Forest.

In some instances, the classifier is a two-way classifier. In some embodiments, a two-way classifier classifies a sample from a subject having HMB from a sample from a subject experiencing normal menstrual bleeding, a sample from a subject having AUB from a sample from a subject experiencing normal menstrual bleeding, or a sample from a subject having a menstrual cycle disorder from a sample from a subject experiencing normal menstrual bleeding. In some instances, the classifier used classifies a subject as not needing treatment for preeclampsia. In some instances, a multi-way classifier is used (e.g., preeclampsia, non-preeclampsia, and indeterminate).

In some embodiments, classifiers and/or classifier probe sets (e.g., antibody sets) is used to either rule-in or rule-out a sample as from a patient experiencing HMB, AUB, or another menstrual cycle disorder. In some embodiments, a classifier is used to classify a sample as being from a healthy subject. In some embodiments, alternatively, a classifier is used to classify a sample as being from an unhealthy subject, such as a subject experiencing HMB, AUB, or another menstrual cycle disorder. In some embodiments, alternatively, or additionally, classifiers is used to either rule-in or rule-out a sample as being from a subject who should be treated for HMB, AUB, or another menstrual cycle disorder.

In some embodiments, the methods, kits, and systems disclosed herein include at least one computer program, or use of the same. In some embodiments, a computer program include a sequence of instructions, executable in the digital processing device's CPU (i.e., processor), written to perform a specified task. In some embodiments, computer readable instructions is implemented as program modules, such as functions, objects, Application Programming Interfaces (APIs), data structures, and the like, that perform particular tasks or implement particular abstract data types. In light of the disclosure provided herein, those of skill in the art will recognize that a computer program is written in various versions of various languages.

In some embodiments, the functionality of the computer readable instructions is combined or distributed as desired in various environments. In some embodiments, the computer program will normally provide a sequence of instructions from one location or a plurality of locations.

Further disclosed herein are systems for classifying (or ruling out a classification) one or more samples and uses thereof. In some embodiments, the system comprises (a) a digital processing device comprising an operating system configured to perform executable instructions and a memory device; (b) a computer program including instructions executable by the digital processing device to classify a sample from a subject comprising: (i) a first software module configured to receive a biomarker expression profile of one or more biomarkers from the sample from the subject; (ii) a second software module configured to analyze the biomarker expression profile from the subject; and (iii) a third software module configured to classify the sample from the subject based on a classification system. In some embodiments, the classification system comprises two classes. In other embodiments, the classification system comprises two or more classes. In some embodiments, at least two of the classes is selected from preeclampsia, non-preeclampsia (e.g., for at least a period of time), normal pregnancy, complicated pregnancy, and gestational hypertension. In some embodiments, analyzing the biomarker expression profile from the subject comprises applying an algorithm. In some embodiments, analyzing the biomarker expression profile comprises normalizing the biomarker expression profile from the subject.

In some embodiments, the methods, kits, and systems disclosed herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device will include an operating system configured to perform executable instructions. In some embodiments, the operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device generally includes a storage and/or memory device. In some embodiments, the storage and/or memory device is one or more physical apparatuses used to store data or programs on a temporary or permanent basis. In some embodiments, the device is volatile memory and requires power to maintain stored information. In some embodiments, the device is nonvolatile memory and retains stored information when the digital processing device is not powered. In further embodiments, the non-volatile memory comprises flash memory. In some embodiments, the non-volatile memory comprises dynamic random-access memory.

In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a display to send visual information to a user will normally be initialized. In some embodiments, displays include a cathode ray tube (CRT, a liquid crystal display (LCD), a thin film transistor liquid crystal display (TFT-LCD, an organic light emitting diode (OLED) display. In various further embodiments, on OLED display is a passive-matrix OLED (PMOLED) or active-matrix OLED (AMOLED) display. In some embodiments, the display is a plasma display, a video projector or a combination of devices such as those disclosed herein.

In some embodiments, the digital processing device would normally include an input device to receive information from a user. In some embodiments, the input device is, for example, a keyboard, a pointing device including, by way of non-limiting examples, a mouse, trackball, track pad, joystick, game controller, or stylus; a touch screen, or a multi-touch screen, a microphone to capture voice or other sound input, a video camera to capture motion or visual input or a combination of devices such as those disclosed herein.

In some embodiments, the methods, kits, and systems disclosed herein include one or more non-transitory computer readable storage media encoded with a program including instructions executable by the operating system to perform and analyze the test described herein; preferably connected to a networked digital processing device. In some embodiments, the computer readable storage medium is a tangible component of a digital device that is optionally removable from the digital processing device. In some embodiments, the computer readable storage medium includes, by way of non-limiting examples, CD-ROMs, DVDs, flash memory devices, solid state memory, magnetic disk drives, magnetic tape drives, optical disk drives, cloud computing systems and services, and the like. In some instances, the program and instructions are permanently, substantially permanently, semi-permanently, or non-transitorily encoded on the media.

In some embodiments, a non-transitory computer-readable storage media is encoded with a computer program including instructions executable by a processor to create or use a classification system. In some embodiments, the storage media comprises (a) a database, in a computer memory, of one or more clinical features of two or more control samples, wherein (i) the two or more control samples is from two or more subjects; and (ii) the two or more control samples is differentially classified based on a classification system comprising two or more classes; (b) a first software module configured to compare the one or more clinical features of the two or more control samples; and (c) a second software module configured to produce a classifier set based on the comparison of the one or more clinical features. In some embodiments, at least two of the classes is selected from preeclampsia, non-preeclampsia, normal pregnancy, complicated pregnancy, and gestational hypertension.

### EXAMPLES

### Example 1: Collection of a Sample Using a Collection Device

A sample can be collected from a subject, for example a menstruating subject, using a collection device. The subject can be experiencing HMB or AUB. The collection device can be placed inside or under the vagina and left in place for a pre-determined amount of time to collect fluid, such as menstrual fluid or cervicovaginal fluid.

For example, a collection device can be a tampon. In such a case, the tampon can be inserted into the vagina with either an applicator or fingers, and left in place for approximately 4, 5, 6, 7, or 8 hours. After the time has elapsed, the tampon can be removed and placed in a vessel such as a collection packet. The vessel containing the tampon containing the sample can be stored or shipped. For example, it can be shipped or mailed to a laboratory for analysis.

### Example 2: Measurement of Gene Expression of a Sample

In an instance of this example, a collection device such as a tampon can be inserted into the vagina of a menstruating subject suspected of having HMB, AUB, or another menstrual cycle disorder. After 8 hours, the tampon can be removed.

The sample can be extracted from the collection device. For example, the tampon can be dissolved or degraded, or the sample can be removed by squeezing or centrifuging.

RNA can be analyzed from the liquid fraction of a sample. In such cases, the sample can be centrifuged (e.g., at 2000×g for 5-7 minutes at 4° C.), and the supernatant can be collected and set aside for analysis.

RNA can be analyzed from the cellular fraction of a sample. In such cases, the sample can be centrifuged (e.g., at 2000×g for 5-7 minutes at 4° C.), the supernatant can be washed away, the cells can be washed with ice cold PBS, and again centrifuged (e.g., at 2000×g for 5-7 minutes at 4° C.).

RNA can be extracted from the sample using an acceptable method, e.g., using Trizol reagent. Once extracted, RNA can be transferred to an Eppendorf tube for further processing.

In some cases, magnetic beads or a silica membrane can be used to purify RNA via selective binding. To isolate RNA using such a technique, a volume of sample (e.g., 3 mL) of specimen can be removed and subjected to an organic phase extraction to remove impurities. The aqueous phase can be removed, and RNA molecules in solution can be precipitated out by the addition of ethanol. The RNA homogenate can be applied to magnetic beads or silica membrane where RNA can be selectively bound. The bound RNA can be washed, and residual genomic DNA can be removed from the membrane or bead solution, such as by treatment of the bound nucleic acid with DNase I. RNA can be eluted using the elution buffer such as one provided in standard nucleic acid extraction kits or using RNase free water.

The extracted RNA can be washed, for example using a wash buffer, which can comprise about 70% ethanol and about 30% water or other acceptable wash buffer. Washing can comprise pipetting a volume (e.g., at least 500 μL) of the wash buffer into the Eppendorf tube containing the RNA, pipetting up and down gently or gently flicking the tube, briefly spinning the Eppendorf tube containing the RNA on a benchtop centrifuge, and removing the wash buffer by pouring or by pipette.

The extracted RNA can be purified. For example, the RNA can be purified by vacuum filtration or by centrifugal filtration. In such cases, the RNA can be solubilized in RNase free water, applying the RNA to a vacuum filter or a centrifugal filter, and using either a vacuum or centrifuge as appropriate to filter the buffer and impurities from the RNA. The RNA can then be eluted into a different Eppendorf tube by applying an elution buffer (e.g., about 50 μL of elution buffer), allowing the RNA to dissolve in the Elution buffer, and using either a vacuum or centrifuge as appropriate to elute the RNA.

The extracted RNA can be measured, for example to determine the amount of RNA in the sample. For example, RNA can be quantified by measuring its absorbance. In one such method, 1 μL of RNA can be loaded onto a NanoDrop spectrophotometer, providing a measurement of the absorbance at 260 nm (A₂₆₀), the absorbance at 280 nm (A₂₈₀), and the absorbance at 230 nm (A₂₃₀). Concentration can be calculated using the A₂₆₀ measurement. The conversion for such a calculation can be A₂₆₀ of 1.0=40 μg/mL. Protein in the sample (e.g., as a contaminant) can be detected at 280 nm. Other contaminants in the sample (e.g., guanidine thiocyanate) can be detected at 230 nm. In some cases, an absorbance reading at 320 nm (A₃₂₀) can be taken to detect light scattering components in the sample, and subtracted from the A₂₆₀ value as a background removing step.

Other methods for quantifying RNA can comprise fluorescent dye-based quantification. In such methods, a nucleic acid sample along with a series of standards of known concentrations can be incubated with a fluorescent dye that can bind to the nucleic acid and undergo a conformational change. Such a reaction can result in an increased fluorescence at a wavelength specific to a dye being used. Fluorescence can be measured using a plate reader or a handheld fluorometer, and a standard curve (e.g., for a plate reader) or a reference standard (e.g., for a handheld fluorometer) can be created by plotting fluorescence against nucleic acid concentrations of the known standards. The fluorescence can be converted to nucleic acid concentration for example by using the linear regression equation that best describes the standard curve.

The extracted RNA can be stored. The RNA can then undergo analysis. Analysis can include PCR amplification of a gene product of interest, such as a gene biomarker, and can also include PCR amplification of a housekeeping gene such as GAPDH or β-actin for normalization of the gene expression results. For example, 1× reaction buffer, 200 μM of dNTPs, 0.2 μM forward primer, 0.2 μM reverse primer, approximately 1,000 ng of template nucleic acid, and 1.25 units of Taq polymerase can be prepared in nuclease free water. PCR thermocycling conditions can follow a standard protocol, such as an initial denaturation step of 95° C. for 30 seconds, followed by 30 cycles of 95° C. for 15-30 seconds, 45° C.-68° C. for 15-60 seconds, and 68° C. for 1 minute per kilobase (amplification), followed by a final extension step of 68° C. for 5 minutes, followed by an optional hold at between 4° C. and 10° C. inclusive. The PCR product can be quantified, e.g., via q-RT-PCR, northern blot, or gel-electrophoresis. The results can be analyzed and compared with normal levels of that gene. In some cases, an alternate method of gene expression quantification can be used, such as an imaging-based method (e.g., in situ hybridization).

### Example 3: Measurement of Cell Types of a Sample

In an instance of this example, a collection device such as a tampon can be inserted into the vagina of a menstruating subject suspected of having HMB, AUB, or another menstrual cycle disorder. After 8 hours, the tampon can be removed.

A sample can be extracted from a collection device such as a tampon. For example, the tampon can be dissolved or degraded, or the sample can be removed by squeezing or centrifuging. Cells can be extracted from the sample using an acceptable method, such as by centrifugation or filtration. In some cases, after cells are extracted, they can be washed 1-3 times with a volume of a buffer such as phosphate buffered saline (PBS) based buffer or a media such as Dulbecco's Modified Eagle Medium (DMEM). Buffer volume can be between 500 μL and 10 mL. In some cases, buffer volume can be about 500 about 1 mL, about 1.5 mL, about 2 mL, about 2.5 mL, about 3 mL, about 4 mL, about 5 mL, about 6 mL, about 7 mL, about 8 mL, about 9 mL, or about 10 mL. In some cases, buffer volume can be at least 500 at least 1 mL, at least 1.5 mL, at least 2 mL, at least 2.5 mL, at least 3 mL, at least 4 mL, at least 5 mL, at least 6 mL, at least 7 mL, at least 9 mL, or at least 10 mL. In some cases, buffer volume can be no more than 500 no more than 1 mL, no more than 1.5 mL, no more than 2 mL, no more than 2.5 mL, no more than 3 mL, no more than 4 mL, no more than 5 mL, no more than 6 mL, no more than 7 mL, no more than 8 mL, no more than 9 mL, or no more than 10 mL.

The cells can be analyzed to determine their cell type. For example, immunohistochemistry or immunofluorescence staining can be used to determine the cell type. In such a protocol, cells can be fixed to a slide, blocked with a blocking buffer such as fetal bovine serum or 5% w/v nonfat dry milk in Tris-buffered saline with Tween 20 (TBST), and stained with one or more antibodies raised against one or more cell surface markers for one or more cell types. For example, a primary antibody in blocking buffer (e.g., between 50 μL and 200 μL) can be incubated on the slide for between 1 and 24 hours, followed by washing (e.g., 3 washes of 5 minutes each in TBST) and subsequent incubation of a labeled secondary antibody in blocking buffer (e.g., between 50 μL and 200 μL) can be incubated on the slide for between 1 hour and 4 hours, followed by washing (e.g., 3 washes of 5 minutes each in TBST). Next, the slides can be imaged, for example using a fluorescence microscope, to detect the one or more antibodies and identify one or more cell types. In some cases, these steps can be performed in a multiwell plate, and the one or more antibodies can be detected using a plate reader. Cell types in the sample can then be quantified, analyzed, and compared with the cell types in a typical sample (e.g., a control sample).

In another instance of this example, cells can be labeled with one or more antibodies raised against one or more cell surface markers for one or more cell types. The antibodies can be labeled, such as by a fluorescent molecule or magnetic molecule. Each different antibody can have a wavelength that is different than other antibodies. Cells can then undergo cell sorting using a method such as fluorescence activated cell sorting (FACS) or magnetic activated cell sorting (MACS) to determine cell type. Cell types in the sample can then be quantified, analyzed, and compared with the cell types in a typical sample (e.g., a control sample).

### Example 4: Measurement of Nucleic Acid Content of a Sample

In an instance of this example, a collection device such as a tampon can be inserted into the vagina of a menstruating subject suspected of having HMB, AUB, or another menstrual cycle disorder. After 8 hours, the tampon can be removed.

A sample can be extracted from a collection device such as a tampon. For example, the tampon can be dissolved or degraded, or the sample can be removed by squeezing or centrifuging.

Nucleic acids (e.g., RNA, DNA, or RNA and DNA) can be extracted from the sample using an acceptable method, e.g., using Trizol reagent. Once extracted, nucleic acid can be transferred to an Eppendorf tube for further processing.

Nucleic acid can be analyzed from the liquid fraction of a sample. In such cases, the sample can be centrifuged (e.g., at 2000×g for 5-7 minutes at 4° C.), and the supernatant can be collected. Supernatant can be collected and set aside for analysis.

Nucleic acid can be analyzed from the cellular fraction of a sample. In such cases, the sample can be centrifuged (e.g., at 2000×g for 5-7 minutes at 4° C.), the supernatant can be washed away, the cells can be washed with ice cold PBS, and again centrifuged (e.g., at 2000×g for 5-7 minutes at 4° C.).

In some cases, magnetic beads or a silica membrane can be used to purify nucleic acid via selective binding. To isolate nucleic acid using such a technique, a volume of sample (e.g., 3 mL) of specimen can be removed and subjected to an organic phase extraction to remove impurities. The aqueous phase can be removed, and nucleic acid molecules in solution can be precipitated out by the addition of ethanol. The nucleic acid homogenate can be applied to magnetic beads or silica membrane where nucleic acid can be selectively bound. The bound nucleic acid can be washed, and residual genomic DNA can be removed from the membrane or bead solution, such as by treatment of the bound nucleic acid with DNase I if desired (e.g., if RNA only is desired), or residual RNA can be removed such as by treatment of the bound nucleic acid with an RNase if desired (e.g., if DNA only is desired). Nucleic acid can be eluted using the elution buffer such as one provided in standard nucleic acid extraction kits or using RNase free water (for RNA purification), DNase free water (for DNA purification), or RNase and DNase free water (for purification of both RNA and DNA).

The extracted nucleic acids can be washed, for example, using a wash buffer, which can comprise about 70% ethanol and about 30% water or other acceptable wash buffer. Washing can comprise pipetting a volume (e.g., at least 500 μL) of the wash buffer into the Eppendorf tube containing the nucleic acids, pipetting up and down gently or gently flicking the tube, briefly spinning the Eppendorf tube containing the nucleic acids on a benchtop centrifuge, and removing the wash buffer by pouring or by pipette.

The extracted nucleic acids can be purified. For example, the nucleic acids can be purified by vacuum filtration or by centrifugal filtration. In such cases, the nucleic acids can be solubilized in RNase free water, DNase free water, or RNase and DNase free water, applying the nucleic acids to a vacuum filter or a centrifugal filter, and using either a vacuum or centrifuge as appropriate to filter the buffer and impurities from the nucleic acids. The nucleic acids can then be eluted into a different Eppendorf tube by applying an elution buffer (e.g., about 50 μL of elution buffer), allowing the nucleic acids to dissolve in the Elution buffer, and using either a vacuum or centrifuge as appropriate to elute the nucleic acids.

The extracted nucleic acids can be measured, for example to determine the amount of nucleic acid in the sample. For example, nucleic acid can be quantified by measuring its absorbance. In one such method, 1 μL of nucleic acid can be loaded onto a NanoDrop spectrophotometer, providing a measurement of the absorbance at 260 nm (A₂₆₀), the absorbance at 280 nm (A₂₈₀), and the absorbance at 230 nm (A₂₃₀). Concentration can be calculated using the A₂₆₀ measurement. The conversion for such a calculation can be A₂₆₀ of 1.0=40 μg/mL for RNA, A₂₆₀ of 1.0=50 μg/mL for double stranded DNA, or A₂₆₀ of 1.0=33 μg/mL for single stranded DNA. Protein in the sample (e.g., as a contaminant) can be detected at 280 nm. Other contaminants in the sample (e.g., guanidine thiocyanate) can be detected at 230 nm. In some cases, an absorbance reading at 320 nm (A₃₂₀) can be taken to detect light scattering components in the sample, and subtracted from the A₂₆₀ value as a background removing step.

Other methods for quantifying RNA can comprise fluorescent dye-based quantification. In such methods, a nucleic acid sample along with a series of standards of known concentrations can be incubated with a fluorescent dye that can bind to the nucleic acid and undergo a conformational change. Such a reaction can result in an increased fluorescence at a wavelength specific to a dye being used. Fluorescence can be measured using a plate reader or a handheld fluorometer, and a standard curve (e.g., for a plate reader) or a reference standard (e.g., for a handheld fluorometer) can be created by plotting fluorescence against nucleic acid concentrations of the known standards. The fluorescence can be converted to nucleic acid concentration for example by using the linear regression equation that best describes the standard curve.

The extracted nucleic acids can be stored. In some cases, the extracted nucleic acids can be dried prior to storage. Drying can comprise air drying, drying under nitrogen, or freeze drying for example. Nucleic acids can be stored at room temperature, at a refrigerated temperature (e.g., about 4° C.)., or freezing (e.g., about −20° C. or about −80° C.). Nucleic acids can be stored for a period of time, e.g., 1 day, 2 days, 3 days, 5 days, 6 days, 7 days, 2 weeks, 3 weeks, 4 weeks, or more.

The nucleic acids can be analyzed, for example, to determine the total amount of nucleic acid in the sample. The analysis can be a qualitative analysis, for example to determine whether a sample has more or less nucleic acid than a standard, control, or other sample, or to determine the presence or absence of nucleic acid in the sample, or to determine the approximate size range of nucleic acid in a sample. The analysis can be a quantitative analysis, for example to determine the quantity of nucleic acid in a sample, either absolutely or relative to a standard such as a standard curve.

In some cases, analysis can comprise gel electrophoresis. Gel electrophoresis can comprise first preparing an agarose gel. The agarose gel can be between 0.7% and 2% agarose in TAE. In some instances, the agarose gel can be 1% agarose in TAE. An appropriate amount of agarose can be added to a volume of TAE. For example, 100 mL TAE with 1 g agarose can yield a 1% TAE gel. The mixture can be heated, for example, in a microwave, for between 1 and 3 minutes, or until the agarose is completely dissolved. The agarose solution can be allowed to cool, for example to about 50° C. and then poured into a mold. In some cases, ethidium bromide can be added to the agarose gel after heating and cooling and prior to pouring, such that the final concentration of ethidium bromide is between about 0.2 μg/mL and 0.5 μg/mL. The sample(s) can be mixed gently with a loading buffer. Loading buffer can be any acceptable buffer, and can comprise a dye. Common loading buffers include Ficoll and Orange G, sucrose and xylene cyanol/bromophenol blue, glycerol and bromophenol blue, and NEB loading dye. The samples can be loaded into the wells in the gel, along with a molecular weight ladder and standard or control samples if desired. In some cases, at least 5 ng of nucleic acid, at least 10 ng of nucleic acid, at least 15 ng of nucleic acid, or at least 20 ng of nucleic acid can be loaded into the gel. In some cases, no more than 75 ng of nucleic acid, no more than 100 ng of nucleic acid, no more than 125 ng of nucleic acid, or no more than 150 ng of nucleic acid can be loaded into the gel. In some cases, between 10 ng of nucleic acid and 100 ng of nucleic acid can be loaded into the gel. The gel can run at between 80 V and 150 V. In some cases, the gel can run at about 70 V, about 80 V, about 90 V, about 100 V, about 110V, about 120 V, about 130 V, about 140 V, or about 150 V. In some cases, where ethidium bromide was added to the gel, the DNA can be observed under ultraviolet light (e.g., about 260 nm, about 300 nm, or about 360 nm) as emitted light (e.g., about 590 nm). In some cases where ethidium bromide is not added to the gel, a dye in the loading buffer can be employed to detect nucleic acid bands, for example under visible or ultraviolet light. In some cases where ethidium bromide is not added to the gel, the gel can be soaked in either ethidium bromide or another dye, and the nucleic acid can be visualized such as by ultraviolet light. In some cases, the nucleic acid bands in the gel can be imaged, and the size or intensity of the bands can be used to quantify the nucleic acid (e.g., relative to a standard, relative to an internal standard, using a standard curve, or relative to a control). In some cases, the presence or absence of a band(s) of a certain size(s) can be determined.

In some cases, analysis can comprise measurement of the absorbance of the sample.

Nucleic acid dyes can be intercalating dyes, minor-groove binding dyes, or other nucleic acid stains and dyes. Examples of nucleic acid dyes can include ethidium bromide, SYBR gold, SYBR green, SYBR safe, Eva green, propidium iodide, crystal violet, dUTP-conjugated probes, 4′,6-diamidino-2-phenylindole, 7-aminoactinomycin D, Hoechst 33258 (33342, 34580), or YOYO-1/DiYO-1/TOTO-1/DiTO-1. For example, a fluorescent dye can be added to a blank, one or more nucleic acid standards, and one or more samples. The dye can be incubated with the nucleic acid to allow binding of the dye to the nucleic acid. The incubation can be performed at room temperature, but might also be performed at another temperature, e.g., 4° C. The blank and standard samples can be measured according to the dye used. For example, a fluorescent dye can be detected using fluorescent spectrophotometry and a regression analysis can be performed to generate a standard curve. For example, if a linear range of standards is used, then a linear regression analysis can be performed. In some cases, a logarithmic range of standards can be used, and a logarithmic regression analysis can be performed. The regression analysis can yield a fit, such as a linear fit or a logarithmic fit. The fit can then be used to calculate the amount of nucleic acid in the sample.

### Example 5: Measurement of Flow Rate of a Sample

In an instance of this example, a collection device such as a tampon can be inserted into the vagina of a menstruating subject suspected of having HMB, AUB, or another menstrual cycle disorder. After 8 hours, the tampon can be removed. In some cases, the collection device can be removed after less than 8 hours or after more than 8 hours. Importantly, the collection device is left in the vagina for a known amount of time.

A sample can be extracted from a collection device such as a tampon. For example, the tampon can be dissolved or degraded, or the sample can be removed by squeezing or centrifuging.

The sample can then be measured to determine the volume. Volume can be measured using a pipette, a graduated cylinder, or by another method. The ratio of the volume collected to the amount of time of sample collection can be the flow rate (e.g., as rate=volume/time). Flow rate can be measured during one menstrual window or during multiple menstrual windows.

In some cases, the flow rate can be measured serially. In such cases, a collection device can be inserted into the vagina of a menstruating subject suspected of having HMB, AUB, or another menstrual cycle disorder. After a set amount of time (e.g., 30 minutes, 1 hour, 2 hours, 3 hours, 4 hours, etc.), the tampon can be removed. The sample can be extracted from a collection device and volume measured as described above. After removal of the collection device, a second collection device can be inserted into the vagina for a set amount of time, after which the second sample can be extracted from the second collection device and volume measured as described above. This process can be repeated 1, 2, 3, 4, 5, 6, 7, 8, 9, 10, or n times. Collection of a sample can occur immediately after collection of the previous sample, or a period of time (e.g., 1 minute, 15 minutes, 30 minutes, 1 hour, 2 hours, 6 hours, 12 hours, 1 day, or another suitable period of time) can elapse between collection of serial samples. Flow rate can be calculated for each of the serially collected samples as the ratio of the volume collected to the amount of time of sample collection. Serially collected samples can be averaged to determine a mean flow rate. In some cases, a maximum flow rate, minimum flow rate, or mode flow rate can be determined. In some cases, flow rate is measured as depicted in Table 6.

TABLE 6
Flow rate in a sample from an individual

| Day 1 | Day 2 | Day 3 | Day 4 |
|---|---|---|---|
| Volume of Menstrual | Volume of Menstrual | Volume of Menstrual | Volume of Menstrual |
| Blood taken on Day 1 in 2 | Blood taken on Day 2 in 2 | Blood taken on Day 3 in 2 | Blood taken on Day 4 in 2 |
| hr time unit | hr time unit | hr time unit | hr time unit |
| Volume of Menstrual | Volume of Menstrual | Volume of Menstrual | Volume of Menstrual |
| Blood taken on Day 1 in 1 | Blood taken on Day 2 in 1 | Blood taken on Day 3 in 1 | Blood taken on Day 4 in 1 |
| hr time unit | hr time unit | hr time unit | hr time unit |
| Volume of Menstrual | Volume of Menstrual | Volume of Menstrual | Volume of Menstrual |
| Blood taken on Day 1 in | Blood taken on Day 2 in | Blood taken on Day 3 in | Blood taken on Day 4 in |
| 45 minute time unit | 45 minute time unit | 45 minute time unit | 45 minute time unit |
| Volume of Menstrual | Volume of Menstrual | Volume of Menstrual | Volume of Menstrual |
| Blood taken on Day 1 in | Blood taken on Day 2 in | Blood taken on Day 3 in | Blood taken on Day 4 in |
| 30 minute time unit | 30 minute time unit | 30 minute time unit | 30 minute time unit |
| Volume of Menstrual | Volume of Menstrual | Volume of Menstrual | Volume of Menstrual |
| Blood taken on Day 1 in | Blood taken on Day 2 in | Blood taken on Day 3 in | Blood taken on Day 4 in |
| 15 minute time unit | 15 minute time unit | 15 minute time unit | 15 minute time unit |

### Example 6: Measurement of Protein Biomarkers of a Sample

In an instance of this example, a collection device such as a tampon can be inserted into the vagina of a menstruating subject suspected of having HMB, AUB, or another menstrual cycle disorder. After 8 hours, the tampon can be removed.

A sample can be extracted from a collection device such as a tampon. For example, the tampon can be dissolved or degraded, or the sample can be removed by squeezing or centrifuging.

Protein can be extracted from the sample using an acceptable method. In some cases, RIPA buffer can be used for protein extraction. RIPA buffer can comprise 150 mM NaCl, 1% Triton X-100, 0.5% Sodium deoxycholate, 0.1% sodium dodecyl sulfate, 50 mM Tris-HCl (titrate to pH 8.)), and 1× protease inhibitor. In some cases, another lysis buffer or extraction buffer can be used.

Protein can be analyzed from the liquid fraction of a sample. In such cases, the sample can be centrifuged (e.g., at 2000×g for 5-7 minutes at 4° C.), and the supernatant can be collected. Supernatant can be collected and set aside for analysis.

Protein can be analyzed from the cellular fraction of a sample. In such cases, the sample can be centrifuged (e.g., at 2000×g for 5-7 minutes at 4° C.), the supernatant can be washed away, the cells can be washed with ice cold PBS, and again centrifuged (e.g., at 2000×g for 5-7 minutes at 4° C.). the PBS can be removed, and ice-cold RIPA buffer can be added to the cell pellet and agitated (e.g., for 30 minutes at 4° C.). The samples can be centrifuged (e.g., at 16000×g for 20 minutes at 4° C.), and the supernatant can be retained for analysis.

In some cases, protein concentration can be normalized across samples prior to analysis. For example, a small volume of lysate can be used to perform a protein estimation assay such as a bicinchoninic acid (BCA) assay. Briefly, standards of known protein concentration can be prepared, e.g., using bovine serum albumin. Standards can range between 2000 μg/mL and 25 μg/mL, as well as a blank sample containing no protein. A working reagent can be prepared by mixing 50 parts of a reagent comprising sodium carbonate, sodium bicarbonate, bicinchoninic acid and sodium tartrate in 0.1 M sodium hydroxide with one part of a second reagent comprising 4% cupric sulfate. 25 μL of each sample or standard can be pipetted onto a microplate, and 200 μL of working reagent can be added to each well. The plate can be mixed thoroughly, covered, and incubated at 37° C. for 30 minutes. The plate can be cooled to room temperature, and the absorbance can be measured at or near 562 nm on a plate reader. A standard curve can be determined, and the concentration of proteins in each sample can be calculated using the standard curve. Other methods for calculating protein concentration can include a Bradford assay or a nanodrop assay. An appropriate volume of lysate can be added to each sample to ensure each sample contains the same protein concentration.

Protein can then be measured. In some cases, protein can be measured by western blotting. A loading buffer can be added to each sample, and each sample can be loaded onto an SDS gel for SDS-PAGE gel electrophoresis to separate the proteins according to size. After electrophoresis, in some cases, the gel can be stained to visualize the protein, for example by Coomassie blue staining or silver staining. After electrophoresis, the protein can be transferred onto a membrane using either a tank transfer or a semi-dry transfer technique. The membrane can be blocked, and immunodetection can be performed using a primary antibody against a protein biomarker, and a fluorescent secondary antibody against the primary antibody. The protein can be detected using radiographic film or a fluorescence-sensitive camera and quantified.

Protein can be measured by ELISA. Briefly, a capture antibody against a protein biomarker of interest can be coated to the bottom of a well of a microplate, and a sample can be incubated in the well to allow binding of protein of interest in the sample to the capture antibody. In some cases, this capture antibody may be absent, and the sample protein can be coated to the bottom of a well of a microplate. A second antibody against the protein biomarker of interest can be incubated in the well, and excess antibody can be washed away. A third antibody against the second antibody can be incubated in the well, such that the third antibody is detectible (e.g., conjugated to HRP or a fluorescent tag). A plate reader can be used to measure the intensity of signal from the well, and using a standard curve, the amount of the protein biomarker in the sample can be calculated.

A proteomic analysis can be performed. Proteomic analysis can comprise detecting proteins in a sample using a mass-spectrometry (MS) method, such as electrospray ionization MS/MS. The results of the protein detection can be evaluated for example using statistical means or using machine learning or deep learning means. In some cases, a differential expression of proteins present during HMB, AUB, or another disease or disorder can be determined using such a method.

The protein can be washed, purified, quantified, and/or stored. The protein can undergo analysis for identification, such as western blotting analysis or ELISA analysis. In some cases, a proteomic analysis can be performed. Protein in the sample can then be compared with protein in a typical sample (e.g., a control sample).

### Example 7: Tampon Collection System and Use

Menstrual fluid (2.5 mL) can be collected into a collection device via a low-absorbency, all-cotton tampon. The tampon is placed in the sample collection device, the lid is snapped shut, and the base of the device is twisted counterclockwise to release a DNA/RNA preservation buffer (7.5 mL) and elute biological material from the tampon by compression forces. Preserved biological specimen is removed from the collection device through an outlet valve at the bottom of the base (6 mL of preserved specimen is removed for downstream processing). Once removed from the device, the preserved specimen can be either stored or immediately processed.

To isolate RNA, 3 mL of specimen are removed and undergo an organic phase extraction. The aqueous phase is removed and RNA molecules in solution are precipitated out by the addition of ethanol. The RNA homogenate is applied to magnetic beads or silica membrane where RNA is selectively bound. The bound RNA is washed, and residual genomic DNA can be removed from the membrane or bead solution by treatment of the bound nucleic acid with DNase I. RNA can be eluted using the elution buffer provided in standard nucleic acid extraction kits.

To isolate DNA, precipitation buffer is added to the remaining 3 mL of specimen and shaken to ensure precipitation of cellular debris and proteins. The DNA remains in solution and is removed and placed into a new tube where proteinase K is added to remove any residual proteins. DNA lysis buffer and ethanol are added to adjust the binding conditions of nucleic acids. The solution is applied to silica membrane or magnetic beads where DNA is selectively bound. The bound DNA is washed and eluted with provided elution buffer found in standard nucleic acid extraction kits.

The tampon collection system of this example is comprised of (1) a tampon for sample collection, (2) a collection packet for storage and transport of the sample and (3) a shipping packet for shipment to a laboratory. In addition, several lab accessories (e.g., a luer lock syringe and vacutainer) are provided for removing the sample from the collection packet after shipment. Each of these components is described in detail below.

The tampon can be an off-the-shelf, 510(k)-cleared, low absorbency or junior tampon that is 100% cotton, free of chemicals, dyes, and synthetic materials. The tampon can be also hypoallergenic, fragrance-free, and chlorine-free. The applicator is made of BPA-free plastic. The tampon provided may be manufactured by the same vendor in order to reduce brand-to-brand variability inherent in tampon formulations.

The collection Packet can be a cylindrical device used to receive the tampon after the tampon has collected the biological sample. It can be designed to allow for stabilization of nucleic acid in the biological sample.

### Example 8: Measurement of Properties of Menstrual Fluid

Menstrual blood and whole blood were collected from 80 subjects experiencing menstrual bleeding. Tampons were inserted into the vaginal cavity, and removed after 2 hours. After removal, each tampon was placed into a vessel as described in Example 7. While in the vessel, each tampon was compressed and menstrual fluid was eluted using RNAgard buffer as a preservation buffer. The vessel was then connected to a vacutainer which facilitated the transfer of the menstrual fluid from the vessel into the vacutainer tube. DNA and RNA were isolated from each sample. To determine the expression of genes in whole blood and menstrual fluid, small RNA sequencing, RNA transcriptional sequencing were performed using the RNA, and 16s microbiome sequencing, and methylation arrays were performed using the DNA. The unique expression of 800 genes from RNA experiments, 49 small RNAs, and 1,000 CpG-methylation sites were measured in the samples. The values of the gene expression were compared between the menstrual blood and the whole blood.

Among menstrual samples analyzed using a tampon collection system as described herein, approximately 800 genes were identified that were differentially expressed in menstrual fluid compared to whole blood. The peak of differentiation between the sample types was seen on heavy flow day when the majority of endometrial lining is shed. These genes fall within molecular pathways of the immune system, extracellular matrix organization, metabolism, and immune signaling cascades. A list of such markers can be found in Table 7 of this disclosure.

TABLE 7
Gene markers in menstrual fluid

| Gene | log₂ | Gene | log₂ | Gene | log₂ | Gene | log₂ |
|---|---|---|---|---|---|---|---|
| Name | change | Name | change | Name | change | Name | change |
| GNB1 | 2.76 | TPM1 | 3.24 | RPL29 | 4.33 | TPT1 | 3.61 |
| RPL22 | 3.19 | RAB8B | 2.57 | TKT | 3.03 | LCP1 | 2.23 |
| ENO1 | 3.16 | HERC1 | 3.38 | ARF4 | 2.56 | LMO7 | −2.51 |
| PLOD1 | 2.81 | PPIB | 2.39 | ARL6IP5 | 2.52 | MYCBP2 | 3.14 |
| EPHA2 | −2.76 | DENND4A | 2.91 | FOXP1 | 3.05 | DNAJC3 | 2.48 |
| CDC42 | 3.24 | RPL4 | 4.58 | CGGBP1 | 3.09 | IPO5 | 3.36 |
| CLIC4 | 3.53 | RPLP1 | 2.02 | PCNP | 3.29 | COL4A2 | 3.09 |
| MED18 | −3.68 | PKM | 4.05 | RPL24 | 3.75 | CUL4A | 2.42 |
| PHACTR4 | −3.6 | MORF4L1 | 3.66 | BBX | 3.28 | RPPH1 | 1.96 |
| EPB41 | 3.13 | BCL2A1 | 2.7 | FSTL1 | 3.49 | PNP | 3.42 |
| PTP4A2 | 3.05 | KIAA1199 | 2.55 | CSTA | −2.24 | HNRNPC | 2.92 |
| KHDRBS1 | 2.45 | RHCG | −3.99 | ITGB5 | 3.25 | ARHGAP5 | 2.22 |
| EIF3I | 3.2 | CIB1 | 2.95 | SEC61A1 | 2.65 | BAZ1A | 3.35 |
| RBBP4 | 3.36 | IQGAP1 | 2.56 | RPN1 | 2.67 | RPS29 | 3.92 |
| SFPQ | 3.2 | HBA2 | −3.3 | CNBP | 3.38 | RPL36AL | 3.41 |
| MACF1 | 2.42 | HBA1 | −3.22 | CDV3 | 2.35 | NIN | 3.87 |
| YBX1 | 4.81 | RPS2 | 3.63 | COPB2 | 2.39 | KTN1 | 3.46 |
| SLC2A1 | 3.29 | PRSS22 | −3.47 | ZBTB38 | 3.42 | SYNE2 | 4.38 |
| RPS8 | 5.44 | PPL | −3.39 | XRN1 | 2.94 | ACTN1 | 2.56 |
| PRDX1 | 2.44 | SNN | 2.45 | PLOD2 | 2.77 | KIAA0247 | −2.43 |
| CMPK1 | 3.62 | GSPT1 | 3.04 | TSC22D2 | −2.66 | TTC9 | −2.74 |
| RNF11 | 2.35 | RPS15A | 3.64 | SERP1 | 2.71 | MAP3K9 | −3.15 |
| NRD1 | 3.3 | ARL6IP1 | 2.16 | SIAH2 | 2.83 | PCNX | 2.71 |
| USP24 | 2.77 | SCNN1B | −2.79 | MBNL1 | 3.37 | TMED10 | 2.17 |
| JAK1 | 2.25 | IL4R | 2.93 | SEC62 | 3.73 | TC2N | 2.48 |
| PDE4B | 2.82 | MVP | 3.37 | TBL1XR1 | 2.85 | PAPOLA | 2.19 |
| SERBP1 | 3.6 | CORO1A | 2.82 | AP2M1 | 3.21 | PPP2R5C | 2.43 |
| SH3GLB1 | 2.78 | PRSS8 | −3.07 | PSMD2 | 3.55 | DYNC1H1 | 3.73 |
| GBP2 | 3.16 | CYLD | 2.8 | EIF4A2 | 2.31 | EIF5 | 2.94 |
| RPL5 | 4.52 | RBL2 | 2.67 | LPP | 2.63 | THBS1 | 4.93 |
| SCARNA2 | 3.02 | GFOD2 | -6 | ATP13A3 | 2.11 | CHAC1 | −3.64 |
| SORT1 | −2.24 | KARS | 2.41 | MUC4 | −4.11 | SNAP23 | 3.27 |
| CAPZA1 | 1.77 | COX4I1 | 3.38 | TFRC | 3.38 | PDIA3 | 3.57 |
| RHOC | 3.41 | CYBA | 3.89 | FYTTD1 | 3.11 | SERF2 | 2.62 |
| HIPK1 | 2.43 | RPL13 | 3.65 | RPL35A | 4.62 | B2M | 4.59 |
| CSDE1 | 3.09 | PRPF8 | 3.08 | MRFAP1 | 3.53 | DUOX2 | −2.94 |
| ATP1A1 | 3.69 | SPNS2 | −4.26 | WDR1 | 2.27 | DUOXA2 | −5.74 |
| MAN1A2 | 3.1 | PFN1 | 2.97 | RPL9 | 4.16 | COPS2 | 2.69 |
| FAM46C | 3.45 | ALOX12 | −4.22 | PDS5A | 3.26 | ARPP19 | 3.95 |
| TXNIP | 3.11 | EIF5A | 3.32 | FRYL | 2.7 | CGNL1 | −4.48 |
| ECM1 | −3.26 | RPL26 | 4.9 | TMEM165 | 2.45 | ANXA2 | 3.31 |
| MCL1 | 1.95 | NCOR1 | 3.16 | IGFBP7 | 2.83 | VPS13C | 3.53 |
| CDC42SE1 | 2.15 | UBB | 3.82 | TMPRSS11B | −4.36 | NRIP1 | 3.27 |
| S100A10 | 2.54 | C17or76-AS1 | 3.86 | MOB1B | 2.82 | APP | 4.18 |
| FLG | −6.1 | RPL23A | 3.78 | PF4 | 3.22 | CCT8 | 3.4 |
| CRNN | −3.25 | RPL23 | 5.02 | PPBP | 4.44 | C21orf7 | 3.73 |
| CRCT1 | −5.66 | RPL19 | 4.55 | CCNG2 | −1.97 | BACH1 | 2.1 |
| LCE3E | −2.94 | NR1D1 | −2.91 | HNRPDL | 3 | MORC3 | 2.21 |
| LCE3D | −6.54 | KRT13 | −3.05 | SNCA | 3.24 | TTC3 | 3.94 |
| SPRR3 | −2.64 | KRT16 | −4.21 | NFKB1 | 3.28 | BRWD1 | 2.73 |
| SPRR2D | −4.86 | EIF1 | 1.69 | UBE2D3 | 2.27 | TMPRSS2 | −3.07 |
| SPRR2A | −4.34 | PTRF | 2.62 | RPL34 | 3.79 | SIK1 | −1.97 |
| SPRR2E | −4.27 | RPL27 | 3.49 | PDE5A | 2.89 | CSTB | −2.63 |
| S100A8 | −2.04 | SLC25A39 | 3.41 | ANXA5 | 3.94 | PFKL | 2.56 |
| S100A6 | 3.39 | ARHGAP27 | −2.42 | KIAA1109 | 3 | COL18A1 | 2.99 |
| RPS27 | 3.83 | CDC27 | 3.05 | SMARCA5 | 2.98 | COL6A2 | 2.86 |
| TPM3 | 3.26 | ITGB3 | 3.52 | RPS3A | 4.56 | MAPK1 | 2.43 |
| ASH1L | 2.8 | MMD | 3.08 | MFAP3L | 3.24 | MIF | 2.86 |
| ARHGEF2 | 2.14 | SRSF1 | 3.18 | HPGD | −3.92 | FBXO7 | 3.68 |
| LMNA | 3.66 | CLTC | 2.63 | ACSL1 | 2.98 | MYH9 | 3.45 |
| CCT3 | 3.34 | MED13 | 2.26 | CCT5 | 3.07 | EIF3D | 2.43 |
| HDGF | 3.46 | DDX5 | 3.58 | SUB1 | 3.46 | LGALS1 | 3.52 |
| TAGLN2 | 4.24 | GNA13 | 3.2 | IL7R | 2.73 | EIF3L | 3.86 |
| COPA | 2.12 | PRKAR1A | 2.46 | RPL37 | 4.82 | DDX17 | 1.81 |
| UHMK1 | 2.83 | RPL38 | 4.22 | IL6ST | 1.92 | JOSD1 | 2.4 |
| PRRC2C | 2.33 | EVPL | −4.66 | MAP3K1 | 2.61 | RPL3 | 4.08 |
| RABGAP1L | 3.13 | 9-Sep | 3.01 | KIF2A | 3.23 | ATF4 | 2.21 |
| TOR1AIP2 | −2.05 | MYL12A | 3.25 | TNPO1 | 2.77 | XRCC6 | 2.69 |
| QSOX1 | −2.65 | MYL12B | 3.5 | BTF3 | 3.32 | ARL8B | 2.64 |
| GLUL | −2.33 | ANKRD12 | 2.3 | VCAN | 3.84 | EMC3 | 3.21 |
| LAMC1 | 3.29 | RAB31 | 3.44 | CHD1 | 3.5 | RPL32 | 4.96 |
| IVNS1ABP | 2.67 | ROCK1 | 2.37 | PJA2 | 3.72 | ANKRD28 | 2.99 |
| TPR | 2.2 | ATP5A1 | 3.52 | CDC42SE2 | 3.1 | UBE2E1 | 2.92 |
| RGS18 | 3.93 | C18or25 | −3.12 | SKP1 | 2.96 | RPL15 | 3.91 |
| NUCKS1 | 3.81 | SMAD2 | 3.5 | PITX1 | −5.48 | TOP2B | 3.46 |
| C1orf116 | −3.73 | RAB27B | 3.24 | TGFB1 | 3.66 | TGFBR2 | 2.33 |
| CD55 | 3.06 | FECH | 3.17 | HSPA9 | 3.62 | CMTM6 | 3.29 |
| CAPN2 | 2.68 | LMAN1 | 2.66 | MATR3 | 4.06 | GOLGA4 | 1.82 |
| ARF1 | 2.25 | CD226 | 2.54 | PAIP2 | 3.31 | RPSA | 4.37 |
| GUK1 | 3.75 | HMHA1 | 2.89 | SPINK5 | −4.55 | RPL14 | 4.33 |
| TOMM20 | 3.22 | GPX4 | 3.42 | GRPEL2 | −2.69 | CTNNB1 | 3.4 |
| LYST | 2.89 | MIDN | −2.91 | CD74 | 3.81 | HIGD1A | 3.12 |
| HNRNPU | 4.39 | RPS15 | 3.61 | RPS14 | 4.77 | GPX1 | 4.1 |
| TRIM58 | 3.15 | OAZ1 | 3.16 | ANXA6 | 3.26 | RHOA | 2.56 |
| PFKP | 3.01 | EEF2 | 3.83 | GM2A | −4.48 | RAD23B | 3.5 |
| NET1 | 2.78 | UBXN6 | 2.83 | SPARC | 4.97 | PTBP3 | 2.82 |
| GDI2 | 2.25 | RPL36 | 2.67 | CYFIP2 | 2.69 | AKNA | 2.55 |
| ATP5C1 | 2.59 | RPS28 | 2.21 | CLINT1 | 2.43 | ATP6V1G1 | 2.77 |
| OPTN | 2.21 | ZNF426 | −2.44 | DOCK2 | 2.75 | STOM | 3.64 |
| FAM107B | 2.62 | CDC37 | 2.8 | NPM1 | 4.7 | PTGS1 | 3.35 |
| RSU1 | 2.92 | ILF3 | 3.27 | ATP6V0E1 | 1.98 | RPL35 | 3.06 |
| VIM | 5.49 | CALR | 3.52 | HNRNPH1 | 3.4 | RPL12 | 4.55 |
| PIP4K2A | 3.47 | RPL18A | 4.18 | CANX | 3.22 | SET | 2.93 |
| YME1L1 | 2.69 | FKBP8 | 3.23 | GNB2L1 | 3.81 | AIF1L | −4.05 |
| ZEB1 | 3.21 | UBA52 | 4.08 | CDYL | 2.43 | SETX | 2.86 |
| ITGB1 | 4.08 | UBA2 | 2.45 | F13A1 | 3.3 | RPL7A | 3.93 |
| HNRNPF | 3.6 | SPINT2 | 3.29 | DEK | 3.53 | GPSM1 | −3.46 |
| NCOA4 | 4.07 | NCCRP1 | −5.27 | C6orf62 | 3.6 | PLCXD1 | −7.68 |
| SRGN | 2.47 | RPS19 | 3.28 | FAM65B | 2.91 | SLC25A6 | 3.61 |
| PPA1 | 2.72 | ARHGEF1 | 2.63 | HIST1H2AC | 3.07 | TMSB4X | 5.17 |
| PSAP | 2.06 | CNFN | −4.46 | HLA-A | 3.05 | RPS6KA3 | 2.53 |
| P4HA1 | 2.78 | VASP | 2.74 | HLA-E | 2.37 | EIF2S3 | 3.19 |
| SYNPO2L | −4.96 | SNRPD2 | 3.05 | TUBB | 4.06 | CYBB | 2.79 |
| VCL | 3.24 | CALM3 | 2.71 | MUC21 | −3.02 | DDX3X | 2.53 |
| RPS24 | 4.67 | GLTSCR2 | 2.29 | HLA-C | 1.97 | SLC9A7 | −2.16 |
| GHITM | 2.75 | FTL | 2.28 | HLA-B | 3.01 | TIMP1 | 3.72 |
| FAM25A | −4.88 | RPL13A | 2.89 | CLIC1 | 2.98 | RBM3 | 3.4 |
| GLUD1 | 3.37 | RPS11 | 5 | CFB | 2.55 | MAGED2 | 3.26 |
| PTEN | 2.85 | VSIG10L | −4.64 | HLA-DRA | 3.63 | ALAS2 | 2.95 |
| PCGF5 | 2.97 | ZNF83 | −2.34 | HLA-DRB1 | 3.09 | MSN | 4.37 |
| TM9SF3 | 3.72 | RPS9 | 3.97 | HLA-DQB1 | 3.07 | OGT | 2.87 |
| GSTO1 | 3.13 | EPS8L1 | −3.45 | HLA-DPA1 | 3.18 | RPS4X | 4.97 |
| SHOC2 | 3.28 | RPL28 | 3.23 | HLA-DPB1 | 2.69 | XIST | 2.68 |
| ACSL5 | 3.52 | RPS5 | 3.4 | RPS18 | 3.59 | ATRX | 3.32 |
| ABLIM1 | −2.21 | RPS7 | 3.86 | RPL10A | 4.81 | SH3BGRL | 3.46 |
| RPLP2 | 3.6 | YWHAQ | 2.04 | SRSF3 | 3.21 | MORF4L2 | 2.91 |
| CTSD | 1.95 | ODC1 | 3.11 | C6orf132 | −3.79 | ACSL4 | 3.45 |
| HBB | −3.57 | PDIA6 | 3.51 | HSP90AB1 | 3.92 | DOCK11 | 2.6 |
| RPL27A | 4.91 | LPIN1 | −2.16 | CRISP3 | −5.57 | PGRMC1 | 3.47 |
| IPO7 | 2.57 | OST4 | 3.55 | EEF1A1 | 4.33 | RPL39 | 3.89 |
| EIF4G2 | 1.51 | LBH | 3.64 | NT5E | 3.26 | LAMP2 | 2.57 |
| COPB1 | 3.2 | BIRC6 | 3.56 | SYNCRIP | 2.58 | STAG2 | 3.22 |
| RPS13 | 4.46 | LTBP1 | 2.79 | UBE2J1 | 2.72 | MBNL3 | 3.87 |
| LDHA | 3.99 | EML4 | 3.26 | AIM1 | −2.89 | FLNA | 4.76 |
| HIPK3 | 2.25 | CALM2 | 4.65 | SNX3 | 2.55 | RPL10 | 4.8 |
| CAPRIN1 | 2.53 | PSME4 | 3.32 | CD164 | 2.96 | MPP1 | 2.92 |
| CD44 | 4.97 | SPTBN1 | 2.46 | SERINC1 | 3.54 | CD24 | 3.78 |
| PTPRJ | 3.15 | RPS27A | 4 | VNN1 | 2.62 | LYZ | 3.32 |
| DDB1 | 2.86 | SMEK2 | 3.23 | RPS12 | 4.97 | NAPIL1 | 3.91 |
| EEF1G | 4.43 | USP34 | 2.75 | SGK1 | 3 | OSBPL8 | 2.52 |
| SLC3A2 | 3.25 | XPO1 | 3.41 | UTRN | 3.25 | BTG1 | 2.31 |
| FAU | 4.06 | ACTR2 | 2.1 | SASHI | −3.63 | SLC25A3 | 3.53 |
| CFL1 | 2.61 | ANXA4 | 3.23 | SYNE1 | 3.43 | HSP90B1 | 2.93 |
| GSTP1 | 2.75 | MXD1 | −3.33 | SOD2 | 2.49 | C12orf75 | 3.22 |
| CCND1 | 2.79 | ZNF638 | 3.01 | WTAP | 2.31 | GLTP | −2.41 |
| CTTN | 2.28 | MOB1A | 3.45 | IGF2R | 2.4 | RPL6 | 3.71 |
| RAB6A | 3.88 | MTHFD2 | 3.02 | QKI | 3.37 | PTPN11 | 3.23 |
| RPS3 | 4.59 | TMSB10 | 5.28 | RNASET2 | 3.62 | RPLP0 | 2.59 |
| PICALM | 2.11 | TGOLN2 | 2.97 | MAFK | 2.88 | RAN | 3.32 |
| BIRC3 | 3.18 | MAL | −5.72 | ACTB | 1.6 | ZMYM2 | 3.3 |
| BIRC2 | 2.61 | RPL31 | 5.18 | EIF2AK1 | 2.7 | USP12 | 3.22 |
| TMEM123 | 4.42 | LIMS1 | 3.92 | HNRNPA2B1 | 2.54 | HMGB1 | 3.74 |
| MMP1 | 3.19 | RANBP2 | 3.86 | CBX3 | 3.43 | HSPH1 | 2.45 |
| ATM | 3.18 | SOWAHC | −2.82 | C7orf41 | 2.65 | UFM1 | 3.23 |
| MPZL2 | 3.07 | ACTR3 | 2.6 | 7-Sep | 3.7 | ELF1 | 3.38 |
| MLL | 2.93 | GYPC | 2.9 | PPIA | 3.08 | TSC22D1 | 3.15 |
| RPS25 | 4.74 | CXCR4 | 3.76 | UPP1 | −2.66 | TGM2 | 2.93 |
| ARHGEF12 | 3.01 | ZEB2 | 2.65 | CHCHD2 | 3.26 | TOP1 | 2.32 |
| HSPA8 | 3 | SP3 | 2.13 | AKAP9 | 2.23 | SRSF6 | 3.26 |
| ETS1 | 4.58 | HNRNPA3 | 2.37 | GNG11 | 3.76 | PKIG | 2.88 |
| APLP2 | 2.13 | NFE2L2 | 2.27 | GNB2 | 3.36 | YWHAB | 3.44 |
| CCND2 | 3.11 | NCKAP1 | 2.37 | SERPINE1 | 2.78 | STK4 | 2.76 |
| SCARNA10 | 2.5 | C2orf88 | 3.35 | PRKAR2B | 2.89 | MMP9 | 2.75 |
| GAPDH | 3.07 | GLS | 2.97 | LAMB1 | 3.04 | FAM210B | 3.25 |
| CHD4 | 3.5 | MYO1B | 2.59 | TES | 2.56 | RTFDC1 | 3.21 |
| RIMKLB | 2.53 | SDPR | 3.5 | CAPZA2 | 3.72 | RBM38 | 3.09 |
| A2ML1 | −3.95 | STK17B | 3.6 | SND1 | 3.22 | RAB22A | 2.4 |
| ARHGDIB | 2.96 | SF3B1 | 2.16 | HILPDA | −3.11 | GNAS | 3.89 |
| LDHB | 3.95 | HSPD1 | 3.65 | CALU | 3.15 | TUBB1 | 3.27 |
| TWF1 | 2.97 | CLK1 | 3.59 | CPA4 | −3.81 | RPS21 | 3.49 |
| ANO6 | 3.33 | FAM126B | −2.17 | MKLN1 | 3.04 | PPDPF | −3.57 |
| TUBA1B | 3.48 | TRAK2 | 3.8 | CALD1 | 3.5 | PTK6 | -5 |
| TMBIM6 | 2.02 | EEF1B2 | 3.98 | JHDM1D | 3.42 | DNAJC5 | −1.8 |
| DAZAP2 | 4.04 | XRCC5 | 3.35 | MKRN1 | 3.17 | MIR3648 | 4.79 |
| KRT80 | −3.24 | RPL37A | 4.21 | ATG9B | −5.16 | MIR3687 | 3.96 |
| KRT4 | −2.74 | TNS1 | 2.86 | INSIG1 | 2.18 | CA2 | 3.06 |
| KRT78 | −4.51 | ARPC2 | 3.33 | CTSB | 2.55 | UQCRB | 3.34 |
| KRT8 | 2.58 | DOCK10 | 2.63 | PCM1 | 3.67 | RPL30 | 4.93 |
| EIF4B | 2.65 | TRIP12 | 3.43 | ASAH1 | 2.82 | EIF3E | 4.22 |
| PFDN5 | 2.6 | SP100 | 3.02 | BNIP3L | 3.67 | EIF3H | 2.83 |
| ITGA5 | 3.22 | NCL | 3.83 | DPYSL2 | 3.48 | FAM83A | −4.29 |
| RPL41 | 2.53 | PTMA | 5.21 | CLU | 2.71 | FBX032 | −2.31 |
| MYL6 | 4.07 | C2orf54 | −3.7 | TMEM66 | 2.38 | PSCA | −3.95 |
| PTGES3 | 3.08 | HDLBP | 2.94 | PCMTD1 | 2.54 | EEF1D | 3.92 |
| NACA | 4.65 | 2-Sep | 3.91 | RPS20 | 4.71 | PLIN2 | 3.4 |
| LRP1 | 2.63 | RASSF2 | 2.48 | SDCBP | 2.01 | RPS6 | 4.4 |
| CTDSP2 | 2.1 | XRN2 | 3.34 | ASPH | 3.31 | VCP | 2.38 |
| USP15 | 2.58 | RBM39 | 2.64 | TRAM1 | 4.17 | RMRP | 4.15 |
| RAP1B | 3.78 | LINC00657 | 2.76 | RPL7 | 4.75 | FAM108B1 | −3.35 |
| CTSL1 | 2.98 | HNRNPK | 3.61 | ISCA1 | 2.61 | VPS13A | 3.37 |
| HEMGN | 3.03 | | | | | | |

### Example 9: Microbial Metagenome of Cervicovaginal and Menstrual Fluid

The human microbiome can present a potential source of novel biomarkers for detection of HMB. The microbiome can be the collection of microorganisms in the body that exists in a mutualistic relationship with the host.

Menstrual blood was collected from 80 subjects experiencing menstrual bleeding. Tampons were inserted into the vaginal cavity, and removed after 2 hours. After removal, each tampon was placed into a vessel as described in example 7. While in the vessel, each tampon was compressed and menstrual fluid was eluted using RNAgard buffer as a preservation buffer. The vessel was then connected to a vacutainer which facilitated the transfer of the menstrual fluid from the vessel into the vacutainer tube. DNA was collected from the samples. 16s microbiome sequencing was performed on the DNA of each sample. The microbial metagenome of cervicovaginal and menstrual fluid was analyzed using the sample collection device to understand the bacterial diversity present in endometriosis compared to healthy controls. Within the population were 5 patients with polycystic ovarian syndrome, 19 with endometriosis (both pre- and post-surgery collected tampons), and 5 healthy and 50 “suspected unhealthy” individuals. 16s microbial sequencing was performed where a region of the ribosomal RNA genomic code was amplified and sequenced, enabling species-level resolution of bacterial composition. This information was used to compare the relative abundance of bacterial species between healthy (broken up into truly healthy and suspected unhealthy), polycystic ovarian syndrome, and endometriosis. The diversity present within each sample (alpha diversity) as well as the diversity present between samples in the same cohort (beta diversity) were assessed.

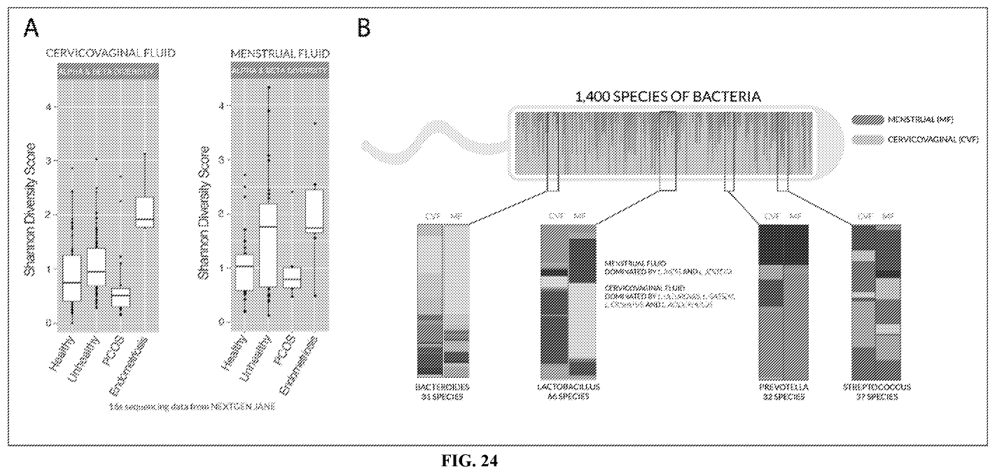

The Shannon Diversity Index was used, which takes into account the abundance of each bacterial species, as well as how evenly that species is represented within the sample or population. There was an increased diversity of bacterial species among patients with endometriosis compared to healthy patients, as shown in FIG. 24. Interestingly, polycystic ovarian syndrome patients showed a significantly reduced diversity of bacterial species within cervicovaginal fluid when compared to endometriosis patients. specific bacterial species were identified that are associated with vaginal and menstrual microbiomes, as well as with endometriosis. On a genus level, both vaginal and menstrual microbiomes were dominated by Lactobacillus. However, when the species level is observed, there is a dramatic shift in the species of bacteria within each genus between vaginal and menstrual samples—most notably there is a shift of Lactobacillus species (a total of 66 species detected): from L. ulturensis, L. gasseri, L. crispatus and L. acidophilus in vaginal samples to L. iners and L. jensenii in menstrual samples, as shown in FIG. 24, panel B. Notably, Propionibacterium acnes, which is present at 15-fold higher levels in our endometriosis patients than in healthy individuals. P. acnes produces high levels of prostaglandin-like substances and porphyrin, both of which have been implicated in inflammation) and dysmenorrhea.

### Example 10: Biological Information Derived from a Sample

Tampon samples from patients were collected on 4 days of menstruation. Tampon collections lasted two hours and were placed into the NGJ device and 20 mL of preservation buffer was released onto the tampon to preserve cells. Tampons were harvested, and fluid was eluted off tampon. Cells were spun at 100 g for 10 minutes to pellet cells and remove preservation buffer. Cells were resuspended in 1×PBS with 2 mM EDTA and 0.5% BSA. Cells were then digested to a single cell suspension by addition of collagenase 4 and DNase I and incubated at 37° C. for 30 minutes with gentle agitation. Cells were then passed through a 70 uM nylon filter to remove any clumps of tissue. Cells were then stained with the following antibodies, and subjected to flow cytometry where percent positive cells were calculated for each antibody to determine percentage of each cell type: CD31-FITC CD34-PE CD326 EPCAM-APC CD24-FITC ANTI-CYTOKERATIN-FITC CD49E-APC CD44-PE SSEA-4-FITC NANOG-APC LGRS-PE. 1 ug of antibody was used to label 1 million cells. Cells were incubated at 4° C. with antibody for 30 minutes, then cells were washed 3 times with 1×PBS with 2 mM EDTA and 0.5% BSA. Unstained and single fluorophore stained cells were counted in order to determine autofluorescence and set proper gating parameters to determine positive cells populations before multiplexed stains were measured.

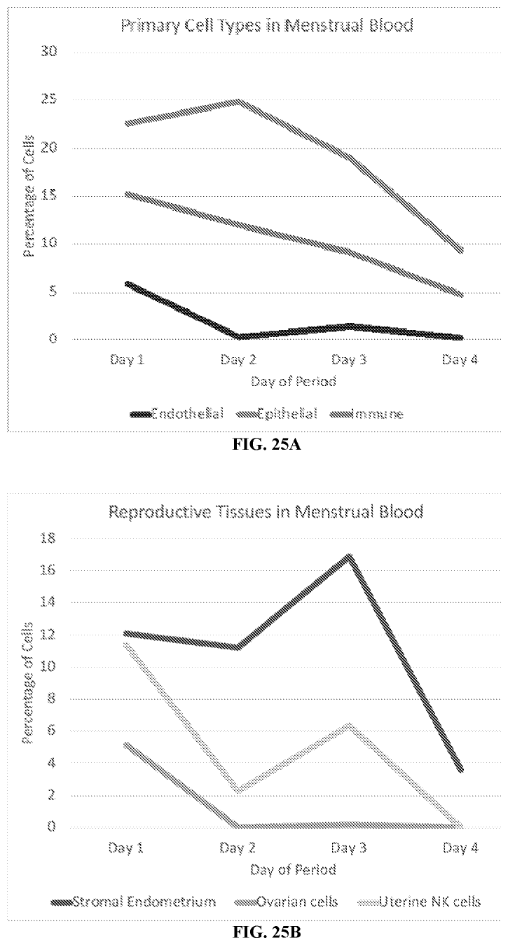

As depicted in FIG. 25, the composition of primary cell types in menstrual blood changes over the days of a cycle. Immune cells were present at higher levels than epithelial or immune cells from days 1-4, as depicted in FIG. 25A. When comparing reproductive tissues in menstrual blood, stromal endometrium was present in the highest amount, followed by uterine NK cells, then ovarian cells, as depicted in FIG. 25B. When comparing stem cells in menstrual blood, SSEA4 positive stem cells were present in higher quantities on day 1 and day 3, while NANOG positive stem cells were present in higher quantities on day 2, as depicted in FIG. 25C.

### Example 11: Determining Red Blood Cell Levels from Menstrual Samples

Heavy flow tampons were collected from two participants for 2 hours and placed into the NGJ device and 20 mL nucleic acid preservation buffer or NaOH were eluted onto tampons to convert hemoglobin to alkaline hematin. Whole blood was also collected, and serially diluted in NaOH to create a standard curve of mL of blood to alkaline hematin intensity values. Samples were centrifuged to remove cell debris and the samples were measured on a spectrophotometer at 500-500 nm wavelengths. Intensities were recorded and the standard curve was used to impute values to the patient samples. The volume of blood from the 2 hour collection was recorded, and microliters of blood present in the menstrual fluid was calculated to the standard whole blood curve.

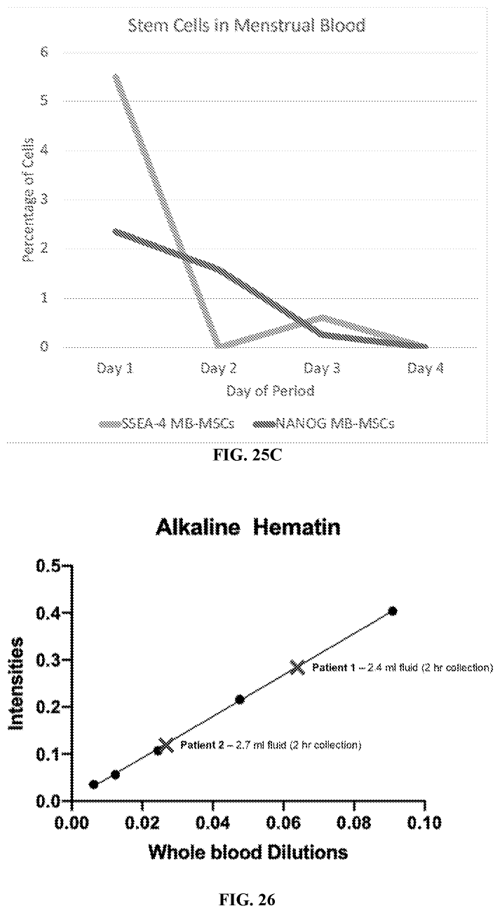

The results are shown in FIG. 26 As indicated by the red X, different patients have different levels of red blood cells in the same volume of menstrual fluid as indicated by alkaline hematocrit levels.

### Example 12: Determining Anemia from Menstrual Samples

Samples are taken on both day 1 and day 2. Samples comprise both whole blood and menstrual blood are taken in a 2 hour time unit. The quantity of hemoglobin gene expression and hematocrit are measured as described herein. The data collection is described in Table 8. Using the data gathered in the samples described above and other factors from the survey described herein, an algorithm is generated that predicts the likelihood of a subject having anemia based on an analysis of menstrual blood samples.

TABLE 8
Protocol for predicting anemia

| Day 1 | | Day 2 | |
|---|---|---|---|
| Quantity of Hemoglobin | Quantity of Hemoglobin | Quantity of Hemoglobin | Quantity of Hemoglobin |
| in Menstrual Blood taken | in Whole Blood taken on | in Menstrual Blood taken | in Whole Blood taken on |
| on Day 1 in 2 hr time unit | Day 1 in 2 hr time unit as | on Day 2 in 2 hr time unit | Day 2 in 2 hr time unit as |
| as defined by levels of | defined by levels of | as defined by levels of | defined by levels of |
| Hemoglobin gene | Hemoglobin gene | Hemoglobin gene | Hemoglobin gene |
| expression | expression | expression | expression |
| Quantity of Hematocrit in | Quantity of Hematocrit in | Quantity of Hematocrit in | Quantity of Hematocrit in |
| Menstrual Blood taken on | Whole Blood taken on | Menstrual Blood taken on | Whole Blood taken on |
| Day 1 in 2 hr time unit as | Day 1 in 2 hr time unit as | Day 2 in 2 hr time unit as | Day 2 in 2 hr time unit as |
| defined by spectrometry | defined by spectrometry | defined by spectrometry | defined by spectrometry |
| readings indicating | readings indicating | readings indicating | readings indicating |
| volume of red blood cells | volume of red blood cells | volume of red blood cells | volume of red blood cells |

### Example 13: Stratification of Populations into Group Based on Phenotypic Annotations and Biological Data

Subjects are annotated using different features, such as those described in the survey provided herein. FIG. 28 provides an example of some of the features used to annotate subjects. The subjects are then stratified into different groups based on the features described.

For instance, the subjects are grouped into 3 sets. Biological features such as flow rate per minute, red blood cell content, and cells present in menstrual blood are measured for each set. FIGS. 28A-28C show theoretical values for each of the biological measures and how annotation features could be used to segregate populations into groups. These values will be used to set normal reference ranges for each subgrouping based on phenotypic annotations and biological data.

The features and the biological data are then integrated using an algorithm. An individual subject can then be compared to the reference range for subjects within a specific subgroup or set. FIG. 29 shows how the averaged value for a subpopulation (dotted line), considered a population baseline would look given phenotypic and biological data. The solid line with pink dot represents an individual patient and how that patient compares to their group's baseline data. This would indicate a health status change that would initiate a patient seeking medical care and further testing to determine and diagnose the changed health state. For example, if FIG. 29 represents total blood loss from period to period, the patient's data would indicate that for two months the patient experienced heavy menstrual bleeding, but then fell with the normal baseline range. An elevated menstrual flow for many months would prompt the patient to seek medical care for the condition. This would prompt the doctor to recommend diet or supplement changes to increase iron levels, or complete further testing to determine if hormonal medication to control menstruation is necessary.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of using a predictive model to identify a condition of heavy menstrual bleeding (HMB), the method comprising using one or more processors in a computer server:
(a) receiving data defining a plurality of subject digital biomarkers, each digital biomarker comprising a response by the subject to an associated inquiry in a computing data storage;
(b) retrieving from computer data storage a combination of independent variables relating to the subject;
(c) using a predictive model to determine a dependent variable representing a subject HMB risk score for the subject based on the combination of independent variables relating to the subject;
(d) determining if the dependent variable representing the subject HMB score is above a threshold; and if the dependent variable representing the subject HMB score is above the threshold, sending information to be displayed.

2. The method of claim 1, wherein the combination of independent variables includes objective data relating to the subject's menstrual bleeding.

3. The method of claim 1, wherein the combination of independent variables includes survey data relating to the subject.

4. The method of claim 3, wherein the survey data comprises at least one independent variable selected from the group consisting of a menstrual cycle phenotype, a physical body characteristic, a disease or condition, a medical treatment, demographic information, lifestyle information, ancestry, sexuality, menstrual management, health care usage and access.

5. The method of claim 1, wherein the combination of independent variables includes health record data relating to the subject.

6. The method of claim 2, wherein the objective data comprises a menstrual flow rate measurement.

7. The method of claim 5, wherein the menstrual flow rate measurement comprises:
(a) collecting menstrual fluid from a subject for a specified duration;
(b) measuring the volume of collected menstrual fluid;
(c) calculating a flow rate from the volume and the specified duration.

8. The method of claim 7, wherein the method further comprises analyzing a biological marker from the collected menstrual fluid.

9. The method of claim 8, wherein the biological marker is selected from a cell-type, a protein, a microorganism, a metabolite, a hematocrit level, or a nucleic acid.

10. The method of claim 8, wherein the biological marker is unique to menstrual fluid.

11. A method for generating a severity assessment of a menstrual bleeding state of a human female subject, the method comprising:
(a) presenting one or more questions to the subject about a first set of attributes related to the subject's menstrual history and a second set of attributes related to a subject's menstrual phenotype, wherein the one or more questions are presented to the subject on a display of a graphic user interface of an input device;
(b) prompting the subject to enter a response to the one or more questions into the input device, wherein the input device transmits the response to a system comprising a processor and a computer-readable memory, wherein the system calculates an assessment score corresponding to menstrual bleeding state of the subject using the response to the one or more questions and stores the assessment score in the memory;
(c) using an assay to perform one or more measurements on a menstrual fluid sample from the subject;
(d) comparing the one or more measurements with one or more predetermined thresholds; and
(e) determine based on the calculated assessment score and the comparison with the one or more predetermined thresholds, the menstrual bleeding state of the subject; and
generating a severity assessment of the subject's menstrual bleeding state.

12. A method of preparation of a biological sample comprising:
(a) identifying a subject;
(b) classifying the subject into a risk group based on one or more digital biomarkers;
(c) collecting menstrual fluid from a subject for a specified duration;
(d) measuring the volume of collected menstrual fluid
(e) calculating a flow rate from the volume and the specified duration.

13. The method of claim 12, wherein the collecting step is performed using a cup, a tampon, or a pad.

14. The method of claim 12, wherein the menstrual fluid is collected in a manner that preserves at least one of intact cells from the vaginal-cervical space, protein, metabolite, DNA or RNA.

15. The method of claim 12, wherein the collecting step comprises contacting the menstrual fluid or a portion thereof with a preserving solution.

16. The method of claim 12-13, further comprising extracting nucleic acid from the menstrual fluid and measuring at least one nucleic acid parameter.

17. The method of claim 12-13, further comprising measuring the presence of level of a metabolite in the menstrual fluid.

18. The method of claim 16, wherein the nucleic acid parameter comprises the amount of nucleic acid, the diversity of nucleic acid, the presence of a miRNA, mRNA expression, copy number.

19. The method of claim 16-18, further comprising separating or isolating one or more cell types from the menstrual fluid.

20. The method of claim 19, wherein the one or more cell types are selected from the group selected from immune cells, ovarian cells, fallopian tube cells, endometrial cells, and cervical cells.

21. The method of claim 13-18, further comprising repeating the collecting step for two or more longitudinal samples from the subject.

22. The method of claim 21, further comprising measuring an individual flow rate for each longitudinal sample and deriving a mean, average or progression of flow rate from the individual flow rates.

23. The method of claim 12-22, wherein the digital biomarker comprises a factor listed in Table 4.

24. A method for detecting a menstrual cycle disorder, comprising:
(a) determining an expression level of one or more markers in a fluid sample obtained from the vaginal cavity of a subject, wherein the one or more markers are selected from Table 1, Table 2, and/or Table 3; and
(b) comparing said expression level to a reference level of said one or more markers;
wherein an increased or decreased expression level of said one or more markers relative to said reference expression level indicates that said subject has said menstrual cycle disorder.

25. The method of claim 24, wherein the fluid sample is obtained from the subject during the subject's menstrual window.

26. The method of claim 24, wherein the reference level is obtained from the subject in a time period outside subject's menstrual window.

27. The method according to any of claims 24-26, wherein the reference level is obtained from a healthy control subject or an average level from a group of healthy control subjects.

28. The method according to any of claims 24-27, wherein the one or more markers are protein expression markers selected from Table 1.

29. The method according to any of claims 24-27, wherein the one or more markers are gene expression markers selected from Table 2.

30. The method according to any of claims 24-27, wherein the one or more markers are gene expression markers selected from Table 3.

31. The method according to any of claims 24-30, wherein said menstrual cycle disorder is heavy menstrual bleeding.

32. The method according to any of claims 24-30, further comprising determining a risk level for the menstrual cycle disorder in the subject.

33. The method of claim 32, wherein the determining a risk level step comprises assessing one or more phenotypic or behavioral characteristics of the subject selected from Table 4 and/or Table 5.

34. The method of claim 24, further comprising stratifying the subject into a treatment group based on the risk level.

35. The method of claim 24, further comprising obtaining two or more fluid samples from the vaginal cavity of the same subject, wherein the two or more fluid samples are from different time points within the subject's menstrual cycle, wherein step (a) is repeated for each of the two or more fluid samples and wherein the expression level for each of the two or more fluid samples are compared in step (b).

36. The method according to any of claims 24-34, wherein the fluid sample comprises blood.

37. The method according to any of claims 24-36, wherein the fluid sample comprises shed endothelial cells and shed epithelial cells.

38. The method of claim 37, wherein the fluid sample comprises a cell type selected from the group consisting of endothelial cells, epithelial cells, immune cells, and stem cells.

39. The method of claim 35, wherein the two or more fluid samples are obtained during the subject's menstrual window.

40. The method of claim 35, wherein at least one of the two or more fluid samples are obtained outside of the subject's menstrual window.

41. The method of claim 1, wherein the one or more markers are selected from the group consisting of HLA-Aa (Major histocompatibility complex class I), IL-6ST (Interleukin 6 signal transducer), APCDD1L (Adenomatosis polyposis coli downregulated 1-like), TBX3 (T-box 3), UBN1a (Ubinuclein), DSPa (Desmoplakin), SDCBP2 (Syndecan binding protein (syntenin)), EZH2 (Enhancer of zeste 2 polycomb repressive complex 2 subunit), UHMK1 (U2AF homology motif (UHM) kinase), NR2C2 (Nuclear receptor subfamily 2, group C, member 2), MIR1282a (microRNA), TMED6a (Transmembrane p24 trafficking protein), VAV3 (Vav 3 guanine nucleotide exchange factor), CDC42BPA (CDC42 binding protein kinase alpha (DMPK-like)), C17orf75 (Chromosome 17 open reading frame), MB21D1 (Mab-21 domain containing 1), PTPRC (Protein tyrosine phosphatase, receptor type C), WISP1 (WNT1 inducible signaling pathway protein 1), CDC27 (Cell division cycle 27), FSD1L (Fibronectin type III and SPRY domain containing 1-like), BPIFB1 ((C20orf114) a BPI fold containing family B, member 1), SCGB3A1a (Secretoglobin, family 3A, member 1) TFF3a (Trefoil factor 3 (intestinal)), SCGB1D2a (Secretoglobin, family 1D, member 2), SCGB2A2a (Secretoglobin, family 2A, member 2), PRODH (Proline dehydrogenase (oxidase) 1), MFF (Mitochondrial fission factor), TSPAN8 (Tetraspanin), CXCL6a (Chemokine (C-X-C motif) ligand 6), SPDYE2 (Speedy/RINGO cell cycle regulator family member E2), FCGBP (Fc fragment of IgG binding protein), IRX6 (Iroquois homeobox 6), ADAM10 (ADAM metallopeptidase domain 10), MUC5ACa (Mucin 5AC, oligomeric mucus/gel-forming), TRHDE-AS1 (TRHDE antisense RNA (LOC283392)), CYP26A1 (Cytochrome P450, family 26, subfamily A, polypeptide 1), SAA2a (Serum amyloid A2), MMEL1 (Membrane metalloendopeptidase-like 1), GRIP2 (Glutamate receptor interacting protein 2), and SAA1a (Serum amyloid A1).

42. The method of claim 41, further comprising determining an increased expression level of one or more markers selected from the group consisting of HLA-Aa (Major histocompatibility complex class I), IL-6ST (Interleukin 6 signal transducer), APCDD1L (Adenomatosis polyposis coli downregulated 1-like), TBX3 (T-box 3), UBN1a (Ubinuclein), DSPa (Desmoplakin), SDCBP2 (Syndecan binding protein (syntenin)), EZH2 (Enhancer of zeste 2 polycomb repressive complex 2 subunit), UHMK1 (U2AF homology motif (UHM) kinase), NR2C2 (Nuclear receptor subfamily 2, group C, member 2), MIR1282a (microRNA), TMED6a (Transmembrane p24 trafficking protein), VAV3 (Vav 3 guanine nucleotide exchange factor), CDC42BPA (CDC42 binding protein kinase alpha (DMPK-like)), C17orf75 (Chromosome 17 open reading frame), MB21D1 (Mab-21 domain containing 1), PTPRC (Protein tyrosine phosphatase, receptor type C), WISP1 (WNT1 inducible signaling pathway protein 1), CDC27 (Cell division cycle 27), and FSD1L (Fibronectin type III and SPRY domain containing 1-like) relative to said reference expression level.

43. The method of claim 41, further comprising determining a decreased expression level of the one or more markers selected from the group consisting of BPIFB1 ((C20orf114) a BPI fold containing family B, member 1), SCGB3A1a (Secretoglobin, family 3A, member 1) TFF3a (Trefoil factor 3 (intestinal)), SCGB1D2a (Secretoglobin, family 1D, member 2), SCGB2A2a (Secretoglobin, family 2A, member 2), PRODH (Proline dehydrogenase (oxidase) 1), MFF (Mitochondrial fission factor), TSPAN8 (Tetraspanin), CXCL6a (Chemokine (C-X-C motif) ligand 6), SPDYE2 (Speedy/RINGO cell cycle regulator family member E2), FCGBP (Fc fragment of IgG binding protein), IRX6 (Iroquois homeobox 6), ADAM10 (ADAM metallopeptidase domain 10), MUC5ACa (Mucin 5AC, oligomeric mucus/gel-forming), TRHDE-AS1 (TRHDE antisense RNA (LOC283392)), CYP26A1 (Cytochrome P450, family 26, subfamily A, polypeptide 1), SAA2a (Serum amyloid A2), MMEL1 (Membrane metalloendopeptidase-like 1), GRIP2 (Glutamate receptor interacting protein 2), and SAA1a (Serum amyloid A1). relative to said reference expression level.

44. The method of claim 41, further comprising determining an expression pattern of said one or more genes or expression products thereof.

45. The method according to any of claims 24-44, wherein the fluid sample is disposed in a sample collector.

46. The method of claim 45, wherein said sample collector is a pad, a tampon, a vaginal cup, a cervical cap, a menstrual disk, a cervical disk, a sponge, or an interlabial pad.

47. The method of claim 45, wherein the sample collector comprises a chamber comprising a buffer for preserving intact cells, DNA, RNA, protein, metabolite(s), or any combination thereof.

48. The method of claim 24, further comprising the step of treating the subject for a heavy menstrual bleeding disorder if the increased or decreased expression level of said one or more markers relative to said reference expression level indicates that said subject has said menstrual cycle disorder.

49. The method of claim 48, wherein the step of treating is selected from the group consisting of a therapeutic agent, a surgical intervention or a combination thereof.

50. The method of claim 49, wherein the therapeutic agent is selected from the group consisting of an antifibrinolytic agent, a combined hormonal contraceptive, a progestogens, a progestogen-releasing intrauterine device, an androgen, a gonadotropin releasing hormone analogue or any combination thereof.

51. The method of claim 49, wherein the surgical intervention is selected from the group consisting of surgical excision, n endometrial ablation, endometrial cryoablation, uterine artery embolization, myomectomy, hysterectomy, and any combination thereof.

52. The method of claim 24, further comprising placing the subject in a non-treatment category if the subject does not have increased or decreased expression level of said one or more markers relative to said reference expression level.

53. A method of producing a desired preparation for assessment of menstrual health, comprising:
(a) receiving a fluid sample collected from the vaginal cavity of a subject, the fluid sample comprising one or more types of cells;
(b) contacting the fluid sample with a buffer solution under conditions suitable to maintain one or more cell types in a substantially intact state;
(c) separating one cell type in the fluid sample from the remaining fluid sample;
(d) determining an expression level of one or more markers in the one cell type, wherein the one or more markers are selected from Table 1 and/or Table 2; and
(e) comparing the expression level with a reference level to assess a level of menstrual health.

54. The method of claim 53, wherein step (b) maintains at least 90%, 95%, or substantially 100% of said one or more types of cells in a substantially intact state.

55. The method of claim 53, wherein the fluid sample is a menstrual fluid sample.

56. The method of claim 53, wherein the one cell type is selected from the group consisting of endothelial cells, epithelial cells, mesenchymal cells, and leukocytes.

57. The method of claim 53 or claim 56, wherein the one cell type is separated based on expression of a cell surface antigen.

58. The method of claim 57, wherein the one cell type is an endothelial cell and the cell surface antigen is selected from the group consisting of CD31/PECAM-1, CD34, CD36/SR-B3, CD39, CD44, CD47, CD54/ICAM-1, CD61, CD62E, CD62P, CD80, CD86, CD93, CD102, CD105, CD106, CD112, CD117, ESAM, ENDOMUCIN, CXCL16, CD121a, CD141, CD142, CD143, CD144, CD146, CD147, CD151, CD160, CD201, CD213a, CD248, CD309, ADAMS 8-17, 33, ADAMTS-13, ADAMTS-18, VWF, TEM8, NOTCH, KLF4 and any combination thereof.

59. The method of claim 57, wherein the one cell type is an epithelial cell and the cell surface antigen is selected from the group consisting of EpCAM, E-cadherin, CD326, and any combination thereof.

60. The method of claim 57, wherein the one cell type is a leukocyte and the cell surface antigen is CD45.

61. The method of claim 57, wherein the one cell type is a mesenchymal cell and the cell surface antigen is selected from the group consisting of N-cadherin, OB-cadherin, alpha-5, beta-1 integrin, alpha-V, beta-6 integrin, Syndecan-1, and any combination thereof.

62. The method according to any of claims 57-61, wherein the method further comprises using an antibody that binds the cell surface antigen to separate the one cell type.

63. The method of according to any of claims 53-62, wherein the fluid sample is disposed in a sample collector.

64. The method of claim 63, wherein said sample collector is a pad, a tampon, a vaginal cup, a cervical cap, a menstrual disk, a cervical disk, a sponge, or an interlabial pad.

65. The method of claim 63 or claim 64, wherein the sample collector comprises the buffer solution.

66. A method for assessing menstrual health in a subject, comprising:
(a) receiving a menstrual fluid sample collected from said subject;
(b) contacting said menstrual fluid sample with a buffer solution under conditions suitable to preserve a sample component selected from the group consisting of intact cells, nucleic acid, protein, and any combination thereof; and
(c) determining a sample component level from the menstrual fluid sample.

67. The method of claim 66, wherein the sample component comprises intact cells, and wherein step (b) maintains at least 90%, 95%, or substantially 100% of said one or more types of cells in a substantially intact state.

68. The method of claim 67, wherein step (c) comprises determining an amount of one or more cell types in the menstrual fluid sample.

69. The method of claim 68, wherein the one or more cell types are selected from the group consisting of leukocytes, erythrocytes, endothelial cells, epithelial cells, stromal cells, stem cells, and any combinations thereof.

70. The method of claim 66, further comprising comparing the amount of one or more cell types to a predetermined threshold.

71. The method of claim 70, wherein an increase in the amount as compared to the predetermined threshold indicates that the subject has a menstrual cycle disorder.

72. The method of claim 66, wherein the sample component comprises nucleic acid.

73. The method of claim 72, wherein the method further comprises determining an amount of nucleic acid present in the menstrual fluid sample.

74. The method of claim 73, further comprising comparing the amount of nucleic acid to a predetermined threshold.

75. The method of claim 74, wherein an increase in the amount as compared to the predetermined threshold indicates that the subject has a menstrual cycle disorder.

76. The method of claim 74, wherein the predetermined threshold is an amount of nucleic acid present in a reference menstrual fluid sample.

77. The method of claim 76, wherein the reference menstrual fluid sample is obtained from a healthy control subject.

78. The method of claim 72, wherein the nucleic acid comprises RNA, and wherein the method further comprises measuring the level of at least one RNA expression marker.

79. The method of claim 78, wherein the nucleic acid comprises miRNA, and wherein the method further comprises measuring the level of at least one miRNA.

80. The method of claim 78 or claim 79 wherein the method comprises comparing the level to a predetermined threshold.

81. The method of claim of claim 66, wherein the sample component comprises protein.

82. The method of claim 81, wherein the method comprises determining the presence or level of at least one protein marker.

83. The method of claim 82, wherein the method comprises comparing the level to a predetermined threshold.

84. The method of claim 66, wherein the sample component comprises a microbial source present in the menstrual fluid sample.

85. The method of claim 84, wherein the microbial source is a bacteria, a fungus, or a virus.

86. The method of claim 84, wherein the method further comprises measuring a microbial source component, and wherein the component is selected from the group consisting of nucleic acid, protein, metabolite, cell, and any combination thereof.

87. The method of claim 84, further comprising measuring the bacterial diversity in the menstrual fluid sample.

88. The method of claim 86 or claim 87, comparing the measure of bacterial diversity to a reference measure of bacterial diversity.

89. The method of according to any of claims 85-88, further comprising determining an amount of bacteria in genus Atopobium, Propionibacterium, Dialister, Porphyromonas, Streptococcus, Dermabacter, Moraxella, Anaerococcus, Peptostreptococcus, Lactobacillus, Prevotella, Campylobacter, Corynebacterium, Facklamia, or Klebsiella.

90. The method of according to any of claims 66-89, wherein said menstrual fluid sample is disposed in a sample collector.

91. The method of claim 90, wherein said sample collector is a pad, a tampon, a vaginal cup, a cervical cap, a menstrual disk, a cervical disk, a sponge, or an interlabial pad.

92. The method of claim 90 or claim 91, wherein the sample collector comprises the buffer solution.

93. The method of claim 92, wherein said buffer solution has a pH greater than 7.

94. The method of claim 92, further comprising, prior to (c), storing said menstrual fluid sample in the presence of said buffer solution for at least 1 day.

95. The method of claim 94, wherein the storage occurs at up to about 4° C.

96. The method of claim 94, wherein the storage occurs at up to about −20° C.

97. The method of claim 94, wherein the storage occurs at room temperature.

98. A method for detecting a menstrual cycle disorder in a subject, comprising:
(a) determining a flow rate of a menstrual fluid sample collected from said subject within a predefined time period; and
(b) comparing said flow rate of said menstrual fluid sample to a predetermined threshold;
wherein an increased flow rate relative to said predetermined threshold indicates that said subject has said menstrual cycle disorder.

99. The method of claim 98, wherein said predetermined threshold is a flow rate of a menstrual fluid sample obtained from a reference subject within said predefined time period.

100. The method of claim 99, wherein said reference subject is a healthy control subject.

101. The method of claim 99, wherein said predefined time period is greater than 15 minutes.

102. The method of claim 99, wherein said predefined time period is less than 2 hours.

103. The method of claim 99, further comprising measuring a volume of said menstrual fluid sample collected from said subject within said predefined time period.

104. The method of claim 103, further comprising comparing said measured volume to a predetermined threshold.

105. The method of claim 103, wherein said predetermined threshold is a volume of a menstrual fluid sample collected from a reference subject within said predefined time period.

106. The method according to any of claims 98-105, wherein said menstrual fluid sample is disposed in a sample collector.

107. The method of claim 106, wherein said sample collector is a pad, a tampon, a vaginal cup, a cervical cap, a menstrual disk, a cervical disk, a sponge, or an interlabial pad.

108. A method for detecting a menstrual cycle disorder, comprising:
(a) determining an expression level of one or more markers in a fluid sample obtained from the vaginal cavity of a subject, wherein the one or more markers are selected from Table 1, Table 2 and/or Table 3;
(b) applying a classifier algorithm to said expression level of one or more markers and a reference level of each of the one or more markers to calculate a metric that quantifies a difference between the expression level and the reference level for each of the one or more markers; and
(c) determining a presence of a menstrual cycle disorder based on the metric.

109. A method of producing a desired preparation for assessment of menstrual health, comprising:
(a) receiving a fluid sample collected from the vaginal cavity of a subject, the fluid sample comprising one or more types of cells;
(b) contacting the fluid sample with a buffer solution under conditions suitable to maintain one or more cell types in a substantially intact state;
(c) separating one cell type in the fluid sample from the remaining fluid sample; and
(d) applying a classifier algorithm to said expression level of one or more markers in the one cell type to calculate a metric that quantifies the difference between said expression level and a reference level to assess a level of menstrual health, wherein the one or more markers are selected from Table 1 and/or Table 2.

110. A method for detecting a menstrual cycle disorder in a subject, comprising:
(a) determining a flow rate of a menstrual fluid sample collected from said subject within a predefined time period; and
(b) applying a classifier algorithm to said flow rate of said menstrual fluid sample to calculate a metric that quantifies a difference between said metric and a predetermined threshold;
wherein an increased flow rate relative to said predetermined threshold indicates that said subject has said menstrual cycle disorder.

111. A method comprising:
(a) obtaining a fluid sample from a vaginal cavity of a subject, wherein the fluid sample is stabilized under conditions which preserve a component of the fluid sample;
(b) determining an expression level of one or more markers in the fluid sample;
(c) comparing said expression level to a reference level of said one or more markers to detect an increased or decreased expression level of the one or more markers relative to the reference expression level, and
(d) determining from the increased or decreased expression level whether the subject has a menstrual cycle disorder.

112. The method of claim 111, wherein the component is preserved for at least 1 day.

113. The method of claim 111, wherein the component is preserved at up to about 4° C.

114. The method of claim 111, wherein the component is preserved at up to about −20° C.

115. The method of claim 111, wherein the component is preserved at room temperature

116. The method of claim 111, wherein the component is selected from the group consisting of a cell, a nucleic acid, a protein, a metabolite, and a microorganism.

117. The method of claim 111, wherein the one or more markers are selected from the group consisting of the markers in Table 4.

118. The method of claim 111, wherein the one or more markers are selected from the group consisting of the markers in Table 5.

119. The method of claim 111, wherein the one or more markers are selected from the group consisting of the markers in Table 1.

120. The method of claim 111, wherein the menstrual cycle disorder is HMB.

121. The method of claim 111, wherein the menstrual cycle disorder is AUB.
